SEQUENCE LISTING (59 sequences)

SEQ ID NO: 1 - DNA, 1308 bp, Homo sapiens
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 2 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 3 - protein, 1161 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 4 - DNA, 3486 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 5 - protein, 995 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 6 - protein, 2988 aa, Artificial Sequence
  synthetic construct
ATGGCTCTTCCTGTTACAGCCCTCCTGCTTCCCCTGGCGCTGCTGTTGCACGCCGCCCGCCCCGACATCCAGATGACCCAGACTACCTCCTCTCTGAGTGCTAGCCTGGGGGACCGCGTAACCATTTCATGCCGAGCCTCCCAGGACATCTCCAAGTACCTGAATTGGTACCAGCAGAAGCCGGACGGCACCGTGAAGCTTCTTATCTACCATACTAGCCGCCTGCACTCTGGCGTCCCATCCAGATTTTCAGGCTCCGGCTCTGGCACCGACTACTCTCTTACCATCTCTAACCTCGAACAGGAGGACATCGCCACCTATTTCTGTCAACAGGGCAACACGCTGCCCTATACCTTCGGCGGTGGCACCAAGCTGGAGATCACCGGCTCTACCTCGGGCAGCGGCAAACCTGGCTCCGGGGAAGGCAGCACCAAGGGAGAGGTGAAGCTGCAGGAGAGCGGACCAGGCCTGGTGGCTCCCTCCCAGTCCCTGTCTGTGACCTGTACCGTATCGGGGGTCTCACTGCCGGACTACGGCGTTAGTTGGATTCGCCAGCCACCCCGCAAGGGTCTGGAGTGGCTGGGCGTTATCTGGGGCTCCGAGACTACCTACTACAATTCCGCGCTGAAGTCGCGCCTGACTATCATCAAGGACAACTCCAAGAGCCAGGTGTTCCTGAAAATGAACTCGTTGCAGACGGATGACACCGCAATCTATTATTGCGCCAAGCACTACTACTACGGCGGCAGCTACGCCATGGATTACTGGGGTCAGGGTACCTCCGTGACCGTGTCCTCGATTGAGGTGATGTATCCACCCCCCTATCTGGACAACGAGAAATCAAATGGCACCATCATCCACGTAAAGGGCAAGCACCTGTGCCCCTCTCCTCTGTTCCCCGGCCCTTCCAAGCCCTTCTGGGTGCTAGTGGTGGTAGGGGGCGTCCTGGCGTGCTACTCCTTGCTGGTCACTGTGGCGTTCATCATCTTCTGGGTCCGGAGCAAGCGTTCTCGCCTGCTGCACAGCGATTACATGAATATGACTCCGCGCCGCCCCGGGCCAACCCGCAAGCACTACCAGCCGTACGCGCCCCCCCGCGACTTTGCCGCCTACCGATCCCGCGTGAAATTTTCACGCAGCGCGGACGCCCCGGCCTATCAGCAGGGACAGAACCAGCTTTACAACGAGCTCAACCTGGGCAGGAGGGAGGAGTACGATGTGCTGGACAAGCGCCGCGGTAGAGACCCGGAGATGGGCGGTAAGCCGCGCAGGAAGAACCCTCAGGAGGGCCTCTACAACGAGCTCCAGAAGGACAAGATGGCCGAGGCTTACTCTGAGATCGGTATGAAAGGCGAGCGGCGCCGTGGCAAAGGCCATGACGGCCTGTACCAGGGTCTCTCGACTGCCACTAAGGATACTTACGACGCACTGCACATGCAGGCCCTGCCCCCGCGCGGATCCGGCGCAACAAACTTCTCTCTGCTGAAACAAGCCGGAGATGTCGAAGAGAATCCTGGACCGACCGAGTACAAGCCCACGGTGCGCCTCGCCACCCGCGACGACGTCCCCAGGGCCGTACGCACCCTCGCCGCCGCGTTCGCCGACTACCCCGCCACGCGCCACACCGTCGATCCGGACCGCCACATCGAGCGGGTCACCGAGCTGCAAGAACTCTTCCTCACGCGCGTCGGGCTCGACATCGGCAAGGTGTGGGTCGCGGACGACGGCGCCGCGGTGGCGGTCTGGACCACGCCGGAGAGCGTCGAAGCGGGGGCGGTGTTCGCCGAGATCGGCCCGCGCATGGCCGAGTTGAGCGGTTCCCGGCTGGCCGCGCAGCAACAGATGGAAGGCCTCCTGGCGCCGCACCGGCCCAAGGAGCCCGCGTGGTTCCTGGCCACCGTCGGAGTCTCGCCCGACCACCAGGGCAAGGGTCTGGGCAGCGCCGTCGTGCTCCCCGGAGTGGAGGCGGCCGAGCGCGCCGGGGTGCCCGCCTTCCTGGAGACCTCCGCGCCCCGCAACCTCCCCTTCTACGAGCGGCTCGGCTTCACCGTCACCGCCGACGTCGAGGTGCCCGAAGGACCGCGCACCTGGTGCATGACCCGCAAGCCCGGTGCCGGCAGCGGCGAGGGCAGAGGCAGCCTGTTAACCTGCGGCGACGTGGAGGAGAACCCAGGTCCTATGTACGACGGGCCGCGCCTGCTGCTGTTGCTGCTTCTGGGGGTGTCCCTTGGAGGTGCCAAGGAGGCATGCCCCACAGGCCTGTACACACACAGCGGTGAGTGCTGCAAAGCCTGCAACCTGGGCGAGGGTGTGGCCCAGCCTTGTGGAGCCAACCAGACCGTGTGTGAGCCCTGCCTGGACAGCGTGACGTTCTCCGACGTGGTGAGCGCGACCGAGCCGTGCAAGCCGTGCACCGAGTGCGTGGGGCTCCAGAGCATGTCGGCGCCGTGCGTGGAGGCCGACGACGCCGTGTGCCGCTGCGCCTACGGCTACTACCAGGATGAGACCACTGGGCGCTGCGAGGCGTGCCGCGTGTGCGAGGCGGGCTCGGGCCTCGTGTTCTCCTGCCAGGACAAGCAGAACACCGTGTGCGAGGAGTGCCCCGACGGCACGTATTCCGACGAGGCCAACCACGTGGACCCGTGCCTGCCCTGCACCGTGTGCGAGGACACCGAGCGCCAGCTCCGCGAGTGCACACGCTGGGCCGACGCCGAGTGCGAGGAGATCCCTGGCCGTTGGATTACACGGTCCACACCCCCAGAGGGCTCGGACAGCACAGCCCCCAGCACCCAGGAGCCTGAGGCACCTCCAGAACAAGACCTCATAGCCAGCACGGTGGCAGGTGTGGTGACCACAGTGATGGGCAGCTCCCAGCCCGTGGTGACCCGAGGCACCACCGACAACCTCATCCCTGTCTATTGCTCCATCCTGGCTGCTGTGGTTGTGGGTCTTGTGGCCTACATAGCCTTCAAGAGGTGGAACAGTTGA

SEQ ID NO: 7 - protein, 1165 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 8 - DNA, 3498 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 9 - protein, 999 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 10 - DNA, 3000 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagttga

SEQ ID NO: 11 - protein, 1155 aa, Artificial Sequence
  synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 12 - DNA, 3468 bp, Artificial Sequence
  synthetic construct
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 13 - protein, 1159 aa, Artificial Sequence
  synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 14 - DNA, 3480 bp, Artificial Sequence
  synthetic construct
atggcgctgcccgtgactgctttgttgcttccattggcgctcctgcttcacgccgctcgccctcaggtgcagctgcagcagagtggccctgagctcgagaagcctggcgccagcgttaagatatcttgcaaggcttccggttattccttcaccggctacaccatgaactgggtcaagcagagccatggaaagtcgttggaatggatcggccttatcaccccctacaacggtgcttcgtcctacaaccagaaatttcgtgggaaggccaccctgacagtcgacaaatccagttccaccgcgtacatggatctgctgtctctgacgagcgaggacagcgccgtgtacttctgtgctcgcggaggctatgatggccggggcttcgactattggggccagggcacgaccgtgaccgtcagctccggtggcggaggatctggtggcggtggatctggcggcggaggtagtgacattgagctgacccagagccctgccatcatgtctgcaagcccaggcgaaaaggtgacaatgacctgctctgcttcgtcctctgtgagctacatgcactggtaccaacagaagtctgggacctccccgaaacgctggatctacgacacctccaagctggcctccggcgttcccgggcgcttcagcggctctggaagtggcaactcctactccctgaccatttcgtccgtggaggctgaagatgacgcaacctactactgtcaacagtggtctggataccccctgaccttcggcgctggaaccaagctggagatcacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactga

SEQ ID NO: 15 - DNA, 20 bp, Artificial Sequence
  synthetic construct
acggaggctaagcgtcgcaa

SEQ ID NO: 16 - DNA, 20 bp, Artificial Sequence
  synthetic construct
cgcttccgcggcccgttcaa

SEQ ID NO: 17 - DNA, 20 bp, Artificial Sequence
  synthetic construct
ttgctcaaatacaaagcctg

SEQ ID NO: 18 - DNA, 20 bp, Artificial Sequence
  synthetic construct
tgagagactccagttgccag

SEQ ID NO: 19 - DNA, 20 bp, Artificial Sequence
  synthetic construct
ggcttccacagacacaccca

SEQ ID NO: 20 - DNA, 20 bp, Artificial Sequence
  synthetic construct
caaccagccagaaattgctg

SEQ ID NO: 21 - protein, 20 aa, Artificial Sequence
  synthetic construct
TTCCAGGGCTCCGAGCCGCG

SEQ ID NO: 22 - protein, 20 aa, Artificial Sequence
  synthetic construct
GTTGTTCTGGAAGTTGAGGA

SEQ ID NO: 23 - protein, 615 aa, Artificial Sequence
  synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQPGDVEENPGPRMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 24 - DNA, 1845 bp, Artificial Sequence
  synthetic construct
atggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctaccagcggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagcggctcccgggccaagagaagcggatccggcgccaccaacttcagcctgctgaagcagcccggagacgtggaagaaaaccctggccctaggatgagcatcggcctgctgtgctgcgccgccctgagcctgctgtgggcaggtccagtgaatgctggtgtcactcagaccccaaaattccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggc

SEQ ID NO: 25 - DNA, 2996 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacag

SEQ ID NO: 26 - protein, 998 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWN

SEQ ID NO: 27 - DNA, 3495 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgac

SEQ ID NO: 28 - protein, 1165 aa, Artificial Sequence; identical to SEQ ID NO: 7
  synthetic construct

SEQ ID NO: 29 - DNA, 2984 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacag

SEQ ID NO: 30 - protein, 994 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSGEGRGSLLTCGDVEENPGPMYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWN

SEQ ID NO: 31 - DNA, 3483 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcagcggcgagggcagaggcagcctgttaacctgcggcgacgtggaggagaacccaggtcctatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgac

SEQ ID NO: 32 - protein, 1161 aa, Artificial Sequence; identical to SEQ ID NO: 3
  synthetic construct

SEQ ID NO: 33 - DNA, 2336 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacag

SEQ ID NO: 34 - protein, 778 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWN

SEQ ID NO: 35 - DNA, 2324 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacag

SEQ ID NO: 36 - protein, 774 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWN

SEQ ID NO: 37 - DNA, 2834 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacggggatccggcgcaacaaacttctctctgctgaaacaagccggagatgtcgaagagaatcctggaccgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatga

SEQ ID NO: 38 - protein, 944 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITH

SEQ ID NO: 39 - DNA, 2822 bp, Artificial Sequence
  synthetic construct
atggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgcggatccggcgcaacaaacttctctctgctgaaacaagccggagatgtcgaagagaatcctggaccgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatga

SEQ ID NO: 40 - protein, 940 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITH

SEQ ID NO: 41 - DNA, 2337 bp, Artificial Sequence
  synthetic construct
atgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagtggatccggcgcaacaaacttctctctgctgaaacaagccggagatgtcgaagagaatcctggaccggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacgg

SEQ ID NO: 42 - protein, 779 aa, Artificial Sequence
  synthetic construct
MYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSGSGATNFSLLKQAGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 2325 bp, Artificial Sequence
  synthetic construct
atgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagtggatccggcgcaacaaacttctctctgctgaaacaagccggagatgtcgaagagaatcctggaccggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgc

SEQ ID NO: 44 - protein, 775 aa, Artificial Sequence
  synthetic construct
MYDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSGSGATNFSLLKQAGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 2835 bp, Artificial Sequence
  synthetic construct
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgacaactactcccgcccccaggcctcctactcccgcgcccaccatagcttcccagccgctatctctccgccctgaagcgtgtaggccagctgcaggtggtgctgtccacacccgcggcttggatttcgcttgcgacatttatatttgggcgccgctcgccggcacctgcggggtcctgctgctgagcctggtgatcacactttactgtaaacgcggaaggaagaagctgctgtacatcttcaagcagcccttcatgcgcccggtgcagactacacaagaggaagatggatgcagctgtcgcttccctgaggaggaggagggtggatgcgagctgcgagtgaaattttcccgcagcgcggacgcacctgcctaccagcagggacagaaccagctgtacaacgagctcaacctaggtcgccgcgaggagtacgacgtgctggacaagcgtcgcgggcgggacccggagatgggcggcaagccccgtcgtaagaaccctcaggagggcctttacaacgagctgcagaaggacaaaatggcggaggcctacagtgagattggtatgaagggcgagcggcgcagaggaaaaggccacgacgggttgtatcagggcctctcgaccgctacgaaggatacgtacgacgcgctacacatgcaggccctgcccccacgg

SEQ ID NO: 46 - protein, 945 aa, Artificial Sequence
  synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 2823 bp, Artificial Sequence
  synthetic construct
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgacggatccggcgcaacaaacttctctctgctgaaacaagccggagatgtcgaagagaatcctggaccggctcttcctgttacagccctcctgcttcccctggcgctgctgttgcacgccgcccgccccgacatccagatgacccagactacctcctctctgagtgctagcctgggggaccgcgtaaccatttcatgccgagcctcccaggacatctccaagtacctgaattggtaccagcagaagccggacggcaccgtgaagcttcttatctaccatactagccgcctgcactctggcgtcccatccagattttcaggctccggctctggcaccgactactctcttaccatctctaacctcgaacaggaggacatcgccacctatttctgtcaacagggcaacacgctgccctataccttcggcggtggcaccaagctggagatcaccggctctacctcgggcagcggcaaacctggctccggggaaggcagcaccaagggagaggtgaagctgcaggagagcggaccaggcctggtggctccctcccagtccctgtctgtgacctgtaccgtatcgggggtctcactgccggactacggcgttagttggattcgccagccaccccgcaagggtctggagtggctgggcgttatctggggctccgagactacctactacaattccgcgctgaagtcgcgcctgactatcatcaaggacaactccaagagccaggtgttcctgaaaatgaactcgttgcagacggatgacaccgcaatctattattgcgccaagcactactactacggcggcagctacgccatggattactggggtcagggtacctccgtgaccgtgtcctcgattgaggtgatgtatccacccccctatctggacaacgagaaatcaaatggcaccatcatccacgtaaagggcaagcacctgtgcccctctcctctgttccccggcccttccaagcccttctgggtgctagtggtggtagggggcgtcctggcgtgctactccttgctggtcactgtggcgttcatcatcttctgggtccggagcaagcgttctcgcctgctgcacagcgattacatgaatatgactccgcgccgccccgggccaacccgcaagcactaccagccgtacgcgcccccccgcgactttgccgcctaccgatcccgcgtgaaattttcacgcagcgcggacgccccggcctatcagcagggacagaaccagctttacaacgagctcaacctgggcaggagggaggagtacgatgtgctggacaagcgccgcggtagagacccggagatgggcggtaagccgcgcaggaagaaccctcaggagggcctctacaacgagctccagaaggacaagatggccgaggcttactctgagatcggtatgaaaggcgagcggcgccgtggcaaaggccatgacggcctgtaccagggtctctcgactgccactaaggatacttacgacgcactgcacatgcaggccctgcccccgcgc

SEQ ID NO: 48 - protein, 941 aa, Artificial Sequence
  synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHDGSGATNFSLLKQAGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - DNA, 2215 bp, Artificial Sequence
  synthetic construct
taggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagctgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 50 - DNA, 1952 bp, Artificial Sequence
  synthetic construct
ctagagcgctgccaccatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagctgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 51 - DNA, 2164 bp, Artificial Sequence
  synthetic construct
ctagcaagctggaggaaaaactgtttcatacagaaggcgtggaggaaaaactgtttcatacagaaggcgtggaggaaaaactgtttcatacagaaggcgtggaggaaaaactgtttcatacagaaggcgtcgcgaattcgcggagactctagagggtatataatggaagctcgatttccagcttggcattccggtactgttggtaaacacctctagagcgctgccaccatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagctgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 52 - DNA, 2214 bp, Artificial Sequence
  synthetic construct
ctagcggggactttccgcttggggactttccgctggggactttccgctggggactttccgctggggactttccgaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgtctagagcgctgccaccatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagctgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 53 - DNA, 2210 bp, Artificial Sequence
  synthetic construct
ctagctgagtcagtgactcagtgagtcagtgactcagtgagtcagtgactcagtgagtcagtgactcaggaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgtctagagcgctgccaccatgtacgacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagaccactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagctgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 54 - DNA, 2711 bp, Artificial Sequence
  synthetic construct
taggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagccgccaccatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 55 - DNA, 2448 bp, Artificial Sequence
  synthetic construct
ctagagccgccaccatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 56 - DNA, 2662 bp, Artificial Sequence
  synthetic construct
ctagcaagctggaggaaaaactgtttcatacagaaggcgtggaggaaaaactgtttcatacagaaggcgtggaggaaaaactgtttcatacagaaggcgtggaggaaaaactgtttcatacagaaggcgtcgcgaattcgcggagactctagagggtatataatggaagctcgatttccagcttggcattccggtactgttggtaaacacctctagagcgctgccaccatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 57 - DNA, 2712 bp, Artificial Sequence
  synthetic construct
ctagcggggactttccgcttggggactttccgctggggactttccgctggggactttccgctggggactttccgaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgtctagagcgctgccaccatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 58 - DNA, 2708 bp, Artificial Sequence
  synthetic construct
ctagctgagtcagtgactcagtgagtcagtgactcagtgagtcagtgactcagtgagtcagtgactcaggaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgtctagagcgctgccaccatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactgacctgcagggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 59 - protein, 21 aa, Artificial Sequence
  synthetic construct
GSGEGRGSLLTCGDVEENPGP

(19) United States
(12) Patent Application Publication
(10) Pub. No.: US 2024/0307444 A1
(43) Pub. Date: Sep. 19, 2024

(54) METHODS AND COMPOSITIONS FOR IMPROVED IMMUNOTHERAPIES

(71) Applicants: New York Genome Center, Inc., New York, NY (US); New York University, New York, NY (US)

(72) Inventors: Neville E. Sanjana, New York, NY (US); Mateusz Legut, Jersey City, NJ (US)

(21) Appl. No.: 18/576,076

(22) PCT Filed: Jun. 30, 2022
(86) PCT No.: PCT/US2022/073294

§ 371 Date: Jan. 2, 2024

Related U.S. Application Data
(60) Provisional application No. 63/320,101, filed on Mar. 15, 2022.
(60) Provisional application No. 63/287,389, filed on Dec. 8, 2021.
(60) Provisional application No. 63/217,014, filed on Jun. 30, 2021.

(51) Int. Cl. A61K 35/17; A61K 39/00; A61P 35/02; C07K 14/705; C07K 14/725; C12N 5/0783; C12N 15/86; G01N 33/50

(52) U.S. Cl. CPC A61K 35/17; A61K 39/4611; A61K 39/4631; A61K 39/464412; A61K 39/464417; A61K 39/464468; A61P 35/02; C07K 14/7051; C07K 14/70578; C12N 5/0636; C12N 15/86; G01N 33/505; A61K 2239/48; A61K 2239/54; C12N 2510/00; C12N 2740/15043; G01N 2500/10

(57) ABSTRACT

Provided herein are nucleic acids, expression cassettes, modified lymphocytes and compositions comprising the same which include a sequence encoding a gene of Table 1. In some embodiments, the gene is LTBR. In certain embodiments, the cell is a T cell. In certain embodiments, the cell further comprises a CAR or engineered TCR. Methods of treatment using the provided compositions are also described.

### STATEMENT OF GOVERNMENT SUPPORT

This invention was made with government support under R00HG008171, DP2HG010099, and R01CA218668 awarded by the National Institutes of Health and D18AP00053 awarded by the Defense Advanced Research Projects Agency. The government has certain rights in the invention.

### BACKGROUND

Cellular immunotherapies with engineered autologous patient T cells redirected against a chosen tumor antigen have yielded great efficacy against blood cancers, resulting in five approvals for chimeric antigen receptors (CARs) by the US Food and Drug Administration (FDA) so far⁶. By contrast, CAR therapy for solid tumors has shown a much lower efficacy overall, owing to the suppression of T cell effector function in the tumor microenvironment. Even for blood malignancies, with the exception of B acute lymphoblastic leukemia, most patients do not experience a durable response, with resistance being primarily due to T cell dysfunction rather than antigen loss⁷. Considerable efforts have been devoted to identifying genes and pathways that contribute to T cell dysfunction^{8,9}. However, comprehensive, genome-wide screens for modulators of T cell function thus far have been limited to loss-of-function screens²⁻⁴.

The advances in CRISPR genome engineering have made it possible to readily knock out every gene in the genome in a scalable and customizable manner. Although its large size makes it challenging (albeit not impossible¹⁰) to deliver Cas9 via lentivirus to primary T cells, alternative approaches have been developed, which rely on transient delivery of Cas9 protein² or mRNA¹¹, or on constitutive Cas9 expression in engineered isogenic mouse strains³. These approaches, however, are not amenable to gain-of-function screens in human cells, which require continuous expression of the transcriptional activator that drives target gene expression.

What is needed is improved compositions and methods for more effective immunotherapies.

### SUMMARY OF THE INVENTION

Provided herein, in one aspect, is a modified lymphocyte comprising an exogenous nucleic acid encoding a gene of Table 1. In certain embodiments, the gene is LTBR. In other embodiments, the gene is LTBR, ADA, IFNL2, IL12B CALML3 MRPL51, DBI GPN3, ITM2A, AHNAK, BATF, GPD1, ATF6B, AHCY, DUPD1, or AKR1C4.

In one embodiment, the lymphocyte comprises an expression cassette comprising an expression control sequence and a nucleic acid encoding a gene of Table 1. In certain embodiments, the gene is LTBR. In other embodiments, the gene is LTBR, ADA, IFNL2, IL12B CALML3 MRPL51, DBI GPN3, ITM2A, AHNAK, BATF, GPD1, ATF6B, AHCY, DUPD1, or AKR1C4.

In certain embodiments, the lymphocyte further comprises a nucleic acid encoding a chimeric antigen receptor (CAR). In certain embodiments, the CAR is Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kyrmriah®), or one of those found in FIG. 19. In certain embodiments, the CAR is a chimeric autoantibody receptor (CAAR). In certain embodiments, the lymphocyte further comprises a nucleic acid encoding a T cell receptor (TCR). In certain embodiments, the TCR is selected from those found in FIG. 17. In certain embodiments, the lymphocyte is a T cell.

In another aspect, a vaccine composition is provided comprising a nucleic acid encoding a gene of Table 1 and a nucleic acid encoding a viral protein. In certain embodiments, the viral protein is a glycoprotein. In certain embodiments, the glycoprotein is a viral spike protein, optionally a coronavirus spike protein. In certain embodiments, the nucleic acid encoding the gene of Table 1 is mRNA, or the nucleic acid encoding the viral spike protein is mRNA, or both. In certain embodiments, the gene is LTBR. In other embodiments, the gene is LTBR, ADA, IFNL2, IL12B CALML3 MRPL51, DBI GPN3, ITM2A, AHNAK, BATF, GPD1, ATF6B, AHCY, DUPD1, or AKR1C4.

In another aspect, an expression cassette comprising a nucleotide sequence encoding a chimeric antigen receptor and a nucleic acid encoding a gene of Table 1 is provided. In certain embodiments, the gene is LTBR. In other embodiments, the gene is LTBR, ADA, IFNL2, IL12B CALML3 MRPL51, DBI GPN3, ITM2A, AHNAK, BATF, GPD1, ATF6B, AHCY, DUPD1, or AKR1C4.

In another aspect, an expression cassette comprising a nucleic acid encoding a T cell receptor and a nucleic acid encoding a gene of Table 1 is provided. In certain embodiments, the gene is LTBR. In other embodiments, the gene is LTBR, ADA, IFNL2, IL12B CALML3 MRPL51, DBI GPN3, ITM2A, AHNAK, BATF, GPD1, ATF6B, AHCY, DUPD1, or AKR1C4.

In another aspect, an expression cassette comprising a nucleic acid encoding a viral protein and a nucleic acid encoding a gene of Table 1 is provided. In certain embodiments, the gene is LTBR. In other embodiments, the gene is LTBR, ADA, IFNL2, IL12B CALML3 MRPL51, DBI GPN3, ITM2A, AHNAK, BATF, GPD1, ATF6B, AHCY, DUPD1, or AKR1C4.

In another aspect, a method of producing a modified lymphocyte comprising introducing an exogenous nucleic acid encoding a gene of Table 1 into the cell. In certain embodiments, the lymphocyte comprises an expression cassette comprising an expression control sequence and a nucleic acid encoding a gene of Table 1. In certain embodiments, the gene is LTBR. In other embodiments, the gene is LTBR, ADA, IFNL2, IL12B CALML3 MRPL51, DBI GPN3, ITM2A, AHNAK, BATF, GPD1, ATF6B, AHCY, DUPD1, or AKR1C4. In certain embodiments, the lymphocyte further comprises a nucleic acid encoding a chimeric antigen receptor (CAR). In other embodiments, the lymphocyte further comprises a nucleic acid encoding an engineered T cell receptor (TCR).

In another aspect, a method of treating cancer in a subject in need thereof is provided. The method includes administering a composition as described herein to a subject in need thereof. In certain embodiments, the subject has a solid tumor. In certain embodiments, the subject has lymphoma, optionally B cell lymphoma, follicular lymphoma, or mantle cell lymphoma. In certain embodiments, the subject has leukemia. In certain embodiments, the subject has multiple myeloma. In certain embodiments, the subject has a virally-driven cancer, optionally Burkitt's lymphoma, liver cancer, Kaposi's sarcoma, cervical cancer, head cancer, neck cancer, anal cancer, oral cancer, pharyngeal cancer, penile cancer, adult T-cell lymphoma, or merkel cell carcinoma.

In another aspect, a method of treating a viral disease in a subject in need thereof is provided. The method includes administering a composition as described herein to a subject in need thereof. In certain embodiments, the disease is HIV. In certain embodiments, the disease is HPV. In certain embodiments, the disease is an autoimmune disorder.

In another aspect, a method of treating an autoimmune disease in a subject in need thereof is provided. The method includes administering a composition as described herein to a subject in need thereof.

In another aspect, a method of increasing proliferation, or T cell effector function including cytokine production and/or secretion, the method comprising administering a composition as described herein to a T cell. In certain aspects, the T cell is obtained from a human prior to treating the T cell to overexpress the gene of Table 1, and the treated T cell is reintroduced into a human. In certain embodiments, the gene is LTBR. In other embodiments, the gene is LTBR, ADA, IFNL2, IL12B CALML3 MRPL51, DBI GPN3, ITM2A, AHNAK, BATF, GPD1, ATF6B, AHCY, DUPD1, or AKR1C4.

In another aspect, a method of increasing the response to a vaccine composition is provided. The method includes co-administering with a vaccine a nucleic acid encoding a gene of Table 1. In certain embodiments, the gene is LTBR. In other embodiments, the gene is LTBR, ADA, IFNL2, IL12B CALML3 MRPL51, DBI GPN3, ITM2A, AHNAK, BATF, GPD1, ATF6B, AHCY, DUPD1, or AKR1C4.

In certain embodiments of any of the methods described herein, the expression of the gene of Table 1 is transient.

In another aspect, a method of identifying a gene that alters the therapeutic function of a modified lymphocyte when exogenously expressed in the modified lymphocyte is provided. The method includes: (a) obtaining a lymphocyte population; (b) transducing the lymphocyte population with a plurality of viral vectors, each viral vector encoding a gene which may be linked to one or more barcodes; (c) stimulating the transduced lymphocytes to induce activation, proliferation, and/or effector function; (d) isolating a transduced lymphocyte from the lymphocyte population of (c); and (e) detecting the presence of the gene and/or the linked barcodes in the isolated lymphocyte; wherein the detected gene is effective to alter the therapeutic function of a modified lymphocyte that expresses the gene. In certain embodiments, the gene is an open-reading frame (ORF) or a nucleotide sequence encoding a non-coding RNA, optionally a microRNA (miRNA) or long non-coding RNA (lncRNA, long ncRNA). In certain embodiments, the lymphocyte population comprises a cell population that has been enriched for one or more of T cells, B cells, NK T cells, NK cells, or a subpopulation thereof, optionally wherein the cells are human. In certain embodiments, the plurality of viral vectors comprises a library of open reading frames (ORFs). In certain embodiments, the viral vector is a retroviral vector or a lentiviral vector. In certain embodiments, stimulating the transduced lymphocytes comprises culturing the lymphocytes with one or more of an antibody, cytokine, an antigen, a superantigen, an antigen presenting cell, a cancer cell, and a cancer cell line. In certain embodiments, stimulation of the transduced lymphocytes comprises TCR stimulation, optionally comprising CD3/CD28 stimulation. In certain embodiments, the method further includes labeling the transduced lymphocytes with a cell proliferation dye, and isolating progeny cells. In certain embodiments, step (d) comprises identifying cells that express one or more cell surface markers and/or one or more effector functions and/or one or more secreted cytokines. In certain embodiments, step (e) comprises obtaining genomic DNA from the isolated lymphocyte and PCR amplification of the gene and/or barcode sequence. In certain embodiments, step (e) further comprises single-cell transcriptome and/or proteome analysis. In certain embodiments, (e) comprises flow cytometric analysis, cell-hashing, single-cell sequencing analysis, single cell RNA sequencing (scRNA-seq), Perturb-seq, CROP-seq, CRISP-seq, ECCITE-seq, or cellular indexing of transcriptomes and epitopes (CITE-seq).

In another aspect, a method of analyzing the effect on an individual cell of overexpression of an ORF of interest is provided. The method includes (a) introducing into the cell an expression cassette comprising a nucleic acid encoding the ORF of interest and overexpressing said ORF; (b) providing a first set of nucleic acids derived from the individual cell and oligonucleotides having a common barcode sequence into a discrete partition, wherein the oligonucleotides are releasably attached to a bead, wherein the first set of nucleic acids comprises endogenous transcriptome mRNA and ORF mRNA; (c) performing RT-PCR to generate a second set of nucleic acids derived from the first set of nucleic acids, wherein said second set of nucleic acids within the partition have attached thereto oligonucleotides that comprise the common nucleic acid barcode sequence, and wherein the RT-PCR is performed using RT-PCR reagents which comprise a primer which specifically anneals to a sequence on the ORF mRNA, that is not a poly A sequence, and wherein the second set of nucleic acids comprises endogenous transcriptome cDNA and ORF cDNA; (d) amplifying the second set of nucleic acids to generate a third set of nucleic acids using PCR reagents which comprise a second primer which specifically anneals to a sequence on the ORF cDNA, that is not a poly A sequence; and (c) detecting and/or sequencing the barcode sequence, transcriptome cDNA, and/or ORF cDNA. In certain embodiments, step (e) further comprises single-cell transcriptome and/or proteome analysis. In certain embodiments, (e) comprises flow cytometric analysis, cell-hashing, single-cell sequencing analysis, single cell RNA sequencing (scRNA-seq), Perturb-seq, CROP-seq, CRISP-seq, ECCITE-seq, or cellular indexing of transcriptomes and epitopes (CITE-seq).

In certain embodiments, the method includes obtaining a portion of the third set of nucleic acids and amplifying the ORF cDNA using a second set of PCR reagents which comprise a third primer which specifically anneals to a sequence on the ORF cDNA, that is not a poly A sequence, to generate a fourth set of nucleic acids.

In certain embodiments, the method includes amplifying the ORF cDNA in the fourth set of nucleic acids using a third set of PCR reagents which comprise a fourth primer which specifically anneals to a sequence on the ORF cDNA, that is not a poly A sequence, to generate a fifth set of nucleic acids; and wherein step (e) comprises fragmenting said third set and said fifth set of nucleic acids, ligating adapters to ends, and subjecting to NGS.

Other aspects and advantages of the invention will be readily apparent from the following detailed description of the invention.

### BRIEF DESCRIPTION OF THE DRAWINGS

FIG. 1A-FIG. 1B show a genome-scale overexpression screen to identify genes that boost the proliferation of primary human T cells. (FIG. 1A) Overview of the pooled ORF screen. CD4⁺ and CD8⁺ T cells were separately isolated from peripheral blood from three healthy donors. The barcoded genome-scale ORF library was then introduced into CD3/CD28-stimulated T cells, followed by selection of transduced cells. After 14 days of culture, T cells were labelled with carboxy fluorescein succinimidyl ester (CFSE) and restimulated to induce proliferation. By comparing counts of specific ORF barcodes before and after cell sorting, we identified ORFs enriched in the CFSElow population. Those genes included LTBR, VSTM1, CD59, IL12B, 1L23A, MAPK3, ADA, and DBI. (FIG. 1B) Robust rank aggregation of genes in both CFSE^{low}CD4⁺ and CFSE^{low}CD8⁺ T cells, based on consistent enrichment of individual barcodes for each gene, are shown.

FIG. 2A-FIG. 2F show overexpression of top-ranked ORFs increases the proliferation, activation and cytokine secretion of CD4⁺ and CD8⁺ T cells. (FIG. 2A) CD4+ and CD8⁺ T cells from screen-independent donors were separately isolated and then transduced with lentiviruses encoding top-ranked ORFs together with a selection marker. After transduction and selection, T cells were restimulated before measurement of proliferation, expression of activation markers and cytokine secretion. (FIG. 2B) Proliferation of T cells transduced with top-ranked genes as the relative proliferation, which is defined as the ratio of stimulated cells to the corresponding unstimulated control, normalized to tNGFR. A minimum of two donors was tested per overexpressed gene, in biological triplicate. Boxes show 25th-75th percentiles with a line at the mean; whiskers extend to maximum and minimum values. DUPD1 is also known as DUSP29. (FIG. 2C) Mean relative proliferation of ORF-transduced T cells in CD4⁺ and CD8⁺ T cells, normalized to tNGFR. Significant genes in both T cell subsets or either of them are marked (Student's two-sided/test P<0.05 and false discovery rate <0.1). (FIG. 2D) Representative expression of CD25 or CD154 after restimulation. The numbers on the histograms correspond to the percentage of gated cells (CD8⁺CD154⁺) or the mean fluorescence intensity (MFI). Dashed lines indicate the gate used to enumerate CD154⁺ cells (CD8⁺) or MFI for control (tNGFR) cells. (FIG. 2E) Secretion of IL-2 and IFNγ after restimulation, normalized to tNGFR. Only genes that significantly increase T cell proliferation in CD4⁺, CD8⁺ or both T cell subsets are shown. A minimum of two donors was tested in triplicate per gene. Boxes show 25th-75th percentiles with a line at the mean; whiskers extend to maximum and minimum values. (FIG. 2F) Intersection between different T cell activation phenotypes that are significantly (P<0.05) improved by a given ORF in CD8⁺ or CD4⁺ T cells.

FIG. 3A-FIG. 3E show single-cell OverCITE-seq identifies shared and distinct transcriptional programs that are induced by gene overexpression in T cells. (FIG. 3A) OverCITE-seq captures overexpression (ORF) constructs, transcriptomes, TCR clonotypes, cell-surface proteins and treatment hashtags in single cells. (FIG. 3B) ORF assignment rate in resting and CD3/CD28-stimulated T cells. (FIG. 3C) Antibody-derived tag sequencing (ADTs; right) yields similar NGFR expression in tNGFR-transduced T cells to flow cytometry (left) with tNGFR-transduced T cells. Untransduced cells (left) or cells assigned a non-tNGFR ORF (right) are shown in grey. (FIG. 3D) Uniform manifold approximation and projection (UMAP) representation of single-cell transcriptomes after unsupervised clustering of OverCITE-seq-captured ORF singlets. The inset in the top left identifies stimulated and resting T cells as given by treatment hashtags. For each cluster, a subset of the top 20 differentially expressed genes is shown. HIST1H1B is also known as H1-5 and HIST1H3C is also known as H3C3. (FIG. 3E) ORF prevalence in two representative clusters. Standardized residual values are from a chi-squared test. ORFs of interest are shown.

FIG. 4A-FIG. 4K show LTBR overexpression improves T cell function through activation of the canonical NF-κB pathway. (FIG. 4A) Differential expression of genes in resting LTBR and tNGFR (negative control) T cells. Genes highlighted in red are those with a twofold or greater change in expression and an adjusted P<0.05. (FIG. 4B) Significantly enriched GO biological processes in LTBR-overexpressing T cells (p<0.05). (FIG. 4C) Cell viability of CD8⁺ T cells transduced with LTBR or tNGFR lentivirus, either restimulated with CD3/CD28 for four days or left unstimulated (n=2 donors with 3 biological replicates each). (FIG. 4D) PD-1 expression on resting LTBR or tNGFR T cells stimulated with a 3:1 excess of CD3/CD28 beads every three days, for up to three rounds of consecutive stimulation. (FIG. 4E) ICAM-1 expression (resting) and IL-2 secretion (activated) by T cells transduced with Flag-tagged LTBR mutants, normalized to wild-type LTBR (n=6 replicates across two experiments). (FIG. 4F) Enrichment of transcription factor motifs in differentially accessible chromatin (top 10 motifs from each comparison). (FIG. 4G) Quantification of phosphorylated RELA (phospho-RELA) in LTBR or tNGFR T cells stimulated with CD3/CD28 antibodies for the indicated periods of time. (FIG. 4H, FIG. 4I) Quantification of phosphorylated IκBα (FIG. 4H) or mature NF-κB2 (FIG. 4I) in resting or CD3/CD28-stimulated (15 min) LTBR or tNGFR cells. (FIG. 4J) IFNγ secretion by stimulated LTBR or tNGFR cells after CRISPR knockout of the indicated genes (n=18, 3 sgRNAs in 2 donors in 3 biological replicates). IFNγ quantities are normalized to corresponding non-targeting (NT) controls (either LTBR or tNGFR) to allow comparisons of the relative effects of gene knockout on T cell activation. (FIG. 4K) Expression levels of core LTBR genes (n=274 genes) in LTBR and tNGFR cells after CRISPR knockout of RELA or RELB (normalized to non-targeting control in LTBR cells). Boxes show 25th-75th percentiles with a line at the median; whiskers extend to 1.5 times the interquartile range. Unpaired two-sided/test P values (FIG. 4C, FIG. 4G-FIG. 4K); not significant (NS) P>0.05, *P<0.05, **P<0.01, ***P<0.001, ****P<0.0001. Error bars, s.e.m.; n=3 biological replicates, unless stated otherwise.

FIG. 5A-FIG. 5I show top-ranked genes improve antigen-specific T cell responses and tumor killing. (FIG. 5A-FIG. 5F) Co-delivery of anti-CD19 CARs and ORFs to T cells from healthy donors. (FIG. 5A) Schematic of tricistronic vector and CAR T cell experiments. (FIG. 5B, FIG. 5C) Secretion of IFNγ (FIG. 5B) and IL-2 (FIG. 5C) after overnight co-incubation of CD8⁺ T cells with Nalm6 cells at a 1:1 ratio (n=3 biological replicates, representative of 2 donors). (FIG. 5D) Nalm6 GFP⁺ cell proliferation (normalized total GFP per well) after co-incubation with T cells co-expressing 19-28z CAR and LTBR or tNGFR (negative control) at the indicated effector-to-target ratios. (FIG. 5E) Quantification of Nalm6 GFP⁺ clearance for T cells co-expressing 19-28z or 18-BBz CARs and top-ranked genes (n=3 biological replicates, representative of 2 donors), normalized to tNGFR at an effector-to-target ratio of 0.25 and after 48 h of co-incubation. (FIG. 5F) 19-BBz CAR T cells co-expressing LTBR or tNGFR were co-incubated at a 1:1 ratio with Nalm6 cells every 3 days for up to 3 rounds of stimulation (n=3 biological replicates). Seven days after repeated antigen stimulation, CAR T cells were re-exposed to Nalm6 cells. IFNγ secretion was measured after overnight incubation. (FIG. 5G) Co-delivery of anti-CD19 CARs and ORFs to total PBMCs from a patient with diffuse large B cell lymphoma. Transduced T cells were incubated alone, or co-incubated with CD19⁺ Nalm6 or CD19⁻ Jurkat cell lines at a 1:1 ratio (n=3 biological replicates, representative of 2 patients). Secretion of IFNγ and IL2 was measured after overnight incubation. For the Nalm6 condition, numbers above indicated column pairs are the fold increase in cytokine secretion by LTBR cells over tNGFR (negative control) cells. (FIG. 5H) Delivery of ORFs to Vγ9Vδ2 T cells. Secretion of IFNγ and IL-2 after overnight co-incubation with the pancreatic ductal adenocarcinoma (PDAC) line Capan-2, pre-treated with zoledronate to boost phosphoantigen accumulation (n=3 biological replicates). Data are mean±s.e.m. where appropriate.

FIG. 6A-FIG. 6M show design of the human ORF library screen in primary T cells. (FIG. 6A) Barcoded vector design for ORF overexpression. (FIG. 6B) Distribution of the number of barcodes per ORF in the library. (FIG. 6C) Vector design for quantifying the effect of different promoters and ORF insert sizes on lentiviral transduction efficiency. EFS-elongation factor-1α short promoter, CMV-cytomegalovirus promoter, PGK-phosphoglycerate kinase-1 promoter. (FIG. 6D) Percentage of positive cells and (FIG. 6E) mean fluorescence intensity (MFI) of rat CD2 (rCD2) expressed from the EFS and CMV promoters, following puromycin selection of transduced primary CD4⁺ T cells. Each data point indicates individual transduction (n=3 biological replicates). Error bars are SEM. (FIG. 6F) Distribution of ORF sizes in the genome-scale library. The size of TCR-rCD2 construct tested in panels FIG. 6D and FIG. 6E is marked. (FIG. 6G) Titration of CD3/CD28 antibodies. T cells were labelled with CFSE, stimulated and incubated for 4 days. Gate for proliferating T cells was set to include cells that proliferated at least twice (third CFSE peak). (FIG. 6H) Expansion of T cells from three healthy donors transduced with the ORF library. (FIG. 6I) Representative CFSE profile of restimulated CD8⁺ and CD4⁺ T cells before the sort. The CFSE^{low} sort gate is marked. (FIG. 6J) Recovery of individual barcodes or corresponding ORFs in transduced T cells and plasmid used for lentivirus production. Respective samples from three donors were computationally pooled together at equal number of reads prior to counting how many barcodes or ORFs were present with a minimum of one read. (FIG. 6K) Enrichment of genes in both CFSE^{low} CD4⁺ and CD8⁺ T cells, calculated by collapsing individual barcodes into corresponding genes. Significantly enriched genes (log₂ fold change higher than 0.5 and adjusted p-value lower than 0.05) are marked in red. Immune response genes of interest are marked. (FIG. 6L) GO biological processes for significantly enriched genes in FIG. 6K. (FIG. 6M) Overlap of significantly enriched genes with differentially expressed genes between CD3/CD28 stimulated and naive T cells⁴¹.

FIG. 7A-FIG. 7J show overexpression of select ORFs in screen independent donors. (FIG. 7A) Histograms of selected ORF expression in T cells after puromycin selection. (FIG. 7B) Quantification of tNGFR expression in transduced CD4⁺ and CD8⁺ T cells. Puromycin selection was complete after 7 days post transduction. To maintain T cells in culture, they were restimulated with CD3/CD28 on days 21 and 42. (FIG. 7C) Correlation between ORF sizes and changes in proliferation relative to tNGFR. Mean log₂ fold-changes are shown. (FIG. 7D) Proliferation of restimulated CD8⁺ or (FIG. 7E) CD4⁺ T cells relative to tNGFR in individual donors (n=3 biological replicates). Mean and SEM are shown. (FIG. 7F, FIG. 7G) Proliferation of T cells transduced with ORFs that significantly improved T cell proliferation (see FIG. 2C) measured by dilution of CellTrace Yellow. Representative CellTrace Yellow histograms and fitted distributions (FIG. 7F) as well as quantifications of the proliferation index (FIG. 7G) are shown (n=3 biological replicates). P values: <0.0001, 0.0008, <0.0001, 0.011, 0.0031, 0.0007, <0.0001, 0.28, 0.004, <0.0001, 0.58, 0.01, 0.0003, <0.0001, 0.036, 0.0049 (left to right). (FIG. 7H) Viability of ORF-transduced T cells 4 days after CD3/CD28 restimulation. Representative data from one donor (out of 4 donors tested) are shown (n=3 biological replicates; CD8 left bars, CD4 right bars). (FIG. 7I, FIG. 7J) Cell cycle analysis of T cells stimulated with CD3/CD28 for 24 h. Gating was performed based on isotype and fluorescence minus one controls. Representative gating (FIG. 7I) as well as (FIG. 7J) quantification of cells in the S-G2-M phases (for stimulated T cells) are shown (n=6 biological replicates from two donors). P values: 1, 0.29, 0.0065, 0.17, 0.0051, 1, 0.13, 0.55, 0.0004, 0.98, 0.0088, 0.68, 0.91, 0.7, 1 (left to right). Statistical significance for panels FIG. 7G and FIG. 7I: one way ANOVA with Dunnett's multiple comparisons test *p<0.05. **p<0.01, ***p<0.001, ****p<0.0001. Error bars indicate SEM.

FIG. 8A-FIG. 8E show functional response of ORF-overexpressing T cells. (FIG. 8A) Quantitative expression of CD25 or CD154 following restimulation. A minimum of two donors was tested in triplicate per gene. Only genes that significant increase T cell proliferation in CD4⁺, CD8⁺ or both T cell subsets are shown. Mean and SEM are shown. CD4+ are shown in top bars; CD8+ cells are shown in bottom bars. (FIG. 8B, FIG. 8C) Sensitivity to antigen dose. T cells were incubated with indicated anti-CD3 antibody concentrations for 24 h and the amount of secreted IFNγ was quantified. Representative dose-response curve fitting (FIG. 8B) and IC₅₀ quantifications (FIG. 8C) are shown (n=2 biological replicates). (FIG. 8D) Quantification of secreted IL-2 and IFNγ in T cells incubated alone or with CD3/CD28 antibodies for 24 h. Representative data from one out of four donors (n=3 biological replicates) are shown. Resting cells are shown by left bars, +CD3/CD28 shown on right. (FIG. 8E) Multiplexed quantification of selected secreted cytokines and chemokines by ORF-transduced T cells after 24 h of CD3/CD28 stimulation. Means of duplicate measurements (from independent samples) z-score normalized to tNGFR are shown.

FIG. 9A-FIG. 9J show OverCITE-seq identifies ORFs and their transcriptional effects. (FIG. 9A) Quality parameters of cells as identified by gel bead barcodes. Negative, singlets and doublets are assigned based on cell hashing. (FIG. 9B) Proportion of stimulated and resting T cells among cells assigned to each ORF. Chi-squared test p-values are shown for ORFs with significantly shifted (uneven) distributions of stimulated and rested cells. (FIG. 9C) Cell-cycle corrected scaled expression of the overexpressed gene in the cells transduced with the respective ORF and negative control (tNGFR). Two-sided Wilcoxon test p-values shown above the violin plots indicate the statistical significance of gene expression level between specific ORF and tNGFR-transduced T cells. Box shows 25-75 percentile with a line at the median; whiskers extend to maximum and minimum values. N=71 (ADA), 147 (AHCY), 190 (AHNAK), 119 (AKR1C4), 124 (ATF6B), 179 (BATF), 137 (CALML3), 189 (CDK1), 129 (CDK2), 236 (CLIC1), 84 (CRLF2), 91 (CXCL12), 88 (CYP27A1), 129 (DBI), 26 (DCLRE1B), 261 (DUPD1), 25 (FOSB), 119 (GPD1), 124 (GPN3), 199 (IFNL2), 60 (IL12B), 70 (IL1RN), 156 (ITM2A), 74 (LTBR), 88 (MRPL18), 167 (MRPL51), 107 (MS4A3), 69 (NFYB), 355 (NGFR), 261 (RAN), 182 (SLC10A7), and 56 (ZNF830) single cells. (FIG. 9D) Expression of all ORF genes by cells assigned each ORF. Each row is z-score normalized. (FIG. 9E) Distribution of individual ORF frequencies in clusters. Numbers of ORF cells and the chi-squared test residuals are displayed. Chi-squared test p-values indicating whether ORF distribution in each cluster significantly differs from overall ORF distribution are shown on top of the plot. Proportions of stimulated and resting T cells in each cluster are shown underneath the cluster label. (FIG. 9F, FIG. 9G) Spearman correlations between transcriptional profiles of selected ORF cells in resting (FIG. 9F) and stimulated (FIG. 9G) populations. (FIG. 9H) Fold change of top differentially expressed genes between cells with the indicated ORFs in resting and stimulated T cells. For each condition, the ORFs with the strongest transcriptional changes (compared to tNGFR cells) are shown. (FIG. 9I) Differential gene expression in stimulated ORF T cells compared to resting T cells. Genes with significant expression changes in at least one ORF are shown (DESeq2 adjusted p<0.05). For all genes, we display log₂ fold-change of each ORF (stimulated) to tNGFR (resting), normalized to log₂ fold-change of tNGFR (stimulated) to tNGFR (resting). Genes of interest in each cluster are labelled. (FIG. 9J) Mean TCR clonotype diversity in ORF cells.

FIG. 10A-FIG. 10M show functional analysis of LTBR overexpression in T cells. (FIG. 10A) LTBR expression in the indicated human primary tissues from the Genotype-Tissue Expression (GTEx) project v8⁷⁵ (n=948 donors). Box shows 25-75 percentile with a line at the median. (FIG. 10B) LTBR expression in peripheral blood mononuclear cells (PBMCs) from 31,021 cells from 2 donors⁷⁶. Cell types indicated are derived from Harmony tSNE clustering of single-cell transcriptomes. (FIG. 10C) Overlap between significantly upregulated genes in LTBR cells compared to tNGFR cells identified in single-cell or bulk RNA-seq. (FIG. 10D, FIG. 10E) TCF1 expression in LTBR or tNGFR transduced T cells. (FIG. 10D) Representative histograms of TCF1 expression and the gate for TCF1+ cells (dashed line) are shown, as well as (FIG. 10E) quantification of TCF1⁺ cells (n=3 biological replicates). (FIG. 10F-FIG. 10H) ICAM-1, CD70, CD74, and MHC-II expression in LTBR and tNGFR T cells. Representative histograms (FIG. 10F), quantification (FIG. 10G) in n=3 donors (CD8⁺) or n=4 donors (CD4⁺) and time course (FIG. 10H) of expression in LTBR and tNGFR cells after CD3/CD28 stimulation (n=3 biological replicates). (FIG. 10I) Differentiation phenotype of NGFR and LTBR transduced T cells (n=4 donors, CD4⁺ and CD8⁺ separately). CM: Central memory. EM: Effector memory. Differentiation was defined based on CD45RO and CCR7 expression (naive: CD45RO^{neg} CCR7⁺, CM: CD45RO⁺ CCR7⁺, EM: CD45RO⁺ CCR7^{neg} effector CD45RO^{neg} CCR 7^{neg}). (FIG. 10J) Representative dot plots of T cell viability after CD3/CD28 stimulation. Viable cells are in the lower left quadrant. (FIG. 10K) Cell viability of CD4⁺ T cells transduced with LTBR or tNGFR lentivirus, either restimulated with CD3/CD28 for four days or left unstimulated (n=2 donors with 3 biological replicates each). (FIG. 10L, FIG. 10M) LTBR and tNGFR cells were stimulated with a 3:1 excess of CD3/CD28 beads every three days for up to three rounds of stimulation. Following repeated stimulation, expression of TIM-3 and LAG-3 (FIG. 10L) was measured in resting cells, and secretion of IFNγ and IL2 (FIG. 10M) was measured in restimulated cells (n=3 biological replicates). Statistical significance for panels FIG. 10E, FIG. 10I, and FIG. 10K: two-sided unpaired t-test; for panel FIG. 10G: two-sided paired t-test. Error bars indicate SEM.

FIG. 11A-FIG. 11K show LTBR ligands and expression of LTBR via mRNA or with deletion and point mutants. (FIG. 11A) IL2 secretion after 24 h stimulation with CD3/CD28 antibodies. Where indicated, recombinant soluble LTA (1 ng/mL) or LIGHT (10 ng/ml) were added together with CD3/CD28 antibodies. CD4⁺ T cells from one donor were tested in triplicate. (FIG. 11B, FIG. 11C) CD4⁺ and CD8⁺ T cells from two donors were co-incubated for 24 h with CD3/CD28 antibodies or recombinant soluble LTA or LIGHT and then IL2 (FIG. 11B) and IFNγ (FIG. 11C) were measured. (n=3 biological replicates). No stimulatin, +CD3/CD28, +LTA and +LIGHT shown from left to right. (FIG. 10D, FIG. 10E) Differentiation phenotype (FIG. 10D) or proliferation (FIG. 10E) after restimulation of tNGFR and LTBR transduced T cells (n=3 biological replicates) incubated either with IL2 alone or with LTA (1 ng/ml) or LIGHT (10 ng/mL) for the duration of culture. CM: Central memory. EM: Effector memory. Unpaired two-sided t-test p values are shown. (FIG. 11F, FIG. 11I) Transient LTBR or tNGFR expression via mRNA nucleofection (FIG. 101F). T cells were either nucleofected with LTBR or tNGFR mRNA (n=3 biological replicates), and the surface expression of LTBR (FIG. 11G), tNGFR (FIG. 11H) or four genes upregulated in LTBR cells (FIG. 11I) was monitored over 21 days. At each timepoint the expression of target genes was normalized to matched tNGFR control. (FIG. 11J) Schematic representation of FLAG-tagged LTBR mutants. (FIG. 11K) LTBR and FLAG expression in T cells transduced with LTBR mutants. Error bars indicate SEM.

FIG. 12A-FIG. 12I show chromatin accessibility in LTBR T cells. (FIG. 12A) Principal component (PC) analysis of global accessible chromatin regions of LTBR and tNGFR T cells, either resting or stimulated with CD3/CD28 for 24 h. (FIG. 12B) Differentially accessible chromatin regions between stimulated and resting tNGFR, stimulated and resting LTBR, resting LTBR and resting tNGFR, and stimulated LTBR and stimulated tNGFR. Numbers of peaks gained/lost are shown (using absolute log₂, fold change of 1 and adjusted p value <0.1 as cut-off). (FIG. 12C, FIG. 12D) Changes in chromatin accessibility (FIG. 12C) for differentially expressed (adjusted p<0.05) genes or in gene expression (FIG. 12D) for differentially accessible (adjusted p<0.05) regions. Two-sided t-test p values are shown. Box shows 25-75 percentile with a line at the median; whiskers extend to 1.5× interquartile range. N=614 genes (FIG. 12C) or genomic regions (FIG. 12D). (FIG. 12E, FIG. 12F) Chromatin accessibility profiles at loci more (FIG. 12E) or less open (FIG. 12F) in LTBR compared to tNGFR cells, resting or stimulated for 24 h. The y-axis represents normalized reads (scale: 0-860 for BATF3, 0-1950 for IL13, 0-1230 for TRAF1, 0-1000 for TNFSF4, 0-300 for PDCD1, 0-2350 for LAG3). (FIG. 12G) Chromatin accessibility in resting or stimulated LTBR and tNGFR cells. Each row represents a peak significantly enriched in LTBR over matched tNGFR control (log₂ fold change >1, DESeq2 adjusted p value <0.05). Peaks were clustered using k-means clustering and selected genes at/near peaks from each cluster are indicated. (FIG. 12H) Correlations for each ATAC sample (biological replicate) based on the bias-corrected deviations. (FIG. 12I) Top transcription factor (TF) motifs enriched in the differentially accessible chromatin regions in resting LTBR cells compared to resting tNGFR cells.

FIG. 13A-FIG. 13P show proteomic and functional genomic assays of NF-κB activation. (FIG. 13A) Phospho-RELA staining by intracellular flow cytometry in LTBR and tNGFR cells. Gating for identification of phospho-RELA+ cells is shown. (FIG. 13B, FIG. 13C) Western blot quantification of key proteins in the NF-κB pathway in LTBR and tNGFR cells, resting or stimulated with CD3/CD28 for 15 min. Representative gels (FIG. 13B) or quantification of band intensity relative to GAPHD (FIG. 13C) are shown (n=3 biological replicates). Unpaired two-sided t test p values are shown. (FIG. 13D) Representation of the LTBR signaling pathway. Each gene is colored based on the differential expression in LTBR over matched tNGFR cells (CD4⁺ and CD8⁺ T cells, resting or stimulated for 24 h). (FIG. 13E-FIG. 13G) Simultaneous gene knockout via CRISPR and ORF overexpression. T cells were transduced with a lentiviral vector co-expressing a single guide RNA (sgRNA) and the LTBR ORF. After transduction, Cas9 protein was delivered via nucleofection. (FIG. 13F) Representative expression of target genes in LTBR cells co-expressing an sgRNA targeting B2M, an essential component of the MHC-I complex, or TRBC1/2, an essential component of the αβ TCR. (FIG. 13G) Quantification of IFNγ after restimulation (n=3 sgRNAs). (FIG. 13H-FIG. 13O) Representative protein-level based quantification of gene knockout efficiency. Representative histograms (FIG. 13H, FIG. 13J, FIG. 13L) and quantification of relative expression levels of LTA, LIGHT, and RELA (FIG. 13I, FIG. 13K, FIG. 13M) are shown (n=3 sgRNAs). Dashed lines represent gates used to enumerate cells expressing a given protein. Representative gel (FIG. 13N) and quantification of RELB expression (FIG. 13O) are shown (n=3 sgRNAs for RELB and 2 non-targeting control sgRNAs). (FIG. 13P) Identification of 274 genes identified as enriched in both CD4⁺ and CD8⁺ T cells transduced with LTBR over matched tNGFR controls (“core LTBR” genes). Error bars indicate SEM.

FIG. 14A-FIG. 14P show co-delivery of ORFs with CD19-targeting CARs. (FIG. 14A) Transduction efficiency of CAR+ORF lentiviral vectors or ORF alone (n=4 biological replicates). (FIG. 14B, FIG. 14C) CAR expression level as determined by staining with anti-mouse Fab F(ab′)₂. Representative histograms (FIG. 14B) and quantification of CAR expression relative to tNGFR (FIG. 14C) is shown for two healthy donors and two patients with diffuse large B cell lymphoma (DLBCL). (FIG. 14D) Expansion curves of CAR+ORF transduced T cells (n=4 biological replicates). (FIG. 14E) LTBR expression in autologous CD14⁺ monocytes and T cells transduced with LTBR alone or CAR+LTBR. (FIG. 14F-FIG. 14I) Expression of ICAM-1 (FIG. 14F), CD70 (FIG. 14G), CD74 (FIG. 14H) and MHC-II (FIG. 14I) by T cells transduced with LTBR ORF only, CAR+LTBR or CAR+tNGFR. All data are normalized to tNGFR only (no CAR). Unpaired two-sided t test p values are shown. (FIG. 14J-FIG. 14M) Expression of exhaustion markers PD-1 (FIG. 14J), TIM-3 (FIG. 14K), LAG-3 (FIG. 14L) and CD39 (FIG. 14M) in CAR+ORF T cells. CD8 left bars, CD4 right bars. (FIG. 14N) Differentiation phenotype of CAR+ORF T cells. CM: Central memory. EM: Effector memory. Differentiation was defined based on CD45RO and CCR7 expression (naive: CD45RO^{neg} CCR7⁺, CM: CD45RO⁺CCR7⁺, EM: CD45RO⁺ CCR7^{neg}, effector CD45RO^{neg} CCR7^{neg}). (FIG. 14O, FIG. 14P) Expression of activation markers CD25 (FIG. 14O) and CD69 (FIG. 14P) in CAR+ORF T cells incubated alone or with Nalm6 cells for 24 h. Error bars indicate SEM. N=3 biological replicates, unless indicated otherwise.

FIG. 15A-FIG. 15P show top-ranked genes from the ORF screen boost antigen-specific T cell responses. (FIG. 15A, FIG. 15B) Co-delivery of anti-CD19 CARs and ORFs to T cells from healthy donors. (FIG. 15A) IFNγ and (FIG. 15B) IL2 secretion after overnight co-incubation of CD4⁺ T cells with Nalm6 cells at 1:1 ratio (n=3 biological replicates, representative of two donors). (FIG. 15C, FIG. 15D) IFNγ (FIG. 15C) or IL-2 (FIG. 15D) secretion by CAR+ORF or ORF only T cells co-incubated for 24 h either alone or with Nalm6 cells. (FIG. 15E) Cytotoxicity of 19-BBz CAR T cells expressing tNGFR or LTBR ORF after co-incubation with Nalm6 GFP cells. (FIG. 15F) Quantification of Nalm6 clearance (relative to Nalm6 co-incubated with untransduced T cells) for CAR+ORF or ORF alone T cells at different effector:target ratios. Unpaired two-sided t-test p values: 0.011, 1.3×10−4, 0.072, 0.02, 0.021, 0.52, 0.087, 1, 0.51 (left to right). (FIG. 15G) Representative images of T cells transduced with 19-28z CAR and NGFR or LTBR, co-incubated with CD19⁺ Nalm6 GFP cells for 48 h at 1:1 ratio. Scale bar: 200 μm. (FIG. 15H-FIG. 15J) Repeated stimulation of CAR+ORF T cells with Nalm6 cells. IL-2 secretion (FIG. 15I), or Nalm6 survival (FIG. 15J), by 19-BBz CAR LTBR or tNGFR T cells re-challenged with Nalm6 after repeated stimulation with Nalm6 cells every three days, for up to three rounds of stimulation. (FIG. 15K) Secretion of cytokines IL2 and IFNγ by CAR/LTBR or CAR/tNGFR T cells from two patients with DLBCL after overnight incubation with Nalm6 target cells. Two-sided paired t-test p value is shown. (FIG. 15L) Representative staining of ORF-transduced T cells endogenously expressing Vγ9Vδ2 TCR. (FIG. 15M) Quantification of ORF-transduced T cells expressing Vγ9Vδ2 TCR. (FIG. 15N, FIG. 15O) IL2 (FIG. 15N) or IFNγ (FIG. 15O) secretion after 24 h co-incubation of ORF transduced Vγ9Vδ2 T cells with leukemia cell lines. (FIG. 15P) IL2 or IFNγ secretion after 24 h co-incubation of ORF transduced Vγ9Vδ2 T cells with BxPC3, a pancreatic ductal adenocarcinoma cell line. Cell lines in panels (FIG. 15N-FIG. 15P) were pre-treated with zoledronic acid prior to co-incubation. Error bars indicate SEM. N=3 biological replicates are shown, unless indicated otherwise.

FIG. 16A-FIG. 16F show top-ranked genes improve antigen-specific CAR T cell responses in solid tumor. (FIG. 16A) Codelivery of anti-mesothelin CARs and ORFs to T cells from healthy donors. (FIG. 16B-FIG. 16D) Secretion of cytokines IFNγ and IL2 by CD4+ and CD8+ T cells co-transduced with anti-mesothelin CARs and ORF, after an overnight co-incubation with a mesothelin-high cell line Capan-2 (FIG. 16B, FIG. 16C) or mesothelin-low cell line BxPC3 (FIG. 16D). No specific cytokine secretion was observed in T cells incubated alone. N=3 biological replicates. Dashed line indicated the level of cytokine secretion in regular CAR T cells (i.e. co-expressing tNGFR). (FIG. 16E, FIG. 16F) Killing of GFP+ mesothelin-high Capan-2 or mesothelin-low BxPC3 after co-incubation with engineered CAR T cells at 1:2 T cell to cancer cell ratio for 48 h. Cancer cell killing was normalized by dividing the integrated GFP signal in wells containing regular CAR T cells (i.e. co-expressing tNGFR) by the integrated GFP signal in specified samples. Ratio above one indicates higher killing, i.e. lower GFP signal in specified samples (and thus lower number of cancer cells) than in matched CAR control (CAR+tNGFR). WT or no CAR=untransduced T cells.

FIG. 17A-FIG. 17D show top-ranked genes improve antigen-specific TCR T cell responses in solid tumor. (FIG. 17A) Codelivery of anti-NY-ESO-1 TCR and ORFs to T cells from healthy donors. (FIG. 17B, FIG. 17C) Secretion of cytokines IFNγ and IL-2 by CD8+ T cells co-transduced with anti-NY-ESO-1 TCR and ORF, after an overnight coincubation with a melanoma cell line A375. No specific cytokine secretion was observed in T cells incubated alone. N=3 biological replicates. Dashed line indicated the level of cytokine secretion in regular TCR T cells (i.e. co-expressing tNGFR). (FIG. 17E, FIG. 17F) Killing of GFP+A375 cells co-incubation with engineered TCR T cells at 1:1 T cell to cancer cell ratio for 48 h. Cancer cell killing was normalized by dividing the integrated GFP signal in wells containing only A375 cells but no T cells by the integrated GFP signal in specified samples. No TCR=untransduced T cells.

FIG. 18A-FIG. 18G provide an overview of OverCITE-seq.

FIG. 19 is a listing of the clinical trials relating to chimeric antigen receptors available on clinicaltrials.gov.

FIG. 20 is a listing of the clinical trials relating to T cell receptors available on clinicaltrials.gov.

FIG. 21 provides exemplary antibody sequences for construction of chimeric antigen receptors.

FIGS. 22A-D demonstrate in vivo efficacy of 19-BB-z CAR T cells co-expressing LTBR against a disseminated leukemia model in NSG mice. FIG. 22A) Experimental design. Female NSG mice (n=4 per group) were inoculated into the tail vein with 5×10⁵ Nalm6-luc cells. Four days later, bioluminescence (BLI) measurement was performed to stage the mice to ensure each group had the same median tumor burden. The following day, untransduced or CAR transduced T cells (CD4:CD8, 1:1) were injected into the tail vein. FIG. 22B) Survival of mice within the duration of the study. Log-rank Mantel-Cox p value is shown. FIG. 22C) Dorsal and ventral total body BLI signal. Individual values for all surviving mice are shown. The lines connect medians of each group. One-way ANOVA with post-hoc Šidác's multiple comparisons test p values between LTBR and tNGFR group are shown. ****p<0.0001. FIG. 22D) Changes in body weight, comparing to the starting (d=0) body weight for each mouse.

FIG. 23 demonstrates survival of LTBR CAR T cells in absence of IL2. Transduced and selected T cells were expanded and cultured in presence of IL2 as described previously. On day 14 post transduction, CAR+LTBR or CAR+tNGFR T cells were washed and split across two conditions: with IL2 and without IL2. Cell viability was then assessed three times a week by direct cell counting, using Trypan Blue exclusion (up to day 23) or flow cytometry with a viability dye (from day 23 onwards). At each timepoint, the number of viable cells in the no IL2 condition was compared to the number of viable cells in the +IL2 condition to determine survival. N=3

FIGS. 24A-F demonstrate LTBR phenotype and function in different media. FIG. 24A-FIG. 24C) CD4 and CD8 T cells from a healthy donor were cultured in a given medium throughout the experiment, including activation, lentiviral transduction, selection and culture. 14 days post transduction, T cells were resuspended in a respective medium without IL2 and stimulated overnight to induce cytokine secretion. The quantity of secreted IFNγ (a) and IL2 (b) was measured by ELISA. The mean quantities of secreted cytokines in all matched conditions are shown in c). FIG. 24D) Expression of CD54 and CD74 in CD4 and CD8 T cells, transduced with tNGFR or LTBR, normalized to untransduced control. FIG. 24E) Ratio of central memory (CM) to effector T cells in CD4 and CD8 T cells, transduced with tNGFR or LTBR. CM: CD45RO+CCR7+, effector: CD45RO+/−CCR7−. FIG. 24F) Expression of PD1, in CD4 and CD8 T cells, transduced with tNGFR or LTBR. **p<0.01

FIG. 25A-C demonstrate overexpression of TNFRSF members in primary T-cells. FIG. 25A) Surface expression of selected TNFRSF members in ORF-transduced and untransduced T-cells. FIG. 25B) CD8 left bars, CD4 right bars. Proliferation of T-cells transduced with TNFRSF members after 4 days of stimulation with CD3/CD28, normalised to tNGFR. FIG. 25C) IFNγ secretion by T-cells transduced with TNFRSF members after 24 h stimulation with CD3/CD28, normalised to tNGFR. CD8 left bars, CD4 right bars.

FIG. 26A-G demonstrate overexpression of constitutively active positive regulators of the NFκB pathway. FIG. 26A,B) IFNγ (a) and IL2 (b) secretion after overnight stimulation of transduced T cells with CD3/CD28. The absolute quantities of secreted cytokines are normalized to LTBR. FIG. 26C-F) Surface expression of representative markers upregulated in LTBR T cells. The expression levels are normalized to LTBR. FIG. 26G) Heatmap summary of phenotypes induced by constitutively active positive regulators of the NFκB pathway, in comparison to LTBR. tNGFR is used as an irrelevant gene.

FIG. 27A-G demonstrate knockout of negative regulators of the NFκB pathway. NT, TNFAP3, and NFKBIA from left to right. FIG. 27A-B) IFNγ (a) and IL2 (b) secretion after overnight stimulation of transduced T cells with CD3/CD28. The absolute quantities of secreted cytokines are normalized to LTBR co-expressing NT sgRNAs. Each dot represents an individual sgRNA. FIG. 27C-F) Surface expression of representative markers upregulated in LTBR T cells. The expression levels are normalized to LTBR co-expressing NT sgRNAs. Each dot represents an individual sgRNA. FIG. 27G) Heatmap summary of phenotypes induced by knockout of the negative regulators of the NFκB pathway, in comparison to LTBR co-expressing NT sgRNAs.

FIG. 28A-F demonstrate transgene positioning for LTBR and CAR co-expression. FIG. 28A) Schematic depiction of the vectors used. FIG. 28B) Expression of LTBR or tNGFR, normalized to the corresponding CAR-puro-gene vector, in CD4 and CD8 T cells. FIG. 28C-E) Cytokine secretion upon overnight co-incubation of CAR T cells with CD19+ target cells Nalm6. FIG. 28F) Cytokine secretion in response to target cells, normalized to the corresponding CAR-puro-gene vector, in CD4 and CD8 T cells.

FIG. 29A-D demonstrate inducible transgene expression in T cells. FIG. 29A) Vector design. FIG. 29B-C) Expression of LTBR (b) and tNGFR (c) in CD4 and CD8 T cells transduced with vectors shown in a. T cells were either left unstimulated (no stim) or stimulated with CD3/CD28 antibodies for 24h. Transgene expression is normalized to the staining intensity in T cells transduced with the promoter-less vector. FIG. 29D) Transgene expression in T cells transduced with the NFκB promoter vector compared to the expression in T cells transduced with the EFS promoter vector.

### DETAILED DESCRIPTION OF THE INVENTION

The engineering of autologous patient T cells for adoptive cell therapies has revolutionized the treatment of several types of cancer¹. However, further improvements are needed to increase response and cure rates. CRISPR-based loss-of-function screens have been limited to negative regulators of T cell functions²⁻⁴ and raise safety concerns owing to the permanent modification of the genome. Here we identify positive regulators of T cell functions through overexpression of around 12,000 barcoded human open reading frames (ORFs). The top-ranked genes increased the proliferation and activation of primary human CD4⁺ and CD8⁺ T cells and their secretion of key cytokines such as interleukin-2 and interferon-γ. In addition, we developed the single-cell genomics method, OverCITE-seq, for high-throughput quantification of the transcriptome and surface antigens in ORF-engineered T cells. The top-ranked ORF-lymphotoxin-β receptor (LTBR)—is typically expressed in myeloid cells but absent in lymphocytes. When overexpressed in T cells, LTBR induced profound transcriptional and epigenomic remodeling, leading to increased T cell effector functions and resistance to exhaustion in chronic stimulation settings through constitutive activation of the canonical NF-κB pathway. LTBR and other highly ranked genes improved the antigen-specific responses of chimeric antigen receptor T cells and γδ T cells, highlighting their potential for future cancer-agnostic therapies⁵. Our results provide several strategies for improving next-generation T cell therapies by the induction of synthetic cell programs.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

It is to be noted that the term “a” or “an”, refers to one or more, for example, “a T cell”, is understood to represent one or more T cell(s). As such, the terms “a” (or “an”), “one or more,” and “at least one” is used interchangeably herein.

As used herein, the term “about” means a variability of plus or minus 10% from the reference given, unless otherwise specified.

Furthermore, “and/or” where used herein is to be taken as specific disclosure of each of the two specified features or components with or without the other. Thus, the term “and/or” as used in a phrase such as “A and/or B” herein is intended to include “A and B.” “A or B.” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991): Qhtsuka et al, J. Biol. Chem. 260:2605-2608 (1985); and Rossolim et al., Mol. Cell. Probes 8:91-98 (1994)).

The terms “nucleic acid sequence,” “nucleotide sequence,” or “polynucleotide sequence” are used interchangeably and refer to a contiguous nucleic acid sequence. The sequence can be either single stranded or double stranded DNA or RNA, e.g., an mRNA.

Nucleic acids described herein can be cloned using routine molecular biology techniques, or generated de novo by DNA synthesis, which can be performed using routine procedures by service companies having business in the field of DNA synthesis and/or molecular cloning (e.g. GeneArt, GenScript, Life Technologies, Eurofins). The nucleic acid sequences encoding aspects of a CRISPR-Cas editing system described herein are assembled and placed into any suitable genetic element, e.g., naked DNA, phage, transposon, cosmid, episome, etc., which transfers the sequences carried thereon to a host cell, e.g., for generating non-viral delivery systems (e.g., RNA-based systems, naked DNA, or the like), or for generating viral vectors in a packaging host cell, and/or for delivery to a host cells in a subject. In certain embodiments, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

“Variants” of proteins or peptides as defined in the context of the present invention may be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g., its specific inhibitory property. “Variants” of proteins or peptides as defined in the context of the present invention may comprise conservative amino acid substitution(s) compared to their native, i.e., non-mutated physiological, sequence. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bonds, e.g., side chains which have a hydroxyl function. This means that e.g., an amino acid having a polar side chain is replaced by another amino acid having a likewise polar side chain, or, for example, an amino acid characterized by a hydrophobic side chain is substituted by another amino acid having a likewise hydrophobic side chain (e.g., serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g., using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam). A variant may also include a non-natural amino acid.

A “variant” of a protein or peptide may have at least 70%, 75%, 80%, 85%, 90%, 95%, 98% or 99% amino acid identity over a stretch of 10, 20, 30, 50, 75, 100 or more amino acids of such protein or peptide, or over the full length of the protein or peptide.

The term “gene” can refer to a segment of DNA involved in producing or encoding a polypeptide chain. It may include regions preceding and following the coding region (leader and trailer) as well as intervening sequences (introns) between individual coding segments (exons).

As used herein, the terms “coding region” and “region encoding” and grammatical variants thereof, refer to an open reading frame (ORF) in a polynucleotide that upon expression yields a polypeptide or protein.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA, and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleic acid sequence encoding an amino acid sequence” includes all nucleic acid sequences that are degenerate versions of each other and that encode the same amino acid sequence. A nucleic acid sequence that encodes a protein or an RNA may also include introns to the extent that the nucleotide sequence encoding the protein may in some versions contain an intron(s).

The term “expression” is used herein in its broadest meaning and comprises the production of RNA, of protein, or of both RNA and protein. Expression may be transient or may be stable.

The terms “expressing” and “overexpression” refer to increasing the expression of a gene or protein. The terms refer to an increase in expression, for example, in increase in the amount of mRNA or protein expressed in a T cell, other lymphocyte or host cell, of at least 10%, as compared to a reference control level, or an increase of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%. Various methods for expression and/or overexpression are known to those of skill in the art, and include, but are not limited to, stably or transiently introducing a heterologous polynucleotide encoding a protein (i.e., a gene set forth in Table 1) to be expressed and/or overexpressed in the cell or inducing expression or overexpression of an endogenous gene encoding the protein in the cell. It is understood that one or more genes set forth in Table 1 can be expressed and/or overexpressed in a cell. It is also understood that two or more genes to be expressed and/or overexpressed in a cell can be selected from one or more of the genes set forth in Table 1.

The term “autologous” refer to any material derived from the same subject to whom it is later to be re-introduced.

The term “exogenous” refers to any material introduced from or produced outside an organism, cell, tissue, or system.

The term “expression vector” refers to a vector comprising a recombinant polynucleotide comprising expression control sequences operatively linked to a nucleotide sequence to be expressed. An expression vector comprises sufficient cis-acting elements for expression: other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, an “expression cassette” refers to a nucleic acid molecule which encodes one or more ORFs or genes, e.g., an effector-enhancing gene, or a CAR or TCR or component thereof. An expression cassette also contains a promoter and may contain additional regulatory elements that control expression of one or more elements of a gene editing system in a host cell. In one embodiment, the expression cassette may be packaged into the capsid of a viral vector (e.g., a viral particle). In one embodiment, such an expression cassette for generating a viral vector as described herein is flanked by packaging signals of the viral genome and other expression control sequences such as those described herein.

The term “regulatory element” or “regulatory sequence” refers to expression control sequences which are contiguous with the nucleic acid sequence of interest and expression control sequences that act in trans or at a distance to control the nucleic acid sequence of interest. As described herein, regulatory elements comprise but are not limited to: promoter; enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Also, see Goeddel; Gene Expression Technology: Methods in Enzymology 185, Academic Press, San Diego, CA (1990). Regulatory sequences include those which direct constitutive expression of a nucleic acid sequence in many types of target cell and those which direct expression of the nucleic acid sequence only in certain target cells (e.g., tissue-specific regulatory sequences).

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The term “constitutive” when referring to a promoter specifies a nucleotide sequence which, when operably linked with a polynucleotide which encodes or specifies a gene product, causes the gene product to be produced in a cell under most or all physiological conditions of the cell. The term “inducible” or “regulatable” when referring to a promoter specifies a nucleotide sequence which, when operably linked with a polynucleotide which encodes or specifies a gene product, causes the gene product to be produced in a cell substantially only when an inducer which corresponds to the promoter is present in the cell. In certain embodiments, the inducible promoter is activated in response to T cell stimulation. In certain embodiments, the promoter is an NFAT, AP1, NFκB, or IRF4 promoter. The term “tissue-specific” when referring to a promoter specifies a nucleotide sequence which, when operably linked with a polynucleotide encodes or specified by a gene, causes the gene product to be produced in a cell substantially only if the cell is a cell of the tissue type corresponding to the promoter. Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

The term “operably linked” or refers to functional linkage between one or more regulatory sequences and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, where necessary to join two protein coding regions, are in the same reading frame.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

In certain embodiments, one or more genes are encoded by a nucleic acid sequence that is delivered to a host cell by a vector or a viral vector, of which many are known and available in the art. In one embodiment, provided is a vector comprising an expression cassette as described herein. In one embodiment, a vector is a non-viral vector. In another embodiment, a vector is a viral vector. A “viral vector” refers to a synthetic or artificial viral particle in which an expression cassette containing a nucleic acid sequence of interest is packaged in a viral capsid or envelope. Examples of viral vectors include but are not limited to lentivirus, adenoviruses, retroviruses (γ-retroviruses and lentiviruses), poxviruses, adeno-associated viruses (AAVs), baculoviruses, herpes simplex viruses. In one embodiment, the viral vector is replication defective. A “replication-defective virus” refers to a viral vector, wherein any viral genomic sequences also packaged within the viral capsid or envelope are replication-deficient, i.e., they cannot generate progeny virions but retain the ability to infect cells.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

In certain embodiments, the vector is a non-viral plasmid that comprises an expression cassette described herein, e.g., naked DNA, naked plasmid DNA, RNA, and mRNA; coupled with various compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

Plasmids, other cloning and expression vectors, properties thereof, and constructing/manipulating methods thereof that can be used in accordance with the present invention are readily apparent to those of skill in the art. In one embodiment, an expression cassette as described herein is engineered into a suitable genetic element (a vector) useful for generating viral vectors and/or for introduction to a host cell, e.g., naked DNA, phage, transposon, cosmid, episome, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY.

The term “transfected” refers to a process by which exogenous nucleic acid is transferred or introduced into the host cell. A “transfected” cell is one which has been transfected with exogenous nucleic acid. The cell includes the primary subject cell and its progeny.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

RNA or DNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

As used herein, the term “subject” means a mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, non-human primate and others. As used herein, the term “subject” is used interchangeably with “patient”.

### Compositions

Provided herein are compositions that include nucleic acids, expression cassettes, and/or lymphocytes which include a coding sequences for a gene which has been shown to enhance T cell survival, proliferation and/or effector function (collectively referred to herein as an “effector-enhancing gene”). In certain embodiments, the effector-enhancing gene comprises any of the genes identified in Table 1, below.

TABLE 1

| gene_{—} | gene_{—} | gene_{—} | gene_{—} | gene_{—} |
|---|---|---|---|---|
| symbol | symbol | symbol | symbol | symbol |
| RPLP1 | PRTN3 | KDSR | RHEX | ACP2 |
| EMC10 | RRAGB | MEI1 | MAGEA12 | PLPPR2 |
| SFTPC | PFKFB4 | C1orf210 | BOLL | BDKRB2 |
| PSMB1 | PDLIM3 | GSTK1 | CLEC19A | IFITM3 |
| SEC61G | GPN3 | SPATS1 | NLRP5 | IDH3B |
| PRAP1 | PRSS48 | ACSM5 | LGALS12 | SYS1 |
| KLK11 | ARMC7 | TAZ | PDXK | IFNA21 |
| TSPAN1 | OR1D5 | IFT81 | PRR19 | ADPRS |
| HDHD2 | UBE2D1 | IL27 | TRAF2 | SULT1C4 |
| CCR4 | MAPK3 | GTF3C6 | PEX11A | ASMTL |
| PAFAH1B3 | LRRC29 | CEP43 | VASH1 | TANK |
| THRA | CD59 | RUVBL2 | TMEM263 | RPL13A |
| SNAPIN | GJB7 | PRND | LRP5L | IL10 |
| CBX8 | MEIS3 | TAFA2 | DAND5 | PSG1 |
| TMEM43 | PKLR | PDK4 | SLC52A1 | TMIGD1 |
| CELA1 | SZRD1 | TMEM44 | COMMD8 | SLC23A3 |
| DBI | GTPBP8 | NRBP2 | NOL6 | CDC42 |
| RIMS3 | HLA-DOA | RTBDN | CA12 | FBXL2 |
| RPL29 | SPPL2C | FKBP1A | CTPS2 | SEMA4G |
| SLC37A4 | LTBR | EDDM3A | CALML3 | ARSJ |
| IGHV7-81 | VSTM1 | TRMT12 | FHIT | WAS |
| FGL1 | EXOSC5 | TFF1 | LTA4H | DPH1 |
| PRSS8 | TIMMDC1 | FNDC8 | UBL4B | H2AC6 |
| HEMK1 | DYRK4 | TLE1 | OR10G3 | ADH4 |
| UBE2A | CRADD | UHMK1 | SNURF | ZNF830 |
| IFNL2 | SPIC | RAD51C | GPR37L1 | RNF11 |
| CZIB | DHX40 | ETFBKMT | TSHB | PIEZO1 |
| HNRNPK | SLC10A7 | TARBP2 | F2RL2 | APTX |
| OXA1L | TIMP4 | LENG1 | SNAPC5 | ALKBH3 |
| KIAA0930 | HSFX3 | CLN6 | TXN | LRRC71 |
| SLC39A1 | TRH | CCDC137 | MID2 | IGSF10 |
| METTL7B | CUTA | GNPDA1 | ADAT2 | H3C10 |
| WDR70 | CDK17 | QTRT1 | RAB8A | MMP2 |
| GLYCTK | HADH | ZCCHC12 | EID3 | ZMYM3 |
| C11orf16 | RAN | FBXO6 | BATF | CD1B |
| ASB6 | ACTL8 | PGAP2 | DCT | RIT2 |
| F2RL1 | LIG3 | SMOC1 | DTNA | GOSR2 |
| AHNAK | OSM | FBXO30 | ELN | CDK2 |
| KRTAP19-5 | NFYB | CST8 | CNOT2 | HMHB1 |
| TPI1 | RNF186 | BAX | CALCB | TRPV2 |
| AHSA1 | RAB24 | S100A8 | MAB21L2 | YPEL4 |
| POLG2 | RPL14 | VWC2L | GRIK2 | CAMKMT |
| SLC39A4 | TMEM107 | PPP1R2B | PLSCR1 | MYL4 |
| CPSF4 | TRIM36 | HEPACAM2 | RASGEF1A | CERS4 |
| RNF114 | TIMM8B | CYP27A1 | GALNT3 | RAP1A |
| DCLRE1B | EMP3 | FCF1 | H2AC14 | ZNF689 |
| PLA2G1B | CCNE1 | TESC | ITM2A | DECR2 |
| HOXA6 | NFIC | RPP30 | GPR3 | TNFAIP8 |
| EXOSC1 | NNAT | EVA1C | ATP5MPL | COPS8 |
| GPHB5 | S100A9 | GLT1D1 | SLC25A48 | DHRSX |
| VRK2 | TMEM134 | KCNA4 | LIN37 | H2BC21 |
| ADAM2 | CENPW | CTRB1 | PDP2 | OR3A2 |
| HLF | POU4F3 | MRPL50 | XIAP | DRAP1 |
| UBE2D4 | S1PR3 | HSD17B7 | SELENOH | RASSF1 |
| KCNJ12 | SPAG11A | IL1RN | SUN5 | S100A7 |
| NCR3 | CHMP2A | APBB1IP | YIF1A | CABLES1 |
| SPRR2B | ECT2 | USP24 | TBRG4 | TP53 |
| NMS | IFRD2 | LHFPL5 | TGIF1 | CCL13 |
| ENHO | PWP1 | HSD11B2 | MASP2 | TUBB |
| APEX2 | MS4A3 | SLC7A11 | MIF4GD | IL31 |
| SRP9 | FANCC | TRIM51 | PATE1 | GLYAT |
| RORA | TOR1AIP2 | OR1N2 | S100A11 | TAAR5 |
| KLK2 | MRFAP1L1 | GPD1 | EDC3 | LRRC25 |
| KLK15 | HCFC2 | CRHBP | OR6B2 | GSK3B |
| HOGA1 | C1D | SYNGR2 | ALDH1A3 | ITIH1 |
| HAAO | CSF3 | SERPINB4 | MRPL17 | CTSE |
| SWSAP1 | CLIC1 | RSKR | ENPP1 | ASCL3 |
| TMEM205 | CDA | PEX12 | GTF2B | CSH1 |
| OR10W1 | PCP2 | SLC1A7 | ANGPT4 | IL23A |
| STK25 | MRPS12 | HHLA3 | CDCP2 | VGLL3 |
| PPP1R8 | SKAP1 | DOK6 | P2RY6 | LAMTOR1 |
| WDFY1 | GPATCH11 | AFTPH | KLRD1 | PODXL2 |
| LARS2 | MORN4 | DDIT4 | POFUT1 | PPIC |
| WDTC1 | GPNMB | STAU2 | PCMTD1 | LSM1 |
| RALB | C5orf24 | TBCCD1 | MAPRE3 | PTPN7 |
| CAPNS2 | PGS1 | TMED9 | CTRB2 | STPG2 |
| AKAP7 | TRIM74 | RPL30 | HAVCR2 | ASB1 |
| SLC4A4 | SSX3 | MC3R | BCL7A | BRWD1 |
| CSRP1 | ANXA9 | FAM216A | SLC25A39 | TPPP3 |
| ZCRB1 | DUPD1 | ZNF232 | PRDX5 | ACSM3 |
| EDA | DCUN1D3 | GGCX | TEX35 | EXOSC4 |
| RASGEF1C | XAGE3 | EIF1AY | SOSTDC1 | PAGE5 |
| POU2F2 | RBIS | SYT10 | REEP4 | MRPL18 |
| NELFE | SH3RF3 | C16orf70 | ANAPC16 | HSPBAP1 |
| TSPAN31 | THNSL1 | CPXM2 | FOSB | TNIP1 |
| FUT10 | RTL8C | PTRH2 | GAK | MAPRE2 |
| DKC1 | GABRB1 | AGAP1 | NPY | MMP16 |
| CGB2 | IFNE | CA2 | SLC25A4 | HUWE1 |
| TM2D2 | CD14 | MATN1 | F11R | IL12B |
| AHCY | ESM1 | PRR15L | ABHD14B | SIRT5 |
| FAM71F2 | TIRAP | WDR1 | AKR1C4 | GALP |
| RHBDD2 | ING3 | PELI2 | CKMT1A | SMIM14 |
| RRAGA | PELI1 | IFTAP | SLC31A2 | MTG1 |
| DPEP2 | GDAP1 | PSMD4 | PPP2R5E | NELFCD |
| BANF2 | PACC1 | CRIP1 | DEFB135 | LRAT |
| FMO4 | CXCL12 | C3AR1 | PAX9 | LCN10 |
| LYPD4 | SUMO3 | DEFB132 | CLEC3A | COX7A2L |
| MGAT4D | PRG3 | VAMP2 | MOB3C | TTC9C |
| NMU | DAB1 | GFAP | MLLT11 | BOD1L1 |
| CCBE1 | SEC31A | CRYBA1 | SLC14A1 | KRTAP3-3 |
| SRGN | CEP112 | HAUS1 | WFDC3 | TMEM126A |
| SDC4 | SAMSN1 | SERAC1 | DOK1 | WDR77 |
| CD244 | PKIB | SLC17A4 | RNASEH2B | RPL21 |
| TBC1D20 | IQCD | GUCA2A | ROGDI | ELF2 |
| LDHC | PHPT1 | ORM2 | ATF6B | PYGB |
| HPCAL1 | LRRC8D | NAGK | WDR62 | HCST |
| SNCB | OR4K17 | SRSF12 | TCEAL7 | NIT1 |
| BNIPL | PSMB6 | OCEL1 | CRLF2 | RALGPS2 |
| OR52W1 | ENPP3 | WDR4 | PLA2G12A | HAPLN3 |
| MRPL51 | SNRK | B3GAT3 | CDK1 | AMIGO3 |
| DMAP1 | NBR1 | TRAPPC6A | GHITM | DYNAP |
| RPL18 | ETFA | MRPL53 | ARL11 | HPRT1 |
| CISH | UHRF2 | KRT14 | POLR2C | OR10X1 |
| TRIM5 | OLFM1 | OTX1 | KIF26B | YARS2 |
| LRTM1 | RAB27B | NEIL2 | UBQLN3 | REEP3 |
| LAMP2 | TNFSF14 | AK3 | ZNF706 | ZDHHC5 |
| TMEM37 | HEATR9 | TMPRSS6 | TMEM143 | DDIT3 |
| FAM180A | RNF5 | LAPTM5 | CTSW | TMEM189 |
| RSPO1 | MKNK1 | GRPEL1 | CDC25C | UBE2M |
| TWIST2 | SMAP1 | HMCES | C2orf73 | PPP2R2B |
| CGGBP1 | BIN3 | GRID1 | FSCB | ACBD4 |
| PHF23 | TAC1 | PRSS37 | THEM4 | BEX3 |
| DEFB134 | SELENOW | INSL5 | PSMA2 | SLC35C2 |
| CHST4 | NACA | EME1 | LAD1 | MPDU1 |
| DCK | PTCHD1 | SPINK9 | SPANXN5 | TRIM47 |
| MRPS21 | STX8 | WDR20 | FAM78A | CLDN14 |
| UTP4 | TRIP6 | JAGN1 | | |
| MRPL49 | CA11 | GPR17 | | |
| MIER2 | DBF4B | LETM1 | | |
| PSMD3 | PFN4 | SLC26A5 | | |
| PIFO | BBS1 | REXO2 | | |
| SLC39A11 | H2BC15 | IL18RAP | | |
| OR13G1 | H6PD | EOLA2 | | |
| INSIG2 | R3HDM4 | DUS1L | | |
| OR2H1 | SNRPA | TMEM182 | | |
| BMPER | TRIM22 | SMU1 | | |
| HP1BP3 | SUPT4H1 | FXYD1 | | |
| QPRT | DIMT1 | CCL17 | | |
| SGCA | TEX38 | STX1A | | |
| CASTOR1 | CCDC127 | UBXN1 | | |
| AGPAT4 | SSMEM1 | POLD3 | | |
| ZDHHC6 | IRF4 | TRPV5 | | |
| C8orf37 | OR2M4 | KRT20 | | |
| C6orf141 | MYL6 | FAM32A | | |
| CD3E | SHFL | HOMER1 | | |
| CA5B | SCG5 | | | |
| TRMT44 | CORO2B | | | |
| PRB3 | PPIG | | | |
| AK2 | RNF185 | | | |
| LMBRD1 | CEP20 | | | |
| EIF4E3 | SPINT4 | | | |
| ADAP2 | MLX | | | |
| CNR2 | BRD2 | | | |
| PLD3 | DERL1 | | | |
| RNF182 | TEX12 | | | |
| STMN2 | SLFN5 | | | |
| PCSK4 | TNFSF18 | | | |
| IRX6 | PHKG1 | | | |
| ADA | PYCR2 | | | |
| SMCP | CALML5 | | | |
| UPP2 | MDFI | | | |
| LMAN2L | CHGB | | | |
| PDCD1 | SPIB | | | |
| RACK1 | C2CD4B | | | |
| PGLYRP1 | ARHGAP28 | | | |
| GNB1L | APCDD1 | | | |
| DDX49 | MRPL15 | | | |
| TMEM68 | WNT7B | | | |

### Expression Cassettes and Cells Containing Same

Provided herein are expression cassettes which include nucleic acid sequences which encode one or more effector-enhancing genes. In certain embodiments, the gene comprises any of the genes identified in Table 1, above, or a fragment or variant thereof. In other embodiments, the gene comprises any of the genes identified in Table 2, below, or a fragment or variant thereof. In certain embodiments, the expression cassette includes more than one effector-enhancing gene, or a fragment or variant thereof. Also provided are host cells which contain the nucleic acids and expression cassettes described herein. In certain embodiments, the host cell is a lymphocyte. As used herein, where reference to a specific gene of Table 1 or Table 2 is mentioned, it is intended that the use of the coding sequence for the full-length protein, a fragment having a deletion or truncation, or a variant having one or more substitutions in the amino acid, is intended. For example, in certain embodiments, the nucleic acid encodes a protein sequence having a deletion or truncation in the N terminus. In certain embodiments, the nucleic acid encodes a protein sequence having a deletion or truncation in the C terminus. In one embodiment, the nucleic acid encodes a protein having of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, or at least 125 amino acids.

In one embodiment, the effector-enhancing gene is LTBR. LTBR, a receptor endogenously expressed by professional antigen presenting cells but not lymphocytes, was identified as a strong synthetic driver of both T-cell proliferation and secretion of key cytokines: IL-2 and IFNγ. Using a multimodal single-cell sequencing approach, it was shown that LTBR induces profound transcriptional changes when overexpressed in T-cells, activating cellular programs involved in antigen presentation and prevention of apoptosis. As describe herein, a platform was developed for testing combinatorial perturbations in T-cells, by co-expressing a gene of interest (e.g. LTBR) together with CRISPR sgRNAs targeting other genes, to map signaling networks in T-cells. Also demonstrated herein is that mRNA delivery of LTBR as an alternative to constitutive lentiviral expression, highlighting the translational potential of our screening approach.

In a certain embodiment, the expression cassette comprises a nucleic acid encoding LTBR, or a fragment thereof. LTBR (lymphotoxin-beta receptor), which encodes for tumor necrosis factor receptor superfamily member 3, is essential for the development and organization of secondary lymphoid tissues and chemokine release. A representative nucleic acid sequence of LTBR can be found at Accession ID NM_002342.3, SEQ ID NO: 1. The full-length amino acid sequence of LTBR is shown in SEQ ID NO: 2.

The LTBR protein can be divided into three regions, or domains: the extracellular domain (amino acids 31-227 of SEQ ID NO: 2); the transmembrane (or helical) domain (amino acids 228-248 of SEQ ID NO: 2); and the cytoplasmic (or intracellular) domain (amino acids 249-435 of SEQ ID NO: 2). The signal peptide of the immature protein is at amino acids 1-30 of SEQ ID NO: 2.

In certain embodiments, the expression cassette comprises a nucleic acid encoding a fragment of LTBR. In certain embodiments, the nucleic acid encodes a protein sequence having a deletion of amino acids 2-31, 32-41, 32-151, 32-180, 393-435, 377-435, 324-377, 297-435, or 262-435 as compared to the native protein (SEQ ID NO: 2). In certain embodiments, the LTBR has a deletion of 378-435, 379-435, 380-435, 381-435, 382-435, 383-435, 384-435, 385-435, 386-435, 387-435, 388-435, 389-435, 390-435, 391-435, or 392-435 as compared to the native protein (SEQ ID NO: 2). In certain embodiments, the nucleic acid encodes a protein sequence having a deletion in the N terminus. In certain embodiments, the nucleic acid encodes a protein sequence having a deletion in the C terminus. In one embodiment, the LTBR is has a deletion of residues 393-435. In certain embodiments, the LTBR has a deletion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, or at least 125 amino acids.

In certain embodiments, the expression cassette comprises a nucleic acid encoding a fragment that is a domain of LTBR. In certain embodiments, the nucleic acid encodes the extracellular domain of LTBR (amino acids 31-227 of SEQ ID NO: 2). In certain embodiments, the nucleic acid encodes the transmembrane domain of LTBR (amino acids 228-248 of SEQ ID NO: 2). In certain embodiments, the nucleic acid encodes the cytoplasmic (or intracellular) domain of LTBR (amino acids 249-435 of SEQ ID NO: 2). In other embodiments, the domain is a variant of one of the LTBR domains, including a variant that has a deletion. Desirable variants of the cytoplasmic domain include those that comprise amino acids 249-378, 249-379, 249-380, 249-381, 249-382, 249-383, 249-384, 249-385, 249-386, 249-387, 249-388, 249-389, 249-390, 249-391, or 249-392 all of SEQ ID NO: 2. Further desirable variants include those that comprise amino acids 249-378, 249-379, 249-380, 249-381, 249-382, 249-383, 249-384, 249-385, 249-386, 249-387, 249-388, 249-389, 249-390, 249-391, or 249-392 all of SEQ ID NO: 2 having 1, 2, 3, 4, 5, 6, 7, 8, 9, 10, 11, 12, 13, 14, or 15 amino acid substitutions as compared to SEQ ID NO: 2.

In other embodiments, the expression cassette comprises a nucleic acid encoding two or more domains of LTBR or fragments thereof. In one embodiment the nucleic acid encodes the cytoplasmic domain (or fragment thereof) and the transmembrane domain of LTBR. In another embodiment, the nucleic acid encodes the cytoplasmic domain (or fragment thereof), transmembrane domain, and extracellular domain of LTBR.

In another embodiment, the expression cassette comprises a nucleic acid encoding AHCY. AHCY encodes the enzyme S-adenosylhomocysteine hydrolase, which catalyzes the reversible hydrolysis of S-adenosylhomocysteine (AdoHcy) to adenosine (Ado) and L-homocysteine (Hcy). A representative nucleic acid sequence of AHCY can be found at Accession ID XM_011528656.3.

In another embodiment, the expression cassette comprises a nucleic acid encoding DUPD1. DUPD1 encodes the enzyme dual specificity phosphatase and pro isomerase domain containing 1 (also referred to as DUSP29—dual specificity phosphatase 29), is able to dephosphorylate phosphotyrosine, phosphoserine and phosphothreonine residues within the same substrate. A representative nucleic acid sequence of DUPD1 can be found at Accession ID XM_011539747.2.

In another embodiment, the expression cassette comprises a nucleic acid encoding AKR1C4. AKR1C4 encodes the enzyme aldo-keto reductase family 1 member C4 (also referred to as 3-alpha-HSD1, CDR, and DD-4), is able to that catalyzes the NADH and NADPH-dependent reduction of ketosteroids to hydroxysteroids. A representative nucleic acid sequence of AKR1C4 can be found at Accession ID NM_001818.5.

In another embodiment, the expression cassette comprises a nucleic acid encoding ATF6B. ATF6B encodes activating transcription factor 6 beta (also referred to as cyclic AMP-dependent transcription factor ATF-6 beta). The processed form of ATF-6 beta acts in the unfolded protein response pathway by activating UPR target genes induced by ER stress. A representative nucleic acid sequence of ATF6B can be found at Accession ID NM_004381.5.

In another embodiment, the expression cassette comprises a nucleic acid encoding ITM2A. ITM2A encodes integral membrane protein 2A (also referred to as protein E25), which binds amyloid-beta. A representative nucleic acid sequence of ITM2A can be found at Accession ID NM_004867.5.

In another embodiment, the expression cassette comprises a nucleic acid encoding AHNAK. AHNAK encodes Neuroblast differentiation-associated protein AHNAK (also referred to as AHNAK nucleoprotein). The encoded protein may play a role in such diverse processes as blood-brain barrier formation, cell structure and migration, cardiac calcium channel regulation, and tumor metastasis. A representative nucleic acid sequence of AHNAK can be found at Accession ID XM_017018270.1.

In another embodiment, the expression cassette comprises a nucleic acid encoding BATF. BATF encodes Basic leucine zipper transcriptional factor ATF-like (also referred to as B-cell-activating transcription factor (B-ATF); and SF-HT-activated gene 2 protein (SFA-2)), which is an AP-1 family transcription factor that controls the differentiation of lineage-specific cells in the immune system. A representative nucleic acid sequence of BATF can be found at Accession ID NM 006399.5.

In another embodiment, the expression cassette comprises a nucleic acid encoding GPD1. GPD1 encodes Glycerol-3-phosphate dehydrogenase [NAD(+)], cytoplasmic (also referred to as GPD-C and GPDH-C). A representative nucleic acid sequence of GPD1 can be found at Accession ID NM_005276.4.

In another embodiment, the expression cassette comprises a nucleic acid encoding GPN3. GPN3 encodes GPN-loop GTPase 3 (also referred to as ATP-binding domain 1 family member C), which is a small GTPase required for proper localization of RNA polymerase II. A representative nucleic acid sequence of GPN3 can be found at Accession ID XM_017019394.1.

In another embodiment, the expression cassette comprises a nucleic acid encoding MRPL51. MRPL51 encodes GPN-loop GTPase 3 (also referred to as ATP-binding domain 1 family member C), which is a small GTPase required for proper localization of RNA polymerase II. A representative nucleic acid sequence of MRPL51 can be found at Accession ID NM 016497.4.

In another embodiment, the expression cassette comprises a nucleic acid encoding DBI. DBI encodes diazepam binding inhibitor (also referred to as ACBD1, ACBP, CCK-RP, EP), a protein that is regulated by hormones and is involved in lipid metabolism and the displacement of beta-carbolines and benzodiazepines. A representative nucleic acid sequence of DBI can be found at Accession ID NM_001282635.3.

In another embodiment, the expression cassette comprises a nucleic acid encoding CALML3. CALML3 encodes calmodulin like 3 (also referred to as CLP), a protein that enhances myosin-10 translation. A representative nucleic acid sequence of CALML3 can be found at Accession ID NM_005185.4.

In another embodiment, the expression cassette comprises a nucleic acid encoding IL12B. IL12B encodes interleukin 12B (also referred to as CLMF, CLMF2, IL-12B, IMD28, IMD29, NKSF, NKSF2), a cytokine that acts on T and natural killer cells, and has a broad array of biological activities. A representative nucleic acid sequence of IL12B can be found at Accession ID NM_002187.3.

In another embodiment, the expression cassette comprises a nucleic acid encoding IFNL2. IFNL2 encodes interferon lambda 2 (also referred to as IL28A; IFNL2a; IFNL3a; IL-28A). This gene, interleukin 28B (IL28B), and interleukin 29 (IL29) are three closely related cytokine genes that form a cytokine gene cluster on a chromosomal region mapped to 19q13. Expression of the cytokines encoded by the three genes can be induced by viral infection. All three cytokines have been shown to interact with a heterodimeric class II cytokine receptor that consists of interleukin 10 receptor, beta (IL10RB) and interleukin 28 receptor, alpha (IL28RA). A representative nucleic acid sequence of IFNL2 can be found at Accession ID NM_172138.2.

In another embodiment, the expression cassette comprises a nucleic acid encoding ADA. ADA encodes adenosine deaminase (also referred to as ADA1; IFNL2a; IFNL3a; IL-28A). This gene encodes an enzyme that catalyzes the hydrolysis of adenosine to inosine in the purine catabolic pathway. A representative nucleic acid sequence of ADA can be found at Accession ID NM_000022.4.

Various isoforms of the genes identified above are known in the art. Some are described in Table 2 below. In another embodiment, an expression cassette is provided which includes the coding sequence for any of the alternative isoforms. Alternative coding sequences accounting to the degeneracy of the genetic code, including codon optimized coding sequences, for these genes can be identified by the person of skill in the art, and utilized as an alternative embodiment of the compositions and methods described herein.

TABLE 2

| Gene | | | Length | | Length | | |
|---|---|---|---|---|---|---|---|
| ID | Symbol | Transcript | (nt) | Protein | (aa) | Isoform | Gene name |
| 100 | ADA | NR_136160.2 | 1431 | | | | adenosine deaminase |
| 100 | ADA | NM_001322050.2 | 1380 | NP_001308979.1 | 228 | 2 | adenosine deaminase |
| 100 | ADA | NM_001322051.2 | 1424 | NP_001308980.1 | 339 | 3 | adenosine deaminase |
| 100 | ADA | NM_000022.4 | 1496 | NP_000013.2 | 363 | 1 | adenosine deaminase |
| 10538 | BATF | NM_006399.5 | 913 | NP_006390.1 | 125 | | basic leucine zipper ATF-like transcription factor |
| 1109 | AKR1C4 | NM_001818.5 | 1192 | NP_001809.4 | 323 | | aldo-keto reductase family 1 member C4 |
| 1388 | ATF6B | NM_001136153.2 | 2617 | NP_001129625.1 | 700 | b | activating transcription factor 6 beta |
| 1388 | ATF6B | NM_004381.5 | 2626 | NP_004372.3 | 703 | a | activating transcription factor 6 beta |
| 1622 | DBI | NM_001282635.3 | 804 | NP_001269564.1 | 104 | 1 | diazepam binding inhibitor, acyl-CoA binding |
| | | | | | | | protein |
| 1622 | DBI | NM_001282636.3 | 718 | NP_001269565.1 | 63 | 7 | diazepam binding inhibitor, acyl-CoA binding |
| | | | | | | | protein |
| 1622 | DBI | NM_001282633.3 | 680 | NP_001269562.1 | 104 | 1 | diazepam binding inhibitor, acyl-CoA binding |
| | | | | | | | protein |
| 1622 | DBI | NM_020548.9 | 675 | NP_065438.1 | 104 | 1 | diazepam binding inhibitor, acyl-CoA binding |
| | | | | | | | protein |
| 1622 | DBI | NM_001178043.4 | 594 | NP_001171514.1 | 97 | 6 | diazepam binding inhibitor, acyl-CoA binding |
| | | | | | | | protein |
| 1622 | DBI | NM_001178041.4 | 690 | NP_001171512.1 | 129 | 5 | diazepam binding inhibitor, acyl-CoA binding |
| | | | | | | | protein |
| 1622 | DBI | NM_001282634.3 | 650 | NP_001269563.1 | 104 | 1 | diazepam binding inhibitor, acyl-CoA binding |
| | | | | | | | protein |
| 1622 | DBI | NM_001178042.4 | 645 | NP_001171513.1 | 104 | 1 | diazepam binding inhibitor, acyl-CoA binding |
| | | | | | | | protein |
| 1622 | DBI | NM_001079862.4 | 564 | NP_001073331.1 | 87 | 3 | diazepam binding inhibitor, acyl-CoA binding |
| | | | | | | | protein |
| 1622 | DBI | NM_001352432.3 | 896 | NP_001339361.1 | 63 | 7 | diazepam binding inhibitor, acyl-CoA binding |
| | | | | | | | protein |
| 1622 | DBI | NM_001178017.3 | 756 | NP_001171488.1 | 148 | 4 | diazepam binding inhibitor, acyl-CoA binding |
| | | | | | | | protein |
| 1622 | DBI | NM_001079863.3 | 698 | NP_001073332.1 | 88 | 2 | diazepam binding inhibitor, acyl-CoA binding |
| | | | | | | | protein |
| 191 | AHCY | XM_011528657.2 | 1436 | XP_011526959.2 | 434 | X1 | adenosylhomocysteinase |
| 191 | AHCY | XM_017027709.2 | 1801 | XP_016883198.1 | 432 | X2 | adenosylhomocysteinase |
| 191 | AHCY | XM_011528658.3 | 1798 | XP_011526960.2 | 434 | X1 | adenosylhomocysteinase |
| 191 | AHCY | XM_011528656.3 | 1803 | XP_011526958.2 | 434 | X1 | adenosylhomocysteinase |
| 191 | AHCY | XM_011528659.1 | 2196 | XP_011526961.1 | 404 | X3 | adenosylhomocysteinase |
| 191 | AHCY | NM_001161766.2 | 2368 | NP_001155238.1 | 404 | 2 | adenosylhomocysteinase |
| 191 | AHCY | NM_001362750.2 | 1774 | NP_001349679.1 | 432 | 1 | adenosylhomocysteinase |
| 191 | AHCY | NM_000687.4 | 2150 | NP_000678.1 | 432 | 1 | adenosylhomocysteinase |
| 191 | AHCY | XM_017027710.2 | 2136 | XP_016883199.1 | 306 | X4 | adenosylhomocysteinase |
| 191 | AHCY | XM_005260317.2 | 2601 | XP_005260374.1 | 404 | X3 | adenosylhomocysteinase |
| 191 | AHCY | NM_001322085.2 | 3092 | NP_001309014.1 | 404 | 2 | adenosylhomocysteinase |
| 191 | AHCY | NM_001322084.2 | 3270 | NP_001309013.1 | 404 | 2 | adenosylhomocysteinase |
| 191 | AHCY | NM_001322086.2 | 3281 | NP_001309015.1 | 434 | 3 | adenosylhomocysteinase |
| 2819 | GPD1 | NM_005276.4 | 2887 | NP_005267.2 | 349 | 1 | glycerol-3-phosphate dehydrogenase 1 |
| 2819 | GPD1 | NM_001257199.2 | 2818 | NP_001244128.1 | 326 | 2 | glycerol-3-phosphate dehydrogenase 1 |
| 282616 | IFNL2 | NM_172138.2 | 951 | NP_742150.1 | 200 | | interferon lambda 2 |
| 338599 | DUSP29 | XM_011539747.2 | 4872 | XP_011538049.1 | 220 | X1 | dual specificity phosphatase 29 |
| 338599 | DUSP29 | NM_001003892.3 | 1135 | NP_001003892.1 | 220 | | dual specificity phosphatase 29 |
| 338599 | DUSP29 | NM_001384909.1 | 1267 | NP_001371838.1 | 220 | | dual specificity phosphatase 29 |
| 338599 | DUSP29 | XM_017016176.1 | 1284 | XP_016871665.1 | 112 | X2 | dual specificity phosphatase 29 |
| 3593 | IL12B | NM_002187.3 | 2364 | NP_002178.2 | 328 | | interleukin 12B |
| 4055 | LTBR | NM_001270987.2 | 2276 | NP_001257916.1 | 416 | 2 | lymphotoxin beta receptor |
| 4055 | LTBR | XM_005253688.2 | 2094 | XP_005253745.1 | 399 | X1 | lymphotoxin beta receptor |
| 4055 | LTBR | XM_006718983.3 | 1127 | XP_006719046.1 | 255 | X2 | lymphotoxin beta receptor |
| 4055 | LTBR | NM_002342.3 | 2134 | NP_002333.1 | 435 | 1 precursor | lymphotoxin beta receptor |
| 51184 | GPN3 | NM_001164372.2 | 1605 | NP_001157844.1 | 323 | 2 | GPN-loop GTPase 3 |
| 51184 | GPN3 | NM_016301.4 | 1457 | NP_057385.3 | 284 | 1 | GPN-loop GTPase 3 |
| 51184 | GPN3 | NM_001164373.2 | 1487 | NP_001157845.1 | 294 | 3 | GPN-loop GTPase 3 |
| 51184 | GPN3 | XM_017019394.1 | 1357 | XP_016874883.1 | 262 | X1 | GPN-loop GTPase 3 |
| 51258 | MRPL51 | NM_016497.4 | 898 | NP_057581.2 | 128 | | mitochondrial ribosomal protein L51 |
| 79026 | AHNAK | NM_024060.4 | 1036 | NP_076965.2 | 149 | 2 | AHNAK nucleoprotein |
| 79026 | AHNAK | NM_001620.3 | 18761 | NP_001611.1 | 5890 | 1 | AHNAK nucleoprotein |
| 79026 | AHNAK | XM_017018270.1 | 18556 | XP_016873759.1 | 5823 | X1 | AHNAK nucleoprotein |
| 79026 | AHNAK | NM_001346445.2 | 18697 | NP_001333374.1 | 5890 | 1 | AHNAK nucleoprotein |
| 79026 | AHNAK | NM_001346446.2 | 18713 | NP_001333375.1 | 5890 | 1 | AHNAK nucleoprotein |
| 810 | CALML3 | NM_005185.4 | 1811 | NP_005176.1 | 149 | | calmodulin like 3 |
| 9452 | ITM2A | NM_001171581.2 | 1484 | NP_001165052.1 | 219 | 2 | integral membrane protein 2A |
| 9452 | ITM2A | NM_004867.5 | 1616 | NP_004858.1 | 263 | 1 | integral membrane protein 2A |

In other embodiments, the expression cassette comprises a nucleic acid encoding a gene selected from the genes of Table 1.

### Engineered T Cell Receptors

The present disclosure provides nucleic acid sequences encoding engineered T cell receptors, e.g., T cell receptors (TCR), TCRs modified as described herein, and chimeric antigen receptors (CAR), for expression in T cells with a nucleic acid sequence encoding a gene that alters T cell effector function. Components of the TCR and CARs are further described herein.

The TCR is a disulfide-linked membrane-anchored heterodimer present on T cell lymphocytes, and the majority of T cells are αβ T cells having a TCR consisting of an alpha (α) chain and a beta (β) chain. Each chain comprises a variable (V) and a constant (C) domain, wherein the variable domain recognizes an antigen, or an MHC-presented peptide. TCRα and TCRβ chains with a known specificity or affinity for specific antigens, e.g., tumor antigens described herein, can be introduced to a T cell using the methods described herein. TCRα and TCRβ chains having a desired, e.g., increased, specificity or affinity for a particular antigen can be isolated using standard molecular cloning techniques known in the art. Other modifications that increase specificity, avidity, or function of the TCRs or the engineered T cells expressing the TCRs can be readily envisioned by the ordinarily skilled artisan, e.g., promoter selection for regulated expression, mutations in the antigen binding regions of the TCRα and TCRβ chains. Any isolated or modified TCRα and TCRβ chain can be operably linked to or can associate with one or more intracellular signaling domains described herein. Signaling can be mediated through interaction between the antigen-bound αβ heterodimer to CD3 chain molecules, e.g., CD3zeta (ζ).

A smaller subset of T cells expresses a TCR having a (γ) gamma chain and a delta (δ) chain. Gamma-delta (γδ) T cells make up 3-10% of circulating lymphocytes in humans, and the Vδ2+ subset can account for up to 95% of γδ T cells in blood. Vδ2+ cells recognize non-peptide epitopes and do not require antigen presentation by major histocompatibility complexes (“MHC”) or human leukocyte antigen (“HLA”). The majority of Vδ2+ T cells also express a Vγ9 chain and are stimulated by exposure to 5-carbon pyrophosphate compounds that are intermediates in mevalonate and non-mevalonate sterol/isoprenoid synthesis pathways. The response to isopentenyl pyrophosphate (5-carbon) is universal among healthy human beings. Another subset of γδ T cells, Vδ1+, make up a much smaller percentage of the T cells circulating in the blood, but are commonly found in the epithelial mucosa and the skin. γδ T cells have several functions, including killing tumor cells and pathogen-infected cells. Stimulation through the γδ TCR improves the capacity for cellular cytotoxicity, cytokine secretion and other effector functions. The TCRs of γδ T cells have unique specificities and the cells themselves occur in high clonal frequencies, thus allowing rapid innate-like responses to tumors and pathogens. See, e.g., Park and Lec, Exp Mol Med. 2021 March; 53(3):318-327, which is incorporated herein by reference.

In certain embodiments, a T cell comprises a nucleic acid sequence encoding a TCR. e.g., a modified TCR that targets a tumor antigen described herein, and a nucleic acid sequence encoding a gene. In any of the embodiments described herein, a TCR can be substituted for a CAR described herein to generate a T cell. An engineered TCR described herein can be substituted for a CAR in any of the embodiments described herein. In certain embodiments, the engineered TCR that targets NY-ESO-1 (SEQ ID NO: 23 and 24) (see, e.g., Thomas et al., NY-ESO-1 Based Immunotherapy of Cancer: Current Perspectives, Front. Immunol., 1 May 2018, which is incorporated herein by reference).

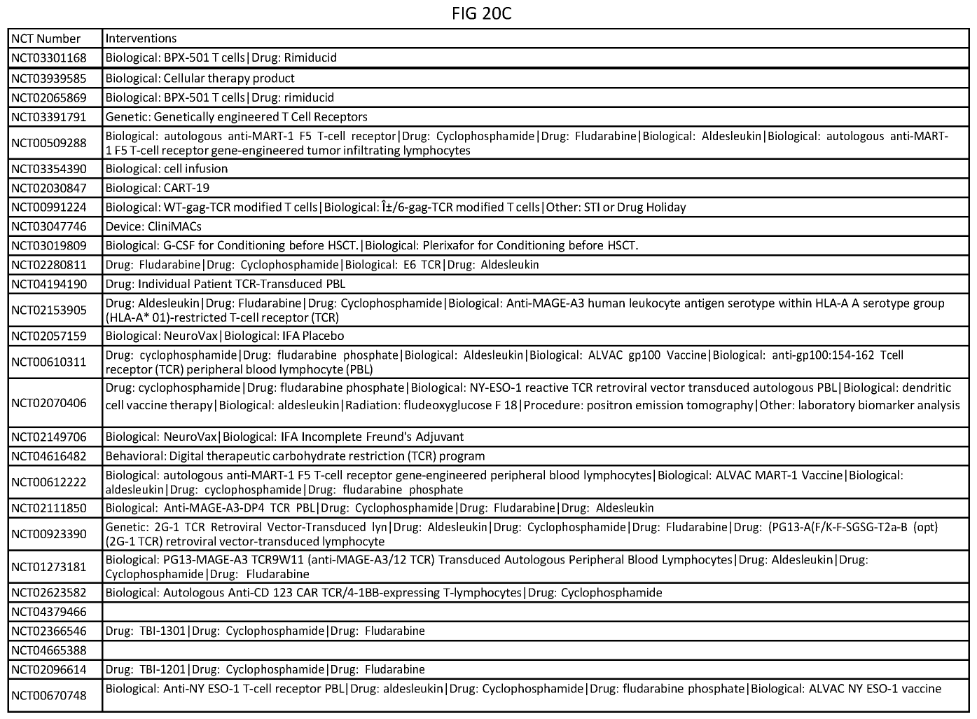

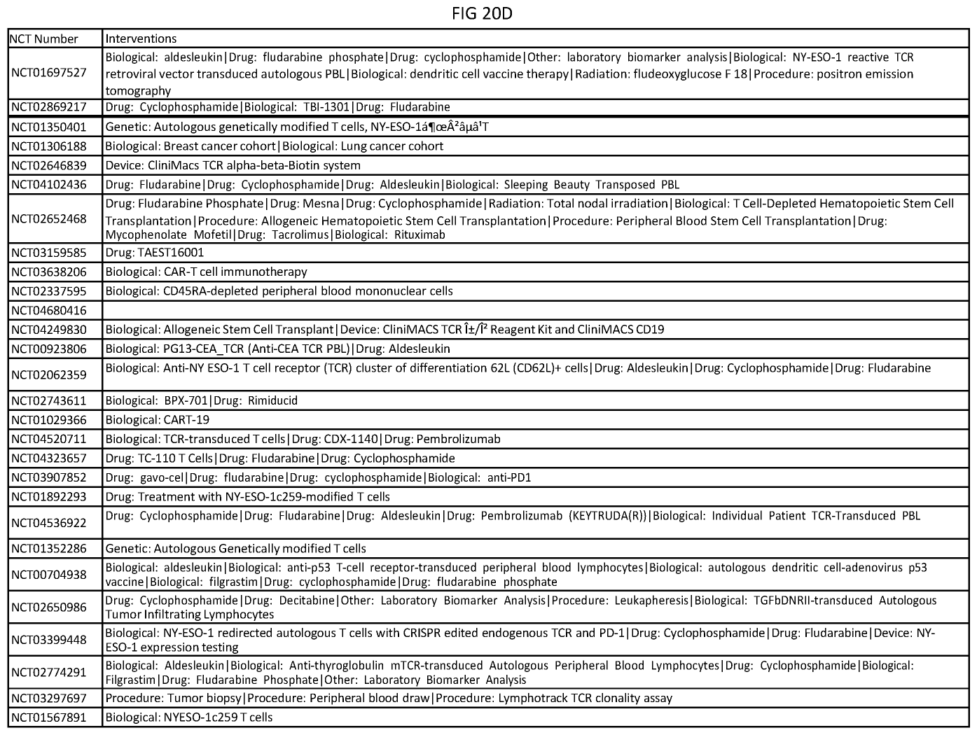

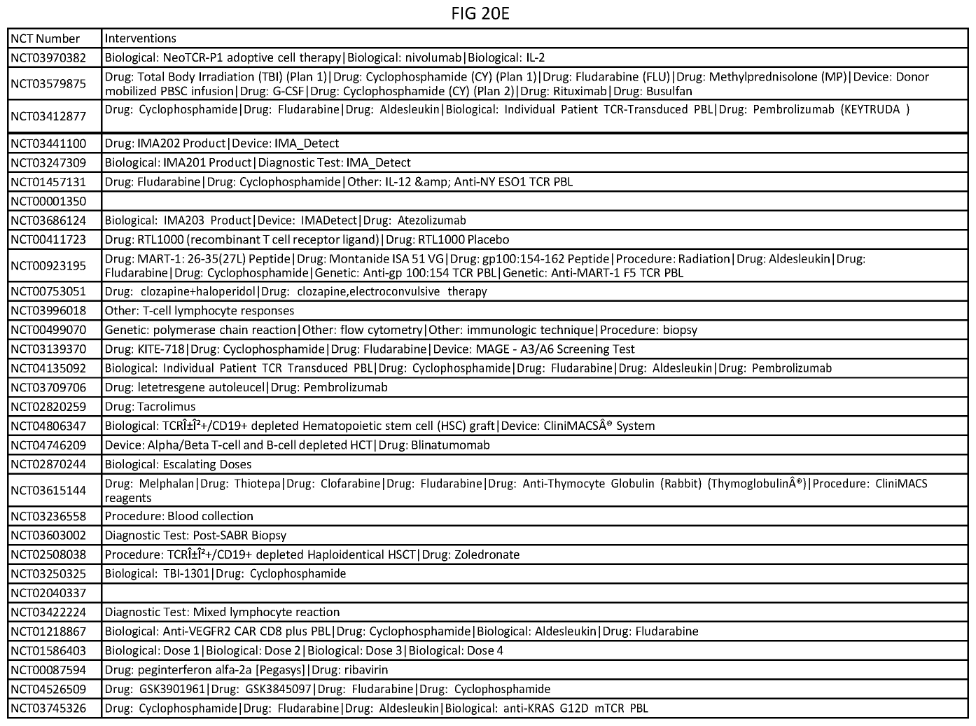

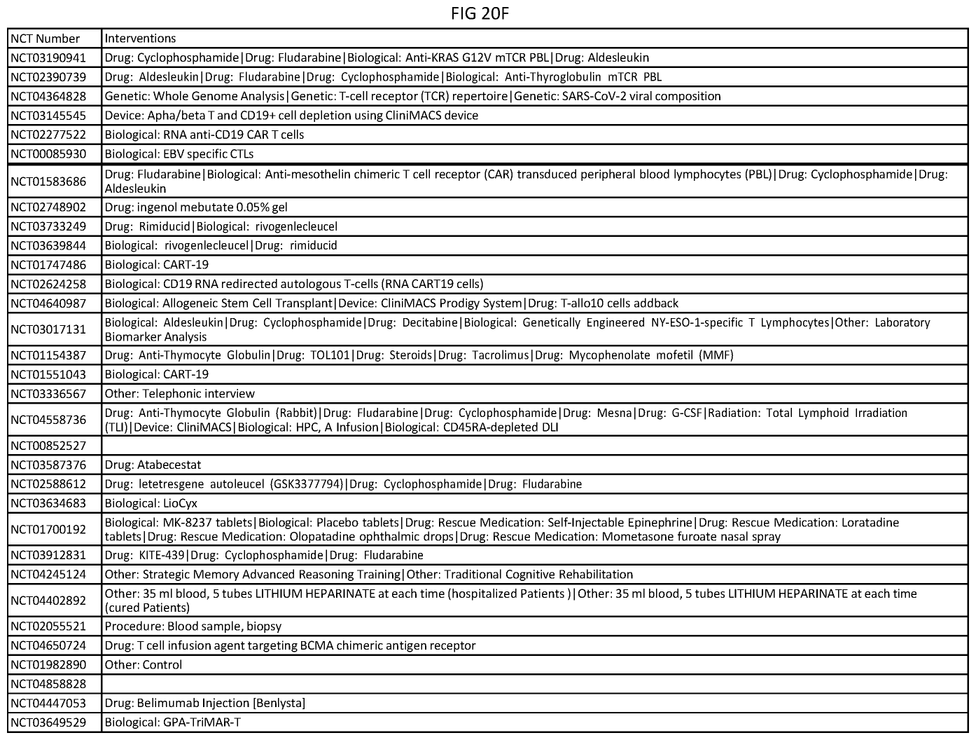

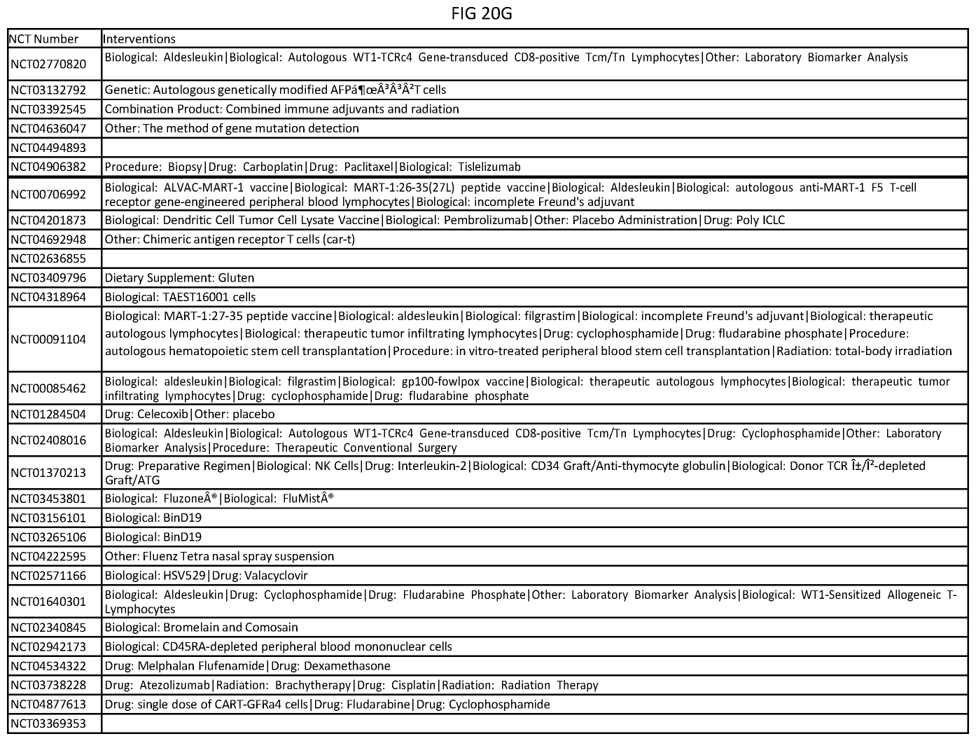

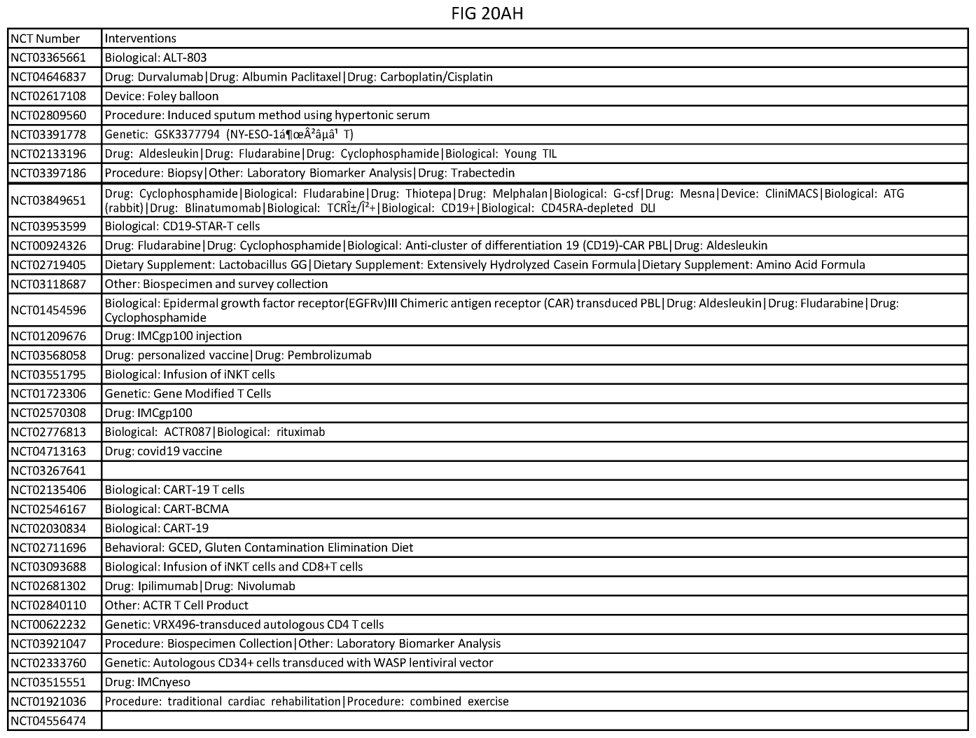

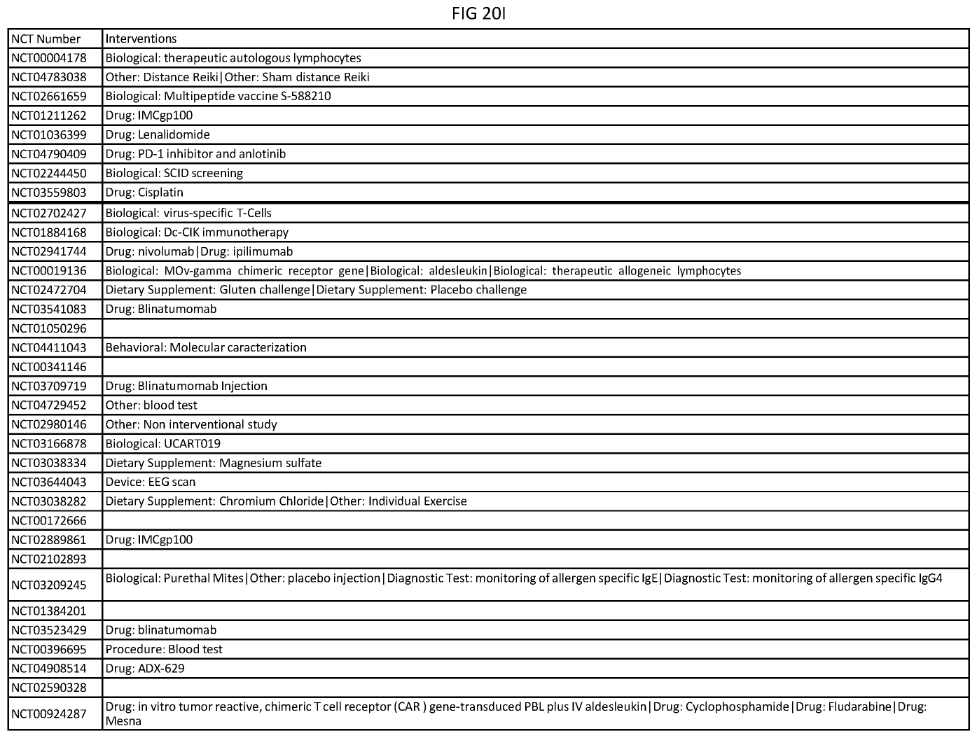

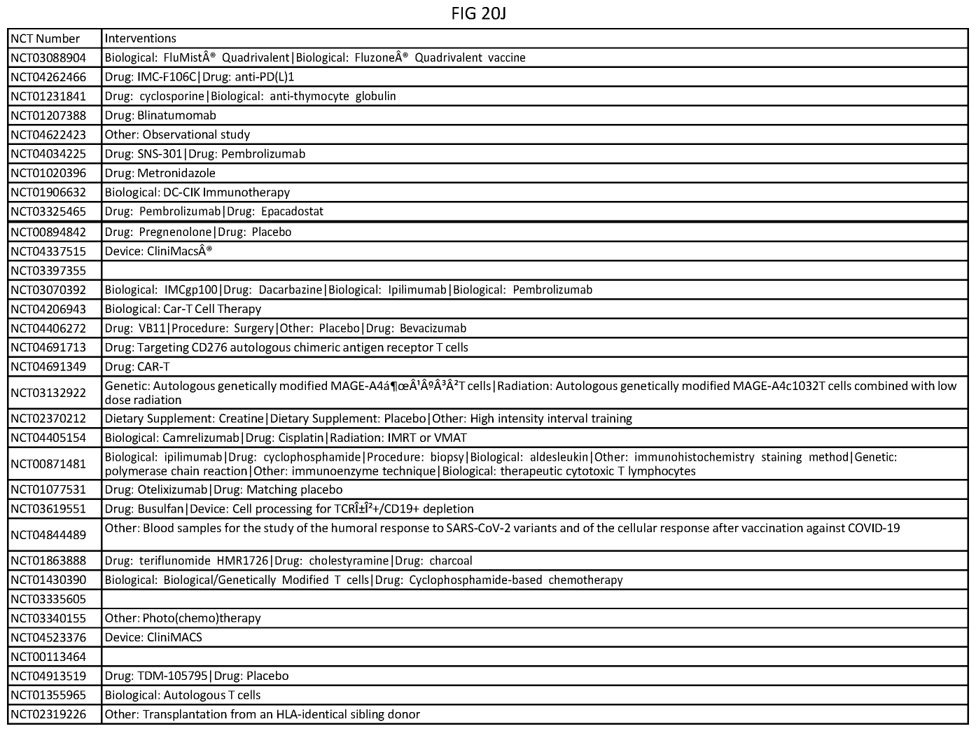

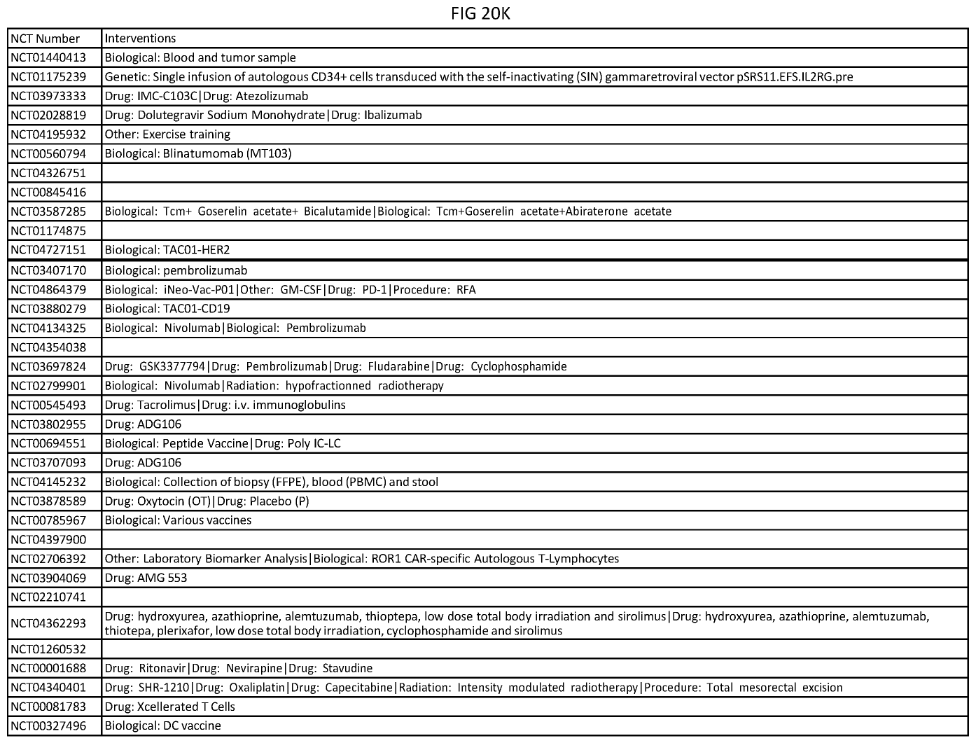

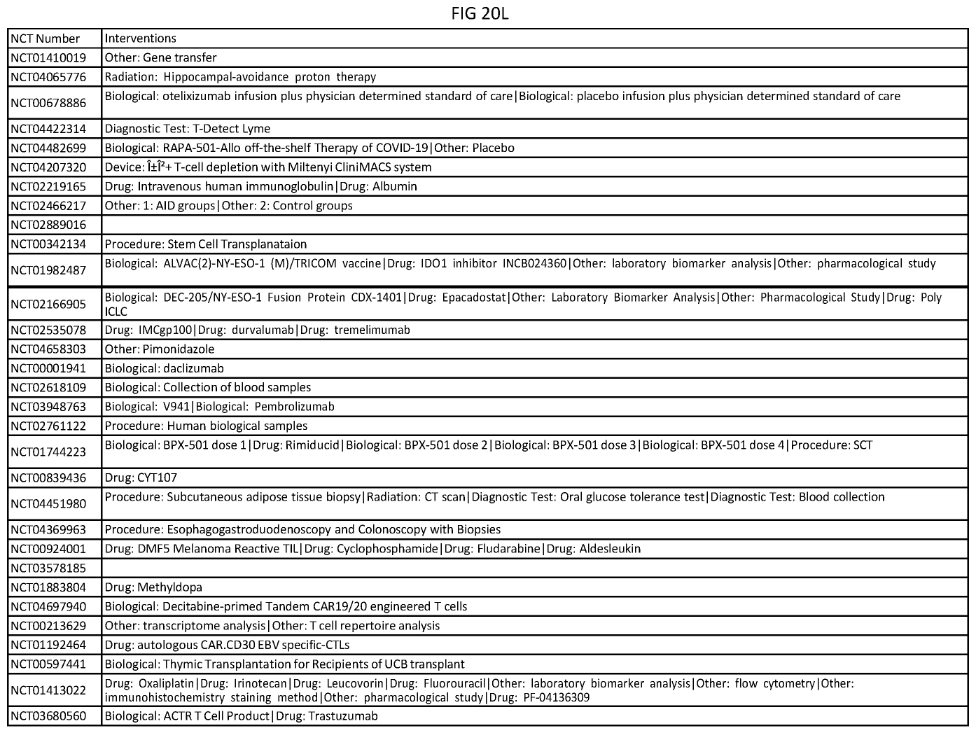

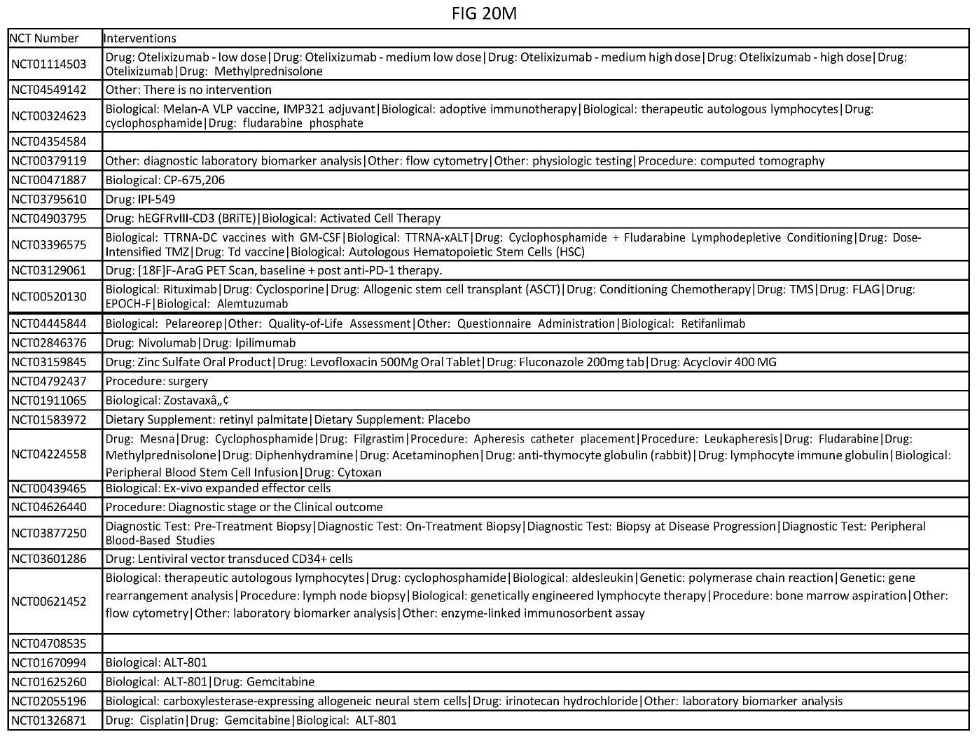

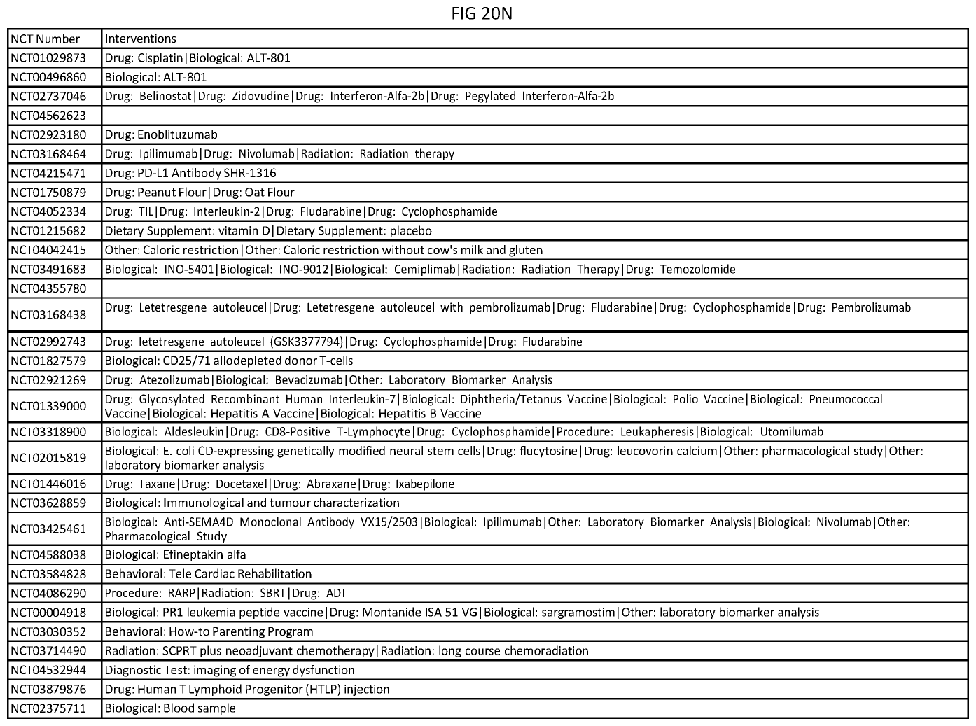

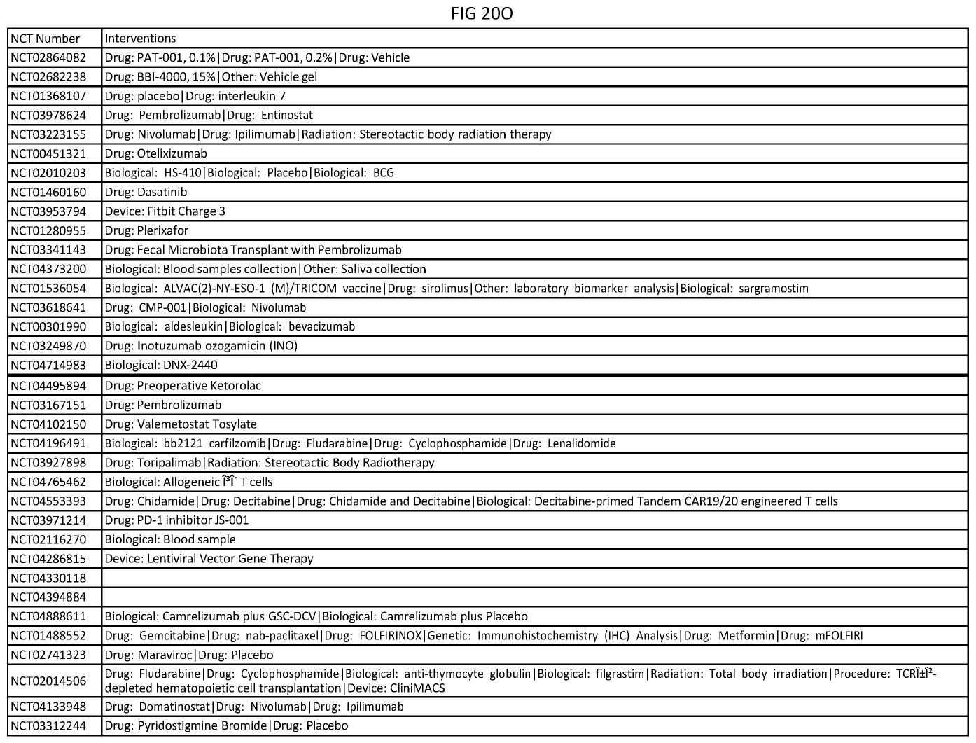

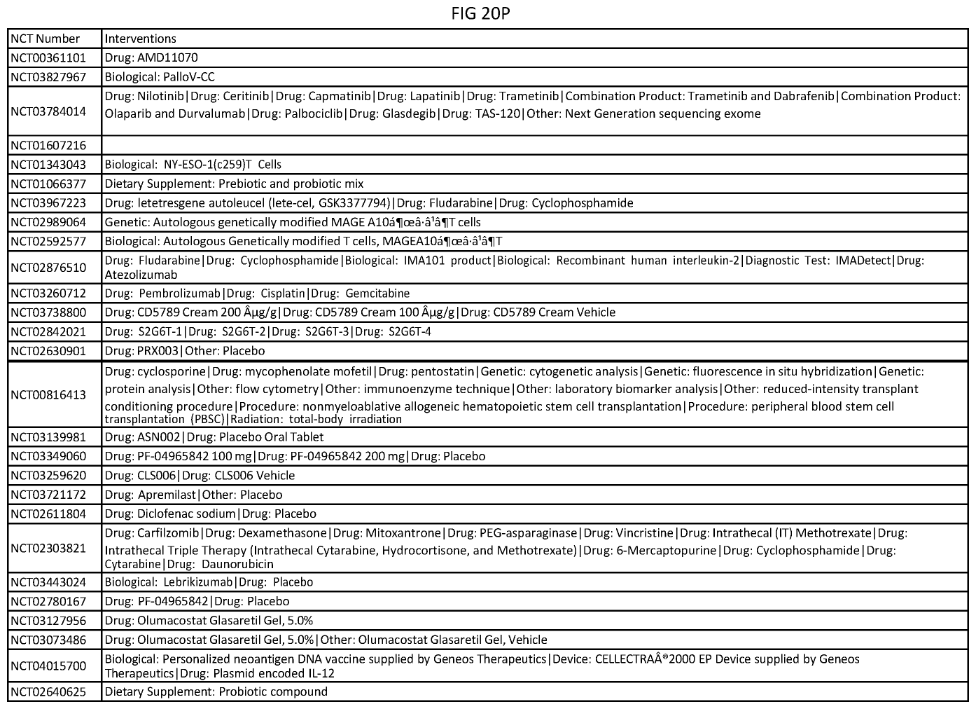

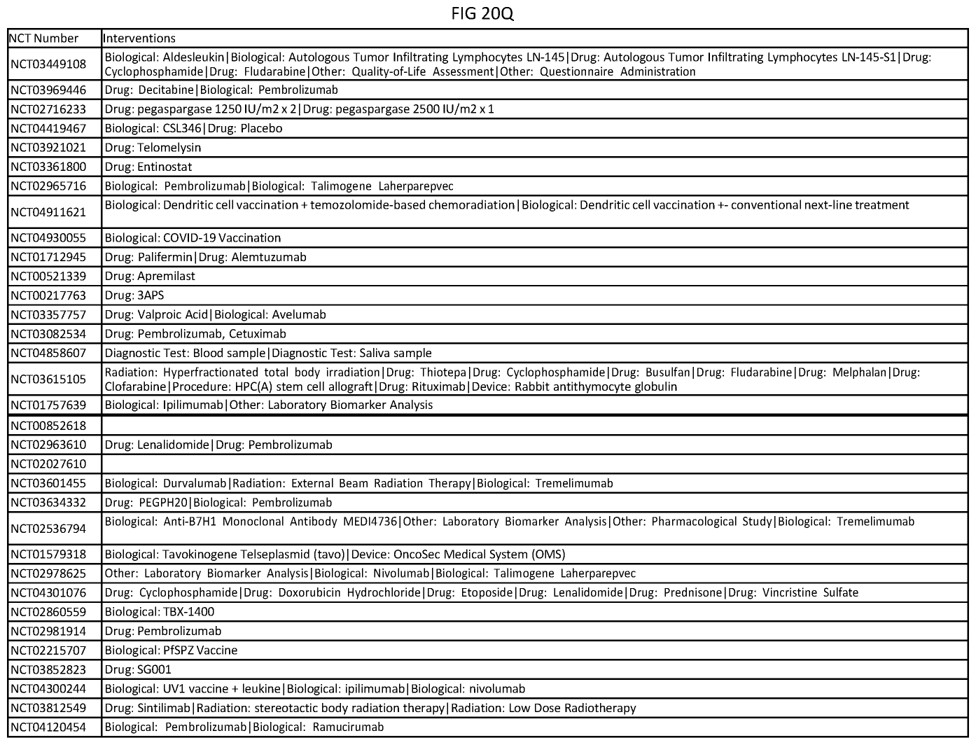

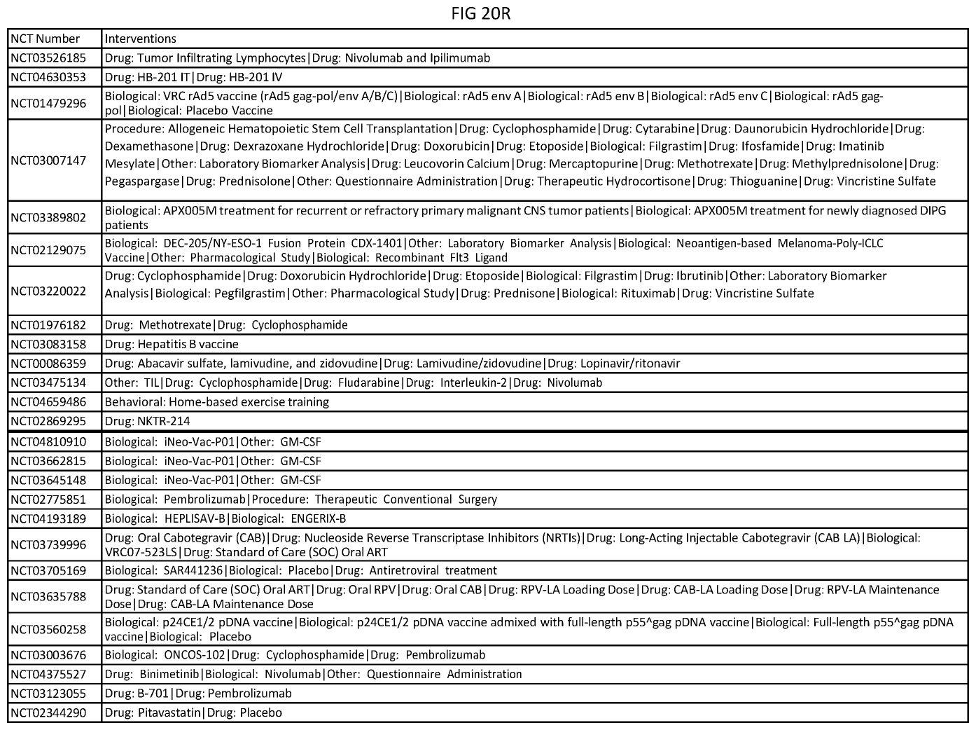

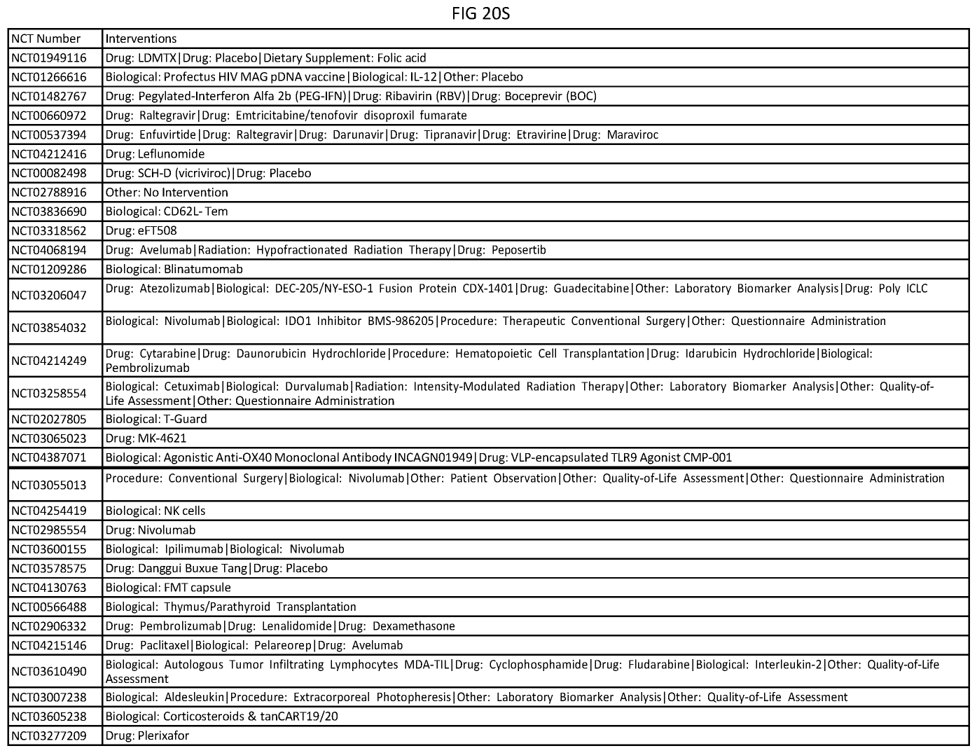

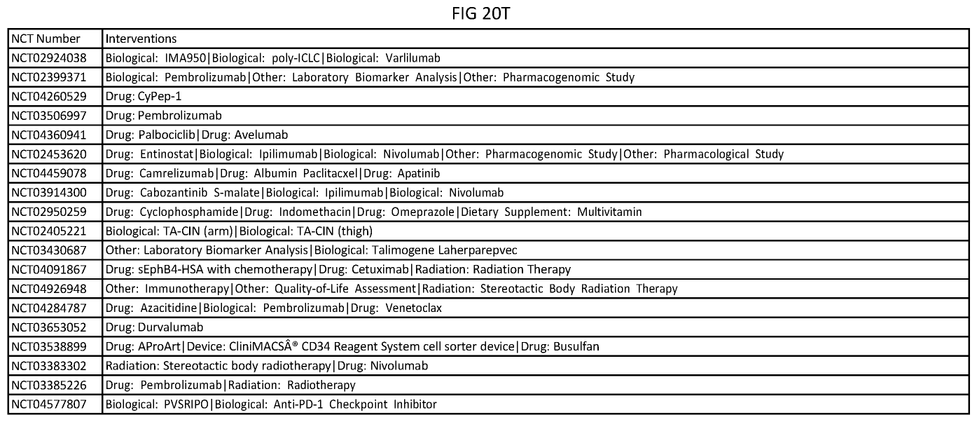

In certain embodiments, the T cell comprises a TCR identified in FIG. 20. In certain embodiments, the TCR targets MART-1. Chodon T, et al, Adoptive transfer of MART-1 T-cell receptor transgenic lymphocytes and dendritic cell vaccination in patients with metastatic melanoma. Clin Cancer Res. 2014 May 1; 20(9):2457-65. doi: 10.1158/1078-0432.CCR-13-3017. Epub 2014 Mar. 14. PMID: 24634374; PMCID: PMC4070853. In other embodiments, the TCR targets MAGE A4. Hong et al, Phase I dose escalation and expansion trial to assess the safety and efficacy of ADP-A2M4 SPEAR T cells in advanced solid tumors. ASCO Meeting Library, 2020 ASCO Virtual Scientific Program, J Clin Oncol 38: 2020 (suppl: abstr 102). In other embodiments, the TCR targets WT1. Chapuis A G, et al. T cell receptor gene therapy targeting WT1 prevents acute myeloid leukemia relapse post-transplant. Nat Med. 2019 July; 25(7): 1064-1072. doi: 10.1038/s41591-019-0472-9. Epub 2019 Jun. 24. PMID: 31235963; PMCID: PMC6982533. In other embodiments, the TCR targets MR1. Crowther, M. D., Dolton, G., Legut, M. et al. Genome-wide CRISPR-Cas9 screening reveals ubiquitous T cell cancer targeting via the monomorphic MHC class I-related protein MR1. Nat Immunol 21, 178-185 (2020). https://doi.org/10.1038/s41590-019-0578-8. In other embodiments, the TCR targets E6. In other embodiments, the TCR targets E7. In other embodiments, the TCR targets KK-LC-1. In other embodiments, the TCR targets NY-ESO-1. In other embodiments, the TCR targets MAGE A3. In other embodiments, the TCR targets GD-2. In other embodiments, the TCR targets P53. In other embodiments, the TCR targets LAGE-A1. In other embodiments, the TCR targets GP100.

### Chimeric Antigen Receptor (CAR)

The term “chimeric antigen receptor” or alternatively a “CAR” refers to a recombinant polypeptide construct comprising at least an extracellular antigen binding domain, a transmembrane domain and a cytoplasmic signaling domain (also referred to as an intracellular signaling domain) comprising a functional signaling domain derived from a stimulatory molecule as defined below. In some embodiments, the stimulatory molecule is TCR zeta, FcR gamma, FcR beta, CD3 gamma, CD3 delta, CD3 epsilon, CD5, CD22, CD79a, CD79b, CD66d, 4-1BB, or CD3-zeta. In certain embodiments, the stimulatory molecule is the zeta chain associated with the T cell receptor complex. In certain embodiments, the stimulatory molecule is 4-1BB. In certain embodiments, the stimulatory molecule is CD28. In certain embodiments, the cytoplasmic signaling domain further comprises one or more functional signaling domains derived from at least one costimulatory molecule as defined below (also referred to as a “costimulatory signaling domain”). In certain embodiments, the costimulatory molecule is chosen from a costimulatory molecule described herein, e.g., OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof. In certain embodiments, the CAR comprises a chimeric fusion protein comprising an extracellular antigen recognition domain, a transmembrane domain and an intracellular signaling domain comprising a functional signaling domain derived from a stimulatory molecule (a primary signaling domain). In certain embodiments, the CAR comprises a chimeric fusion protein comprising an extracellular antigen recognition domain, a transmembrane domain and an intracellular signaling domain comprising a functional signaling domain derived from a co-stimulatory molecule (a costimulatory signaling domain) and a functional signaling domain derived from a stimulatory molecule (a primary signaling domain). In certain embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In certain embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In certain embodiments, the CAR comprises an optional leader sequence at the amino-terminus (N-ter) of the CAR fusion protein. In certain embodiments, the CAR further comprises a leader sequence at the N-terminus of the extracellular antigen binding domain, wherein the leader sequence is optionally cleaved from the scFv domain during cellular processing and localization of the CAR to the cellular membrane.

The present disclosure provides nucleic acid sequences, e.g., DNA or RNA constructs, encoding a CAR, wherein the CAR comprises an antibody fragment that binds to a disease-associated antigen. In certain embodiments embodiment, the sequence encoding the antibody fragment is contiguous with, and in the same reading frame as a nucleic acid sequence encoding an intracellular domain. The intracellular domain comprises, a costimulatory signaling region and/or a zeta chain. The costimulatory signaling region refers to a portion of the CAR comprising the intracellular domain of a costimulatory molecule.

In certain embodiments, the CAR construct comprises an optional leader sequence, an extracellular antigen binding domain, a hinge, a transmembrane domain, and an intracellular stimulatory domain. In certain embodiments, the CAR construct comprises an optional leader sequence, an extracellular antigen binding domain, a hinge, a transmembrane domain, an intracellular costimulatory domain and an intracellular stimulatory domain.

In certain embodiments, the expression cassette includes that encodes, in addition to the effector enhancing gene, one or more components of a chimeric antigen receptor. For example, in one embodiment, a single expression cassette is provided which includes the coding sequence for the effector enhancing gene and coding sequences for a chimeric fusion protein comprising an extracellular antigen binding domain, a transmembrane domain and an intracellular signaling domain.

In certain embodiments, the CAR targets CD19. In one embodiment, the CAR is axicabtagene ciloleucel. In another embodiment, the CAR is Brexucabtagene autoleucel. In another embodiment, the CAR is Tisagenlecleucel. In another embodiment, the CAR is Lisocabtagene maraleucel. In another embodiment, the CAR is Idecabtagene vicleucel.

In one embodiment, an expression cassette is provided, comprising coding sequences for LTBR and axicabtagene ciloleucel. In another embodiment, an expression cassette is provided, comprising coding sequences for LTBR and Brexucabtagene autoleucel. In another embodiment, an expression cassette is provided, comprising coding sequences for LTBR and Tisagenlecleucel. In another embodiment, an expression cassette is provided, comprising coding sequences for LTBR and Lisocabtagene maraleucel. In another embodiment, an expression cassette is provided, comprising coding sequences for LTBR and Idecabtagene vicleucel.

In another embodiment, an expression cassette is provided, comprising coding sequence for AHCY, DUPD1, AKR1C4, ATF6B, ITM2A, AHNAK, BATF, GPD1, GPN3, MRPL51, DBI, CALML3, IL12B, IFNL2, or ADA and axicabtagene ciloleucel. In another embodiment, an expression cassette is provided, comprising coding sequences for AHCY, DUPD1, AKR1C4, ATF6B, ITM2A, AHNAK, BATF, GPD1, GPN3, MPRL51, DBI, CALML3, IL12B, IFNL2, or ADA and Brexucabtagene autoleucel. In another embodiment, an expression cassette is provided, comprising coding sequences for AHCY, DUPD1, AKR1C4, ATF6B, ITM2A, AHNAK, BATF, GPD1, GPN3, MRPL51, DBI, CALML3, IL12B, IFNL2, or ADA and Tisagenlecleucel. In another embodiment, an expression cassette is provided, comprising coding sequences for AHCY, DUPD1, AKR1C4, ATF6B, ITM2A, AHNAK, BATF, GPD1, GPN3, MRPL51, DBI, CALML3, IL12B, IFNL2, or ADA and Lisocabtagene maraleucel. In another embodiment, an expression cassette is provided, comprising coding sequence for AHCY, DUPD1, AKR1C4, ATF6B, ITM2A, AHNAK, BATF, GPD1, GPN3, MRPL51, DBI, CALML3, IL12B, IFNL2, or ADA and Idecabtagene vicleucel.

In another embodiment, an expression cassette is provided, comprising a coding sequence for any of the genes of Table 1 and axicabtagene ciloleucel. In another embodiment, an expression cassette is provided, comprising coding sequences for any of the genes of Table 1 and Brexucabtagene autoleucel. In another embodiment, an expression cassette is provided, comprising coding sequences for any of the genes of Table 1 and Tisagenlecleucel. In another embodiment, an expression cassette is provided, comprising coding sequences for any of the genes of Table 1 and Lisocabtagene maraleucel. In another embodiment, an expression cassette is provided, comprising coding sequences for any of the genes of Table 1 and Idecabtagene vicleucel.

In certain embodiments, the CAR targets mesothelin. In certain embodiments, the CAR targets ROR1. In certain embodiments, the CAR targets B7-H3. In certain embodiments, the CAR targets CD33. In certain embodiments, the CAR targets EGFR806. In certain embodiments, the CAR targets IL13Rα2. In certain embodiments, the CAR targets GD2. In certain embodiments, the CAR targets HER2. In certain embodiments, the CAR targets Glypican 3. In certain embodiments, the CAR targets CD7. In certain embodiments, the CAR targets NY-ESO-1. In certain embodiments, the CAR targets CD30. In certain embodiments, the CAR targets MAGE-A1. In certain embodiments, the CAR targets LMP2. In certain embodiments, the CAR targets PD1. In certain embodiments, the CAR targets mutant KRAS G12V. In certain embodiments, the CAR targets CD20. In certain embodiments, the CAR targets CD22. In certain embodiments, the CAR targets CD171. In certain embodiments, the CAR targets CD123. In certain embodiments, the CAR targets CD38. In certain embodiments, the CAR targets CD10. In certain embodiments, the CAR targets BAFFR. In certain embodiments, the CAR targets PSMA. In certain embodiments, the CAR targets mucin (TnMUC1). Posey A D Jr, et al, Engineered CAR T Cells Targeting the Cancer-Associated Tn-Glycoform of the Membrane Mucin MUC1 Control Adenocarcinoma. Immunity. 2016 Jun. 21; 44(6): 1444-54. doi: 10.1016/j.immuni.2016.05.014. PMID: 27332733; PMCID: PMC5358667. In certain embodiments, the CAR targets CD70. See, Srinivasan et al, 1972 Investigation of ALLO-316: A Fratricide-Resistant Allogeneic CAR T Targeting CD70 As a Potential Therapy for the Treatment of AML, 62^{nd} ASH Annual Meeting and Exposition, Dec. 5-8, 2020. In certain embodiments, the CAR targets TRIB1C. Maciocia P M, et al. Targeting the T cell receptor β-chain constant region for immunotherapy of T cell malignancies. Nat Med. 2017 December; 23(12): 1416-1423. doi: 10.1038/nm.4444. Epub 2017 Nov. 13. PMID: 29131157.

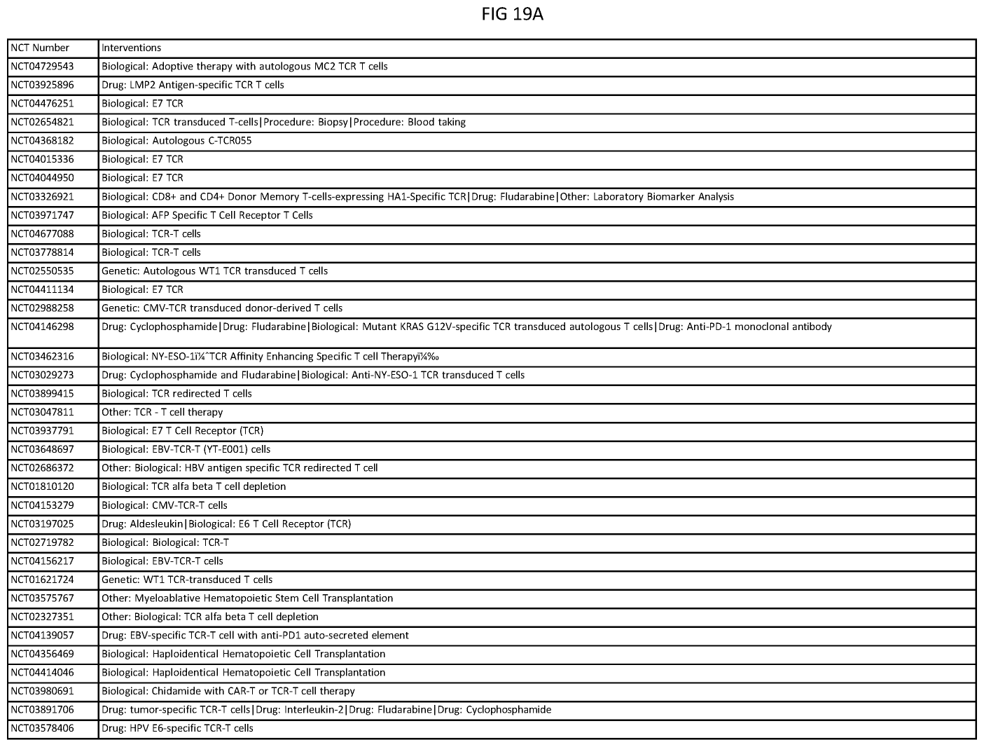

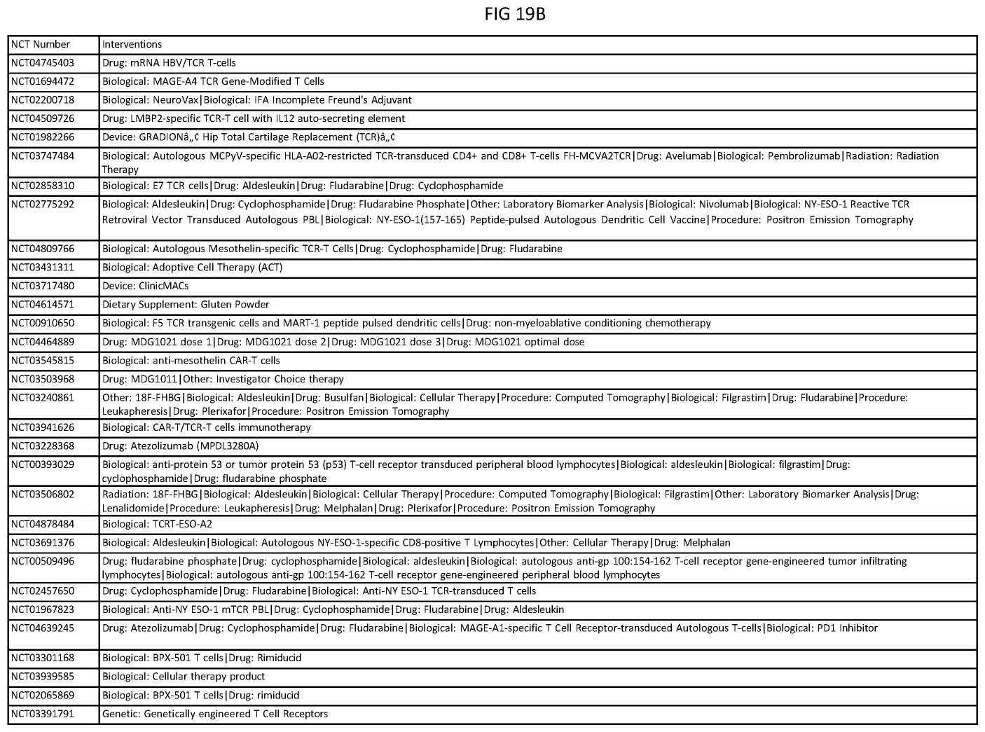

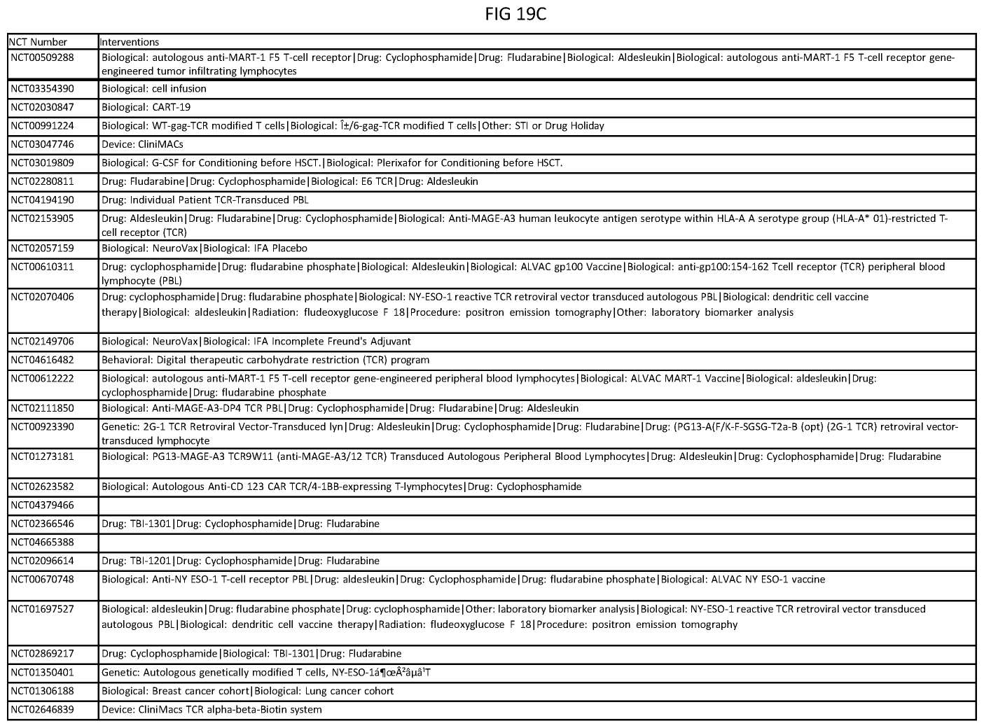

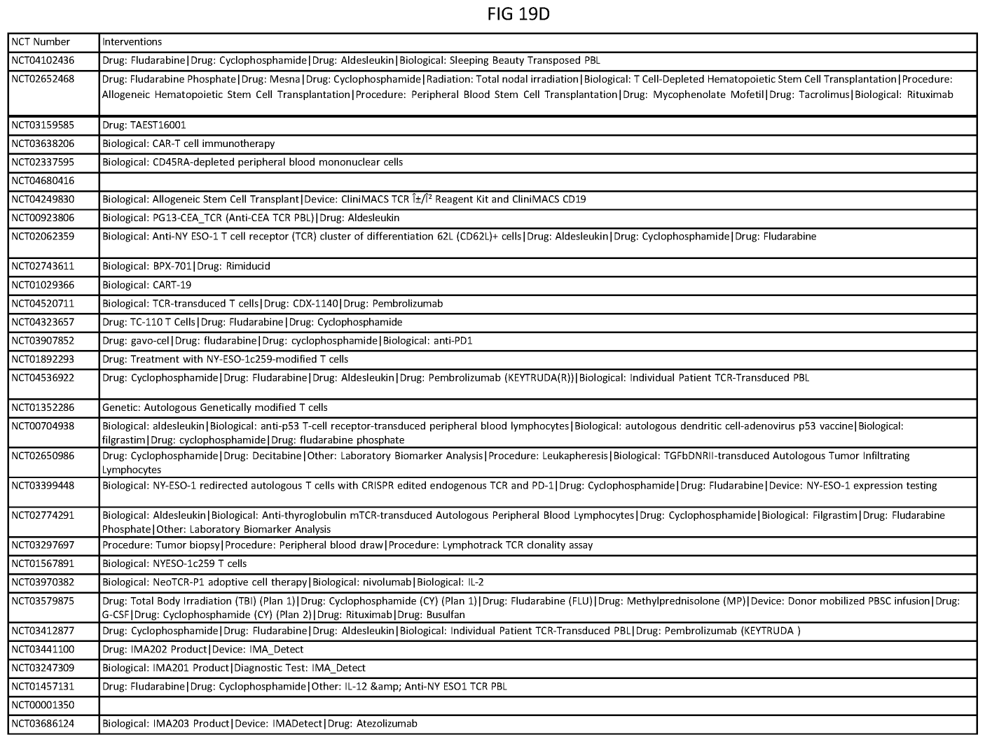

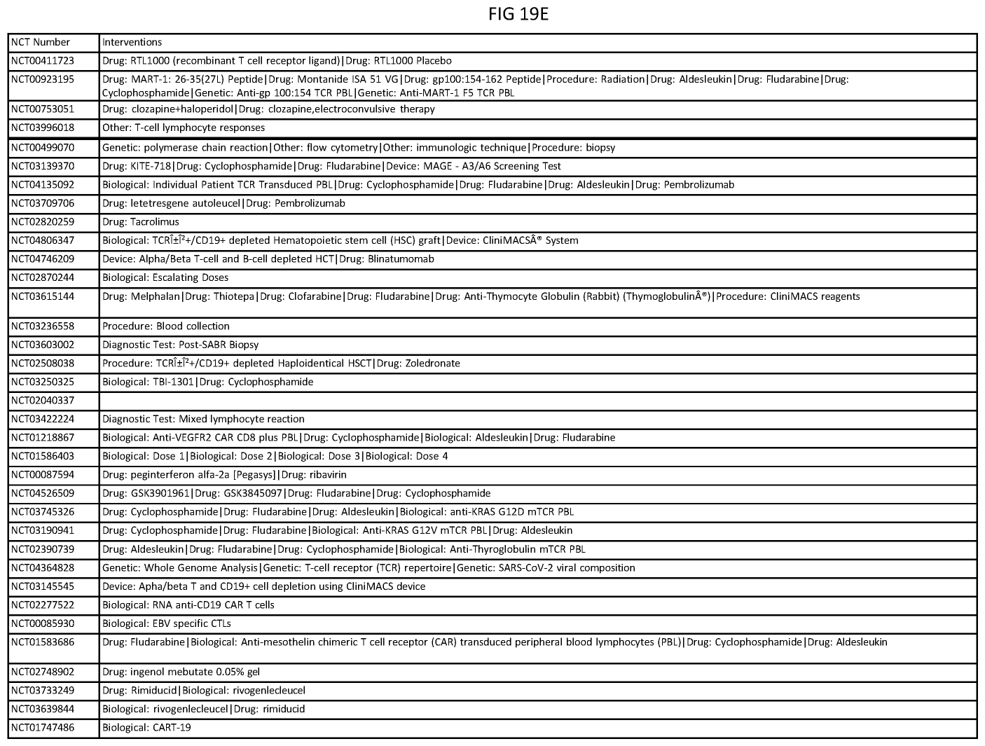

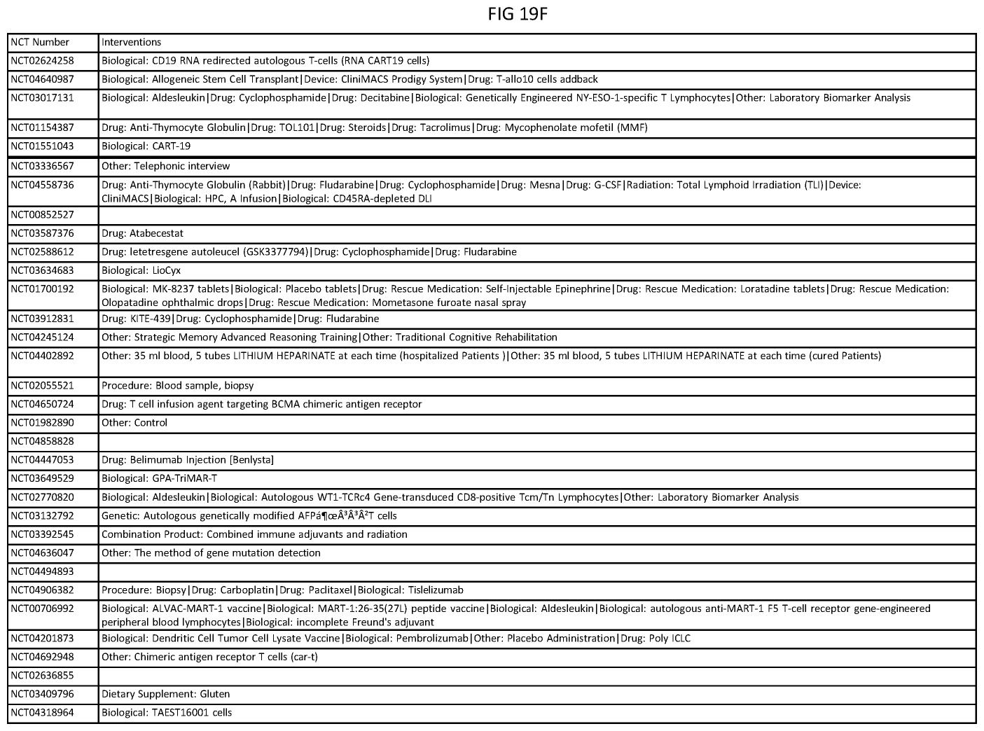

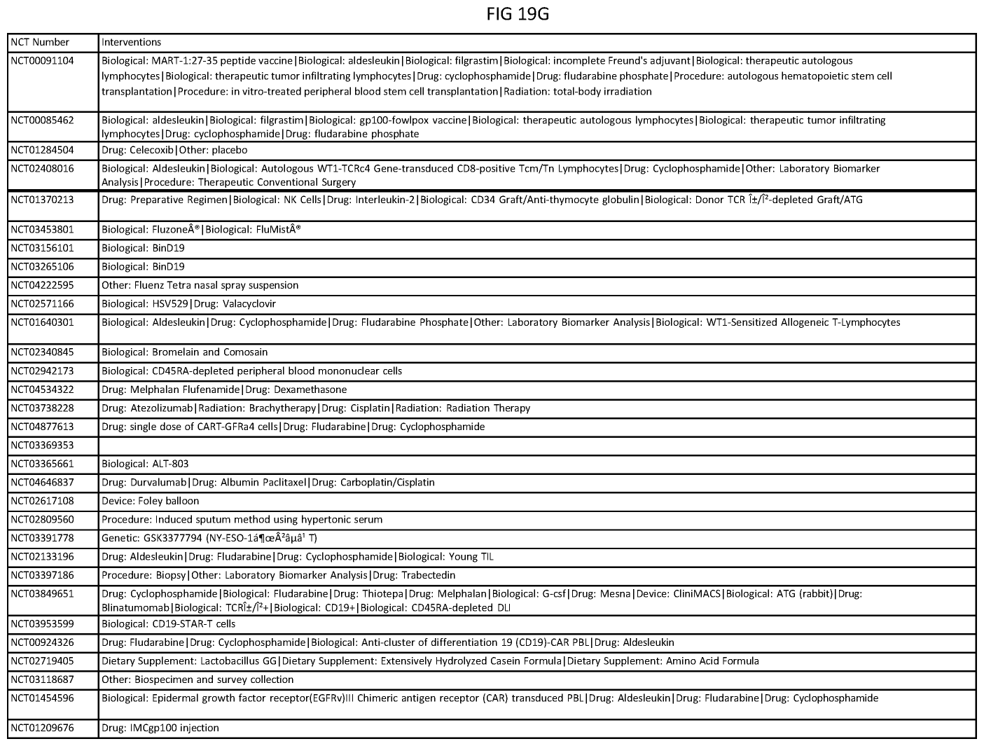

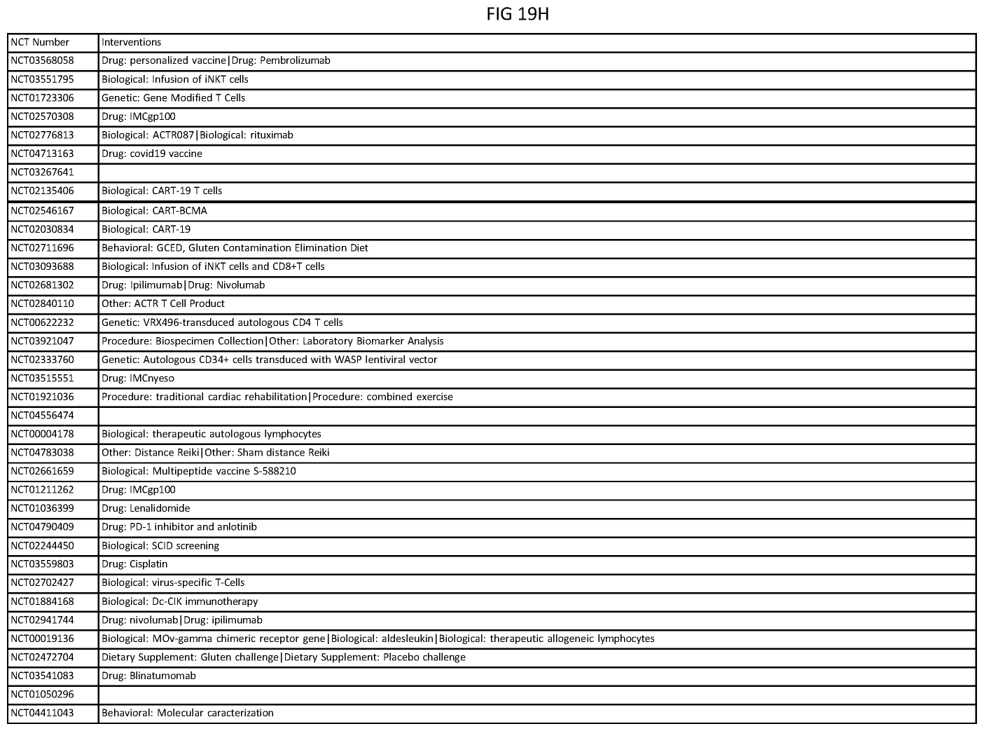

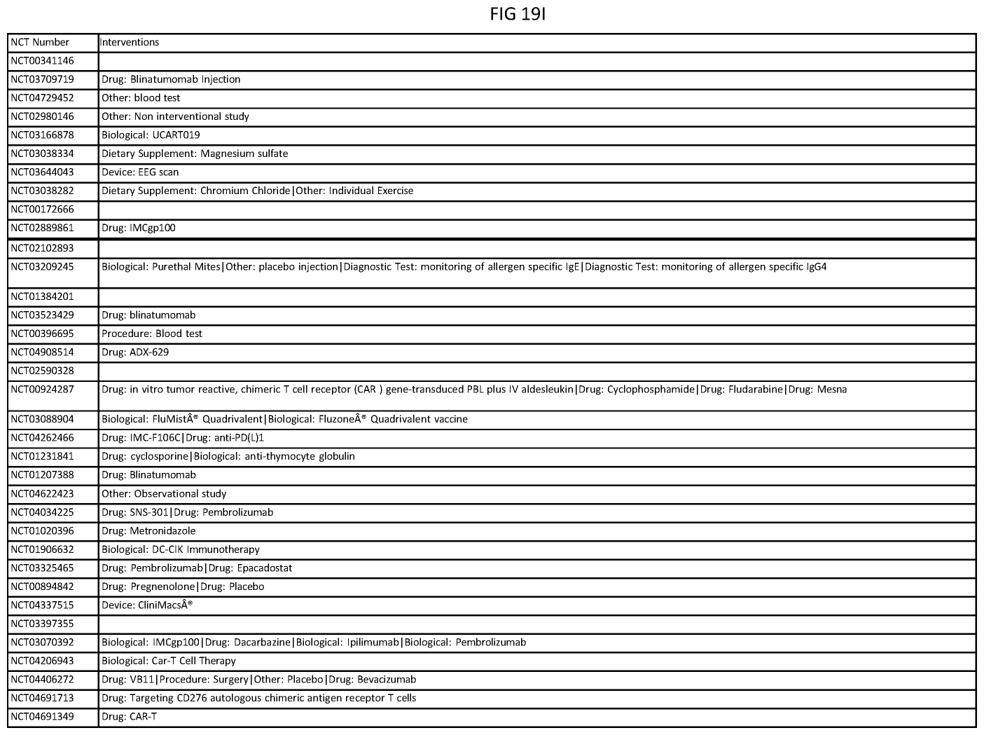

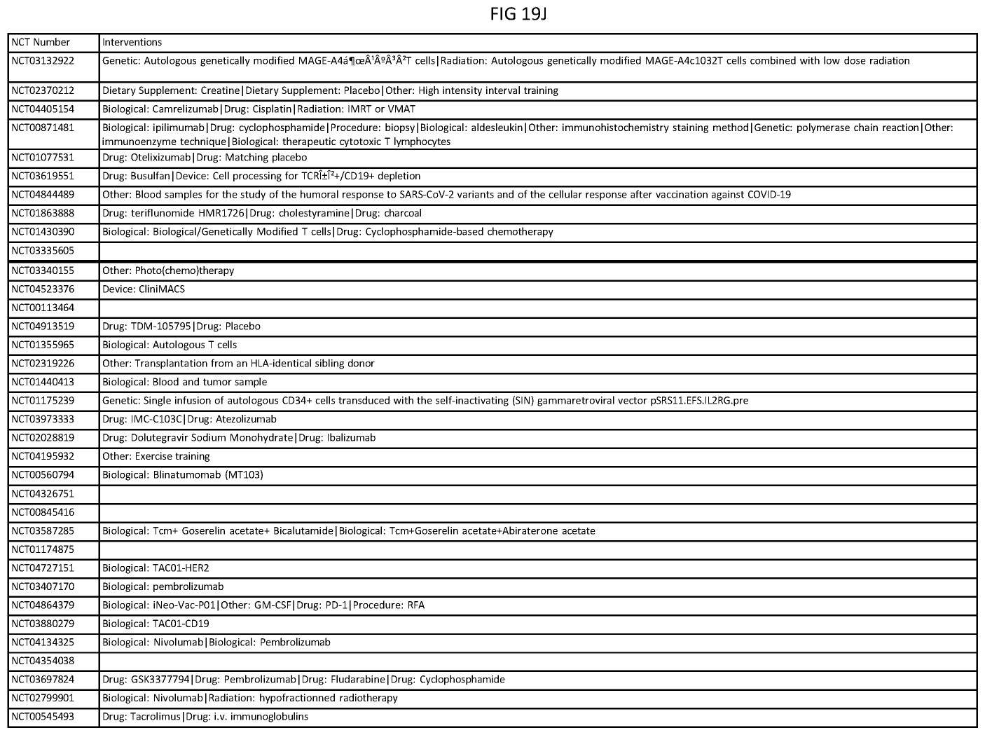

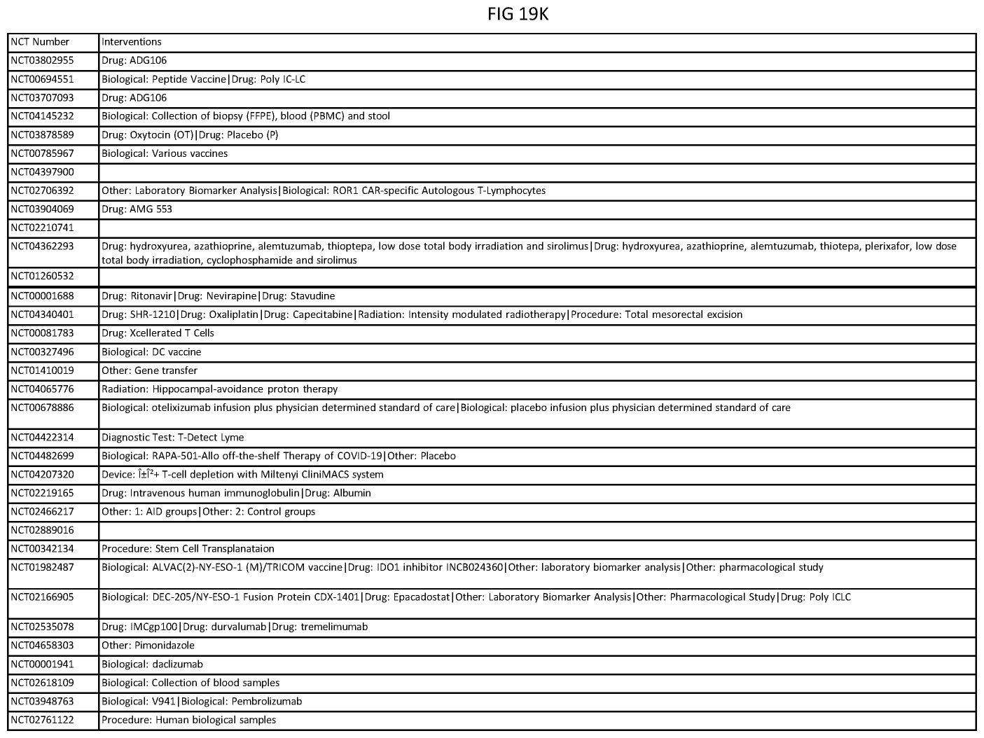

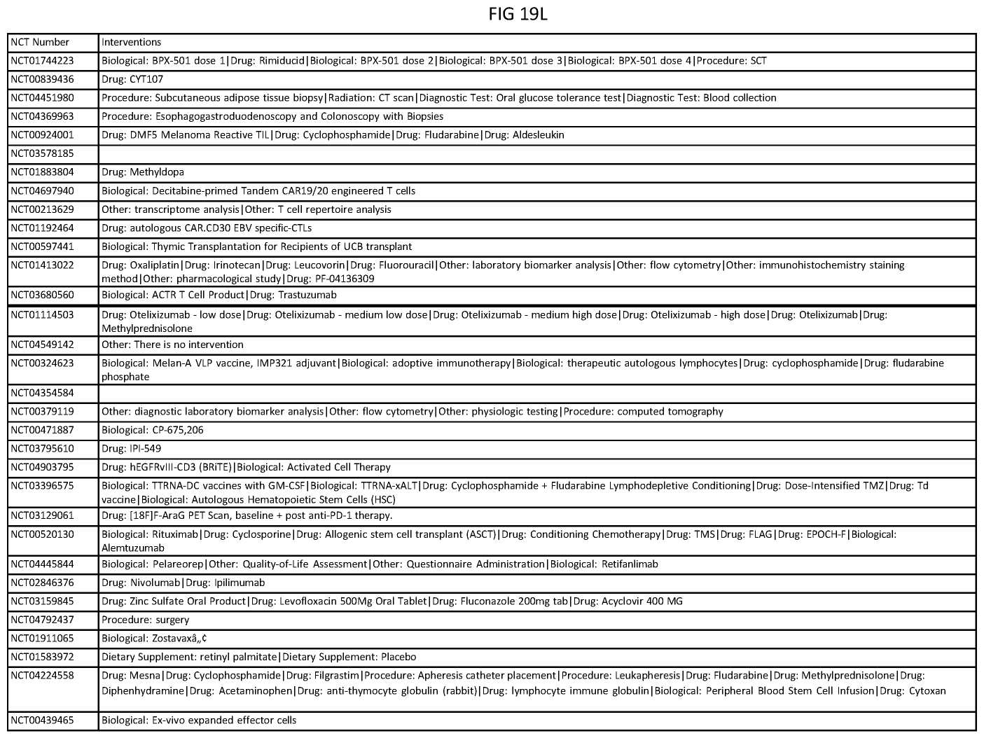

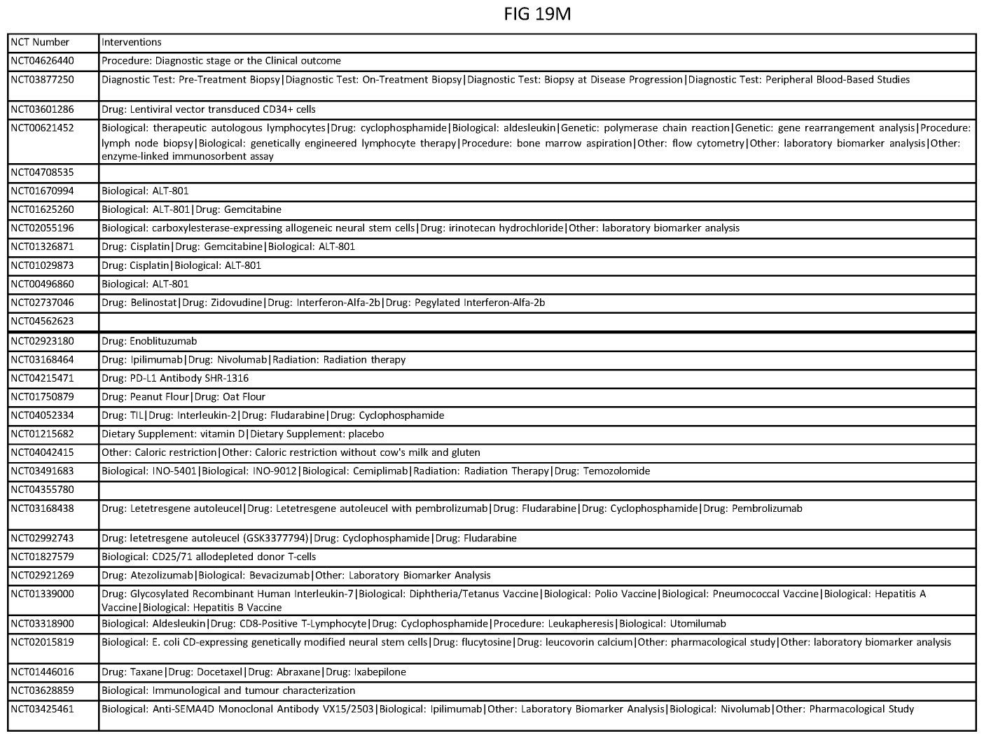

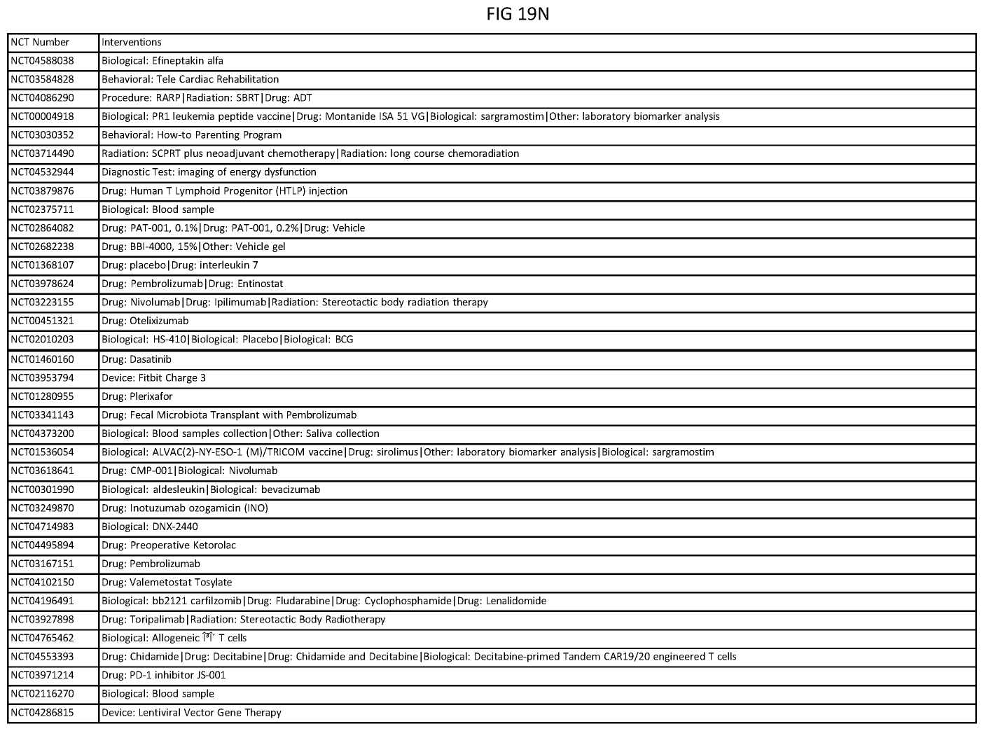

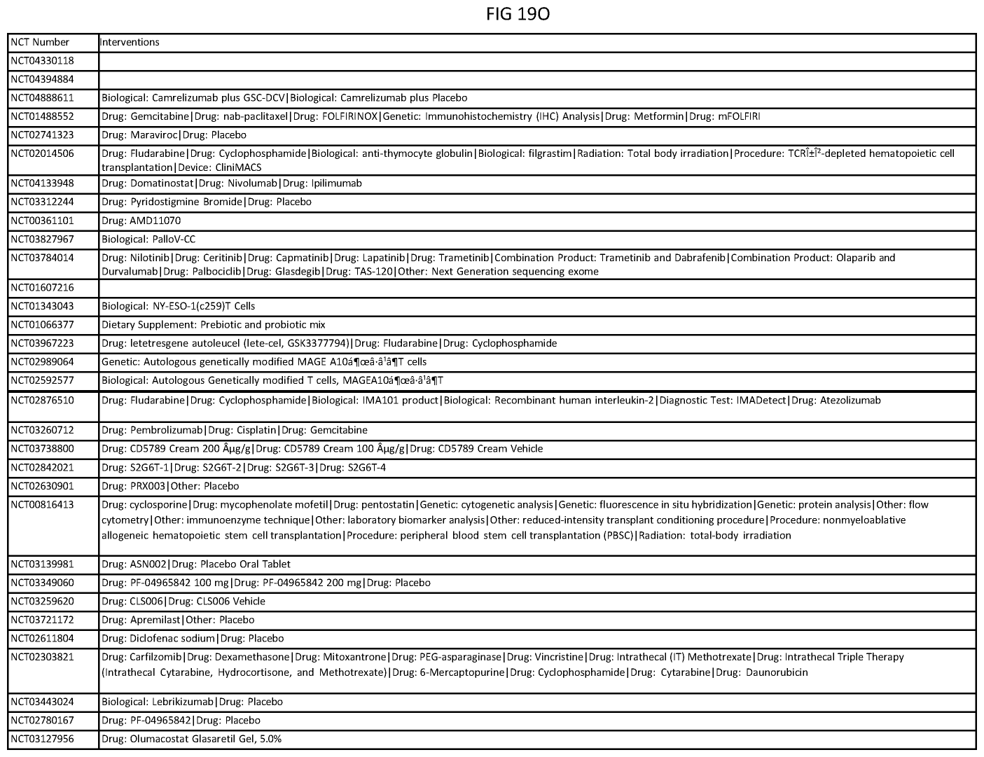

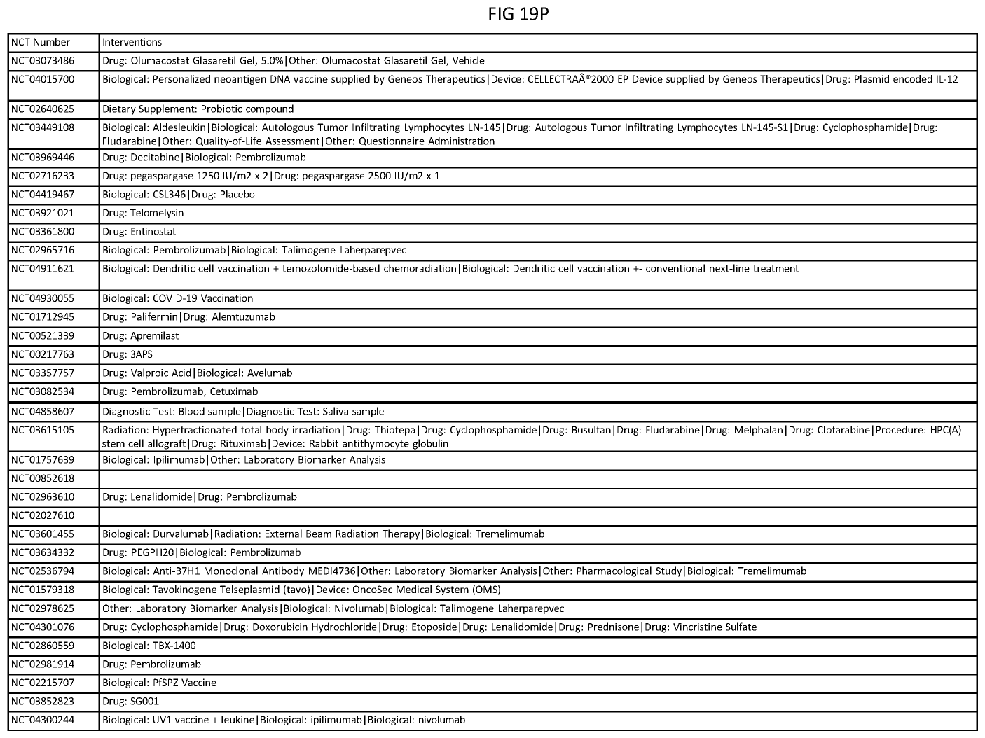

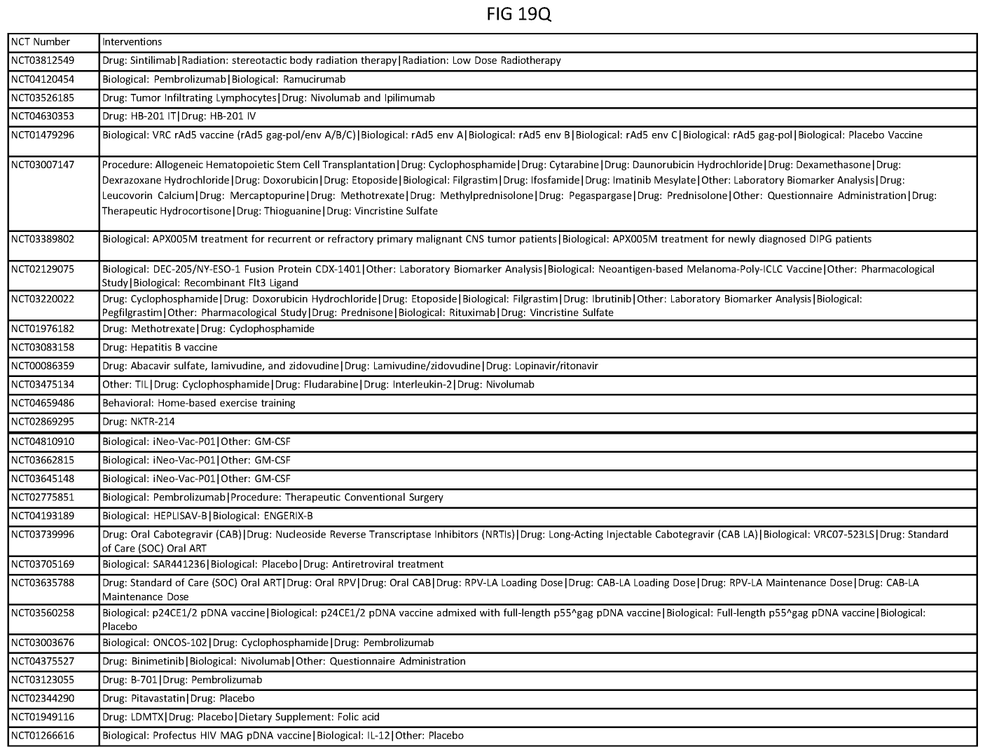

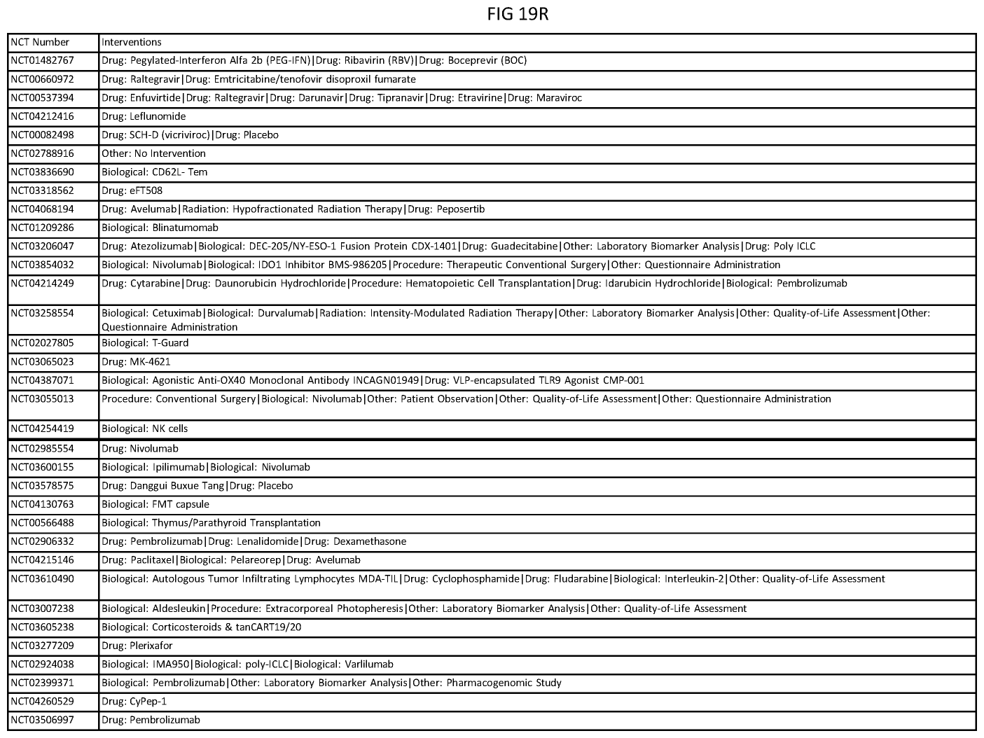

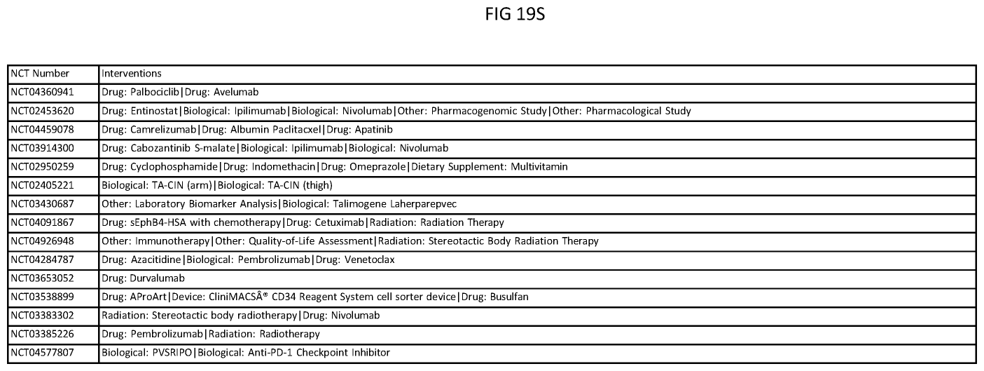

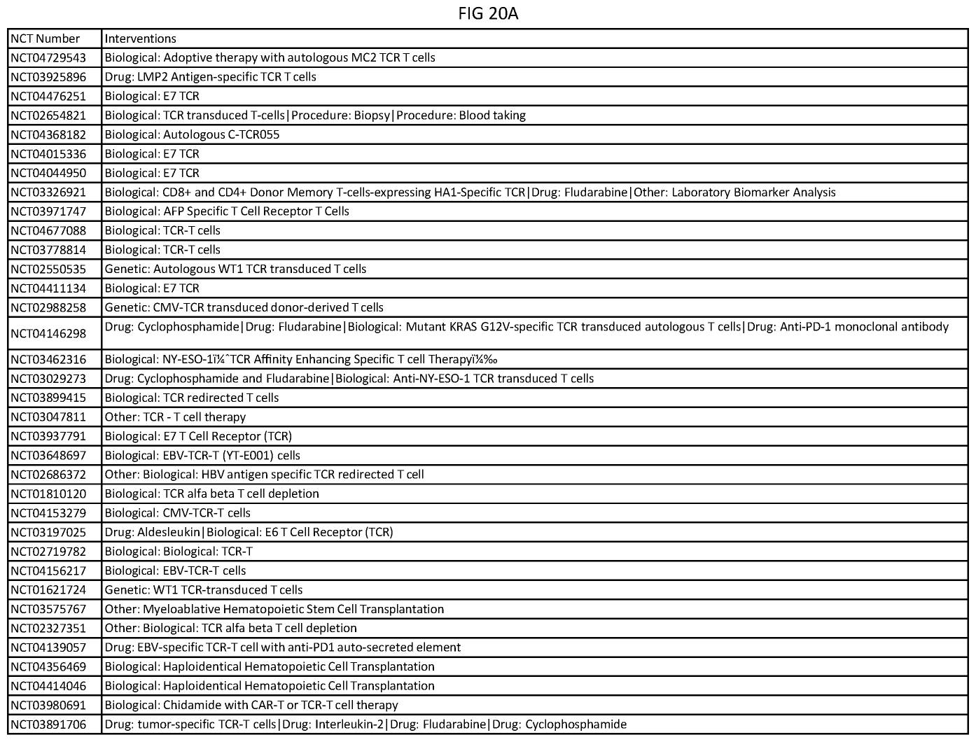

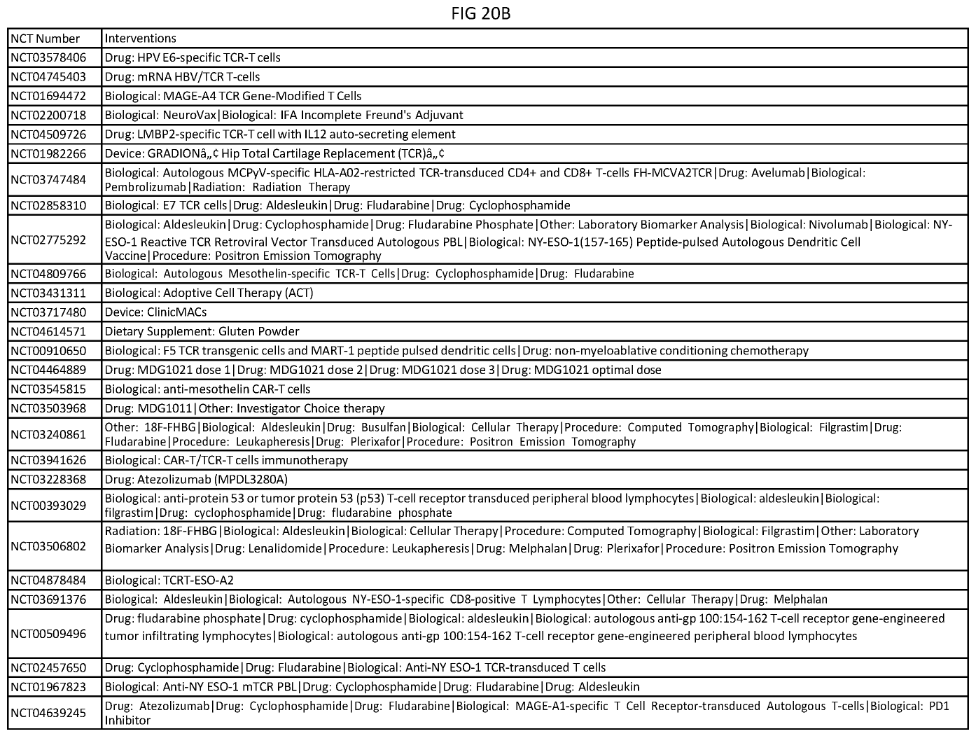

Various other chimeric antigen receptors are known in the art or may be designed by the person of skill. Such CARs include those currently being tested clinically, such as those identified in FIG. 19. The clinical trial information can be found at ClinicalTrials.gov using the provided NCT number. In alternative embodiments, an expression cassette is provided, comprising coding sequences for any of the genes of Table 1 and a CAR identified in FIG. 19. In another embodiment, an expression cassette is provided, comprising coding sequences for LTBR and a CAR identified in FIG. 19.

Other chimeric antigen receptors include those useful for treatment for autoimmune disease, such as are chimeric autoantigen receptors (CAAR). Such CAARs include DSG3-CAART and MuSK-CAART. Others may be known in the art or may be designed by the person of skill. In alternative embodiments, an expression cassette is provided, comprising coding sequences for any of the genes of Table 1 and a CAAR. In another embodiment, an expression cassette is provided, comprising coding sequences for LTBR and a CAAR. In another embodiment, an expression cassette is provided, comprising coding sequences for AHCY, DUPD1, AKR1C4, ATF6B, ITM2A, AHNAK, BATF, GPD1, GPN3, MRPL51, DBI, CALML3, IL12B, IFNL2, or ADA, and a CAAR.

Exemplary sequences for the CARs and TCRs described herein are provided in Example 2, 3, and 4 below. Other exemplary antibody sequences, useful in the construction of CARs are provided in FIG. 21.

In certain, the expression cassette comprises coding sequences for a gene of Table 1 and a follicle stimulating hormone immunoreceptor, such as that described by Powell et al, WO 2016/073456, which is incorporated herein by reference.

In certain embodiments, the expression cassette includes, in addition to the effector enhancing gene, one or more components comprising an engineered T cell receptor (TCR). For example, in one embodiment, a single expression cassette is provided which includes coding sequences for the effector enhancing gene and coding sequences for an engineered TCR comprising TCR alpha and beta chains. In one embodiment, an expression cassette is provided, comprising coding sequences for LTBR and a TCR. In another embodiment, an expression cassette is provided, comprising coding sequences for AHCY, DUPD1, AKR1C4, ATF6B, ITM2A, AHNAK, BATF, GPD1, GPN3, MRPL51, DBI, CALML3, IL12B, IFNL2, or ADA, and a TCR. In another embodiment, an expression cassette is provided, comprising coding sequences for any of the genes of Table 1 and a TCR.

Various other engineered T cell receptors are known in the art or may be designed by the person of skill. Such TCRs include those currently being tested clinically, such as those identified in FIG. 20. In alternative embodiments, an expression cassette is provided, comprising coding sequences for any of the genes of Table 1 and a TCR identified in FIG. 20. In another embodiment, an expression cassette is provided, comprising coding sequences for LTBR and a TCR identified in FIG. 20.

In certain embodiments of the nucleic acids provided herein, the effector enhancing gene is provided in an expression cassette along with the components for the CAR or TCR. In other embodiments, the effector enhancing gene is provided in an expression cassette separate from the components for the CAR or TCR.

In certain embodiments, it is desirable to downregulate or silence certain other genes in conjunction with expression of the effector-enhancing gene and CAR or TCR. Such genes include, for example, genes of the NFκB pathway, such as TNFAIP3 and NFKBIA. Compositions and methods for downregulation or silencing of genes are known in the art, and include, e.g., siRNA, miRNA, CRISPR/CAS, etc. In certain embodiments, an sgRNA is provided targeting the gene of interest, in conjunction with delivery of a CAS protein, such as described in Example 10.

In other embodiments, a composition is provided which includes a nucleic acid encoding an effector enhancing gene and a nucleic acid encoding a viral protein. Desirable viral proteins include glycoproteins such as spike proteins, E2 proteins, E1 proteins, and haemaglutinin. In one embodiment, the viral protein is a coronavirus spike protein. There are at least 16 different HAs including subtypes H1 through H16. H1, H2 and H3 are found on human influenza viruses. Another HA of interest is H5 found on the avian flu virus H5N1. For example, the viral protein may comprise any HA from subtype H1 through H16. Other suitable viral glycoproteins include, but are not limited to, Dengue virus envelope glycopolypeptide, hepatitis C virus envelope glycopolypeptide E1, hepatitis C virus envelope glycopolypeptide E2, hantavirus envelope glycopolypeptide G1, hantavirus envelope glycopolypeptide G2. The hantavirus envelope glycopolypeptides G1 and G2 are optionally from the Andes, Hantaan or Sin Nombre strain of hantavirus. Viral glycopolypeptides also include human cytomegalovirus glycopolypeptide B, human cytomegalovirus glycopolypeptide H, human herpesvirus-8 glycopolypeptide B, human herpesvirus-8 glycopolypeptide H, human metapneumovirus glycopolypeptide F, human metapneumovirus glycopolypeptide G, human parainfluenzavirus humagglutinin-neuraminidase, human parainfluenzavirus fusion glycopolypeptides, Nipah virus glycopolypeptide F, Nipah virus glycopolypeptide G, respiratory syncytial virus glycopolypeptide F, respiratory syncytial virus glycopolypeptide G, Severe Acute Respiratory Syndrome (SARS) virus spike glycopolypeptide, West Nile virus envelope glycopolypeptide and HIV-1 envelope glycopolypeptide. The HIV-1 envelope glycopolypeptide is optionally YU2 Env, SF162 Env, Env from HIV-1 B strain, Env from HIV-1 C strain and Env from HIV-1 M strain. In certain embodiments, the coding sequence for the effector-enhancing gene and/or the viral protein is/are provided as mRNA.

As used herein, the term “expression cassette” refers to a nucleic acid molecule which encodes one or more biologically useful nucleic acid sequences (e.g., a gene cDNA encoding a protein, enzyme or other useful gene product, mRNA, etc.) and regulatory sequences operably linked thereto which direct or modulate transcription, translation, and/or expression of the nucleic acid sequence(s) and its gene product(s). Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region comprising a polyadenylation site, among other elements. Thus, in addition to the coding sequence for the effector enhancing gene (and/or the CAR or TCR) the expression cassette may also include expression control sequences.

The expression control sequences include a promoter. In some embodiments, its it is desirable to utilize a promoter having high transcriptional activity. Certain strong constitutive promoters are known in the art and include, without limitation, the CMV promoter, the EF-1α promoter, EFS promoter, CBG promoter, CB7 promoter, hPGK, RPBSA, WAS promoter, etc. Alternatively, other promoters, such as regulatable (inducible) promoters [see, e.g., WO 2011/126808 and WO 2013/049493, incorporated by reference herein], or a promoter responsive to physiologic cues may be utilized. In certain embodiments it is desirable to utilize a system whereby LTBR (or other effector-enhancing gene) is expressed only after a T cell encounters its target cell and receives signal through an antigen receptor (CAR or TCR); and once the target cells are cleared and antigen receptors no longer transmit signals, the effector-enhancing gene expression decays back to the background level Thus, in certain embodiments, it is desirable to utilize a promoter that is responsive to activation of the T cell. Such promoters include, without limitation NFAT, NFκB and AP1 promoters.

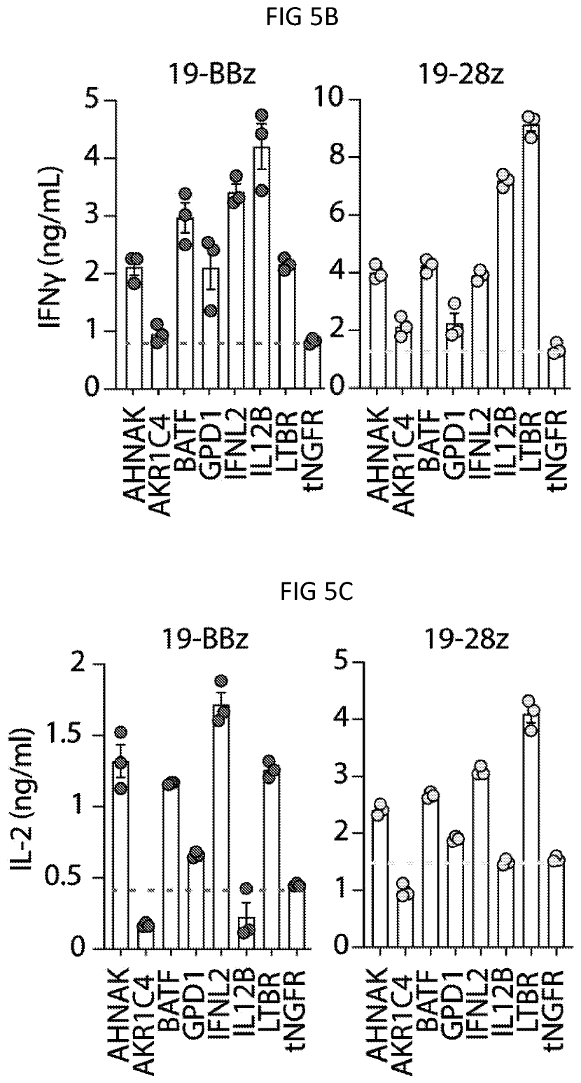

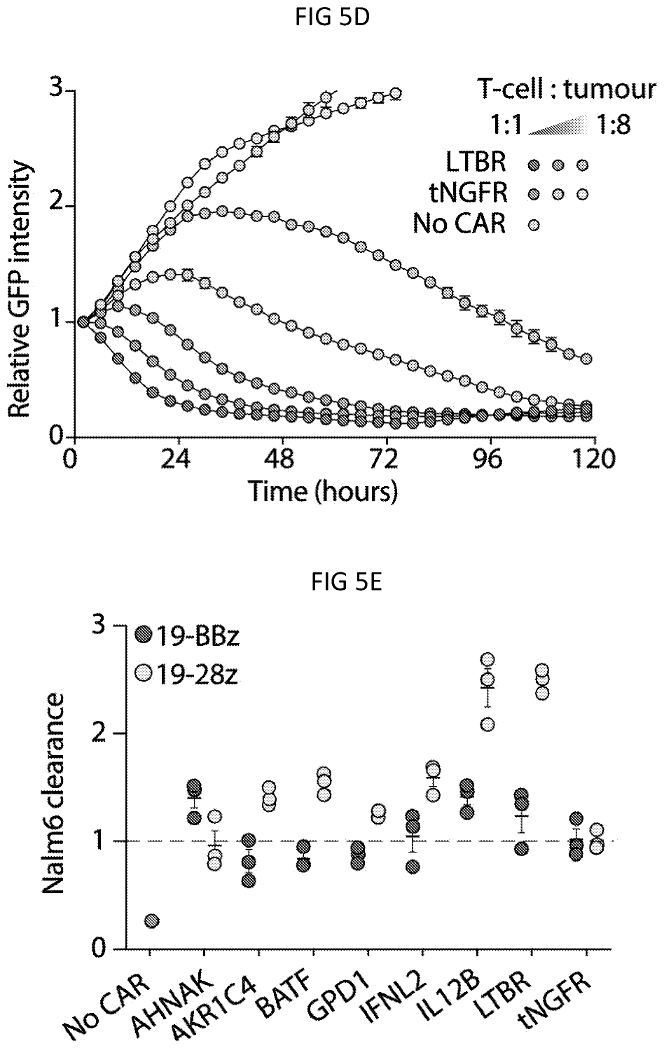

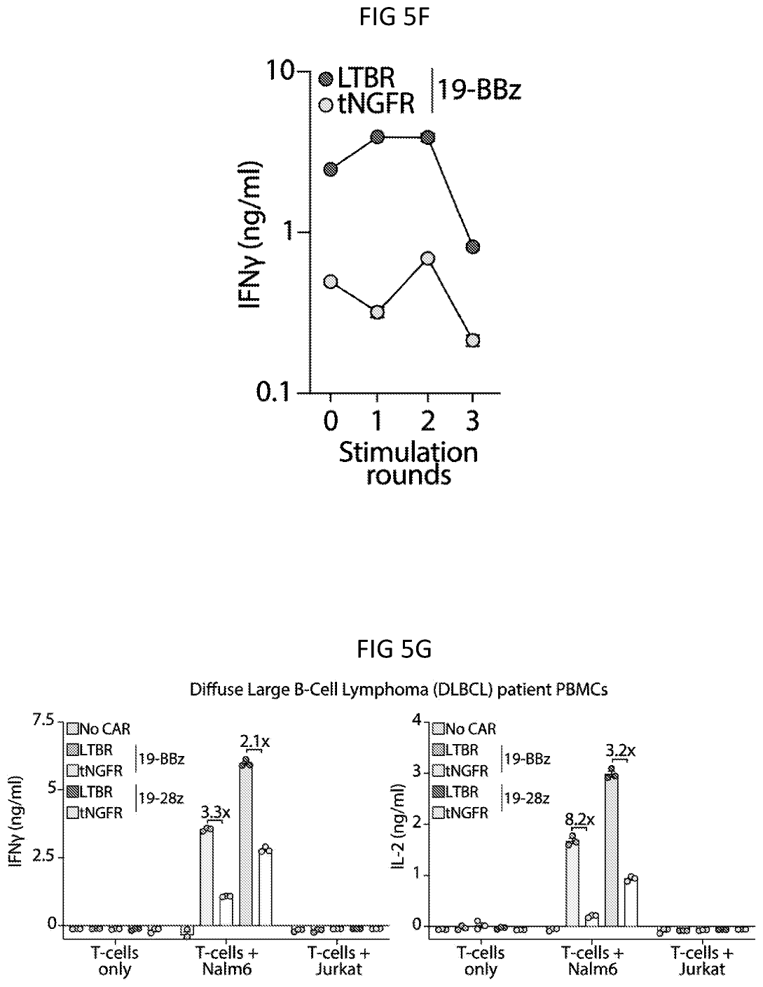

The expression cassette may also include, in certain embodiments, one or more IRES or 2A sequence to allow for expression of multiple coding sequences from the same expression cassette. As exemplified herein, in one embodiment, a CAR directed to CD19 is provided with an ORF directed to one of the genes identified in Table 1, e.g., LTBR. See, FIG. 5A, in a lentiviral vector which includes 2A sequences. An exemplary P2A sequence is shown in SEQ ID NO: 59. Construction of such cassettes and vectors are known in the art, and are described herein in the Examples. See, e.g., Sack et al. Profound Tissue Specificity in Proliferation Control Underlies Cancer Drivers and Aneuploidy Patterns. Cell. 2018 Apr. 5; 173(2): 499-514.e2 and Yang et al, A public genome-scale lentiviral expression library of human ORFs, Nat Methods. 2011 August; 8(8): 659-661, which are incorporated herein by reference.

The positioning of the coding sequences for the various components of the constructs can be varied. For example, it is, in certain aspects, desirable to position the effector-enhancing gene coding sequence upstream of the CAR coding sequence. In other embodiments, it is desirable to position the effector-enhancing gene coding sequence downstream of the CAR coding sequence. In other embodiments, a selection marker gene is included in the construct.

Provided herein, in certain aspects, are compositions which include modified lymphocytes which comprise a nucleic acid and/or expression cassette as described herein. In one embodiment, the host lymphocyte is a T cell. In another embodiment, the host lymphocyte is a natural killer (NK) cell. In certain embodiments, the composition comprises a population of cells which includes a mixed population of lymphocytes (e.g., alpha beta T cells and NK T cells). In other embodiments, the composition comprises cells which includes a population which is enriched for a particular lymphocyte population.

As used herein, the phrase “T cell” refers to a lymphocyte that expresses a T cell receptor molecule. T cells include human alpha beta (αβ) T cells and human gamma delta (γδ) T cells. T cells include, but are not limited to, naive T cells, stimulated T cells, primary T cells, cultured T cells, immortalized T cells, helper T cells, cytotoxic T cells, memory T cells, regulatory T cells, natural killer T cells, combinations thereof, or subpopulations thereof. T cells can be CD4+, CD8+, or CD4+ and CD8+. T cells can also be CD4−, CD8−, or CD4− and CD8−. T cells can be helper cells, for example helper cells of type T_{H}1, T_{H}2, T_{H}3, T_{H}9, T_{H}17, or T_{FH}. T cells can be cytotoxic T cells. T cells can also be regulatory T cells. Regulatory T cells (Tregs) can be FOXP3+ or FOXP3−. T cells can be alpha/beta T cells or gamma/delta T cells. In some cases, the T cell is a CD4+CD25^{hi}CD127^{lo} regulatory′ T cell. In some cases, the T cell is a regulatory T cell selected from the group consisting of type 1 regulatory (Tr1), T_{H}3, CD8+CD28−, Treg17, and Qa-1 restricted T cells, or a combination or sub-population thereof. In some cases, the T cell is a FOXP3⁺ T cell. In some cases, the T cell is a CD4⁺CD25^{lo}CD127^{hi} effector T cell. In some cases, the T cell is a CD4⁺CD25^{lo}CD127^{hi}CD45^{hi}CD45RO− naive T cell. In certain embodiments the T cell is a Vγ9Vδ2 T cell. In some embodiments, the T cell expresses a viral antigen. In other embodiments, the T cell expresses a cancer antigen. A T cell can be a recombinant T cell that has been genetically manipulated.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

### Methods

### Methods of Making Modified Host Cells

Also provided herein are methods of making the modified cells and compositions containing modified cells as described herein. Methods of modifying cells, e.g., lymphocytes, to introduce an exogenous sequence, such as an expression cassette or expression vector comprising a coding sequence for an effector enhancing gene, a CAR or TCR, or more than one of these sequences, are known in the art. For example, see, e.g., WO 2016/109410 A2, which is incorporated herein by reference. In certain embodiments, more than one exogenous sequence is introduced.

By the term “modified” as used herein, is meant a changed state or structure of a molecule or cell of the invention. Molecules may be modified in many ways, including chemically, structurally, and functionally. Cells may be modified through the introduction of nucleic acids. Modifying can refer to altering expression of a gene in a lymphocyte, for example, by introducing an exogeneous nucleic acid that encodes the gene.

The lymphocytes provided herein can be genetically modified, e.g., by transfection, transduction, or electroporation, to express a nucleic acid sequence encoding a gene, as described herein. Depending on the clinical context, e.g., patient's condition or condition to be treated, prolonged or permanent expression of the gene and/or, e.g., for robust and long-lasting CAR activity, e.g., anti-tumor activity, may be desirable. In such embodiments, the lymphocytes are genetically modified, e.g., transduced, e.g., virally transduced, using vectors comprising nucleic acid sequences encoding a gene disclosed herein to confer a desired effector function. In other embodiments, transient expression of the gene is desirable. In such embodiments, the use of, e.g., mRNA or a regulatable promoter to express the effector-enhancing gene, may be used.

Methods of introducing and expressing genes into a cell are known in the art. In the context of an expression vector, the vector can be readily introduced into a host cell, e.g., mammalian, bacterial, yeast, or insect cell by any known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. Sec, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of targeted delivery of nucleic acids are available, such as delivery of polynucleotides with targeted nanoparticles or other suitable sub-micron sized delivery system. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant nucleic acid sequence in the host cell, a variety of assays may be performed. Such assays include, for example, Southern and Northern blotting, RT-PCR and PCR, biochemical assays, such as detecting the presence or absence of a particular peptide, e.g., by immunological means (ELISAs and Western blots).

In certain embodiments, an expression vector is provided which includes the coding sequence for an effector-enhancing gene. In other embodiments, the expression vector includes the coding sequence for one or more components of a CAR or TCR. In other embodiments, a separate expression vector is provided which includes the coding sequence for one or more components of a CAR or TCR. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. In one embodiment, the expression vector is a lentivirus. If more than one expression vector is utilized, each expression vector may be individually selected from amongst those known in the art.

Provided herein is a method of making a population of immune effector cells (e.g., T cells, NK cells) that are modified to express an effector-enhancing gene as described herein, and, optionally, to express a CAR or TCR. Methods for making such immune cells include introducing an exogenous nucleic acid encoding a gene selected from those of Table 1 into the cell. In the example described below, a method of making modified T cells is described for convenience. However, alternative embodiments are envisioned using other kinds of immune cells, e.g., NK T cells or NK cells. Suitable methods are known in the art.

Briefly, an exemplary method includes providing a population of immune effector cells (e.g., T cells), and optionally, removing T regulatory cells, e.g., CD25+ T cells, from the population. In certain embodiments, the population of immune effector cells are autologous to the subject who the cells will be administered to for treatment. In certain embodiments, the population of immune effector cells comprises autologous Vγ9Vδ2 T cells. In certain embodiments, the population of immune effector cells are allogeneic to the subject who the cells will be administered to for treatment. In one embodiment, the T regulatory cells, e.g., CD25+ T cells, are removed from the population using an anti-CD25 antibody, or fragment thereof, or a CD25-binding ligand, e.g., IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein. In one embodiment, the T regulatory cells, e.g., CD25+ T cells, are removed from the population using an anti-CD25 antibody molecule, or fragment thereof. In another embodiment, CD25+ cells are not removed.

Another exemplary method includes providing a population of immune effector cells (e.g., T cells), and enriching the population for CD8+ cells and/or CD4+ cells. In one embodiment, population is enriched for CD8+ and/or CD4+ T cells using an anti-CD8 and/or anti-CD4 antibody, or fragment thereof, or a CD8-binding ligand and/or CD4-binding ligand. In one embodiment, the anti-CD4 and/or anti-CD8 antibody, or fragment thereof, or anti-CD4 and/or anti-CD8-binding ligand is conjugated to a substrate, e.g., a bead, or is otherwise coated on a substrate, e.g., a bead.

In certain embodiments, the method further comprises delivering to a cell one or more vectors comprising a nucleic acid encoding a gene selected from those of Table 1, e.g., LTBR, and optionally, a CAR or TCR. In certain embodiments, the vector is selected from DNA, a RNA, a plasmid, a lentivirus vector, adenoviral vector, or a retrovirus vector. In certain embodiments embodiment, a cell from a population of T cells is transduced with a vector once, e.g., within one day after population of immune effector cells are obtained from a blood sample from a subject, e.g., obtained by apheresis. In certain embodiments, the method further comprises generating a population of RNA-engineered cells transiently expressing exogenous RNA from the population of T cells. The method comprises introducing an in vitro transcribed RNA or synthetic RNA into a cell from the population, where the RNA comprises a nucleic acid encoding a gene of Table 1, e.g., LTBR. For example, in certain embodiments, the population of T cells may be transduced with a vector that comprises a nucleic acid encoding a CAR and then the same population of cells may be introduced an mRNA encoding a gene of Table 1, e.g., LTBR.

In one embodiment, modified cells described herein are expanded. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded in culture for 3 or 4 days, and the resulting cells are more potent than the same cells expanded in culture for 9 days under the same culture conditions. Potency can be defined, e.g., by various T cell functions, e.g., proliferation, target cell killing, cytokine production, activation, migration, or combinations thereof.

### Methods of Treatment

Also provided herein, in certain aspects, are methods of treating cancer in a subject. In certain embodiments, the method includes administering to the subject a cell that expresses an effector-enhancing gene as described herein such that the cancer is treated in the subject. In certain embodiments, the cell further expresses a CAR. In other embodiments, the cell further expresses a TCR. As used herein in describing the methods of treatment, LTBR is utilized as an exemplary effector-enhancing gene, for convenience. However, in alternative embodiments, other genes of Table 1 are utilized. In certain embodiments, the method includes obtaining cells from a patient, modifying the cells as described herein, and administering the cells to the patient.

An example of a cancer that is treatable by the effector-enhancing gene-expressing cell (e.g., LTBR CART or LTBR TCR-T cell) is a hematological cancer. In certain embodiments, the hematologic cancer includes but is not limited to leukemia (such as acute myelogenous leukemia, chronic myelogenous leukemia, acute lymphoid leukemia, chronic lymphoid leukemia and myelodysplastic syndrome) and malignant lymphoproliferative conditions, including lymphoma (such as multiple myeloma, non-Hodgkin's lymphoma, Burkitt's lymphoma, and small cell- and large cell-follicular lymphoma). In other embodiments, a hematologic cancer can include minimal residual disease, MRD, e.g., of a leukemia, e.g., of AML or MDS. In certain embodiments, the cancer is a solid tumor. In certain embodiments, the cancer is pancreatic cancer, melanoma, multiple myeloma, sarcoma, or lung cancer. In another embodiment, the cancer is a cancer associated with follicle stimulating hormone. Such cancers include breast cancer, lung cancer, prostate cancer, colorectal cancer, esophageal cancer, stomach cancer, bladder cancer, pancreatic cancer, kidney cancer, cervical cancer, liver cancer, ovarian cancer, and testicular cancer. In another embodiment, the cancer is a cancer included in the listings in FIG. 19 or FIG. 20.

In certain embodiments, the CAR is selected from Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kyrmriah®).

In another embodiment, the subject has a virally-driven cancer. In certain embodiments, the virally-driven cancer is selected from the following:

TABLE 3

| Virus | Conditions |
|---|---|
| Epstein-Barr Virus (EBV) | Burkitt's Lymphoma |
| Hepatitis B Virus (HBV) | Liver Cancer |
| Hepatitis C Virus (HCV) | Liver Cancer |
| Human Herpesvirus 8 (HH8) | Kaposi's Sarcoma |
| Human Papillomavirus (HPV) | Cervical Cancer, Head |
| | and Neck Cancers, Anal, |
| | Oral, Pharyngeal, |
| | and Penile Cancers |
| Human T-cell Lymphotropic | Adult T-cell Leukemia |
| Virus 1 (HTLV) | |
| Merkel Cell Polyomavirus | Skin Cancer (Merkel |
| | Cell Carcinoma) |

In another aspect, methods of treating an autoimmune disease in a subject are provided. Autoimmune diseases are conditions arising from abnormal immune attack to the body, and they substantially increase the morbidity, mortality and healthcare costs worldwide. As T cells play a key role in the process of autoimmune diseases, engineered T-cell therapy has emerged and is also regarded as a potential approach to overcome current roadblocks in the treatment of autoimmune diseases. Either self-reactive or autoantibodies play a key role in the process of autoimmune diseases. Thus, engineering T cells to express a chimeric autoantibody receptor (CAAR) is a strategy for treatment for autoimmune disease. In one embodiment, the CAR comprises a CAAR. See, e.g., Zhang et al, Chimeric antigen receptor T-cell therapy beyond cancer: current practice and future prospects, Immunotherapy, 2020 September; 12(13): 1021-1034. doi: 10.2217/imt-2020-0009. Epub 2020 Jul. 30, which is incorporated herein by reference. Autoimmune diseases include Pemphigus vulgaris (PV) (e.g., DSG3-CAAR-T) and lupus (e.g., MuSK-CAAR-T)). Other autoimmune diseases include type 1 diabetes, autoimmune thyroid disease, rheumatoid arthritis (RA), inflammatory bowel disease, colitis, systemic lupus erythematosus, and multiple sclerosis (MS). See, e.g., Chen et al, Immunotherapy Deriving from CAR-T Cell Treatment in Autoimmune Diseases, Journal of Immunology Research Volume 2019. Dec. 31, 2019, which is incorporated herein by reference.

In one aspect, the methods comprise administering to the subject in need thereof an effective amount of an effector-enhancing gene-expressing cell (e.g., LTBR CART or LTBR TCR-T cell) described herein in combination with an effective amount of another therapy. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

An effector-enhancing gene-expressing cell (e.g., LTBR CART or LTBR TCR-T cell) described herein and the at least one additional therapeutic agent can be administered simultaneously, in the same or in separate compositions, or sequentially. For sequential administration, the effector-enhancing gene-expressing cell (e.g., LTBR CART or LTBR TCR-T cell) described herein can be administered first, and the additional agent can be administered second, or the order of administration can be reversed.

The effector-enhancing gene-expressing cell (e.g., LTBR CART or LTBR TCR-T cell) and/or other therapeutic agents, procedures or modalities can be administered during periods of active disorder, or during a period of remission or less active disease. The effector-enhancing gene-expressing cell (e.g., LTBR CART or LTBR TCR-T cell) can be administered before the other treatment, concurrently with the treatment, post-treatment, or during remission of the disorder.

When administered in combination, the effector-enhancing gene-expressing cell (e.g., LTBR CART or LTBR TCR-T cell) and the additional agent (e.g., second or third agent), or all, can be administered in an amount or dose that is higher, lower or the same than the amount or dosage of each agent used individually, e.g., as a monotherapy. In certain embodiments, the administered amount or dosage of the effector-enhancing gene-expressing cell (e.g., LTBR CART or LTBR TCR-T cell), the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the effector-enhancing gene-expressing cell (e.g., LTBR CART or LTBR TCR-T cell), the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, an effector-enhancing gene-expressing cell (e.g., LTBR CART or LTBR TCR-T cell) described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxan, fludarabine, cyclosporin, FK506, rapamycin, mycophenolic acid, steroids, FR901228, cytokines, irradiation, or a peptide vaccine, such as that described in Izumoto et al. 2008 J Neurosurg 108:963-971.

In certain instances, an effector-enhancing gene-expressing cell (e.g., LTBR CART or LTBR TCR-T cell) as described herein are combined with other therapeutic agents, such as other anti-cancer agents, anti-allergic agents, anti-nausea agents (or anti-emetics), pain relievers, cytoprotective agents, and combinations thereof.

In certain embodiments, an effector-enhancing gene-expressing cell (e.g., LTBR CART or LTBR TCR-T cell) herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide), or a checkpoint inhibitor (e.g., a PD-1 or PD-L1 inhibitor, e.g., Pembrolizumab (Keytruda), Nivolumab (Opdivo), Cemiplimab (Libtayo), Atezolizumab (Tecentriq), Avelumab (Bavencio), Durvalumab (Imfinzi)).

General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (Dauno Xome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Treatment with a combination of a chemotherapeutic agent and an effector-enhancing gene-expressing cell (e.g., LTBR CART or LTBR TCR-T cell) described herein can be used to treat a hematologic cancer described herein, e.g., AML. In embodiments, the combination of a chemotherapeutic agent and an effector-enhancing gene-expressing cell (e.g., LTBR CART or LTBR TCR-T cell) is useful for targeting, e.g., killing, cancer stem cells, e.g., leukemic stem cells, e.g., in subjects with AML. In embodiments, the combination of a chemotherapeutic agent and an effector-enhancing gene-expressing cell (e.g., LTBR CART or LTBR TCR-T cell) is useful for treating minimal residual disease (MRD). MRD refers to the small number of cancer cells that remain in a subject during treatment, e.g., chemotherapy, or after treatment. MRD is often a major cause for relapse. The present invention provides a method for treating cancer, e.g., MRD, comprising administering a chemotherapeutic agent in combination with an effector-enhancing gene-expressing cell (e.g., LTBR CART or LTBR TCR-T cell), e.g., as described herein.

In certain embodiments, the chemotherapeutic agent is administered prior to administration of the effector-enhancing gene-expressing cell (e.g., LTBR CART or LTBR TCR-T cell). In chemotherapeutic regimens where more than one administration of the chemotherapeutic agent is desired, the chemotherapeutic regimen is initiated or completed prior to administration of effector-enhancing gene-expressing cell (e.g., LTBR CART or LTBR TCR-T cell). In embodiments, the chemotherapeutic agent is administered at least 1 day, 2 days, 3 days, 4 days, 5 days, 6 days, 7 days, 8 days, 9 days, 10 days, 11 days, 12 days, 13 days, 14 days, 15 days, 20 days, 25 days, or 30 days prior to administration of the effector-enhancing gene-expressing cell (e.g., LTBR CART or LTBR TCR-T cell). In embodiments, the chemotherapeutic regimen is initiated or completed at least 1 day, 2 days, 3 days, 4 days, 5 days, 6 days, 7 days, 8 days, 9 days, 10 days, 11 days, 12 days, 13 days, 14 days, 15 days, 20 days, 25 days, or 30 days prior to administration of the effector-enhancing gene-expressing cell (e.g., LTBR CART or LTBR TCR-T cell).

When “an immunologically effective amount,” “an anti-tumor effective amount,” “a tumor-inhibiting effective amount,” or “effective amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). As used herein, the term “effective amount” of an agent is that amount sufficient to effect beneficial or desired results, for example, clinical results, and, as such, an “effective amount” depends upon the context in which it is being applied. For example, in the context of administering an agent that treats a tumor, an effective amount of an agent is, for example, an amount sufficient to reduce or decrease a size of a tumor or to inhibit a tumor growth, as compared to the response obtained without administration of the agent. The term “effective amount” can be used interchangeably with “effective dose,” “therapeutically effective amount,” or “therapeutically effective dose.”

Also provided herein is a method of vaccinating a subject with a combination vaccine including at least two nucleic acid sequences encoding at least one effector-enhancing gene and at least one viral protein. In one embodiment, the effector-enhancing gene is LTBR. In another embodiment, the viral protein is a coronavirus spike protein. Some embodiments provide vaccines comprising an RNA polynucleotide having an open reading frame encoding a effector-enhancing gene, an RNA polynucleotide having an open reading frame encoding a effector-enhancing gene a viral protein, and a pharmaceutically acceptable carrier or excipient, formulated within a cationic lipid nanoparticle (LNP). The vaccines described herein (e.g., LNP-encapsulated mRNA vaccines) produce prophylactically- and/or therapeutically-efficacious levels, concentrations and/or titers of antigen-specific antibodies in the blood or serum of a vaccinated subject. Sec, e.g., US 2018/0311336A1, which is incorporated herein by reference in its entirety.

As used herein, the term “treatment,” and variations thereof such as “treat” or “treating.” refers to clinical intervention in an attempt to alter the natural course of the individual being treated and can be performed either for prophylaxis or during the course of clinical pathology. Desirable effects of treatment include, but are not limited to, preventing or reducing the occurrence or recurrence of disease, alleviation of symptoms, diminishment of any direct or indirect pathological consequences of the disease, decreasing the rate of disease progression, amelioration or palliation of the disease state, and remission or improved prognosis. In certain embodiments, compositions described herein are used to delay development of a disease or to slow the progression of a disease.

Likewise, as used herein, the term “treatment of cancer” or “treating cancer” can be described by a number of different parameters including, but not limited to, reduction in the size of a tumor in an animal having cancer, reduction in the growth or proliferation of a tumor in an animal having cancer, preventing metastasis or reducing the extent of metastasis, and/or extending the survival of an animal having cancer compared to control. In certain embodiments, treatment results in a reduced risk of distant recurrence or metastasis.

The term “cancer” as used herein refers to any disease, condition, trait, genotype or phenotype characterized by unregulated cell growth or replication. In certain embodiments, administration of the compositions disclosed herein, e.g., according to the methods disclosed herein, treats a cancer. In certain embodiments, the cancer is selected from the group consisting of adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, hepatocellular carcinoma (HCC), non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor, secondary cancers caused by cancer treatment, and any combination thereof. In certain embodiments, the cancer is one of those listed in FIG. 19 or FIG. 20.

### Screening Methods

Disclosed herein are methods of performing gain-of-function screens to identify genes that alter lymphocyte activity. The genes include, for example, those that may not be typically expressed by lymphocytes or genes that expressed only in a specific lymphocyte population or context (e.g., following exposure to antigen). The ability to identify genes whose expression is altered by stimulation or not typically expressed has certain advantages of the methods that rely on tailored, biased libraries, include those based on RNA sequencing. In particular, the methods disclosed facilitate the translation of gain-of-function studies to many different physiological and pathological contexts and modifying difficult-to-engineer cell types.

In certain embodiments, a method of identifying a gene that alters the therapeutic function of a modified lymphocyte when exogenously expressed in the modified lymphocyte is provided, wherein the method includes (a) obtaining a lymphocyte population; (b) transducing the lymphocyte population with a plurality of viral vectors, each viral vector encoding a gene linked to a barcode; (c) stimulating the transduced lymphocytes to induce activation, proliferation, and/or effector function; (d) isolating a transduced lymphocyte from the lymphocyte population of (c); and (c) detecting the presence of the gene and/or the linked barcode in the isolated lymphocyte. The gene identified according to the method is effective to alter the therapeutic function of a modified lymphocyte that expresses the gene. Lymphocytes expressing the gene and methods for delivery of the lymphocyte to a subject are provided herein.

While the screening methods exemplified herein utilize lentiviral-mediated delivery of an open reading frame (ORF) library (see Sack et al. Cell. 2018 Apr. 5; 173(2):499-514.2, which is incorporated herein by reference), the methods extend to approaches that screen expression of non-coding sequences, such as non-coding RNAs (e.g., microRNAs (miRNAs) and long noncoding RNAs (lncRNA, long ncRNAs)), as well as alternative ORFs, micro ORFs, upstream ORFs, and the like (i.e., small protein-coding elements in the genome). Thus, the term “gene” as used herein refers to sequences that both encode a protein and those that do not that.

The term “barcode” or “barcode sequence” as used herein refers to a nucleotide sequence that corresponds to and allows for detection and/or identification of an expressed gene. The barcode typically comprises four or more nucleotides. In some embodiments, the barcode comprises 4, 5, 6, 7, 8, 9, 10, 11, 2, 13, 14, or 15 nucleotides. In some embodiments, the barcode comprises 8 to 15 nucleotides. As used herein, the terms “barcoded gene”, “barcoded ORF”, and the like refers to a nucleic acid that has an appended barcoded sequence, whether the barcode is linked directly to the 5′ or 3′ end of the ORF or separated by 1 or more nucleotides at the 5′ or 3′ end of the ORF.

While certain aspects are described herein with reference to T cells, the disclosed methods can identify altered functional responses in other lymphocyte populations including, but not limited to, NK T cells, NK cells, B cells, γδ T cells and combinations and subpopulations thereof. In certain embodiments, the lymphocyte population is a cell population that has been enriched for one or more of T cells, B cells, NK T cells, NK cells, Vγ9Vδ2 T cells, or a subpopulation thereof. The cells may be obtained from one or more subjects from biological samples (blood, tissue, etc.) using a variety of isolation or purification methods know in the art for obtaining and/or enriching cell sample having lymphocytes or a population/sub-population of lymphocytes, including e.g., Ficoll gradient separation, positive and negative selection techniques using antibodies, tetramers, etc. either by magnetic separation or flow cytometry. In certain embodiments, the lymphocyte population is enriched for one or more of CD4+ T cells, CD8+ T cells, αβ T cells, and γδ T cells. In certain embodiments, the screening methods include a lymphocyte having a functional antigen-specific receptor expressed on the lymphocyte surface. In certain embodiments, the lymphocyte population comprises a CAR-expressing or engineered TCR-expressing lymphocyte. In certain embodiments, the lymphocyte population is an immortalized cell line. In a further, embodiment, the cell line has been modified to express a CAR or an engineered TCR. In certain embodiments, the lymphocyte population an NK cell line (e.g. NK-92 or a modified variant). In certain embodiments, the lymphocyte population is derived from a subject or subject having a certain disease or demographic profile. Such disease includes a cancer, such as those described herein, or an infectious disease.

The screening methods disclosed herein require expressing in a lymphocyte population a collection of barcoded genes. Methods for introducing nucleic acids into the cells include viral and non-viral mediated. In certain, the method utilizes a barcoded ORF library that is delivered to a population of cells using a suitable retroviral or lentiviral vector (see, e.g., Sack et al. Cell. 2018 Apr. 5; 173(2): 499-514.e2 and Yang et al. Nat Methods. 2011 Jun. 26; 8(8):659-61, which are incorporated herein by reference). In certain embodiments, at least 20%, at least 30%, at least 40%, at least 50%, at least 60%, at least 70%, at least 80%, or at least 90% of the lymphocyte population is transduced with the vector that delivers genes to be screened. While higher efficiencies of transduction are advantageous, particularly when analyzing smaller or rarer lymphocyte populations, in certain embodiments the methods include one or more selection steps that further enrich the cell population for cells that express genes to be screened. For example, a drug resistance gene (e.g., a puromycin resistance gene) can be included in the gene delivery vector to facilitate selection of transduced cells. In certain embodiments, the gene encoding construct includes a selection gene (e.g., a fluorescent protein, GFP) that facilitates the further isolation or enrichment of transduced cells using flow cytometry.

In certain embodiments, the methods include delivery of a barcoded gene operably linked to a promoter and/or other regulatory elements. The selection of a promoter can depend on factors such as the target cell type (i.e., the lymphocyte population), desired level of gene expression, and/or duration of expression. Suitable primers are provided herein. In certain embodiments, the selection of a promoter determines the efficiency or effectiveness of the method and confer surprising advantages. In certain embodiments, the promoter is an elongation factor-1α short (EFS) promoter. In certain embodiments, the promoter is an elongation factor-1α short (EFS) promoter. In certain embodiments, the promoter is a cytomegalovirus (CMV) promoter. In certain embodiments, a CMV promoter is preferred where it provides higher levels of expression than an EFS promoter. In certain embodiments, the promoter is a phosphoglycerate kinase-1 (PGK) promoter. In yet further embodiments, the promoter is an inducible promoter.

In certain embodiments, the methods provided require the assessment of one or more effector functions following stimulation of a lymphocyte population, wherein changes in the effector function(s) are indicative of an altered response due to the expression of an exogenous gene included in the screen. The term “stimulation” as used herein refers to a primary response induced by binding of a stimulatory molecule of a lymphocyte with its cognate ligand thereby mediating a signal transduction even. In certain embodiments, the method includes stimulation that induces signal transduction via the TCR/CD3 complex. Stimulation can mediate the altered expression of certain molecules, such as downregulation of TGF-β, and/or reorganization of cytoskeletal structures, and the like.

The term “stimulatory molecule,” refers to a molecule expressed by an immune cell, e.g., a T cell, a NK cell, or a B cell, that provide the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with a peptide or polyclonal crosslinking, and which leads to a response, including, but not limited to, proliferation, activation, differentiation, and resistance to apoptosis.

The methods disclosed can be adapted to stimulate cells according to various means, including using polyclonal and non-specific stimulation. In certain embodiments, cells are stimulated using an antibody, bound or soluble, that is specific for an epitope on the lymphocyte cell surface. In certain embodiments, an antibody binds and crosslinks antigen receptors on the surface of a cell. In certain embodiments, lymphocytes are stimulated in antigen specific manner. In certain embodiments, the method comprises stimulating the lymphocyte population with one or more of culturing the lymphocytes with one or more of an antibody, a cytokine, an antigen, a superantigen, an antigen presenting cell, a cancer cell, and a cancer cell line. In certain embodiments, the lymphocyte population includes T cells that are activated via incubation with anti-CD3 and anti-CD28 antibodies. The lymphocytes may also be cultured with cytokines that promote activation, proliferation, differentiation, apoptosis, and/or survival.

The term “antigen presenting cell,” refers to an immune system cell such as an accessory cell (e.g., a B-cell, a dendritic cell, and the like) that displays foreign antigens complexed with major histocompatibility complexes (MHCs) on their surfaces. T cells may recognize these complexes using their T cell receptors (TCRs). APCs process antigens and present them to T-cells. In certain embodiments, the antigen presenting cell induces a response in lymphocyte that expresses a CAR or engineered TCR. In certain embodiments, the antigen presenting cell is a cancer cell line.

In certain embodiments, stimulation of the lymphocytes identifies cells with altered responses as a result of expression of an exogenous gene. Altered responses include, but are not limited to, changes in the extent of proliferation, survival, apoptosis, phenotypic changes (e.g., surface markers, size), production and/or secretion of cytokines or chemokines, and cytotoxic potential. In certain embodiments, proliferation of lymphocytes is determined by labelling the cells with a dye (e.g., CFSE or CellTrace) prior to stimulation. In certain embodiments, expression of markers on lymphocytes, including one or more of CD69, CD25, OX40L (CD154), ICAM-1, CD70, CD74, CD54, MHC-II, CD137, CD44, CD62L, CCR7, CD107a, PD1, TIM3, LAG3, CD80, CD86. TIGIT, VISTA, B7-H3, BTLA, and SIGLEC15 is determined to identify and/or isolate cells that are stimulated. In certain embodiments, production and/or secretion of cytokines or chemokines including one or more of IL-2, IL-12, IL-23, IFNγ, TNFα, GM-CSF, IL7, IL15, IL12, IL18, IL21, IL23, LTA, IL4, IL5, IL6, IL10, IL13, TGFbeta, IL17, LTA, LIGHT, CCL3, CCL4, CCL5, MCP-3, CXCL9, MIP1α, IL8, PDGF-AA, IP10, IL22, IL3, MCP-1, IL9, MDC, sCD40L, and M-CSF is determined to identify and/or isolate cells that are stimulated. In certain embodiments, expression of the markers, cytokines, and/or chemokines is determined by flow cytometry to facilitate sorting of cells based on expression, including relative levels of expression. In certain embodiments, the lymphocytes express proteins that are indicative of cytotoxic potential. In certain embodiments, the expression of perforin and/or granzyme is determined.

Following isolation of stimulated lymphocytes, identification of the expressed gene is determined by PCR amplification of the gene and/or barcode sequence. In certain embodiments, PCR is performed on genomic DNA (gDNA) obtained from the lymphocytes. In certain embodiments, a reverse transcription step is performed to generate cDNA form the cell transcriptome and/or from an exogenous gene and barcode mRNA transcript. The amplified DNA products are then sequenced to identify an exogenous gene expressed in the isolated lymphocyte and/or to quantify the relative expression of an exogenous gene in a population of isolated lymphocytes. In certain embodiments, the disclosed screening methods include RNA and/or DNA sequencing of isolated lymphocytes using techniques that include, but are not limited to, whole transcriptome analysis, whole genome analysis, barcoded sequencing of whole or targeted regions of the genome, and combinations thereof. In certain embodiments, RNA and/or DNA sequencing is performed in combination with proteome analysis. In certain embodiments, the methods include detection of cell surface or intracellular proteins using, e.g., flow cytometry. In certain embodiments, the methods, comprise detection or identification the barcoded gene in combination with profiling additional molecular modalities using methods described in the art, including for example single-cell sequencing analysis (e.g., 10× Genomics Multiome platform), single-cell RNA-sequencing (scRNA-seq) (See, e.g., Haque et al. A practical guide to single-cell RNA-sequencing for biomedical research and clinical applications, Genome Medicine, 9, Article number: 75 (2017); Hwang et al. Single-cell RNA sequencing technologies and bioinformatics pipelines. Exp Mol Med. 2018 Aug. 7; 50(8):96), cell-hashing (Sec, e.g., Stoeckius et al. Cell Hashing with barcoded antibodies enables multiplexing and doublet detection for single cell genomics. Genome Biol. 2018; 19: 224), Perturb-Seq. (See, e.g., Dixit et al. Perturb-seq: Dissecting molecular circuits with scalable single cell RNA profiling of pooled genetic screens. Cell. 2016 Dec. 15; 167(7): 1853-1866.e17), CROP-seq (See, e.g., Datlinger et al. Pooled CRISPR screening with single-cell transcriptome readout Nat Methods. 2017 March; 14(3):297-301), CRISP-seq (See, e.g., Jaitin et al. Dissecting Immune Circuits by Linking CRISPR-Pooled Screens with Single-Cell RNA-Seq Cell. 2016 Dec. 15; 167(7): 1883-1896.e15), Expanded CRISPR-compatible CITE-seq (ECCITE-seq) (See, e.g., Mimitou et al. Multiplexed detection of proteins, transcriptomes, clonotypes and CRISPR perturbations in single cells. Nat Methods. 2019 May; 16(5):409-41), and cellular indexing of transcriptomes and epitopes-seq (CITE-seq) (Sec, e.g., Stoeckius et al. Simultaneous epitope and transcriptome measurement in single cells. Nat Methods. 2017 September; 14(9):865-868).

### OverCITE-seq

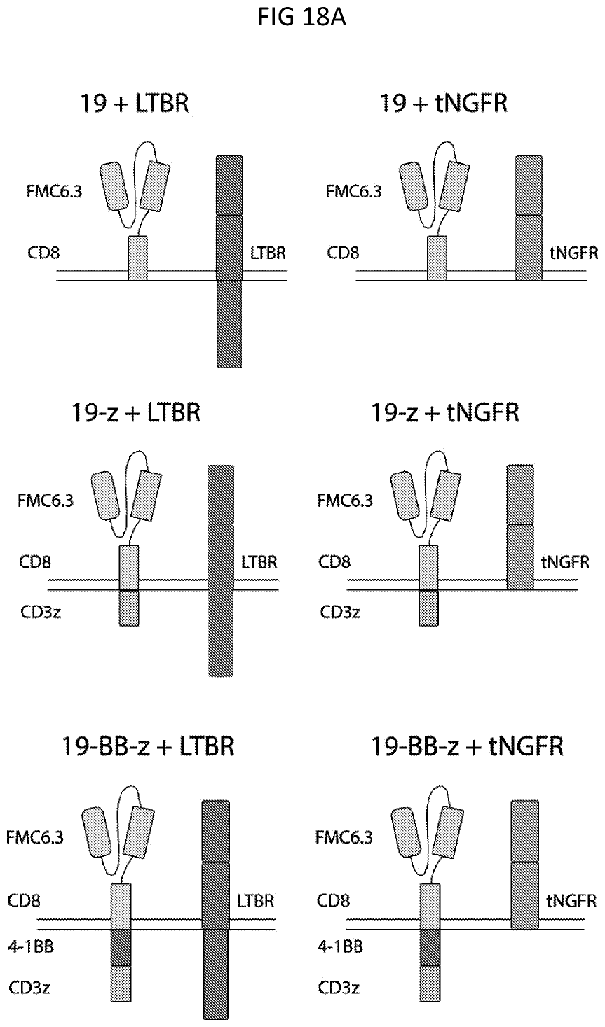

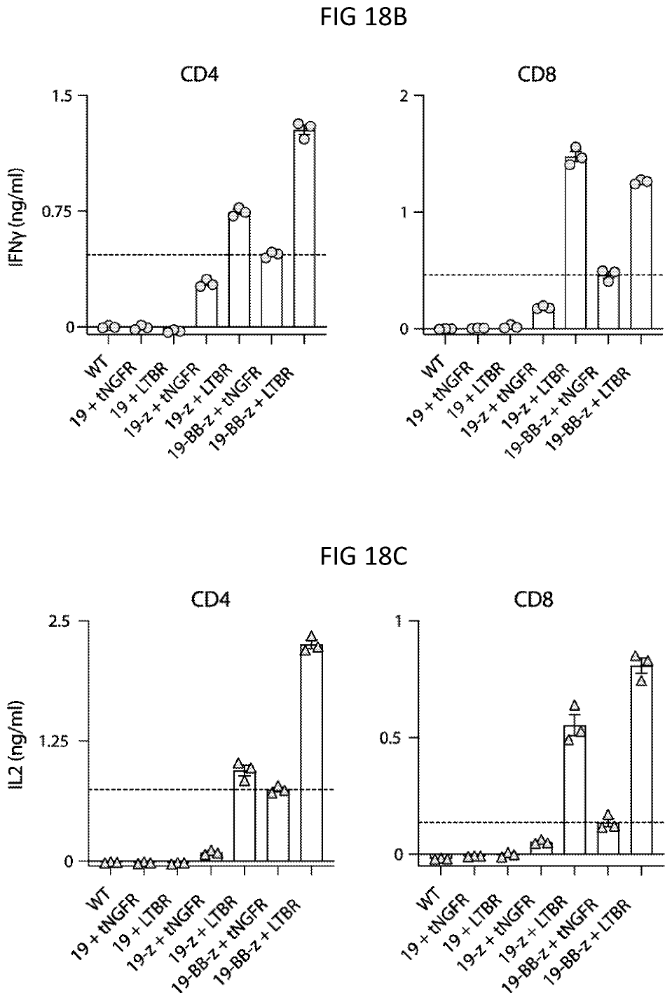

To gain a more comprehensive view of the mechanisms of action of individual genes, and to provide a multidimensional description of the phenotypic changes they induce, described herein is a single-cell sequencing strategy with direct ORF capture. An overview of an embodiment of this aspect is provided in FIG. 18A. As referred to herein, the term “open reading frame mRNA” or ORF mRNA refers to the not only the coding sequence of the gene of interest, but also includes, in some embodiments, downstream sequences which may include a barcode and/or a selection marker, e.g., puromycyin. This approach, termed OverCITE-seq (Overexpression-compatible CITE-seq) builds on previous approaches developed for quantifying surface antigens and CRISPR perturbations and allows for high-throughput, single-cell analysis of a pool of T-cells with different ORFs. In brief, mRNA from lentivirally integrated ORFs is specifically reverse transcribed by a primer binding to a constant sequence of the transcript downstream of the ORF and barcoded, along with the cell transcriptome, during template switching. The resulting cDNA pool is then split for separate construction of gene expression and ORF expression libraries.

Thus, in another aspect, a method of analyzing the effect on an individual cell of overexpression of an ORF of interest is provided. The method includes introducing into the cell an expression cassette comprising a nucleic acid encoding the ORF of interest and overexpressing said ORF in the cell. Overexpression of the ORF may be accomplished through the use of a strong promoter, such as CMV, EF-1a, CAG, PKG, etc. Such promoters are known in the art.

A first set of nucleic acids derived from the individual cell is provided along with oligonucleotides having a common barcode sequence into a discrete partition, wherein the oligonucleotides are attached to a bead. The barcode sequence provides a unique identifier such that, upon characterization of those nucleic acids, they may be attributed as having been derived from the same cell. That is, the oligonucleotides are partitioned such that as between oligonucleotides in a given partition, the nucleic acid barcode sequences contained therein are the same, but as between different partitions, the oligonucleotides have differing barcode sequences. The nucleic acid barcode sequences can include from 6 to about 20 or more nucleotides within the sequence of the oligonucleotides. In some cases, the length of a barcode sequence may be 6, 7, 8, 9, 10, 11, 12, 13, 14, 15, 16, 17, 18, 19, 20 nucleotides or longer. In some cases, the length of a barcode sequence may be at least 6, 7, 8, 9, 10, 11, 12, 13, 14, 15, 16, 17, 18, 19, 20 nucleotides or longer. In some cases, the length of a barcode sequence may be at most 6, 7, 8, 9, 10, 11, 12, 13, 14, 15, 16, 17, 18, 19, 20 nucleotides or shorter.

The first set of nucleic acids comprises both endogenous transcriptome mRNA and ORF mRNA. In some embodiments, the nucleic acids are released from the individual cell in the discrete partition. In some embodiments, the nucleic acids comprise ribonucleic acid (RNA), such as, for example, messenger RNA (mRNA). As used herein, in some embodiments, the partitions refer to containers or vessels (such as wells, microwells, tubes, through ports in nanoarray substrates, e.g., BioTrove nanoarrays, or other containers). In some embodiments, however, the compartments or partitions comprise partitions that are flowable within fluid streams. These partitions may be comprised of, e.g., microcapsules or micro-vesicles that have an outer barrier surrounding an inner fluid center or core, or they may be a porous matrix that is capable of entraining and/or retaining materials within its matrix. In some embodiments, however, these partitions comprise droplets of aqueous fluid within a non-aqueous continuous phase, e.g., an oil phase. See, e.g., US 2015/0376609A1 which is incorporated herein by reference in its entirety. In some embodiments, the cells may be partitioned along with lysis reagents in order to release the contents of the cells within the partition. In such cases, the lysis agents can be contacted with the cell suspension concurrently with, or immediately prior to the introduction of the cells into the partitioning junction/droplet generation zone. In addition to the lysis agents co-partitioned with the cells described above, other reagents can also be co-partitioned with the cells, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids.

Additional reagents may also be co-partitioned with the cells, such as endonucleases to fragment the cell's DNA, DNA polymerase enzymes and dNTPs used to amplify the cell's nucleic acid fragments and to attach the barcode oligonucleotides to the amplified fragments. Additional reagents may also include reverse transcriptase enzymes, including enzymes with terminal transferase activity, primers and oligonucleotides, and switch oligonucleotides (also referred to herein as “switch oligos”) which can be used for template switching. In some cases, template switching can be used to increase the length of a cDNA. In one example of template switching, cDNA can be generated from reverse transcription of a template, e.g., cellular mRNA, where a reverse transcriptase with terminal transferase activity can add additional nucleotides, e.g., polyC, to the cDNA that are not encoded by the template, such, as at an end of the cDNA. Switch oligos can include sequences complementary to the additional nucleotides, e.g. polyG. The additional nucleotides (e.g., polyC) on the cDNA can hybridize to the sequences complementary to the additional nucleotides (e.g., polyG) on the switch oligo, whereby the switch oligo can be used by the reverse transcriptase as template to further extend the cDNA. Switch oligos may comprise deoxyribonucleic acids, ribonucleic acids, modified nucleic acids including locked nucleic acids (LNA), or any combination. Such reagents are known in the art. Sec, e.g., Chromium Next GEM Single Cell 5′ Reagent Kits v2 User Guide (available from assets.ctfassets.net/an68im79xiti/1Ap6qQvsq80oQAGacem7RD/6f895796e18c38a14ef1e8b8ff79a82c/CG000331_ChromiumNextGEMSingleCell5-v2_RevB.pdf), which is incorporated herein by reference in its entirety.

The method further includes performing RT-PCR to generate a second set of nucleic acids derived from the first set of nucleic acids that comprises endogenous transcriptome cDNA and ORF cDNA, wherein said second set of nucleic acids within the partition have attached thereto oligonucleotides that comprise the common nucleic acid barcode sequence. The RT-PCR reagents which include an oligonucleotide primer which specifically anneals to a sequence on the ORF mRNA that is not a poly A sequence. In some embodiments, the oligonucleotide primer which specifically anneals to a sequence on the ORF mRNA, anneals to the mRNA on a portion of the transcript that is common to the sequences of a library, e.g., a coding sequence for a resistance marker, e.g., puromycin. This allows for amplification of many ORF sequences from an ORF library using a common set of reagents. In some embodiments, generating one or more second nucleic acid sequences includes subjecting the nucleic acids to reverse transcription under conditions that yield the one or more second nucleic acid sequences. In some embodiments, the reverse transcription occurs in the discrete partition. In some embodiments, the oligonucleotides are provided in the discrete partition and include a poly-T sequence. In some embodiments, the reverse transcription comprises hybridizing the poly-T sequence to at least a portion of each of the nucleic acids and extending the poly-T sequence in template directed fashion. In some embodiments, the oligonucleotides include an anchoring sequence that facilitates hybridization of the poly-T sequence. In some embodiments, the oligonucleotides include a random priming sequence that can be, for example, a random hexamer. In some embodiments, the reverse transcription comprises hybridizing the random priming sequence to at least a portion of each of the nucleic acids and extending the random priming sequence in template directed fashion.

The method further includes amplifying the second set of nucleic acids to generate a third set of nucleic acids using PCR reagents which comprise a second primer which specifically anneals to a sequence on the ORF cDNA, that is not a poly A sequence.

In certain embodiments, the method includes obtaining a portion of the third set of nucleic acids and amplifying the ORF cDNA using a second set of PCR reagents which comprise a third primer which specifically anneals to a sequence on the ORF cDNA, that is not a poly A sequence, to generate a fourth set of nucleic acids

In certain embodiments, the method includes amplifying the ORF cDNA in the fourth set of nucleic acids using a third set of PCR reagents which comprise a fourth primer which specifically anneals to a sequence on the ORF cDNA, that is not a poly A sequence, to generate a fifth set of nucleic acids.

The third, and optionally, fifth sets of nucleic acids are then fragmented, adapters are attached to both ends of the fragments and subjected to next generation sequencing (NGS) using standard techniques known in the art. Sec, e.g., Kanzi et al, Next Generation Sequencing and Bioinformatics Analysis of Family Genetic Inheritance, Front. Genet., 23 Oct. 2020| https://doi.org/10.3389/fgene.2020.544162, which is incorporated herein by reference. See also, US 2018-0251825 A1 and US 2015-0376609 both of which are incorporated herein by reference.

In certain embodiments, the methods comprise detection or identification of the barcoded sequence in combination with profiling additional molecular modalities using methods described in the art, including for example single-cell sequencing analysis (e.g., 10× Genomics Multiome platform), single-cell RNA-sequencing (scRNA-seq) (See, e.g., Haque et al. A practical guide to single-cell RNA-sequencing for biomedical research and clinical applications, Genome Medicine, 9, Article number: 75 (2017); Hwang et al. Single-cell RNA sequencing technologies and bioinformatics pipelines. Exp Mol Med. 2018 Aug. 7; 50(8):96), cell-hashing (See, e.g., Stoeckius et al. Cell Hashing with barcoded antibodies enables multiplexing and doublet detection for single cell genomics. Genome Biol. 2018; 19: 224), Perturb-Seq. (Sec, e.g., Dixit et al. Perturb-seq: Dissecting molecular circuits with scalable single cell RNA profiling of pooled genetic screens. Cell. 2016 Dec. 15; 167(7): 1853-1866.e17), CROP-seq (See, e.g., Datlinger et al. Pooled CRISPR screening with single-cell transcriptome readout Nat Methods. 2017 March; 14(3):297-301), CRISP-seq (See, e.g., Jaitin et al. Dissecting Immune Circuits by Linking CRISPR-Pooled Screens with Single-Cell RNA-Seq Cell. 2016 Dec. 15; 167(7): 1883-1896.c15), Expanded CRISPR-compatible CITE-seq (ECCITE-seq) (See, e.g., Mimitou et al. Multiplexed detection of proteins, transcriptomes, clonotypes and CRISPR perturbations in single cells. Nat Methods. 2019 May; 16(5):409-41), and cellular indexing of transcriptomes and epitopes-seq (CITE-seq) (See, e.g., Stoeckius et al. Simultaneous epitope and transcriptome measurement in single cells. Nat Methods. 2017 September; 14(9):865-868).

With regard to the description of the inventions provided herein, it is intended that each of the compositions described, is useful, in another embodiment, in the methods of the invention. In addition, it is also intended that each of the compositions herein described as useful in the methods, is, in another embodiment, itself an embodiment of the invention.

### Specific Embodiments

1. A modified lymphocyte comprising an exogenous nucleic acid encoding LTBR.

2. The modified lymphocyte according to embodiment 1, wherein the nucleic acid encoding LTBR encodes an intracellular domain, or fragment or variant thereof.

3. The modified lymphocyte according to embodiment 2, wherein the LTBR intracellular domain comprises amino acids 249 to 435 of SEQ ID NO: 2, or a fragment, deletion, or variant thereof.

4. The modified lymphocyte according to embodiment 2 or 3, wherein the LTBR intracellular domain has a deletion in at least amino acids 393 to 435.

5. The modified lymphocyte according to any one of embodiments 1 to 4, wherein the lymphocyte comprises an expression cassette comprising an expression control sequence and the nucleic acid encoding LTBR.

6. The modified lymphocyte according to any one of embodiments 1 to 5, wherein the lymphocyte further comprises a nucleic acid encoding a chimeric antigen receptor (CAR).

7 The modified lymphocyte according to embodiment 5, wherein the expression cassette further comprises the nucleic acid encoding the CAR.

8. The modified lymphocyte according to embodiment 1 or 5, wherein the lymphocyte further comprises a nucleic acid encoding a T cell receptor (TCR).

9. The modified lymphocyte according to embodiment 8, wherein the expression cassette further comprises the nucleic acid encoding the TCR.

10. The modified lymphocyte according to any one of embodiments 1 to 9, wherein the expression control sequence comprises an EF-1α, EFS, or CMV promoter.

11. The modified lymphocyte according to embodiment 1, wherein the exogenous nucleic acid encoding LTBR is mRNA.

12. The modified lymphocyte according to any one of embodiments 1 to 11, wherein the lymphocyte is a T cell.

13. The modified lymphocyte according to any one of embodiments 1 to 12, wherein the lymphocyte is an alpha beta T or gamma delta T cell, optionally a Vγ9Vδ2 T cell.

14. The modified lymphocyte according to any one of embodiments 1 to 12, wherein the lymphocyte is an NK cell.

15. The modified lymphocyte according to any one of embodiments 1 to 12, wherein the lymphocyte is an NK T cell.

16. The modified lymphocyte according to any one of embodiments 6, 7, 10, or 12 to 15, wherein the CAR is Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kymriah®), or one of those found in FIG. 19.

17. A vaccine composition comprising a nucleic acid encoding LTBR and a nucleic acid encoding a viral protein.

18. The vaccine composition comprising according to embodiment 17, wherein the nucleic acid encoding LTBR encodes an LTBR intracellular domain, or fragment or variant thereof.

19. The modified lymphocyte according to embodiment 18, wherein the LTBR intracellular domain comprises amino acids 249 to 435 of SEQ ID NO: 2, or a fragment, deletion, or variant thereof.

20. The modified lymphocyte according to any embodiment 18 or 19, wherein the LTBR intracellular domain has a deletion in at least amino acids 393 to 435.

21. The vaccine composition according to any one of embodiment 17 to 20, wherein the viral protein is a glycoprotein.

22. The vaccine composition of embodiment 21, wherein the glycoprotein is a viral spike protein.

23. The vaccine composition of embodiment 22, wherein the viral spike protein is a coronavirus spike protein.

24. The vaccine composition according to any one of embodiments 17 to 23, wherein the nucleic acid encoding LTBR is mRNA, or the nucleic acid encoding the viral spike protein is mRNA, or both.

25. An expression cassette comprising a nucleotide sequence encoding a chimeric antigen receptor (CAR) and a nucleic acid encoding LTBR.

26. An expression cassette comprising a nucleic acid encoding a T cell receptor and a nucleic acid encoding LTBR.

27. An expression cassette comprising a nucleic acid encoding a viral protein and a nucleic acid encoding LTBR.

28. The expression cassette according to any one of embodiments 25 to 27, wherein the nucleic acid encoding LTBR encodes an LTBR intracellular domain, or fragment or variant thereof.

29. The expression cassette according to embodiment 28, wherein the LTBR intracellular domain comprises amino acids 249 to 435 of SEQ ID NO: 2, or a fragment, deletion, or variant thereof.

30. The expression cassette according to embodiment 28 or 29, wherein the LTBR intracellular domain has a deletion in at least amino acids 393 to 435.

31. A composition comprising a modified lymphocyte comprising the expression cassette to any one of embodiments 25 to 30.

32. A method of producing a modified lymphocyte comprising introducing an exogenous nucleic acid encoding LTBR into the cell.

33. The method according to embodiment 32, wherein the nucleic acid encoding LTBR encodes an LTBR intracellular domain, or fragment or variant thereof.

34. The method according to embodiment 32 or 33, wherein the LTBR intracellular domain comprises amino acids 249 to 435 of SEQ ID NO: 2, or a fragment, deletion, or variant thereof.

35. The method according to any one of embodiments 32 to 34, wherein the LTBR intracellular domain has a deletion in at least amino acids 393 to 435.

36. The method according to any one of embodiments 32 to 35, wherein the lymphocyte comprises an expression cassette comprising an expression control sequence and the nucleic acid encoding LTBR.

37. The method according to any one of embodiments 32 to 36, wherein the lymphocyte further comprises a nucleic acid encoding a chimeric antigen receptor (CAR).

38. The method according to embodiment 37, wherein the expression cassette further comprises the nucleic acid encoding the CAR.

39. The method according to any one of embodiments 32 to 36, wherein the lymphocyte further comprises a nucleic acid encoding an engineered T cell receptor (TCR).

40. The method according to embodiment 39, wherein the expression cassette further comprises the nucleic acid encoding the TCR.

41. The method according to any one of embodiments 36 to 40, wherein the expression control sequence comprises an EF-1α (full-length or shortened) or CMV promoter.

42. The method according to any one of embodiments 32 to 42, wherein the exogenous nucleic acid encoding LTBR is mRNA.

43. The method according to any one of embodiments 32 to 42, wherein the lymphocyte is a T cell, optionally a CD4+ T cell, a CD8+ T cell, or a Treg cell.

44. The method according to any one of embodiments 32 to 43, wherein the lymphocyte is an alpha beta T cell.

45. The method according to any one of embodiments 32 to 43, wherein the lymphocyte is a gamma delta T cell, optionally a Vγ9Vδ2 T cell.

46. The method according to any one of embodiments 32 to 42, wherein the lymphocyte is an NK cell.

47. The method according to any one of embodiments 32 to 42, wherein the lymphocyte is an NK T cell.

48. The method according to any one of embodiments 37, 38, or 41 to 47, wherein the CAR is Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kymriah®), a chimeric autoantibody receptor, or one of those found in FIG. 19.

49. The method according to any one of embodiments 32 to 48, wherein the lymphocyte is nucleofected with mRNA encoding LTBR.

50. A method of treating cancer in a subject in need thereof, the method comprising administering the modified lymphocyte according to any one of embodiments 1 to 16, the expression cassette according to any one or embodiments 25 to 30, or the composition according to embodiment 31 to the subject.

51. The method according to embodiment 50, wherein the subject has lymphoma.

52. The method according to embodiment 50, wherein the subject has a solid tumor.

53. The method according to embodiment 50, wherein the subject has leukemia.

54. The method according to embodiment 50, wherein the subject has multiple myeloma.

55. The method according to embodiment 50, wherein the subject has a virally-driven cancer.

56. The method according to embodiment 55, wherein the subject has HPV.

57. The method according to embodiment 50, wherein the subject has a cancer that is Burkitt's lymphoma, liver cancer, Kaposi's sarcoma, cervical cancer, head cancer, neck cancer, anal cancer, oral cancer, pancreatic cancer, ovarian cancer, melanoma, pharyngeal cancer, penile cancer, adult T-cell lymphoma, or merkel cell carcinoma.

58. A method of treating a viral disease in a subject in need thereof, the method comprising administering a composition according to any one of embodiments 1 to 16 to the subject.

59. The method according to embodiment 58, wherein the disease is HIV or HPV.

60. A method of treating an autoimmune disorder in a subject in need thereof, the method comprising administering the modified lymphocyte according to any one of embodiments 1 to 16, the expression cassette according to any one or embodiments 25 to 30, or the composition according to embodiment 31 to the subject.

61. A method of increasing proliferation, or T cell effector function including cytokine production and/or secretion, the method comprising introducing the expression cassette according to any one or embodiments 25 to 30 into the T cell.

62. The method according to embodiment 61, wherein the T cell is obtained from a human prior to treating the T cell to overexpress LTBR, and the treated T cell is reintroduced into a human.

63. A method of increasing the response to a vaccine composition comprising co-administering to a subject a vaccine comprising a nucleic acid encoding LTBR.

64. The method according to embodiment 63, wherein the nucleic acid encoding LTBR encodes an LTBR intracellular domain, or fragment or variant thereof.

65. The method according to embodiment 64, wherein the LTBR intracellular domain comprises amino acids 249 to 435 of SEQ ID NO: 2, or a fragment, deletion, or variant thereof.

66. The method according to any embodiment 64 or 65, wherein the LTBR intracellular domain has a deletion in at least amino acids 393 to 435.

67. The method according to any one of embodiments 63 to 66, wherein the expression of LTBR is transient.

68. A modified lymphocyte comprising an exogenous nucleic acid encoding a gene of Table 1.

69. The modified lymphocyte according to embodiment 68, wherein the lymphocyte comprises an expression cassette comprising an expression control sequence and a nucleic acid encoding the gene of Table 1.

70. The modified lymphocyte according to embodiment 68 or 69, wherein the lymphocyte further comprises a nucleic acid encoding a chimeric antigen receptor (CAR).

71. The modified lymphocyte according to embodiment 70, wherein the expression cassette further comprises the nucleic acid encoding the CAR.

72. The modified lymphocyte according to embodiment 68 or 69, wherein the lymphocyte further comprises a nucleic acid encoding a T cell receptor (TCR).

73. The modified lymphocyte according to embodiment 72, wherein the expression cassette further comprises the nucleic acid encoding the TCR.

74. The modified lymphocyte according to any one of embodiments 68 to 73, wherein the expression control sequence comprises an EF-1α, EFS, or CMV promoter.

75. The modified lymphocyte according to embodiment 68, wherein the exogenous nucleic acid encoding the gene of Table 1 is mRNA.

76. The modified lymphocyte according to any one of embodiments 68 to 75, wherein the lymphocyte is a T cell.

77. The modified lymphocyte according to any one of embodiments 68 to 75, wherein the lymphocyte is an alpha beta T cell.

78. The modified lymphocyte according to any one of embodiments 68 to 75, wherein the lymphocyte is a gamma delta T cell, optionally a Vγ9Vδ2 T cell.

79. The modified lymphocyte according to any one of embodiments 68 to 75, wherein the lymphocyte is an NK cell.

80. The modified lymphocyte according to any one of embodiments 68 to 75, wherein the lymphocyte is an NK T cell.

81. The modified lymphocyte according to any one of embodiments 70, 71, or 74 to 80, wherein the CAR is Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kyrmriah®), or one of those found in FIG. 19.

82. A vaccine composition comprising a nucleic acid encoding a gene of Table 1 and a nucleic acid encoding a viral protein.

83. The vaccine composition of embodiment 82, wherein the viral protein is a glycoprotein.

84. The vaccine composition of embodiment 83, wherein the glycoprotein is a viral spike protein.

85. The vaccine composition of embodiment 84, wherein the viral spike protein is a coronavirus spike protein.

86. The vaccine composition of any one of embodiments 82 to 85, wherein the nucleic acid encoding the gene of Table 1 is mRNA, or the nucleic acid encoding the viral spike protein is mRNA, or both.

87. An expression cassette comprising a nucleotide sequence encoding a chimeric antigen receptor and a nucleic acid encoding a gene of Table 1.

88. An expression cassette comprising a nucleic acid encoding a T cell receptor and a nucleic acid encoding a gene of Table 1.

89. An expression cassette comprising a nucleic acid encoding a viral protein and a nucleic acid encoding a gene of Table 1.

90. A composition comprising a modified lymphocyte comprising the expression cassette of any one of embodiments 87 to 89.

91. A method of producing a modified lymphocyte comprising introducing an exogenous nucleic acid encoding a gene of Table 1 into the lymphocyte.

92. The method according to embodiment 91, wherein the lymphocyte comprises an expression cassette comprising an expression control sequence and a nucleic acid encoding the gene of Table 1.

93. The method according to embodiment 91 or 92, wherein the lymphocyte further comprises a nucleic acid encoding a chimeric antigen receptor (CAR).

94. The method according to embodiment 93, wherein the expression cassette further comprises the nucleic acid encoding the CAR.

95. The method according to embodiment 91 or 92, wherein the lymphocyte further comprises a nucleic acid encoding an engineered T cell receptor (TCR).

96. The method according to embodiment 95, wherein the expression cassette further comprises the nucleic acid encoding the TCR.

97. The method according to any one of embodiments 91 to 97, wherein the expression control sequence comprises an EF-1α (full-length or shortened) or CMV promoter.

98. The method according to any one of embodiments 91, wherein the exogenous nucleic acid encoding the gene of Table 1 is mRNA.

99. The method according to any one of embodiments 91 to 98, wherein the lymphocyte is a T cell, optionally a CD4+ T cell, CD8+ T cell, or a Treg cell.

100. The method according to any one of embodiments 91 to 99, wherein the lymphocyte is an alpha beta T cell.

101. The method according to any one of embodiments 91 to 98, wherein the lymphocyte is a gamma delta T cell.

102. The method according to any one of embodiments 91 to 98, wherein the lymphocyte is an NK cell.

103. The method according to any one of embodiments 91 to 98, wherein the lymphocyte is an NK T cell.

104. The method according to any one of embodiments 93, 94, or 97 to 103, wherein the CAR is Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kyrmriah®), or one of those found in FIG. 19.

105. The method according to any one of embodiments 91 to 104, wherein the lymphocyte is nucleofected with mRNA encoding the gene of Table 1.

106. A method of treating cancer in a subject in need thereof, the method comprising administering the 68 to 81, the expression cassette according to any one of embodiments 87 to 89, or the composition according to embodiment 90 to the subject.

107. The method according to embodiment 106, wherein the subject has lymphoma.

108 The method according to embodiment 106, wherein the subject has a solid tumor.

109 The method according to embodiment 106, wherein the subject has leukemia.

110. The method according to embodiment 106, wherein the subject has multiple myeloma.

111. The method according to embodiment 106, wherein the subject has a virally-driven cancer.

112. The method according to embodiment 111, wherein the subject has HPV.

113. The method according to embodiment 106, wherein the subject has a cancer that is Burkitt's lymphoma, liver cancer, Kaposi's sarcoma, cervical cancer, head cancer, neck cancer, anal cancer, oral cancer, pharyngeal cancer, penile cancer, adult T-cell lymphoma, or merkel cell carcinoma.

114. A method of treating a disease in a subject in need thereof, the method comprising administering a composition according to any one of embodiments 82 to 86 to the subject.

115. The method according to embodiment 114, wherein the disease is HIV.

116 The method according to embodiment 114, wherein the disease is HPV.

117. The method according to embodiment 114, wherein the disease is an autoimmune disorder.

118. A method of increasing proliferation, or T cell effector function including cytokine production and/or secretion, the method comprising introducing the composition according to any one of embodiments 87 to 89 into the T cell.

119. The method according to embodiment 118, wherein the T cell is obtained from a human prior to treating the T cell to overexpress the gene of Table 1, and the treated T cell is reintroduced into the human.

120. A method of increasing the response to a vaccine composition comprising co-administering with a vaccine a nucleic acid encoding a gene of Table 1.

121. The method according to any one of embodiments 118 to 120, wherein the expression of the gene of Table 1 is transient.

122. The modified lymphocyte, composition, expression cassette, or method according to any one of embodiments 68 to 121, wherein the gene of Table 1 is LTBR, ADA, IFNL2, IL12B CALML3 MRPL51, DBI GPN3, ITM2A, AHNAK, BATF, GPD1, ATF6B, AHCY, DUPD1, or AKR1C4.

123. A method of identifying a gene that alters the therapeutic function of a modified lymphocyte when exogenously expressed in the modified lymphocyte, the method comprising:
(a) obtaining a lymphocyte population;
(b) transducing the lymphocyte population with a plurality of viral vectors, each viral vector encoding a gene which may be linked to one or more barcodes;
(c) stimulating the transduced lymphocytes to induce activation, proliferation, and/or effector function;
(d) isolating a transduced lymphocyte from the lymphocyte population of (c); and
(e) detecting the presence of the gene and/or the linked barcodes in the isolated lymphocyte;
wherein the detected gene is effective to alter the therapeutic function of a modified lymphocyte that expresses the gene.

124. The method according to embodiment 123, wherein the gene is an open-reading frame (ORF) or a nucleotide sequence encoding a non-coding RNA, optionally a microRNA (miRNA) or long non-coding RNA (lncRNA, long ncRNA).

125. The method according to embodiment 123 or 124, wherein the lymphocyte population comprises a cell population that has been enriched for one or more of T cells, B cells, NK T cells, NK cells, or a subpopulation thereof, optionally wherein the cells are human.

126. The method according to embodiment 125, wherein the lymphocyte population is enriched for one or more of CD4+ T cells, CD8+ T cells, αβ T cells, and γδ T cells.

127. The method according to any one of embodiments 123 to 126, wherein the lymphocyte population comprises a CAR T cell.

128. The method according to any one of embodiments 123 to 126, wherein the lymphocyte population comprises a lymphocyte comprising an engineered TCR expressed on its surface.

129. The method according to any one of embodiments 123 to 128, wherein the lymphocyte population comprises a cell line.

130. The method according to any one of embodiments 123 to 129, wherein the plurality of viral vectors comprises a library of open reading frames (ORFs).

131. The method according to any one of embodiments 123 to 130, wherein the viral vectors comprise a retroviral vector or a lentiviral vector.

132. The method according to any one of embodiments 123 to 131, wherein (b) comprises transducing at least 20%, at least 30%, at least 40%, at least 50%, at least 60%, at least 70%, at least 80%, or at least 90% of the lymphocyte population.

133. The method according to any one of embodiments 123 to 132, wherein the viral vectors comprise an expression cassette having an elongation factor-1α short (EFS) promoter, cytomegalovirus (CMV) promoter, or phosphoglycerate kinase-1 (PGK) promoter.

134. The method according to any one of embodiments 123 to 133, wherein the viral vectors comprise a nucleotide sequence that encodes a selection gene or marker.

135. The method according to any one of embodiments 123 to 134, wherein the modified lymphocyte is an γδ T cell or αβ T cell.

136. The method according to any one of embodiments 123 to 134, wherein the modified lymphocyte is T cell that expresses a Vγ9- or Vγ9Vδ2 TCR.

137. The method according to any one of embodiments 123 to 136, wherein the modified lymphocyte is a CAR T cell.

138. The method according to any one of embodiments 123 to 136, where in the modified lymphocyte has been selected based on expression of an endogenous antigen receptor, optionally a TCR.

139. The method according to any one of embodiments 123 to 138, wherein stimulating the transduced lymphocytes comprises culturing the lymphocytes with one or more of an antibody, cytokine, an antigen, a superantigen, an antigen presenting cell, a cancer cell, and a cancer cell line.

140. The method according to any one of embodiments 123 to 138, wherein stimulation of the transduced lymphocytes comprises TCR stimulation, optionally comprising CD3/CD28 stimulation.

141. The method according to any one of embodiments 123 to 140, further comprising labeling the transduced lymphocytes with a cell proliferation dye, and isolating progeny cells.

142. The method according to any one of embodiments 123 to 141, wherein (d) comprises identifying cells that express one or more cell surface markers and/or one or more effector functions and/or one or more secreted cytokines.

143. The method according to embodiment 142, wherein the one or more cell surface markers comprise CD69, CD25, OX40L (CD154), ICAM-1, CD70, CD74, CD54, MHC-II, CD137, CD44, CD62L, CCR7, CD107a, PD1, TIM3, LAG3, CD80, CD86. TIGIT, VISTA, B7-H3, BTLA, and SIGLEC15.

144. The method according to embodiment 123 to 143, wherein the one or more effector functions comprise cytokine or chemokine production and/or secretion, optionally wherein the cytokine or chemokine is one or more of IL-2, IL-12, IL-23, IFNγ, TNF, GM-CSF, IL7, IL15, IL12, IL18, IL21, IL23, LTA, IL4, IL5, IL6, IL10, IL13, TGFbeta, IL17, LTA, LIGHT, CCL3, CCL4, and CCL5.

145. The method according to any one of embodiments 123 to 144, wherein the one or more effector functions comprises cytotoxic potential, optionally wherein cytotoxic potential is identified by expression of perforin and/or granzyme.

146. The method according to any one of embodiments 123 to 145, wherein (e) comprises obtaining genomic DNA from the isolated lymphocyte and PCR amplification of the gene and/or barcode sequence.

147. The method according to any one of embodiments 123 to 146, wherein (e) further comprises single-cell transcriptome and/or proteome analysis.

148. The method according any one of embodiments 123 to 147, wherein (e) comprises flow cytometric analysis, cell-hashing, single-cell sequencing analysis, single cell RNA sequencing (scRNA-seq), Perturb-seq, CROP-seq, CRISP-seq, ECCITE-seq, or cellular indexing of transcriptomes and epitopes (CITE-seq).

149. A method of analyzing the effect on an individual cell of overexpression of an ORF of interest, comprising:
(a) introducing into the cell an expression cassette comprising a nucleic acid encoding the ORF of interest and overexpressing said ORF;
(b) providing a first set of nucleic acids derived from the individual cell and a first oligonucleotide having a first barcode sequence into a discrete partition, wherein the oligonucleotide is attached to a bead, wherein the first set of nucleic acids comprises endogenous transcriptome mRNA and ORF mRNA;
(c) performing RT-PCR to generate a second set of nucleic acids derived from the first set of nucleic acids, wherein said second set of nucleic acids within the partition have attached thereto first oligonucleotides that comprise the first nucleic acid barcode sequence, and wherein the RT-PCR is performed using RT-PCR reagents which comprise a primer which specifically anneals to a sequence on the ORF mRNA, that is not a poly A sequence, and wherein the second set of nucleic acids comprises endogenous transcriptome cDNA and ORF cDNA; and
(d) amplifying the second set of nucleic acids to generate a third set of nucleic acids using PCR reagents which comprise a second primer which specifically anneals to a sequence on the ORF cDNA, that is not a poly A sequence; and
(e) detecting and/or sequencing the barcode sequence, transcriptome cDNA, and/or ORF cDNA.

150. The method of embodiment 149, further comprising (d′) obtaining a portion of the third set of nucleic acids and amplifying the ORF cDNA using a second set of PCR reagents which comprise a third primer which specifically anneals to a sequence on the ORF cDNA, that is not a poly A sequence, to generate a fourth set of nucleic acids.

151. The method of embodiment 150, further comprising (d″) amplifying the ORF cDNA in the fourth set of nucleic acids using a third set of PCR reagents which comprise a fourth primer which specifically anneals to a sequence on the ORF cDNA, that is not a poly A sequence, to generate a fifth set of nucleic acids.

152. The method according to any one of embodiments 149 to 151, further comprising contacting the cell of (a) with a construct comprising an antibody or antibody fragment attached to the first oligonucleotide.

153. The method according to any one of embodiments 149 to 152, further comprising single-cell transcriptome and/or proteome analysis.

154. The method according to any one of embodiments 149 to 153, wherein (e) comprises flow cytometric analysis, cell-hashing, single-cell sequencing analysis, single cell RNA sequencing (scRNA-seq), Perturb-seq, CROP-seq, CRISP-seq, ECCITE-seq, or cellular indexing of transcriptomes and epitopes (CITE-seq).

The following examples are provided for the purpose of illustration only and the invention should in no way be construed as being limited to this example but rather should be construed to encompass any and all variations that become evident as a result of the teachings provided herein.

### EXAMPLES

### Example 1: Materials and Methods

### Isolation and Culture of Primary Human T Cells

Standard buffy coats containing peripheral blood from de-identified healthy donors were collected by and purchased from the New York Blood Center under an IRB-exempt protocol. All donors provided informed consent. Peripheral blood mononuclear cells (PBMC) were isolated from buffy coats using Lymphoprep (Stemcell) gradient centrifugation. For most assays, CD8⁺ and CD4⁺ were isolated sequentially from the same donor. First, CD8⁺ T cells were isolated by magnetic positive selection using the EasySep Human CD8 Positive Selection Kit II (Stemcell). Then, CD4⁺ T cells were isolated from the resulting flowthrough by negative magnetic selection using the Easy Sep Human CD4⁺ T cell Isolation Kit (Stemcell). γδ T cells were isolated by magnetic negative selection using the EasySep Human Gamma/Delta T cell Isolation Kit (Stemcell). Immediately after isolation, T cells were resuspended in T cell medium, which consisted of Immunocult-XF T cell Expansion Medium (Stemcell) supplemented with 10 ng ml⁻¹ recombinant human IL-2 (Stemcell).

Activation of T cells was performed with Immunocult Human CD3/CD28 T cell Activator (Stemcell) using 25 μl per 10⁶ cells per ml. Typically, T cells were transduced with concentrated lentivirus 24 h after isolation. For some experiments, T cells were electroporated with in-vitro-transcribed mRNA 24 h after isolation or with Cas9 protein 48 h after isolation. At 72 h after isolation, lentivirally transduced T cells were selected with 2 μg ml⁻¹ puromycin.

Every 2-3 days, T cells were either split or had the medium replaced to maintain a cell density of 1×10⁶-2×10⁶ cells per ml. Lentivirally transduced T cells were maintained in medium containing 2 μg ml⁻¹ puromycin for the duration of culture. T cells were used for phenotypic or functional assays between 14 and 21 days after isolation, or cryopreserved in Bambanker Cell Freezing Medium (Bulldog Bio). γδ T cells were further purified before functional assays using anti-Vγ9 PE antibody (Biolegend) and anti-PE microbeads (Miltenyi Biotec) according to the manufacturer's recommendations, in the presence of dasatinib, a protein kinase inhibitor, to prevent activation-induced cell death resulting from TCR cross-linking⁴². PBMCs from patients with diffuse large B cell lymphoma were obtained from the Perlmutter Cancer Center under a protocol approved by the Perlmutter Cancer Center Institutional Review Board (S14-02164).

### Vector Design and Molecular Cloning

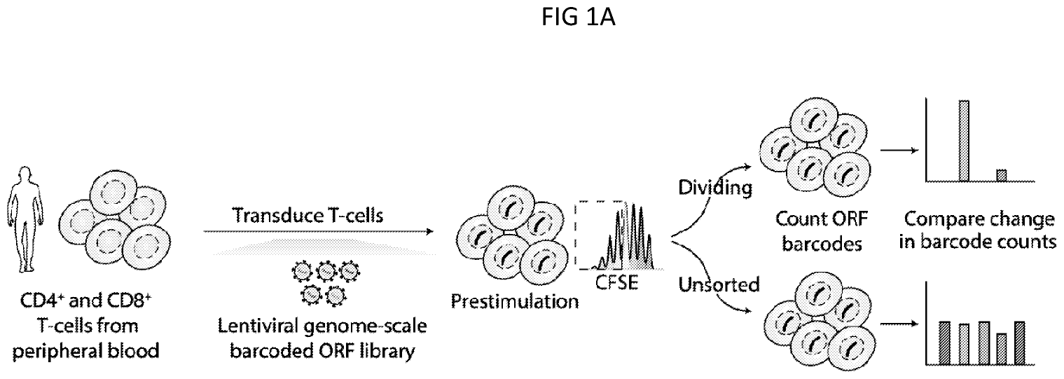

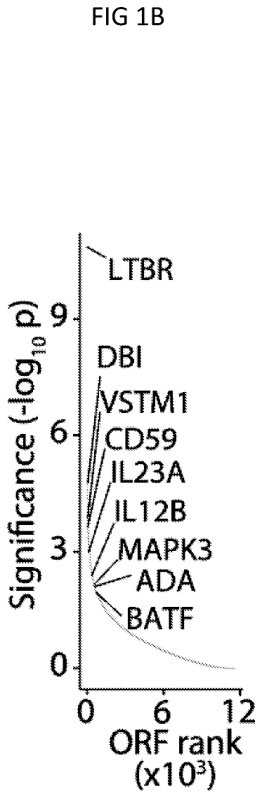

All vectors used were cloned using Gibson Assembly (NEB). For the experiments shown in FIG. 1A-FIG. 1B, we used the lentiviral backbone from the pHAGE plasmid¹⁴. For all other experiments, the backbone from lentiCRISPRv2 (Addgene 52961) was used. ORFs were PCR-amplified for cloning from the genome-scale library used in the screen.

After adding Gibson overhangs by PCR, ORFs and P2A-puro were inserted into XbaI- and EcoRI-cut lentiCRISPRv2. The sgRNA cassette was removed from lentiCRISPRv2 using PacI and NheI digest. For LTBR overexpression and knockout experiments, the sgRNA cassette was not removed. CARs were synthesized as gBlocks (IDT). For CAR-ORF cloning, CAR-P2A-puro-T2A (partial) were first inserted into XbaI- and EcoRI-cut lentiCRISPRv2. For subsequent ORF insertion, the plasmid was cut with HpaI located within the partial T2A and EcoRI. The following vectors were deposited to Addgene: pOT_01 (lenti-EFS-LTBR-2A-puro, Addgene 181970), pOT_02 (lenti-EFS-tNGFR-2A-puro, Addgene 181971), pOT_03 (lenti-EFS-FMC6.3-28z-2A-puro-2A-LTBR, Addgene 181972), pOT_04 (lenti-EFS-FMC6.3-BBz-2A-puro-2A-LTBR, Addgene 181973), pOT_05 (lenti-EFS-FMC6.3-28z-2A-puro-2A-tNGFR, Addgene 181974) and pOT_06 (lenti-EFS-FMC6.3-BBz-2A-puro-2A-tNGFR, Addgene 181975).

### Nuclease and CRISPR Guide RNA Design

All sgRNAs were designed using the GUIDES webtool⁴³. We selected guides that target initial protein-coding exons (with the preference for targeting protein family domains enabled in GUIDES) as well as minimizing off-target and maximizing on-target scores. For Cas9 nuclease nucleofection, we used purified sNLS-SpCas9-sNLS nuclease (Aldevron).

### Preparation of ORF Library Plasmids for Paired-End Sequencing

We re-amplified a previously described genome-scale ORF library.¹⁴ using Endura electrocompetent cells (Lucigen). The identity of ORFs and matched barcodes was confirmed by paired-end sequencing. In brief, the plasmid was first linearized with I-SceI meganuclease, which cuts downstream of the barcode. Then, the linearized plasmid was tagmented using TnY transposase⁴⁴. Then, the fragmented plasmid was amplified in a PCR reaction, using a forward primer binding to a handle introduced by TnY and a reverse primer binding to a sequence downstream of the barcode. All transposons and PCR primer oligonucleotides were synthesized by IDT. The resulting amplicon was sequenced on a NextSeq 500. The forward read (containing the ORF) was mapped to GRCh38.101 CDS transcriptome annotations using STAR v.2.7.3a (map quality ≥10)⁴⁵. Using the paired-end read, we also captured the 24 nucleotide barcode downstream of the constant plasmid sequence. We tabulated ORF-barcode combinations and further curated this table by eliminating any spurious pairs that might be due to sequencing or PCR error. Specifically, a permutation test was performed to identify the maximum number of ORF-barcode combinations expected by random chance, after which we only kept ORF-barcode combinations with a count that exceeded this maximum number. We excluded all non-coding elements from the reference and then collapsed barcodes that were within a Levenshtein distance less than 2.

### Cell Culture

HEK293 FT cells were obtained from Thermo Fisher Scientific and cultured in Dulbecco's modified Eagle's medium (DMEM) supplemented with 10% Serum Plus-II (Thermo Fisher Scientific). Nalm6, Jurkat and BxPC3 cells were obtained from ATCC and cultured in RPMI-1640 supplemented with 10% Serum Plus-II. Capan-2 cells were obtained from ATCC and cultured in McCoy's medium supplemented with 10% Serum Plus-II. For γδ co-incubation experiments, cell lines were pre-treated with 50 uM zoledronic acid (Sigma) for 24 h. Cell lines were routinely tested for mycoplasma using MycoAlert PLUS (Lonza) and found to be negative. Cell lines were not authenticated in this study.

### Lentivirus Production

We produced lentivirus by co-transfecting third-generation lentiviral transfer plasmids together with packaging plasmid psPAX2 (Addgene 12260) and envelope plasmid pMD2.G (Addgene 12259) into HEK293 FT cells, using polyethyleneimine linear MW 25000 (Polysciences). After 72 h, we collected the supernatants, filtered them through a 0.45-μm Steriflip-HV filter (Millipore) and concentrated the virus using Lentivirus Precipitation Solution (Alstem). Concentrated lentivirus was resuspended in T cell medium containing IL-2 and stored at −80 C°.

### Pooled ORF Library Screening

For pooled ORF library screening, CD4⁺ and CD8⁺ T cells were isolated from a minimum of 500×10⁶ PBMCs from 3 healthy donors. The amount of lentivirus used for transduction was titrated to result in 20-30% transduction efficiency, to minimize the probability of multiple ORFs being introduced into a single cell. The cells were maintained in T cell medium containing 2 μg ml−1 puromycin and counted every 2-3 days to maintain a cell density of 1×10⁶-2×10⁶ cells per ml. On day 14 after isolation, T cells were collected, counted, labelled with 5 uM CFSE (Biolegend) and stimulated with CD3/CD28 Activator (Stemcell) at 1.56 μl per 1×10⁶ cells. An aliquot of cells representing 1,000× coverage of the library was frozen down at this step to be used as a pre-stimulation control. After 4 days of stimulation, cells were collected and an aliquot of cells representing 1,000× coverage of the library was frozen down to be used as a pre-sort control. The remaining cells were stained with LIVE/DEAD Violet cell viability dye (Thermo Fisher Scientific), and CFSE^{low} cells (corresponding to the bottom 15% of the distribution) were sorted using a Sony SH800S cell sorter. Genomic DNA was isolated, and two rounds of PCR to amplify ORF barcodes and add Illumina adaptors were performed⁴⁶.

### Pooled ORF Screen Analysis

For most of the analyses, equal numbers of reads from all three donors were combined per bin before trimming and alignment. The barcodes were mapped to the reference library after adaptor trimming with Cutadapt v.1.13 (-m 24 -e 0.1 --discard-untrimmed) using Bowtie v. 1.1.2 (-v 1 -m 1 --best --strata)^{47,48}. All subsequent analyses were performed in RStudio v.1.1.419 with R 4.0.0.2. To calculate individual barcode enrichment, barcode counts were normalized to the total number of reads per sample (with pseudocount added) and log₂-transformed. To calculate ORF enrichment, raw barcode counts were first collapsed by genes before normalization and log₂ transformation.

We performed enrichment analyses at both the barcode and gene level. Statistical analysis on barcode enrichment was performed using MAGeCK⁴⁹, comparing CFSE^{low} samples to corresponding inputs (pre-stimulation), using CD4⁺ and CD8⁺ as replicates. Statistical analysis on ORF enrichment was performed using DESeq2⁵⁰. We obtained raw gene counts by collapsing barcodes into corresponding genes. CFSE^{low} samples were compared to corresponding inputs (both pre-stimulation and pre-sort), using CD4⁺ and CD8⁺ as replicates. GO enrichment (biological process) on genes passing DESeq2 criteria (log₂-transformed fold change >0.5, P_{adj}<0.05) was performed using the topGO package⁵¹. For the genes enriched in the CFSE^{low} screen (DESeq2 analysis), we overlapped these genes with differentially expressed genes after CD3/CD28 stimulation using data from the Database of Immune Cell eQTLs, Expression, Epigenomics (DICE: https://dice-database.org/)⁴¹. For differentially expressed genes, we used the following DICE datasets: ‘T cell, CD4, naive’ versus ‘T cell, CD4, naive [activated]’, ‘T cell, CD8, naive’ versus ‘T cell, CD8, naïve [activated]’. Significant differential expression was as given in the DICE dataset (P_{adj}<0.05).

### Proliferation Assays

Transduced T cells were collected at day 14 after isolation, counted and plated at 2.5×10⁴ cells per well in a round bottom 96-well plate, in 2 sets of triplicate wells per transduction. One set of triplicate wells was cultured in Immunocult-XF T cell Expansion Medium supplemented with 10 ng ml⁻¹ IL-2 and another set of triplicate wells was further supplemented with 1.56 μl CD3/CD28 Activator per 1 ml of medium. The cells were cultured for 4 days, and then were collected and stained with LIVE/DEAD Violet cell viability dye. Before flow cytometric acquisition, the cells were resuspended in D-PBS with 10% v/v Precision Counting Beads (Biolegend). For quantification, the number of viable cell events was normalized to the number of bead events per sample. Then, for each ORF the normalized number of viable cells in wells supplemented with CD3/CD28 Activator was divided by the mean number of viable cells in control wells to quantify T cell proliferation. To enable comparisons between donors and CD4⁺/CD8⁺ T cells, the proliferation of T cells transduced with a given ORF was finally normalized to the proliferation of a matched tNGFR control.

In addition to the counting beads assay, we also measured proliferation using a dye dilution assay. For this assay, transduced T cells were collected at day 14 after isolation, washed with D-PBS and then labelled with 5 uM CellTrace Yellow (CTY) in D-PBS for 20 min at room temperature. The excess dye was removed by washing with a fivefold excess of RPMI-1640 supplemented with 10% Serum Plus-II. The labelled cells were then plated at 2.5×10⁴ cells per well on a round bottom 96-well plate. One set of triplicate wells was cultured in supplemented Immunocult-XF T cell Expansion Medium (that is, without IL-2) and another set of triplicate wells was supplemented with 10 ng ml⁻¹ IL-2 and 1.56 μl CD3/CD28 Activator per 1 ml of medium. The cells were cultured for 4 days, and then were collected and stained with LIVE/DEAD Violet cell viability dye. For quantification of the proliferation index, events were first gated on viable T cells in FlowJo (Treestar) and exported for further analysis in R/RStudio using the flow Fit and flow Core packages⁵². Unstimulated cells were used to determine the parent population size and position to account for differences in staining intensity between different samples. These fitted parent population parameters were then used to fit the CTY profiles of matched stimulated samples, modelled as Gaussian distributions assuming log₂-distanced peaks as a result of cell division and dye dilution. Fitted CTY profiles were inspected visually for concordance with the original CTY profiles and used to calculate the proliferation index. The proliferation index is defined as the sum of cells in all generations divided by the computed number of parent cells present at the beginning of the assay.

### Flow Cytometry for Cell-Surface and Intracellular Markers

For CD25 (IL2RA) and CD154 (CD40L) quantification, T cells were restimulated with CD3/CD28 Activator (6.25 μl per 10⁶ cells) for 6 h (CD154 staining in CD8⁺) or for 24 h before staining (CD25 staining in both CD4⁺ and CD8⁺, and CD154 staining in CD4⁺). For Ki-67 and 7-amino-actinomycin D (7-AAD) staining, T cells were rested overnight in Immunocult-XF T cell Expansion Medium without IL-2 and then activated with CD3/CD28 Activator (25 μl per 10⁶ cells) for 24 h. In other cases, T cells were stained without stimulation. For detection of secreted proteins, T cells were stimulated for 24 h with CD3/CD28 Activator (25 μl per 10⁶ cells) (LTA, LIGHT), and protein transport inhibitors brefeldin A (5 μg ml⁻¹) and monensin (2 μM) were included for the last 6 h of stimulation (IL12B, LTA, LIGHT).

First, the cells were collected, washed with D-PBS and stained with LIVE/DEAD Violet cell viability dye for 5 min at room temperature in the dark, followed by surface antibody staining for 20 min on ice. After surface antibody staining (where applicable) the cells were washed with PBS and acquired on a Sony SH800S cell sorter or taken for intracellular staining. For intracellular staining, the cells were resuspended in an appropriate fixation buffer. The following fixation buffers were used for specific protein detection: Fixation Buffer (Biolegend) for IL12B and MS4A3 staining; True-Nuclear Transcription Factor Fix (Biolegend) for BATF, TCF1 and FLAG staining; and FoxP3/Transcription Factor Fixation Reagent, (eBioscience) for Ki-67. After resuspension in the fixation buffer, cells were incubated at room temperature in the dark for 1 h. Following the incubation, the cells were washed twice in the appropriate permeabilization buffer. The following permeabilization buffers were used: Intracellular Staining Permeabilization Wash Buffer (Biolegend) for IL12B and MS4A3 staining; True-Nuclear Perm Buffer (Biolegend) for BATF, TCF1 and FLAG staining; and FoxP3/Transcription Factor Permeabilization Buffer (eBioscience) for Ki-67. After permeabilization, the cells were stained with the specific antibody or isotype control for 30 min in the dark at room temperature. Finally, the cells were washed twice in the appropriate permeabilization buffer and acquired on a Sony SH800S flow cytometer. For cell-cycle analysis, the cells were further stained with 0.5 g ml⁻¹ 7-AAD for 5 min immediately before acquisition. Gating was performed using appropriate isotype, fluorescence minus one and biological controls. Typically, 5,000-10,000 live events were recorded per sample.

### Flow Cytometry Detection of Phosphorylated Proteins

T cells were rested for 24 h in in Immunocult-XF T cell Expansion Medium without IL-2 before detection of phosphorylated proteins. The rested cells were stimulated with CD3/CD28 Activator (25 μl per 10⁶ cells) for the times indicated in the corresponding figure. Immediately after the stimulation period, the cells were fixed with a 1:1 volume ratio of the pre-warmed Fixation Buffer (Biolegend) for 15 min at 37° C. and washed twice with the cell staining buffer (D-PBS+2% FBS). As per the manufacturer's protocol, the cells were resuspended in the residual volume and permeabilized in 1 ml of pre-chilled True-Phos Perm Buffer (Biolegend) while vortexing. The cells were incubated in the True-Phos Perm Buffer for 60 min at −20° C. After permeabilization the cells were washed twice with the cell staining buffer and stained with anti-CD4, anti-CD8, anti-RELA and anti-phospho-RELA antibodies (or isotype controls) for 30 min at room temperature. After staining, the cells were washed twice in the cell staining buffer and acquired on a Sony SH800S cell sorter. Gating was performed on CD4⁺ or CD8⁺ cells, and the levels of RELA and phospho-RELA were determined using appropriate isotype and biological controls.

### Western Blot Detection of Proteins and Phosphorylated Proteins

T cells expressing tNGFR or LTBR, resting or stimulated for 15 min with CD3/CD28 Activator (25 μl per 10⁶ cells), were collected, washed with 1×D-PBS and lysed with TNE buffer (10 mM Tris-HCl, pH 7.4, 150 mM NaCl, 1 mM EDTA, 1% Nonidet P-40) in the presence of a protease inhibitor cocktail (Bimake B14001) and a phosphatase inhibitor cocktail (Cell Signaling Technologies 5872S) for 1 h on ice. Cell lysates were spun for 10 min at 10,000 g, and the protein concentration was determined with the BCA assay (Thermo Fisher Scientific). Equal amounts of cell lysates (25 mg) were denatured in Tris-Glycine SDS Sample buffer (Thermo Fisher Scientific) and loaded on a Novex 4-12 or 4-20% Tris-Glycine gel (Thermo Fisher Scientific). The PageRuler pre-stained protein ladder (Thermo Fisher Scientific) was used to determine the protein size. The gel was run in 1× Tris-Glycine-SDS buffer (IBI Scientific) for about 120 min at 120 V. Proteins were transferred on a nitrocellulose membrane (BioRad) in the presence of prechilled 1× Tris-Glycine transfer buffer (Thermo Fisher Scientific) supplemented with 20% methanol for 100 min at 100 V.

Immunoblots were blocked with 5% skimmed milk dissolved in 1×PBS with 1% Tween-20 (PBST) and incubated overnight at 4° C. separately with the following primary antibodies: rabbit anti-GAPDH (0.1 mg ml⁻¹, Cell Signaling, 2118S), mouse anti-IKKα (1:1,000 dilution, Cell Signaling, 3G12), rabbit anti-IKKβ (1:1,000 dilution, Cell Signaling, D30C6), rabbit anti-NF-κB p65 (1:1,000 dilution, Cell Signaling, D14E12), rabbit anti-phospho-NF-κB p65 Ser536 (1:1,000 dilution, Cell Signaling, 93H1), mouse anti-IκBα (1:1,000 dilution, Cell Signaling, L35A5), rabbit anti-phospho-IκBα Ser32 (1:1,000 dilution, Cell Signaling, 14D4), rabbit anti-NF-κB p100/p52 (1:1,000 dilution, Cell Signaling, 4882) and rabbit anti-RELB (1:1,000 dilution, Cell Signaling, C1E4). After the primary antibody, the blots were incubated with IRDye 680RD donkey anti-rabbit (0.2 mg ml⁻¹, LI-COR 926-68073) or with IRDye 800CW donkey anti-mouse (0.2 mg ml⁻¹, LI-COR 926-32212). The blots were imaged using Odyssey CLx (LI-COR) and quantified using ImageJ v. 1.52.

### Quantification of Cytokine Secretion

For measurement of secreted IFNγ and IL-2, T cells were first collected and rested for 24 h in medium without IL-2. Then, they were counted, plated at 2.5×10⁴ cells per well in a round bottom 96-well plate and incubated in medium without IL-2, with or without CD3/CD28 Activator (25 μl per 10⁶ cells) for 24 h. Then, cell supernatants were collected, diluted and used for cytokine quantification with an enzyme-linked immunosorbent assay (Human IL-2 or IFNγ DuoSet, R&D Systems), using an Infinite F200 Pro (Tecan) plate reader. Multiplexed quantification of secreted cytokines and chemokines in resting or stimulated T cells was performed using the Human Cytokine/Chemokine 48-Plex Discovery Assay Array (Eve Technologies).

### T Cell Killing Assays

CD19⁺ Nalm6 cells were first transduced with a lentiviral vector encoding EGFPd2PEST-NLS and a puromycin resistance gene⁵³. The transduced cells were kept in puromycin selection throughout the culture, to maintain stable EGFP expression, and puromycin was only removed from the medium before the killing assay. T cells were transduced with a vector encoding a CAR specific for CD19, using either a CD28 stalk, CD28 transmembrane and CD28 signaling domain or CD8 stalk and CD8 transmembrane domain with 4-1BB signaling domain, and CD3ζ signaling domain⁵⁴. Fourteen days after transduction, transduced T cells were combined with 5×10⁴ Nalm6 GFP⁺ cells in triplicate at indicated effector:target ratios in a flat 96-well plate pre-coated with 0.01% poly-1-ornithine (EMD Millipore) in Immunocult medium without IL-2. The wells were then imaged using an Incucyte SX1, using 20× magnification and acquiring 4 images per well every 2 h for up to 120 h. For each well, the integrated GFP intensity was normalized to the 2 h time point, to allow the cells to fully settle after plating.

In Vitro mRNA Preparation

The template for in vitro transcription was generated by PCR from a plasmid encoding LTBR or tNGFR with the resulting amplicon including a T7 promoter upstream of the ORF. The purified template was then used for in vitro transcription with capping and poly-A tailing using the HiScribe T7 ARCA mRNA Kit with Capping (NEB).

### Primary T Cell Nucleofection

Activated T cells were nucleofected with in-vitro-transcribed mRNA at 24 h after activation or with Cas9 protein at 48 h after activation. The cells were collected, washed twice in PBS and resuspended in P3 Primary Cell Nucleofector Solution (Lonza) at 5×10⁵ cells per 20 μl. Immediately after resuspension, 1 ug mRNA or 10 ug Cas9 (Aldevron) were added (not exceeding 10% v/v of the reaction) and the cells were nucleofected using the E0-115 program on a 4D-Nucleofector (Lonza). After nucleofection the cells were resuspended in pre-warmed Immunocult medium with IL-2 and recovered at 37° C. with 5% CO2 for 20 min. After recovery, the cells were plated at 1×10⁶ cells per ml and used in downstream assays.

### OverCITE-Seq Sample Preparation and Sequencing

For single-cell sequencing, CD8⁺ T cells were individually transduced with ORFs and kept, separately, under puromycin selection for 14 days. Then, transduced cells were combined and split into two conditions: one was cultured for 24 h only in the presence of IL-2; the other was further supplemented with 6.25 μl CD3/CD28 Activator per 10⁶ cells. After stimulation, the cells were collected, counted and resuspended in staining buffer (2% BSA+0.01% Tween-20 in PBS) at 2×10⁷ cells per ml. Then, 10% (v/v) Human TruStain FcX Fc Receptor Blocking Solution (Biolegend) was added, and the cells were incubated at 4° C. for 10 min. After Fc receptor blocking, the cell concentration was adjusted to 5×10⁶ cells per ml and the stimulated and unstimulated cells were split into 4 conditions each. Each condition received a different oligonucleotide-conjugated (barcoded) cell hashing antibody to allow for pooling of different conditions in the same 10× Genomics Chromium lane²³. After 20 min co-incubation on ice, the cells were washed 3 times with staining buffer and counted using Trypan blue exclusion. Cell viability was typically around 95%.

Then, cells stained with different hashing antibodies were pooled together at equal numbers and stained with the following oligonucleotide-conjugated (barcoded) antibodies for quantification of cell surface antigens: CD11c (0.1 μg), CD14 (0.2 μg), CD16 (0.1 μg), CD19 (0.1 μg), CD56 (0.2 μg), CD3 (0.2 μg), CD45 (0.01 μg), CD45RA (0.2 μg), CD45RO (0.2 μg), CD4 (0.1 μg), CD8 (0.1 μg), CD25 (0.25 μg), CD69 (0.25 μg) and NGFR (0.25 μg) (TotalSeq-C, Biolegend). The cells were stained for 30 min on ice, washed 3 times with staining buffer, resuspended in PBS and filtered through a 40-μm cell strainer. The cells were then counted and the concentration was adjusted to 1×10⁶ ml⁻¹. For loading into the 10× Genomics Chromium, 3×10⁴ cells were combined with Chromium Next GEM Single Cell 5′ v2 Master Mix (10× Genomics) supplemented with a custom reverse primer binding to the puromycin resistance cassette for boosting ORF transcript capture at the reverse transcription stage. The custom reverse primer was added at a 1:3 ratio to the poly-dT primer included in the Master Mix.

For cDNA amplification, additive primers for amplification of sample hashing and surface antigen barcodes were included²³, as well as a nested reverse primer binding to the puromycin resistance cassette downstream of the ORF. Following cDNA amplification, SPRI beads were used for size selection of resulting PCR products: small-size (fewer than 300 bp) sample hashing and surface antigen barcodes were physically separated from larger cDNA and ORF amplicons for downstream processing. Sample hashing and surface antigen barcodes were also processed²². Amplified cDNA was then separated into three conditions, for construction of the gene expression library, αβ TCR library and ORF library. The ORF library was processed similarly to the αβ TCR library, using nested reverse primers binding downstream of the ORF. The quality of produced libraries was verified on BioAnalyzer using the High Sensitivity DNA kit (Agilent). The libraries were sequenced on a NextSeq 500. For the gene expression library, more than 25,000 reads per cell were generated. For other libraries, more than 5,000 reads per cell were generated.

### OverCITE-Seq Data Analysis

Gene expression unique molecular identifier (UMI) count matrices and TCR clonotypes were derived using 10× Genomics Cell Ranger 3.1.0. Hashtag oligo (HTO) and antibody UMI count matrices were generated using kallisto v.0.46.0⁵⁵ and bustools v.0.39.3⁵⁶. ORF reads were first aligned to plasmid references using Bowtie2 v.2.2.8⁵⁷ and indexed to the associated ORF, after which kallisto and bustools were used to generate UMI count matrices. All modalities were normalized using a centred log ratio (CLR) transformation. Cell doublets and negatives were identified using the HTODemux⁵⁸ function and then excluded from downstream analysis. The UMI cut-off quantile for HTODemux was optimized to maximize singlet recovery using grid search with values between 0 and 1. ORF singlets were identified using MULTIseqDemux⁵⁹. We then excluded cells with low-quality gene expression metrics and removed cells with fewer than 200 unique RNA features or greater than 5% of reads mapping to the mitochondrial transcriptome.

Count matrices were then loaded into and analyzed with Seurat v.4.0.1⁶⁰. Cell cycle correction and scaling of gene expression data was performed using the CellCycleScoring function with default genes, followed by scaling the data using the ScaleData function. Principal component (PC) optimization of the scaled and corrected data was then performed using JackStraw⁶¹, in which we selected all PCs up to the first non-significant PC to use in clustering. Clustering of cells was performed using a shared nearest neighbor (SNN)-based clustering algorithm and visualized using UMAP dimensional reduction⁶² to project cluster PCs into 2D space. Cluster marker analysis was performed using the FindAllMarkers function with the hypothesis set defined as positive and negative markers present in at least 25% of cluster cells and with a log₂-transformed fold change threshold of 0.25 as compared to non-cluster cells. Differential expression analysis of ORFs was performed using DESeq2⁵⁰ to identify genes up and downregulated in ORF-expressing cells as compared to NGFR (control) cells, with differential expression defined as those with q<0.1 calculated using the Storey method⁶³.

### Bulk RNA-Seq and Analysis

CD4⁺ and CD8⁺ LTBR- or tNGFR-transduced T cells were stimulated for 24 h with CD3/CD28 Activator (25 μl per 10⁶ cells) or left unstimulated (n=3 biological replicates). Total RNA was extracted using the Direct-zol RNA purification kit (Zymo). The 3′-enriched RNA-seq library was prepared as described before⁶⁴. In brief, RNA was reverse-transcribed using SMARTScribe Reverse Transcriptase (Takara Bio) and a poly(dT) oligo containing a partial Nextera handle. The resulting cDNA was then PCR-amplified for 3 cycles using OneTaq polymerase (NEB) and tagmented for 5 min at 55° C. using homemade transposase TnY⁴⁴. Immediately afterwards, the tagmented DNA was purified on a MinElute column (Qiagen) and PCR-amplified using One Taq polymerase and barcoded primers for 12 cycles. The PCR product was purified using a dual (0.5×-0.8×) SPRI clean-up (Agencourt) and the size distribution was determined using Tapestation (Agilent). Samples were sequenced on a NextSeq 500 (Illumina) using a v2.5 75-cycle kit (paired end). Paired-end reads were aligned to the transcriptome (human Ensembl v.96 reference⁶⁵) using kallisto v.0.46.0⁵⁵ and loaded into RStudio 1.1.419 with R 4.0.0.2 using the tximport package⁶⁶. Differential gene expression analysis was performed using DESeq2⁵⁰. GO enrichment (biological process) on genes passing DESeq2 criteria (log₂-transformed fold change >1, Padj<0.05) was performed using the topGO package⁵¹.

### ATAC-Seq Library Preparation

CD8⁺ LTBR and tNGFR T cells were stimulated for 24 h with CD3/CD28 Activator (25 μl per 10⁶ cells) or left unstimulated (n=2 biological replicates). We performed bulk ATAC-seq as previously described⁴⁴. In brief, cell membranes were lysed in the RSB buffer (10 mM Tris-HCL pH 7.4, 3 mM MgCl₂, 10 mM NaCl) with 0.1% IGEPAL freshly added. After pipetting up and down, nuclei were isolated by centrifugation at 500 g for 5 min at 4° C. After discarding the supernatant, the nuclei were resuspended in the Tagmentation DNA (TD) Buffer⁴⁴ with homemade transposase TnY protein⁴⁴ and incubated at 37° C. for 30 min. After purification on a MinElute column (Qiagen), the tagmented DNA was PCR-amplified using a homemade Pfu X7 DNA polymerase⁴⁴ and barcoded primers for 12 cycles. The PCR product was purified via a 1.5×SPRI clean-up (Agencourt) and checked for a characteristic nucleosome banding pattern using TapeStation (Agilent). Samples were sequenced on a NextSeq 500 (Illumina) using the v2.5 75-cycle kit (single end).

### ATAC-Seq Analysis

Single-end reads were aligned to the Gencode hg38 primary assembly.⁶⁷ using Bowtie2 v.2.4.4⁵⁷. We then used SAMtools v. 1.9⁶⁸ to filter out alignments with low-mapping quality (MAPQ <30) and subsequently to sort and index the filtered BAM files⁶⁸. Read duplicates were removed using Picard v.4.1.8.1⁶⁹. Peaks were called using MACS3 v.3.0.0⁷⁰ with default parameters (-g 2.7e9 -q 0.05).

To construct the union feature space (‘union peaks’) used for much of the downstream analyses, we began by performing intersections on pairs of biological replicate narrow Peak files using BEDTools v.2.29.0 (using bedtools intersect), keeping only those peaks found in both replicates⁷¹. After marking the shared peaks between replicates, we used bedtools merge to consolidate the biological replicates at each shared peak (at least 1 bp overlap). In this new peak BED file, each shared peak includes all sequence found under the peak in either of the biological replicates. Next, we took the union of each of these peak files (LTBR resting, LTBR stimulated, tNGFR resting, tNGFR stimulation); we combined any peaks with at least 1 bp overlap. Using the union peaks, we generated a peak read count matrix (union peaks×ATAC samples), in which each entry in the matrix corresponds to the number of reads overlapping that peak in the specified sample—we term this the per-peak ATAC matrix. The overlapping reads are taken directly from the BAM files (converted to BED) that provide an alignment for each sample. Thus, the matrix includes a column for each biological replicate. Although samples had minimal differences in aligned reads, we normalized each entry in the matrix by the number of reads that overlapped the TSS regions in each sample. In this manner, any difference in read or alignment depth between samples would be normalized appropriately. In addition to the per-peak ATAC matrix, we also constructed a per-gene ATAC matrix as follows: we assigned a gene's total ATAC reads as the sum of normalized reads from the per-peak ATAC matrix for all peaks within 3 kb of a gene's start or end coordinates.

We imported these two ATAC matrices (per-peak and per-gene) into R v.4.1.1 for gene and peak enrichment analysis using DESeq2 v.1.32.0. For comparison between ATAC-seq and RNA-seq, we used a statistical threshold of adjusted P value <0.05 and either log₂-transformed fold change >0 (for increases in ATAC or RNA) or log₂-transformed fold change <0 (for decreases in ATAC or RNA). For transcription factor-motif analysis we used Chrom-VAR v. 1.14.072 as follows: For each of the test versus control conditions, we constructed Summarized Experiment objects using column and sample subsets of the per-peak matrix and the union feature space. We used the matchMotifs function to annotate transcription factormotifs. We computed enrichment deviations between test and control conditions using the computeDeviations function.

To produce read pile-up tracks at specific genomic loci, we pooled de-duplicated reads from biological replicates (BAM) using samtools merge. We converted these pooled-replicate BAM files to bigWig files by using the bamCoverage function from deeptools v.3.4.2 and setting the scaleFactor to the relative number of TSSs found in the pooled biological replicates compared to all other sample aggregates⁷³. Using the bigWig files, read pileups were plotted with py Genome Tracks v.3.6⁷⁴.

Finally, we performed k-means clustering on ATAC peaks near genes with increased chromatin accessibility. First, using DEseq2 on the ATAC per-gene matrix, we identified genes with log₂-transformed fold change >1 and adjusted P value <0.05 (that is, genes with increased chromatin accessibility) in either of two comparisons: (1) LTBR stimulated versus tNGFR stimulated; (2) LTBR resting versus tNGFR resting. After identifying these genes, we isolated all accessibility peaks in the per-peak ATAC matrix within 3 kb of the gene body; this subset of peaks from the per-peak ATAC matrix was used as input for the clustering. Then, using deeptools (computeMatrix and plotHeatmap functions) on this subset of ATAC peaks, we performed k-means clustering with k=4 clusters and 6 kb read windows.

### Statistical Analysis

Data between two groups were compared using a two-tailed unpaired Student's t-test or the Mann-Whitney test as appropriate for the type of data (depending on the normality of the distribution). Unless otherwise indicated, a P value less than or equal to 0.05 was considered statistically significant for all analyses, and not corrected for multiple comparisons. In cases in which multiple comparison corrections were necessary, we adjusted the P value using the Benjamini-Hochberg method. All group results are represented as mean±s.e.m., if not stated otherwise. Statistical analyses were performed in Prism (GraphPad) and RStudio (Rstudio PBC). Flow cytometry data were analyzed using FlowJo v. 10.7.1 (Treestar).

### Example 2: Genome-Scale Screen for Synthetic Drivers of T-Cell Proliferation

We performed a genome-scale gain-of-function screen in primary human CD4⁺ and CD8⁺ T cells, using a lentiviral library of barcoded human ORFs. We show that T cells with the strongest proliferation phenotypes are enriched for both known and unknown regulators of the immune response, many of which are not typically expressed by peripheral T cells. We validate top-ranked ORFs in cells from screen-independent donors and further demonstrate that these ORFs not only drive T cell proliferation but also increase the expression of activation markers and the secretion of key proinflammatory cytokines. To gain more comprehensive insight into the mechanism of action of these genes, we develop a single-cell sequencing approach coupled with direct ORF capture. We identify LTBR—one of the top-ranked ORFs not expressed by lymphocytes—as a key driver of profound transcriptional and epigenetic remodeling through increased NF-κB signaling, which results in a marked increase in the secretion of proinflammatory cytokines and resistance to apoptosis. Finally, we show that top-ranked ORFs potentiate antigen-specific T cell functions, in the context of CD19-directed CAR T cells and broadly tumor-reactive γδ T cells from healthy donors and patients with blood cancer.

### Genome-Scale ORF Screen in T Cells

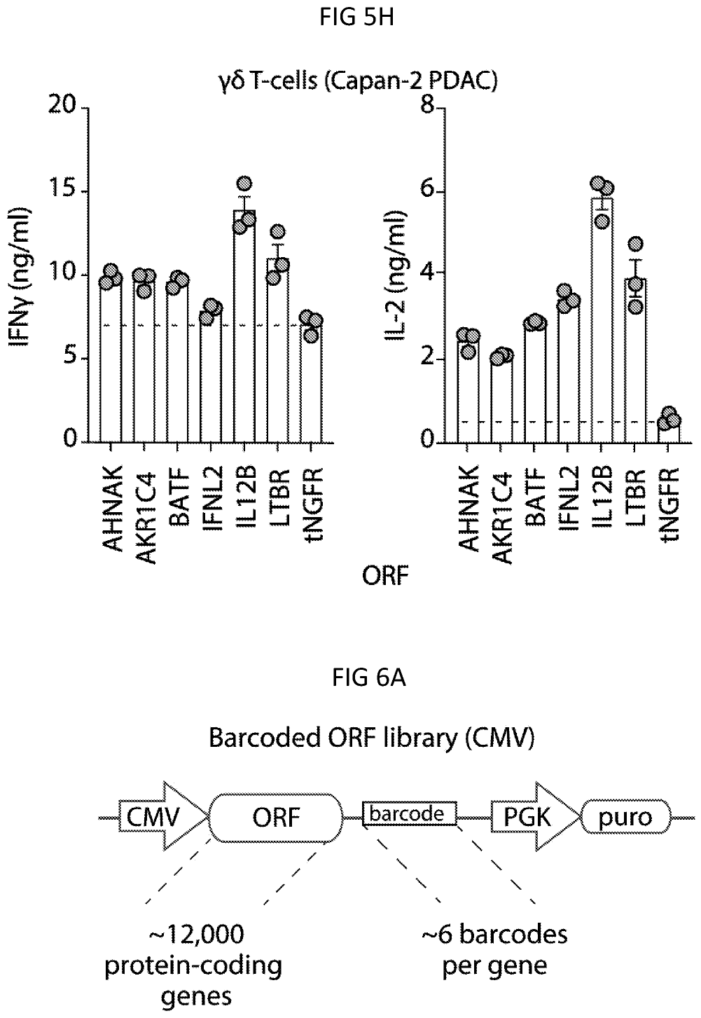

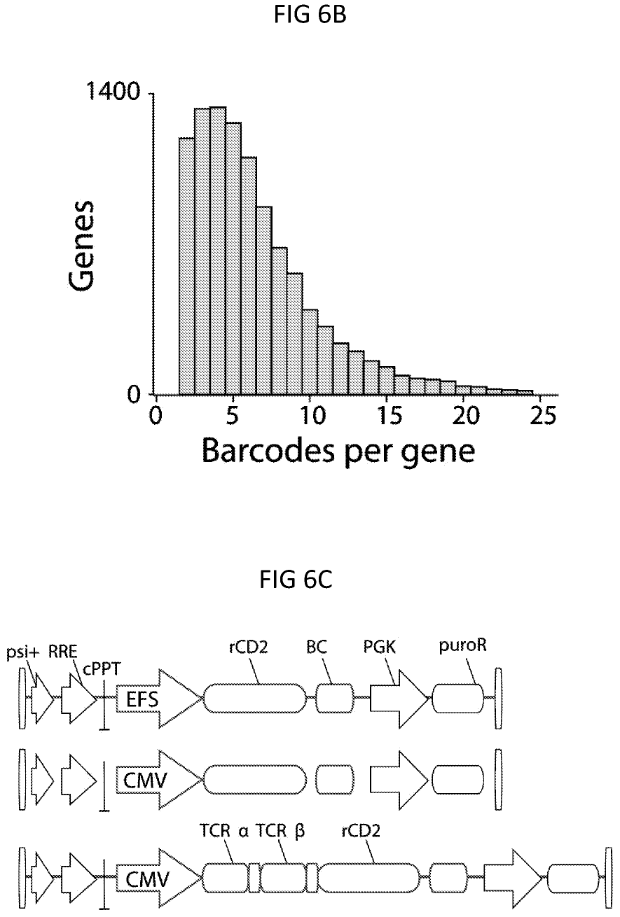

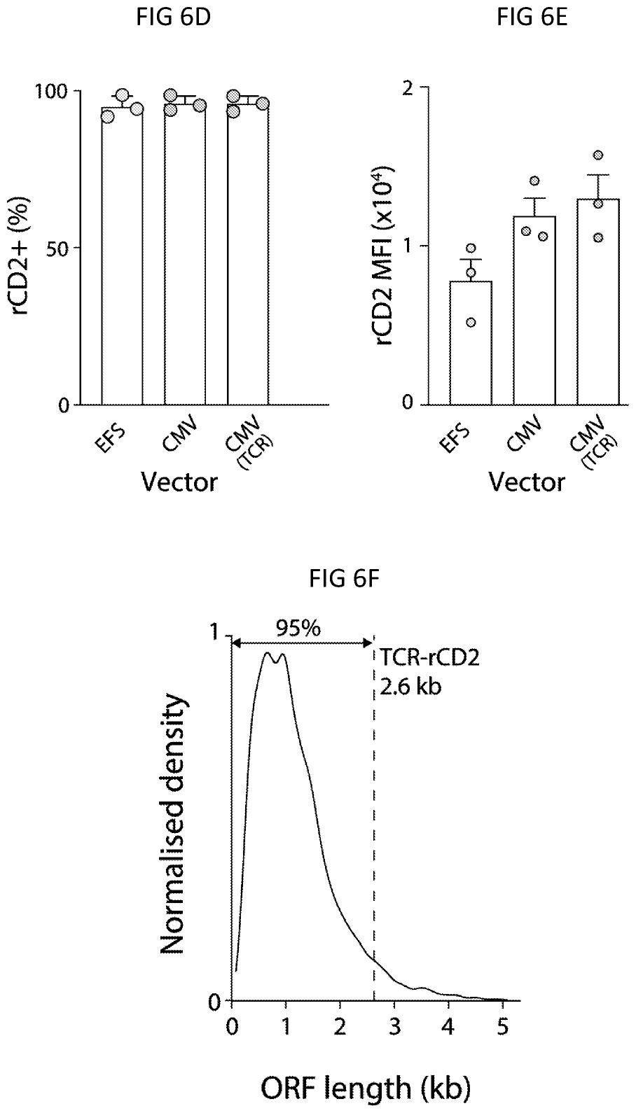

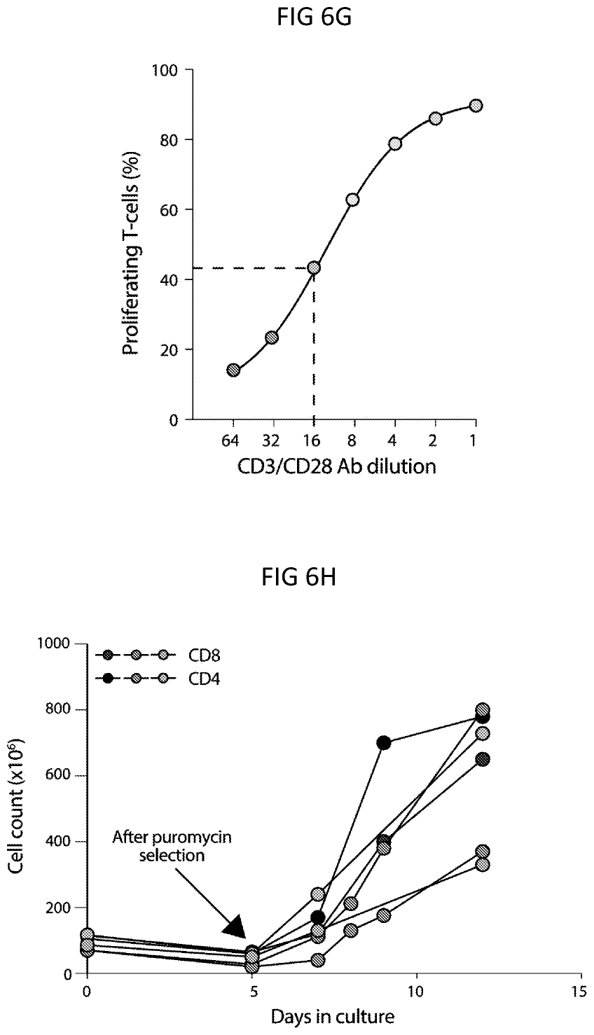

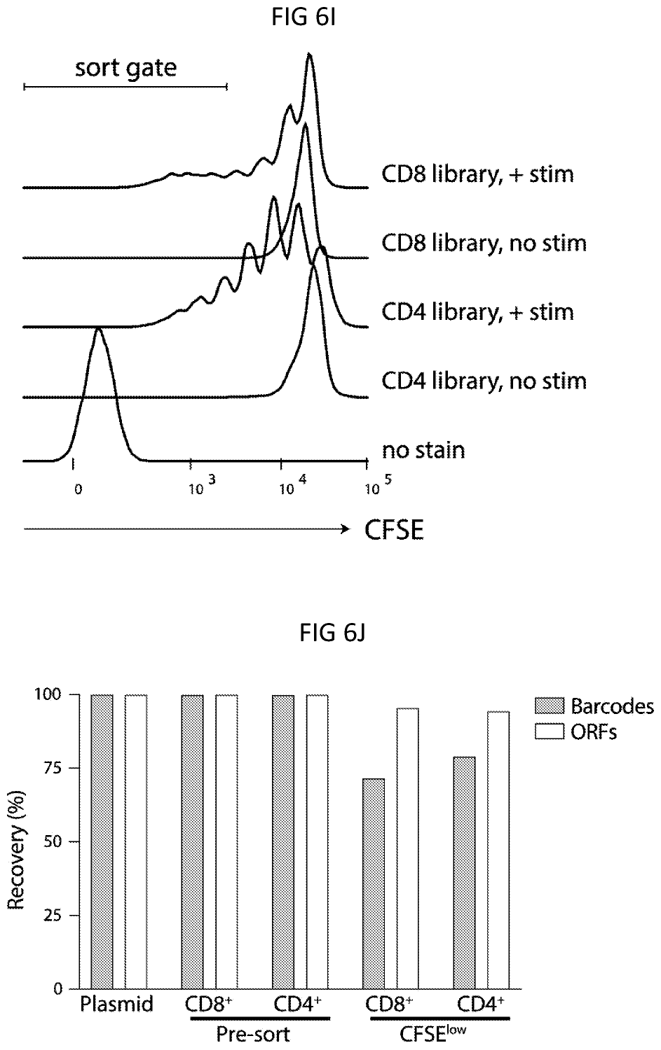

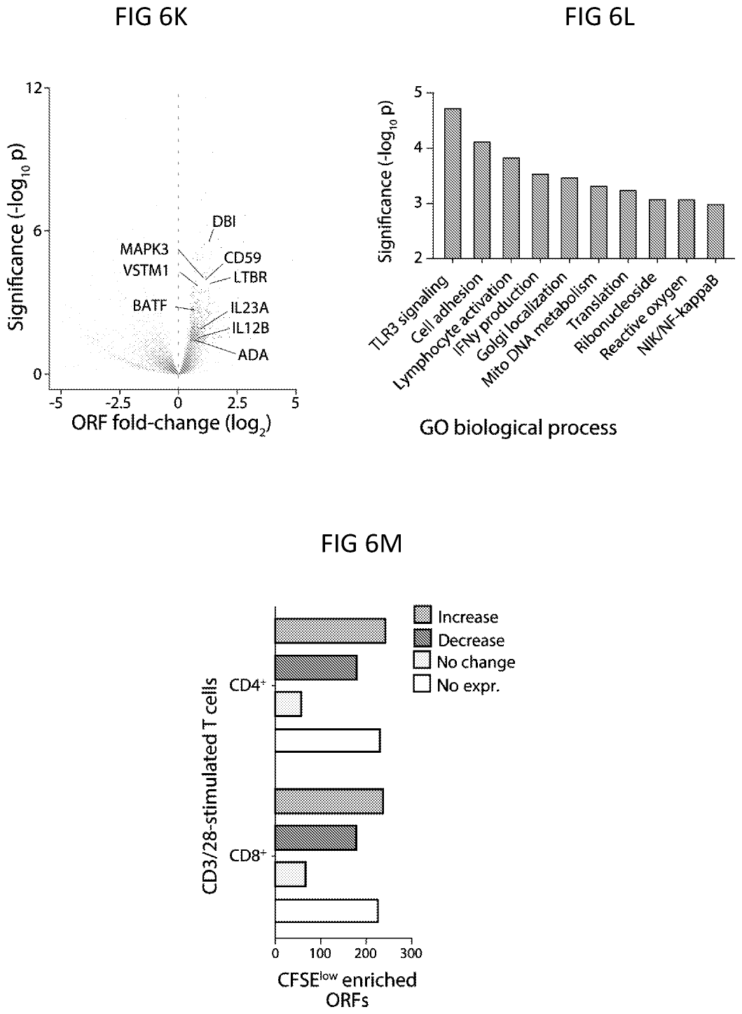

To avoid relying on constitutive expression of large bacterial proteins or chromatin accessibility in the vicinity of target genes¹³, we decided to use a lentiviral library of human ORFs; this library contains nearly 12,000 full-length genes, with around 6 barcodes per gene¹⁴ (FIG. 1A, FIG. 6A-FIG. 6F). Previously, genome-scale loss-of-function screens in human T cells have focused on either CD4⁺ or CD8⁺ T cells. However, both CD4⁺ and CD8⁺ T cells are required for durable tumor control in adoptive therapies^{15,16}, as further exemplified by FDA approvals of anti-CD19 CAR T cells with a defined 1:1 CD4⁺ and CD8⁺ ratio¹. Thus, we decided to use the ORF library to discover genes that boost the proliferation of both CD4⁺ and CD8+ T cells in response to T cell receptor (TCR) stimulation (FIG. 1A, FIG. 6G-FIG. 6I).

We transduced the lentiviral ORF library into CD4⁺ and CD8⁺ T cells from three healthy donors, and after a brief period in culture (14 days) we restimulated the cells to identify drivers of proliferation in response to TCR stimulation. We were able to capture the majority of individual ORF barcodes, and nearly all ORFs, including the largest ones (FIG. 6J). Comparing the relative frequencies of genes in the most highly proliferative cells to unsorted cells, we found an enrichment of genes that are known to participate in immune processes among the top-ranked ORFs (FIG. 6K). We identified MAPK3 (encoding ERK1), a critical mediator of T cell functions¹⁷, the co-stimulatory molecule CD59¹⁸, the transcription factor BATF, and cytokines that are known to promote T cell proliferation, such as IL12B and IL23A¹⁹. In fact, two recent studies showed that overexpression of IL12B and BATF boosts proliferation, cytotoxicity and cytokine secretion in CAR T cells^{19,20}.

Each ORF in the library is linked to an average of six DNA barcodes (FIG. 6B). To increase confidence in our top-ranked ORFs from the pooled screen, we assessed the enrichment of individual barcodes corresponding to a given ORF in proliferating CD4⁺ and CD8⁺ cells (FIG. 6B, FIG. 6C). For the majority of ORFs, multiple individual barcodes for each gene were enriched in the highly proliferating population, thus suggesting that the observed enrichment does not stem from spurious clonal outgrowth or PCR bias. Surprisingly, the most significantly enriched gene was lymphotoxin-β receptor (LTBR), a gene that is broadly expressed in stromal and myeloid cells but completely absent in lymphocytes.

Overall, the enriched ORFs spanned a range of diverse biological processes. Among the top-enriched Gene Ontology (GO) biological processes were lymphocyte proliferation, interferon-γ (IFNγ) production and NF-κB signaling (FIG. 6L). We observed that enriched ORFs showed only a slight preference for genes endogenously upregulated by T cells during stimulation with CD3 and CD28 (CD3/CD28), and in fact were represented in all classes of differential expression (FIG. 6M). This result highlights the capacity of the pooled ORF screen to discover genes that enable T cell proliferation but that are not expressed normally during CD3/CD28-mediated activation and proliferation. For subsequent validation, we decided to test a broad range of ORFs that function in diverse pathways of relevance to T cell fitness, and that showed different modes of endogenous regulation. Neutral genes (MHC-I complex and cell-type specific differentiation markers) are included for comparison. Genes were identified on the basis of differential expression in CD3/CD28-stimulated and resting T cells (upregulated: IL1RN, NFYB, BATF, AHNAK, CLIC1, RAN, DBI, GPD1, GPN3, AHCY, HOMER1, MRPL18, MRPL51, LIG3, ZNF830, HLA-A: downregulated: FOSB, ATF6B, SLC10A7, CDK2, ADA, CD19; no change: CDK1, DCLRE1B, B2M: no expression: IFNL2, LTBR, CXCL12, CRLF2, IL12B, CALML3, CYP27A1, AKR1C4, DUPD1, NGFR)⁴¹.

### Top ORFs Enhance T Cell Functions

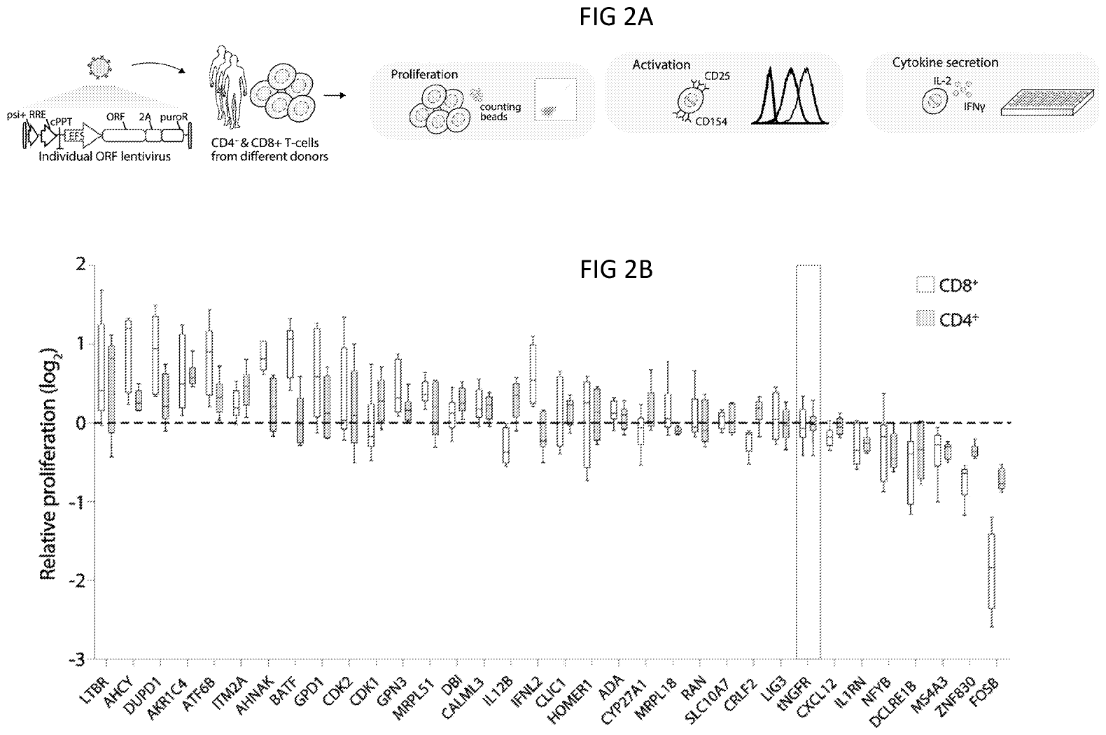

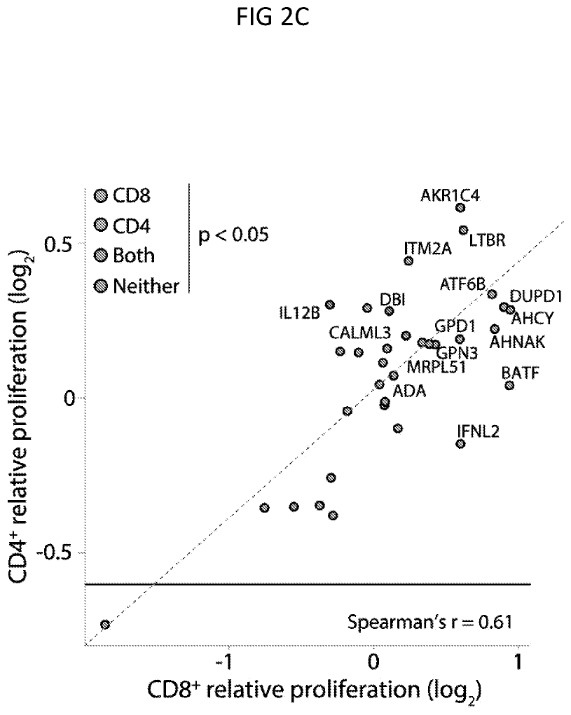

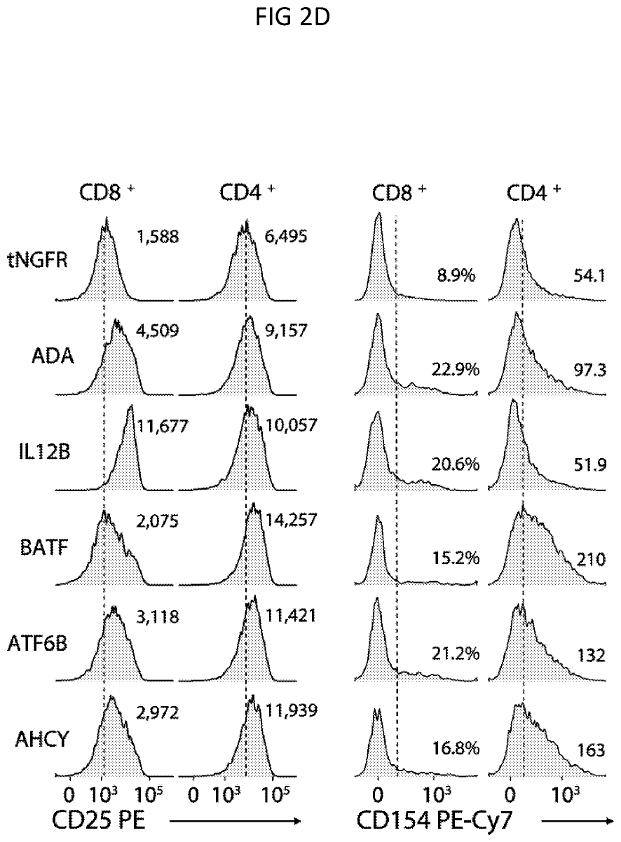

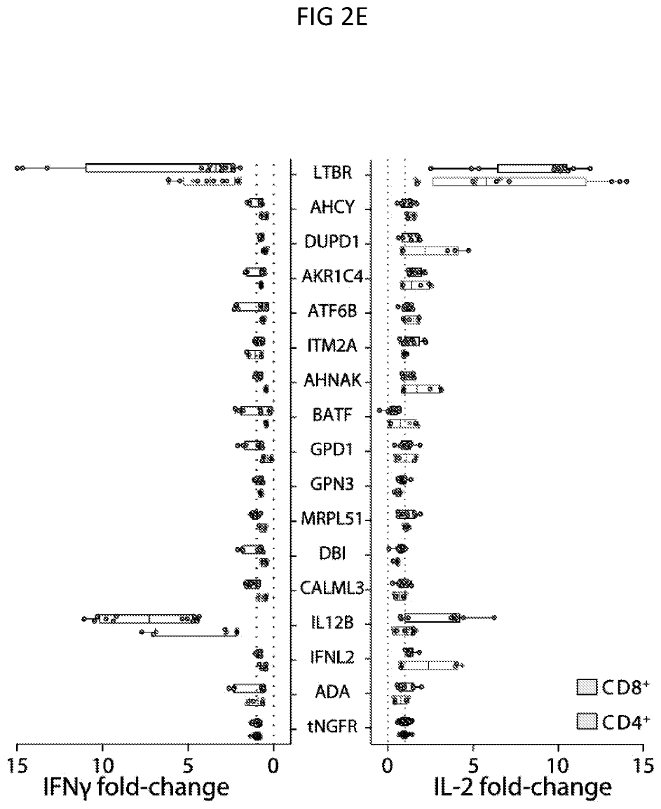

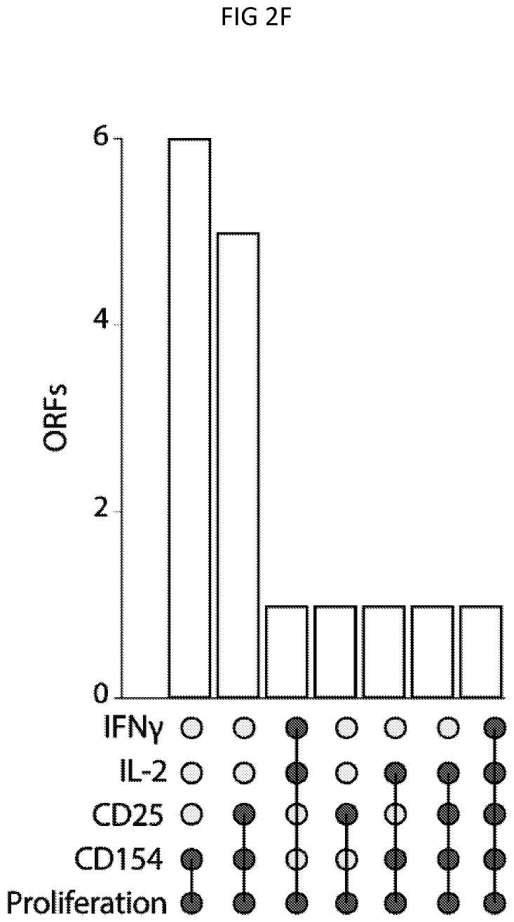

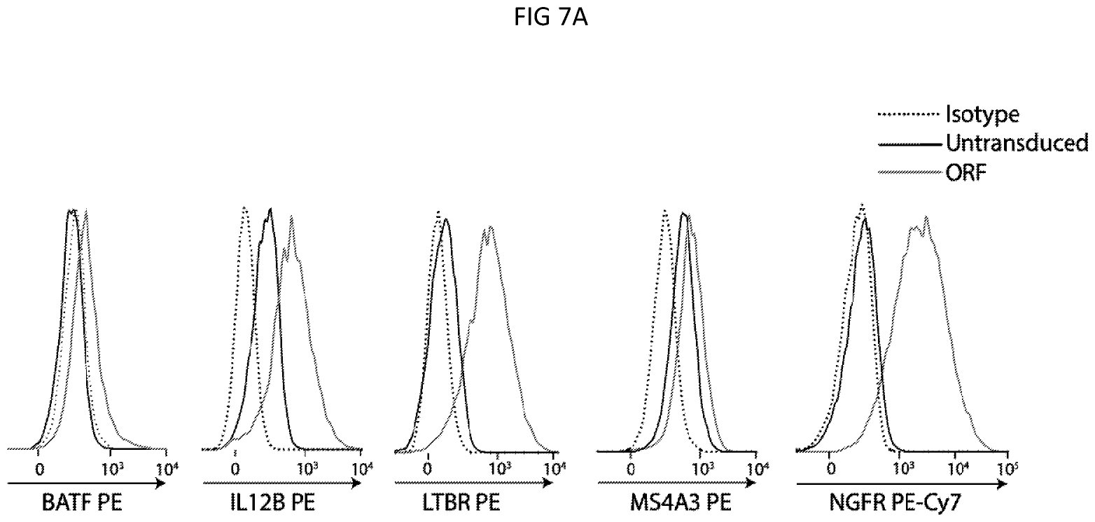

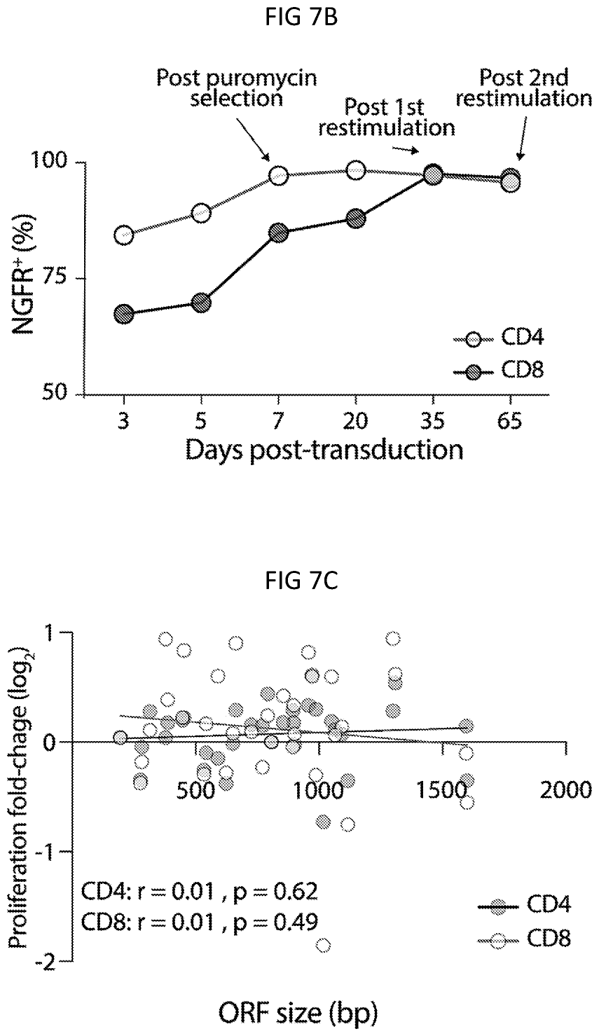

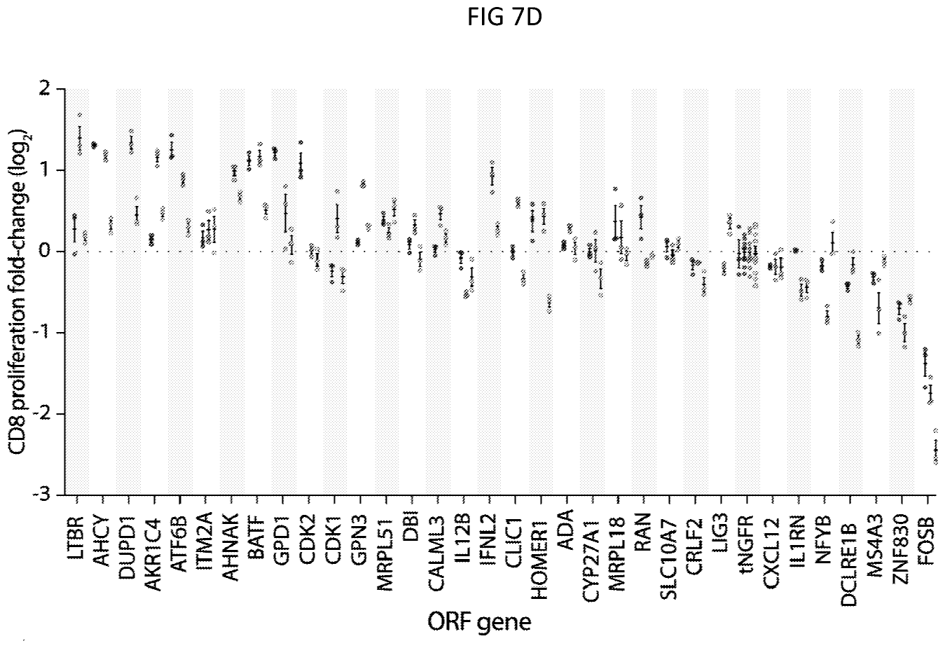

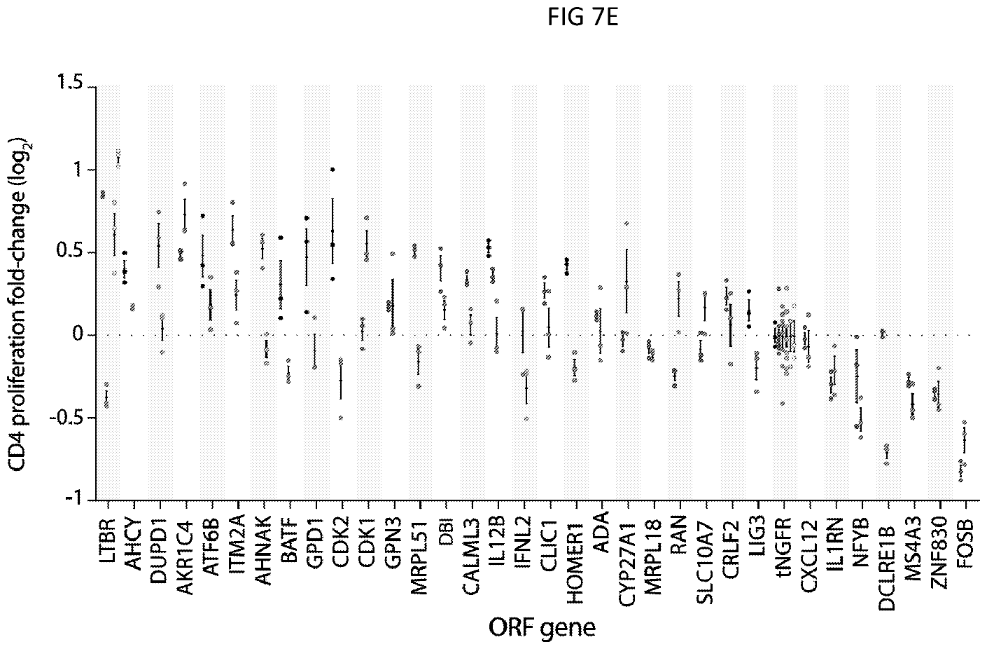

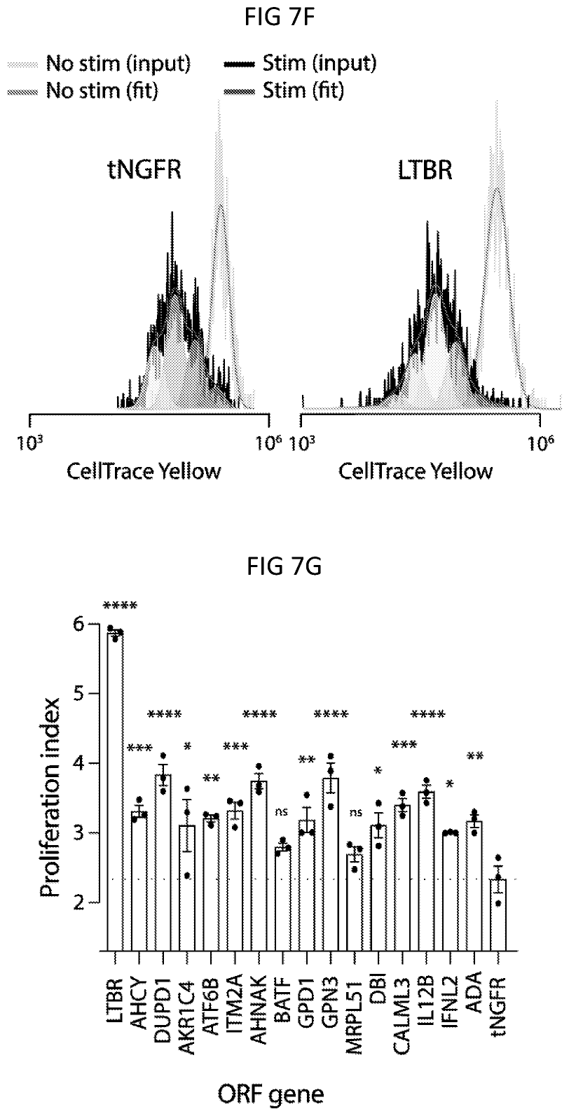

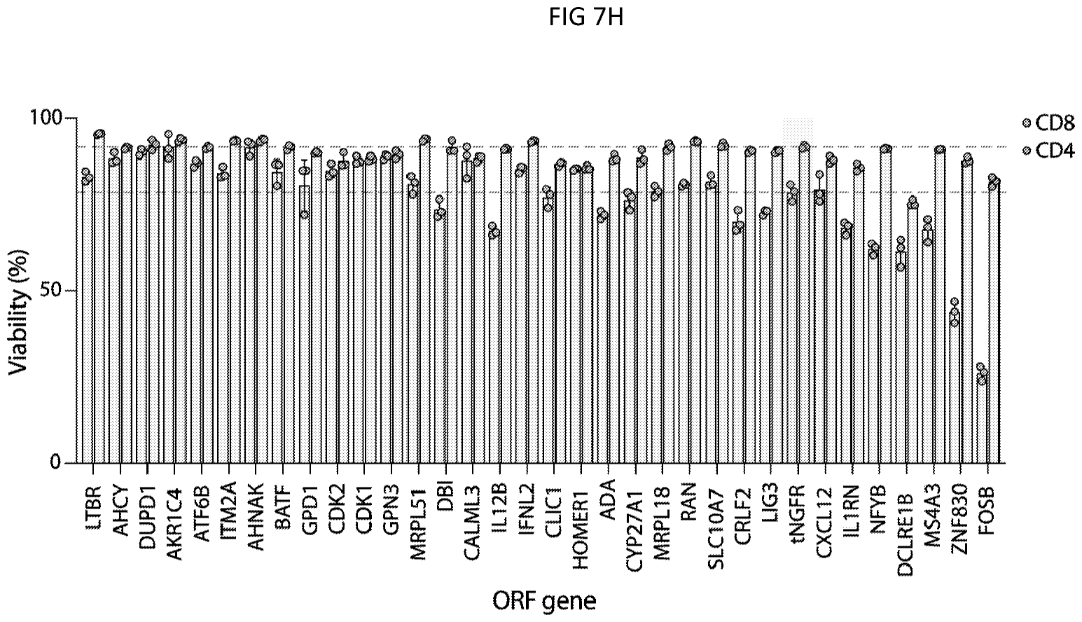

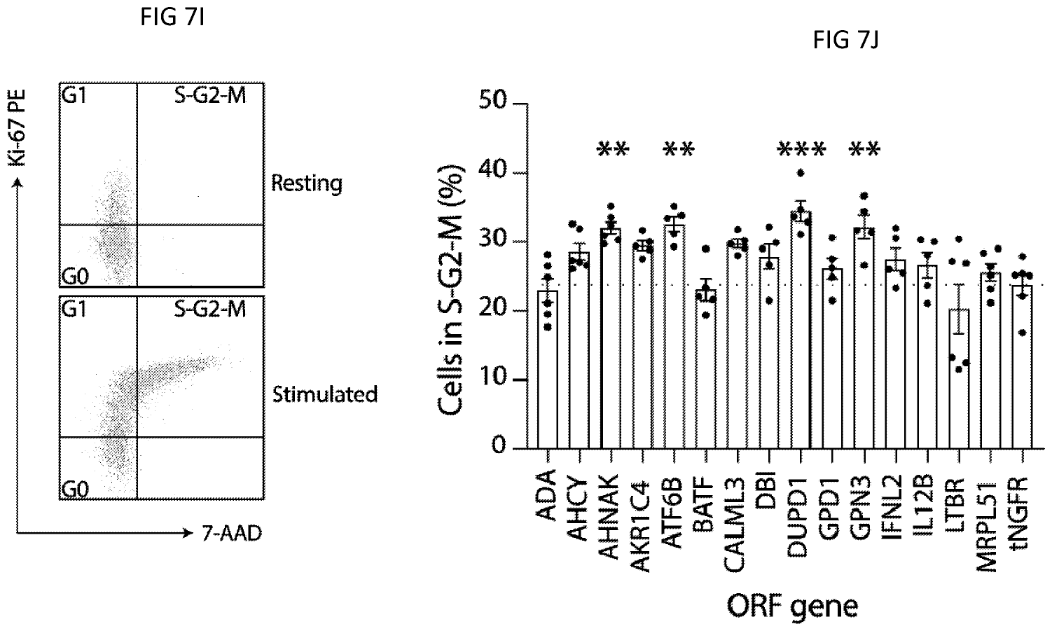

To validate the top-ranked ORFs and understand their effect on other relevant aspects of T cell function, we subcloned 33 ORFs from the library into a vector co-expressing a P2A-linked puromycin resistance gene from the same promoter. We chose a truncated nerve growth factor receptor (tNGFR), lacking its intracellular domain, as a control that has no effect on T cell phenotype²¹. CD4⁺ and CD8⁺ populations were separately isolated from several screen-independent healthy donors and transduced with individual ORFs (FIG. 2A). Using flow cytometry on representative ORFs, we confirmed that they were stably and uniformly expressed in both subsets of T cells for the duration of the experiment (FIG. 7A, FIG. 7B).

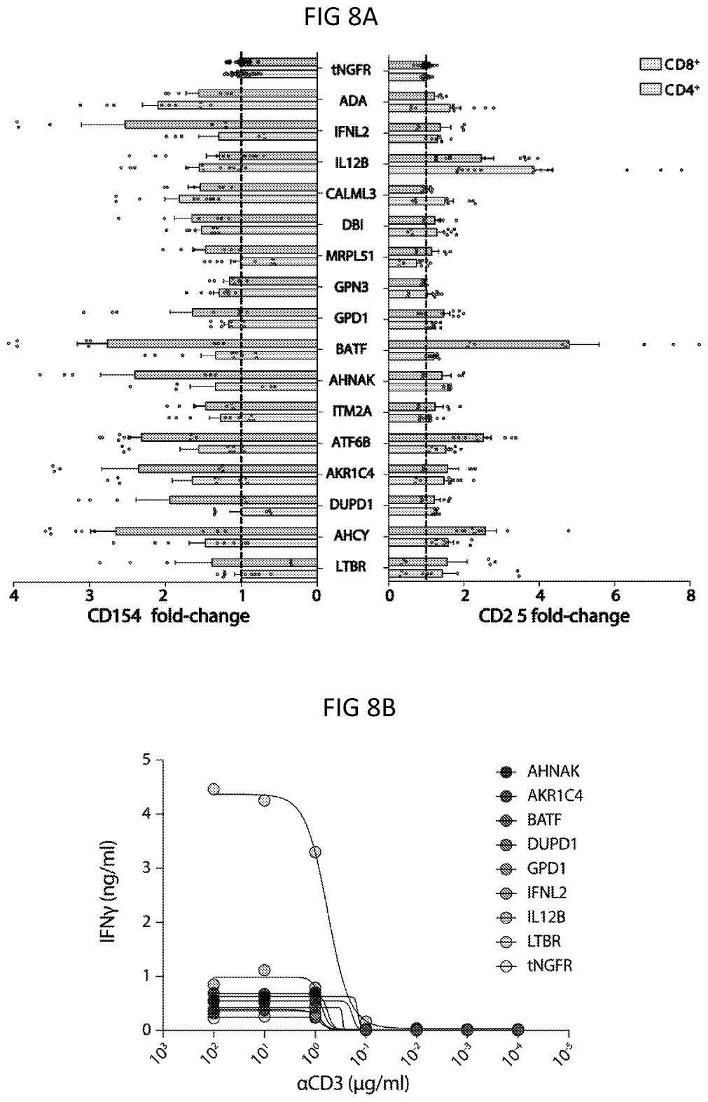

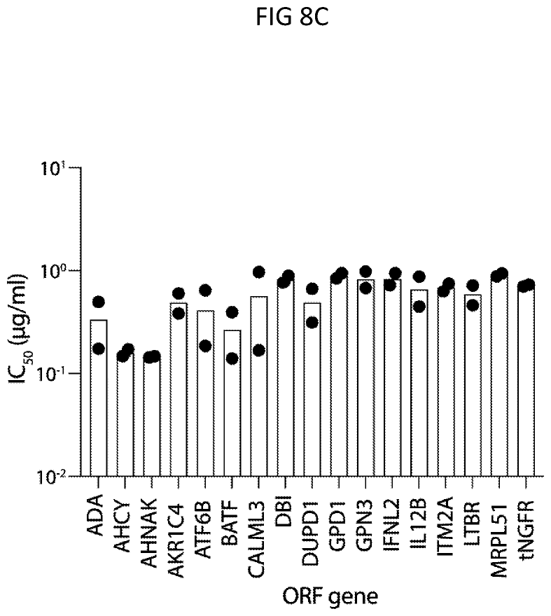

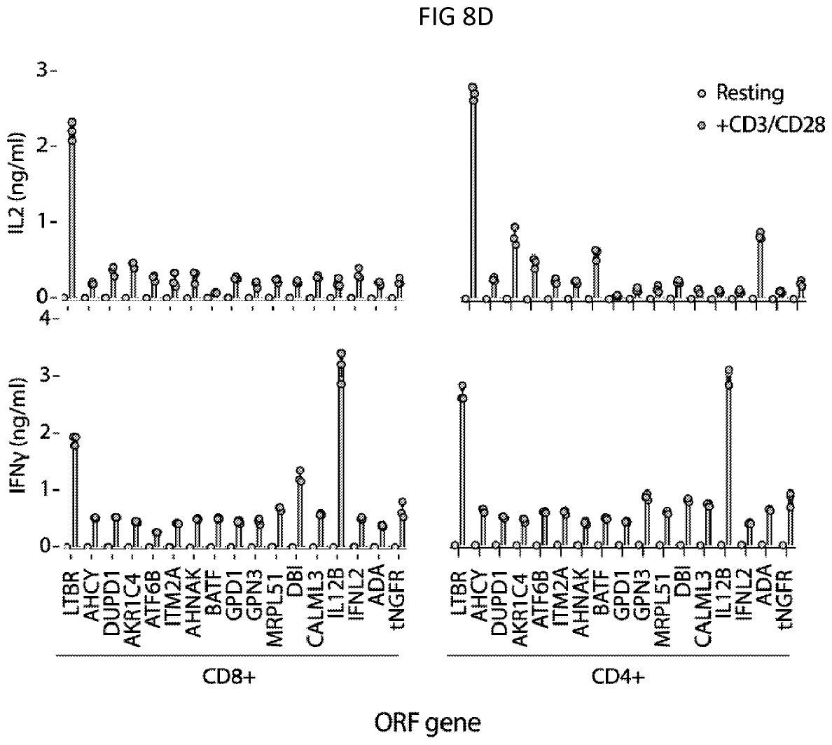

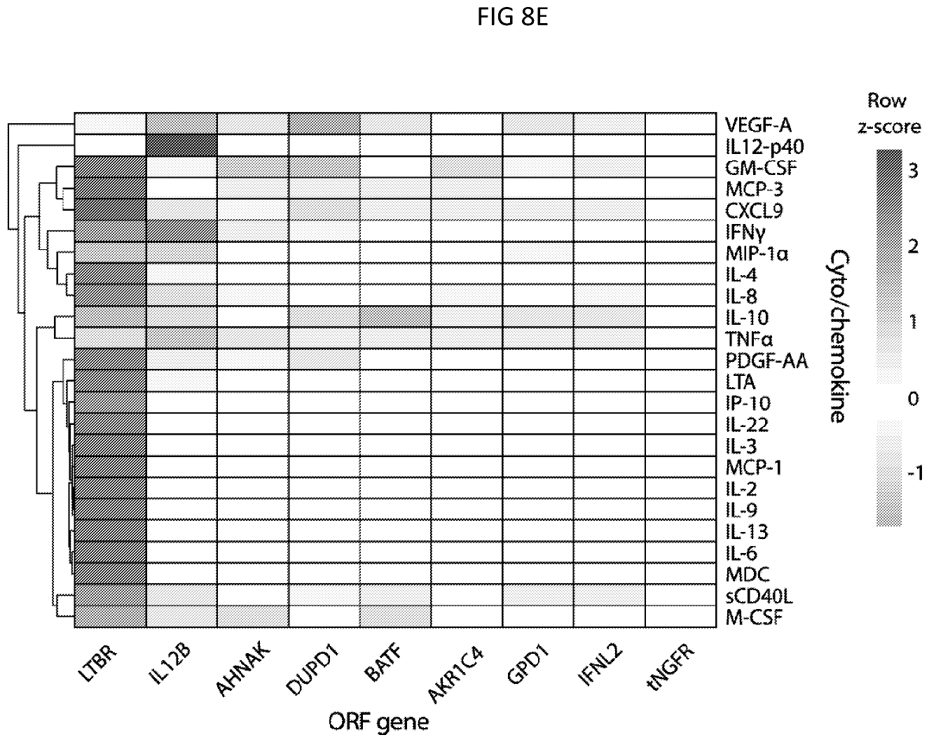

Fourteen days after isolation, we restimulated the cells and measured the relative increase in cell numbers. We found that 16 tested ORFs significantly improved cell proliferation compared with tNGFR, and that proliferation was well correlated between CD4⁺ and CD8⁺ cells (Spearman's r=0.61, P=0.002) (FIG. 2B, FIG. 2C, FIG. 7C-FIG. 7H). Having established that the top ORFs improve T cell proliferation, we next tested whether there is also a change in other T cell phenotypes and functions, such as increased cell cycle entry, expression of the activation markers IL2RA (CD25) and CD40L (CD154), and cytokine secretion. Most of the ORFs tested showed no difference in cycling (FIG. 21, FIG. 2J), but showed higher expression of both CD25 and CD154 in T cells after stimulation (FIG. 2D, FIG. 8A), further corroborating their effect in improving the magnitude of T cell responses.

Finally, we measured the secretion of the cytokines interleukin-2 (IL-2) and IFNγ after restimulation with CD3/CD28 (FIG. 2E, FIG. 8B-FIG. 8E). Although our screen was not designed to identify genes that modulate cytokine secretion, several ORFs could both improve T cell proliferation and boost IL-2 or IFNγ secretion (FIG. 2F). The strongest effect was observed for LTBR, which increased the secretion of both these cytokines in CD4⁺ and CD8⁺ T cells by more than fivefold.

### Single-Cell Analysis of ORF Phenotypes

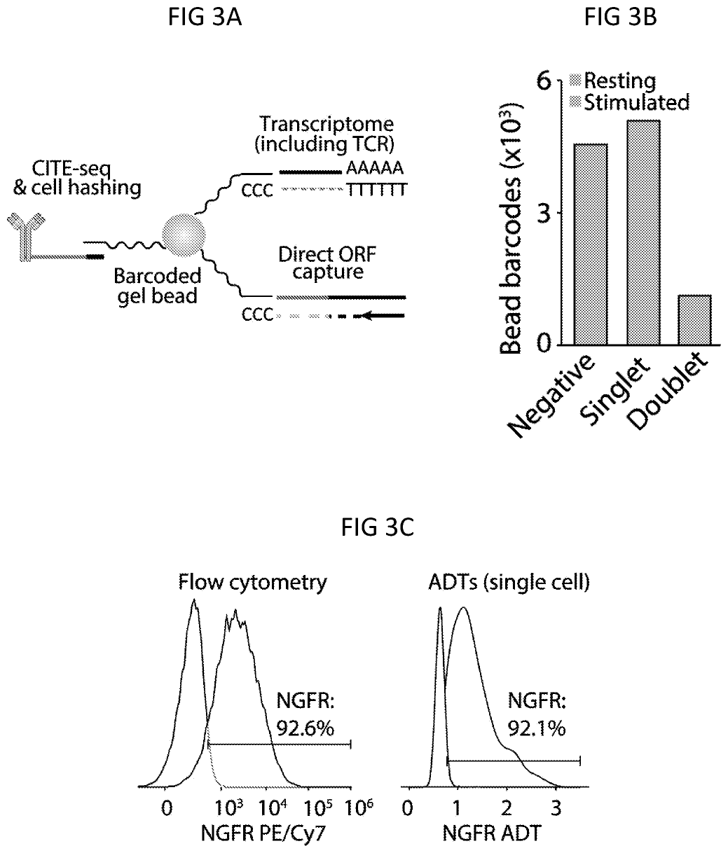

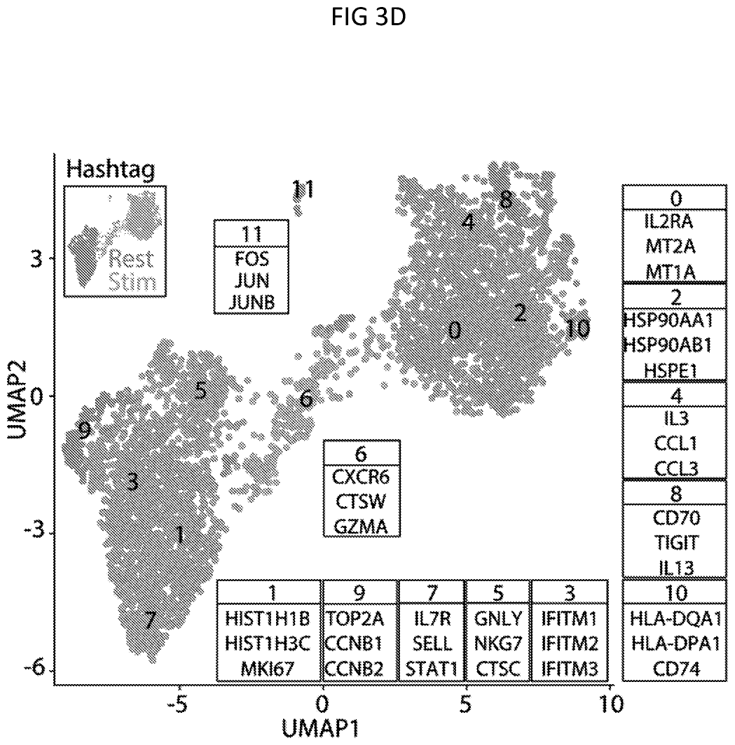

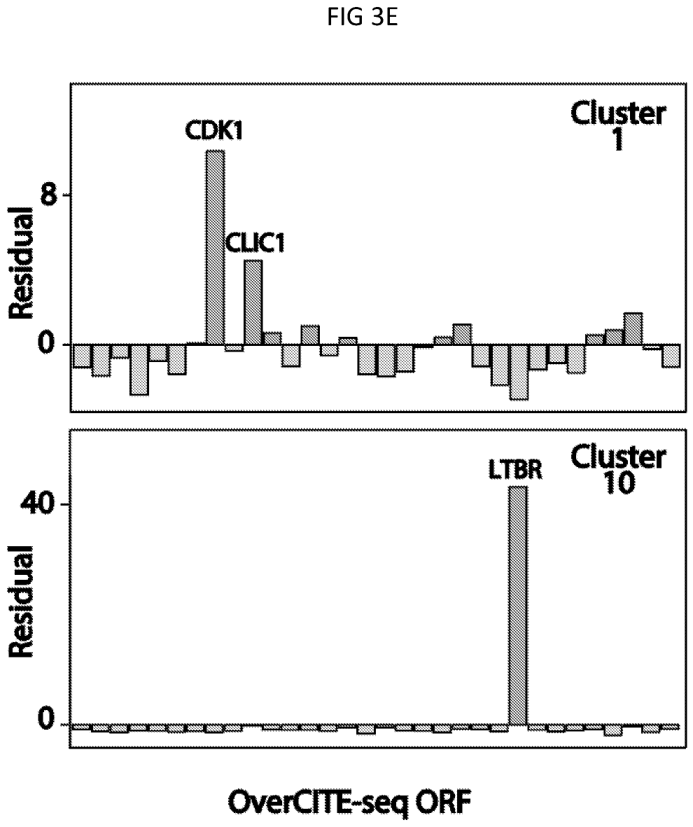

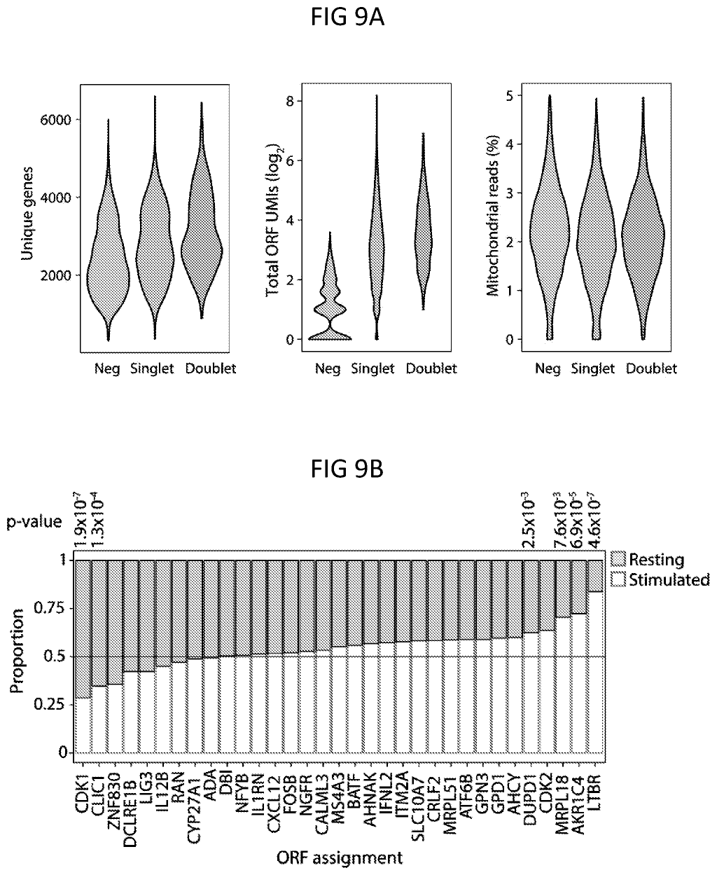

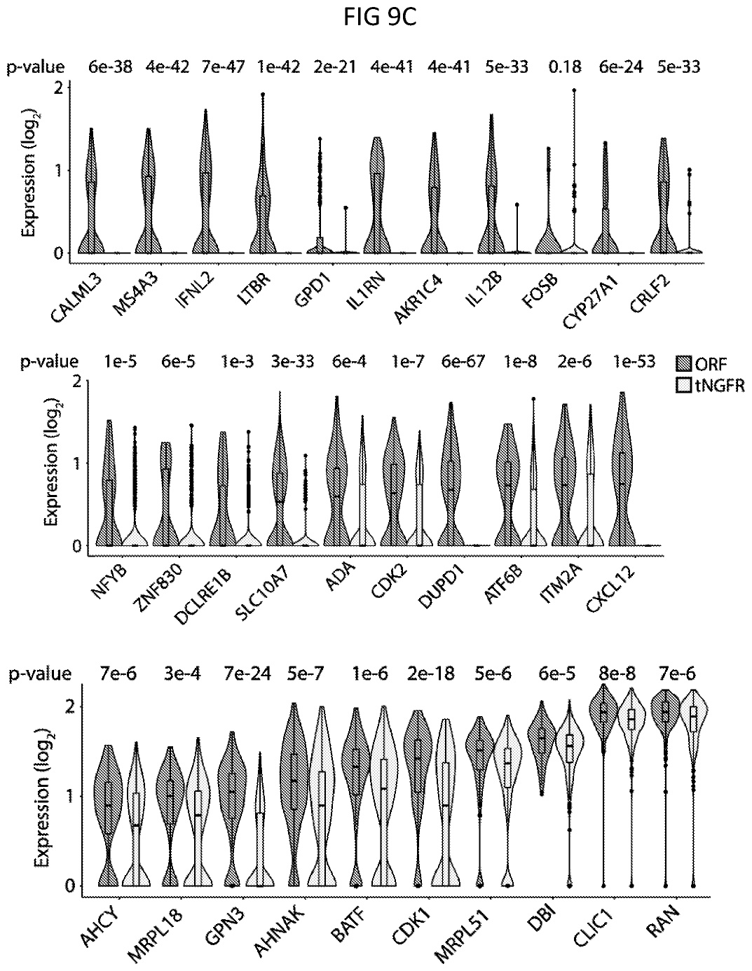

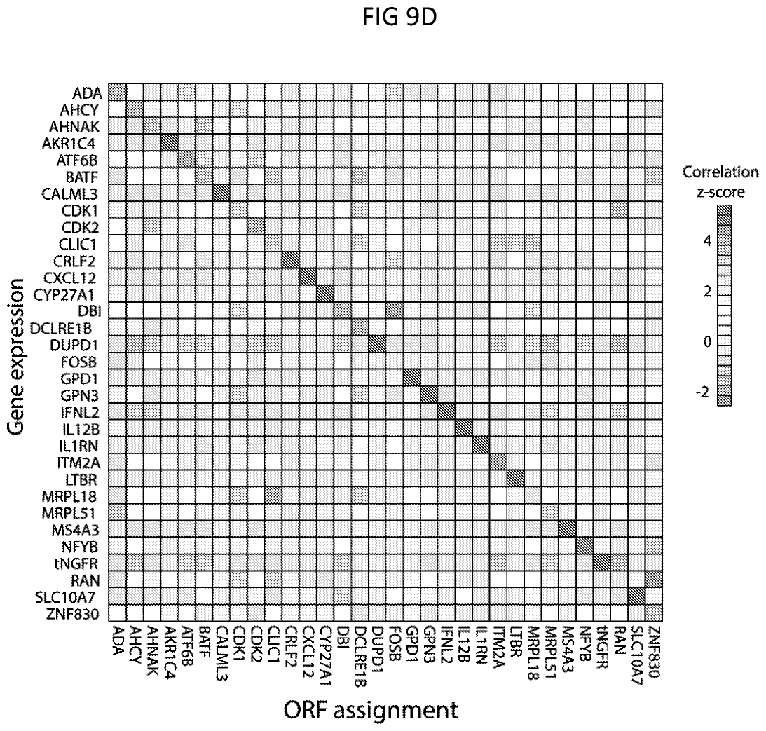

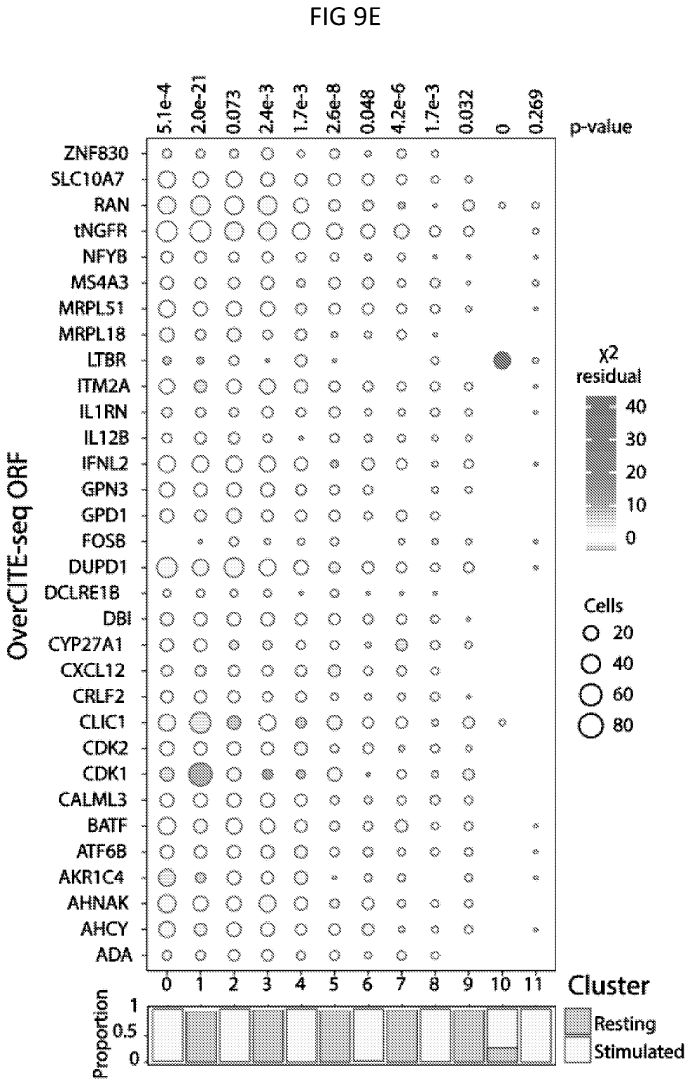

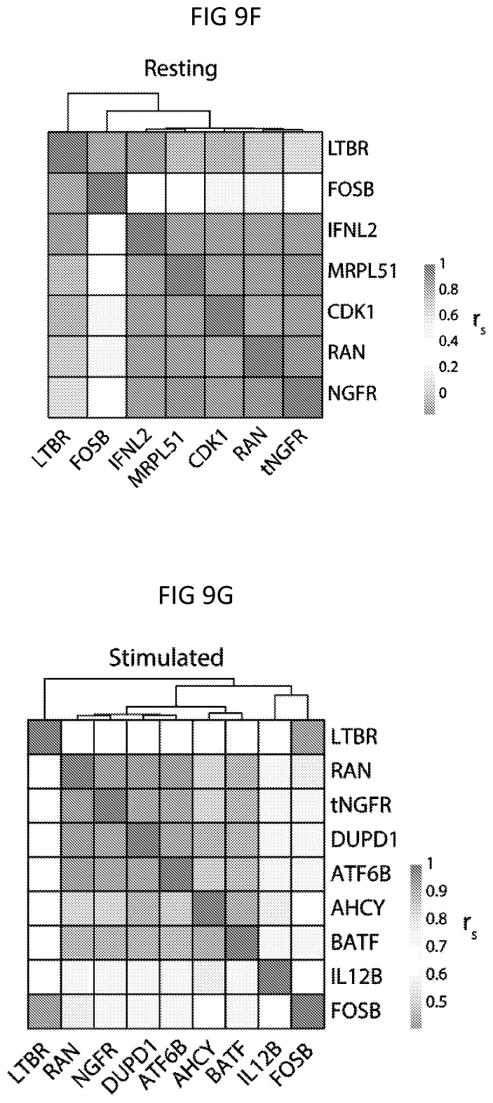

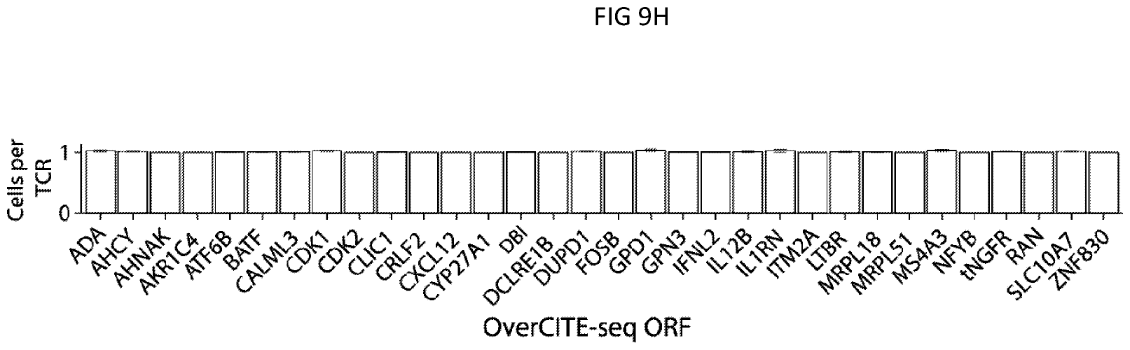

Building on our quantification of how each ORF affects proliferation, activation and cytokine release, we next sought to better understand the underlying mechanisms that drive these changes in cell state. To gain a more comprehensive view of the mechanisms of action of individual ORFs, and to provide a multidimensional characterization of the phenotypic changes they induce, we developed a single-cell sequencing strategy with direct ORF capture. This approach, OverCITE-seq (Overexpression-compatible Cellular Indexing of Transcriptomes and Epitopes by Sequencing) extends previous approaches that we have developed for quantifying surface antigens²² and CRISPR perturbations²³, and allows for high-throughput, single-cell analysis of a pool of T cells with different ORFs. In brief, mRNA from lentivirally integrated ORFs is reverse-transcribed by a primer binding to a constant sequence of the transcript downstream of the ORF and barcoded, along with the cell transcriptome, during template switching. The resulting cDNA pool is then split for separate construction of gene expression and ORF expression libraries (FIG. 3A, FIG. 3B, FIG. 9A).

We optimized and applied OverCITE-seq to a pool of around 30 ORFs transduced into CD8⁺ T cells from a healthy donor. The cell pool was either left unstimulated (‘resting’) or stimulated with CD3/CD28 antibodies to mimic TCR activation. To gain confidence in how well ORFs are assigned to each single cell, we leveraged the fact that the protein produced by the control gene, tNGFR, is expressed on the cell surface and can thus be captured with a DNA-barcoded antibody.²³. The proportion of cells designated as tNGFR positive was consistent when measured by CITE-seq or flow cytometry (FIG. 3C). An analysis of the entire ORF pool showed that single cells assigned with a given ORF had overall the strongest expression of the corresponding gene (FIG. 9B-FIG. 9D), indicating that our ORF capture strategy reliably assigned a genetic perturbation to each single cell.

Unsupervised clustering showed clear separation for stimulated and resting T cells. Within these activation-driven super-clusters we could observe individual clusters associated with a particular cell state or function, such as cell cycle (clusters 1 and 9), macromolecule biosynthesis (cluster 2), type I IFN signaling (cluster 3), cytotoxicity (cluster 6), T cell activation and proliferation (cluster 10), and stress response and apoptosis (cluster 11) (FIG. 3D). Although in many cases several ORFs contributed to a given cluster phenotype (FIG. 9E), we observed a notable enrichment of two ORFs, CDK1 and CLIC1, in cluster 1, characterized by the increased expression of genes that are responsible for chromosome condensation in preparation for cell cycle (FIG. 3E). An even stronger enrichment was observed for cluster 10, which was almost exclusively composed of cells expressing LTBR.

To investigate the mechanisms of genetic perturbations with the strongest transcriptional changes, we looked at the transcriptomic profiles of CD3/CD28-stimulated ORF T cells compared to unstimulated control T cells (FIG. 9F-FIG. 9I). This approach allowed us to identify gene modules that are shared between perturbations or that are perturbation-specific. For example, LTBR and CDK1 showed the strongest enrichment of genes involved in RNA metabolism and cell cycle (CDK4, HSPA8 and BTG3), as well as in the tumor necrosis factor (TNF) signaling pathway (TNFAIP3, TRAF1 and CD70). FOSB appeared to drive an opposite program to LTBR in terms of genes involved in TCR signaling (CD3D, CD3E, LAPTM5 and LAT), cytokine responses (GATA3 and TNERSF4) and the NF-κB pathway (NFKB2, NFKBIA and UBE2N). Finally, we determined that the observed phenotypes were a result of a genetic perturbation rather than an outgrowth of a single clone because virtually every single cell expressed a unique TCR clonotype (FIG. 9J). This result highlights the utility of OverCITE-seq's multimodal capture approach, yielding each T cell's transcriptome, clonotype, cell surface proteome, cell hashing (for treatment or stimulation conditions) and lentiviral ORF identity.

### LTBR Improves Multiple T Cell Functions

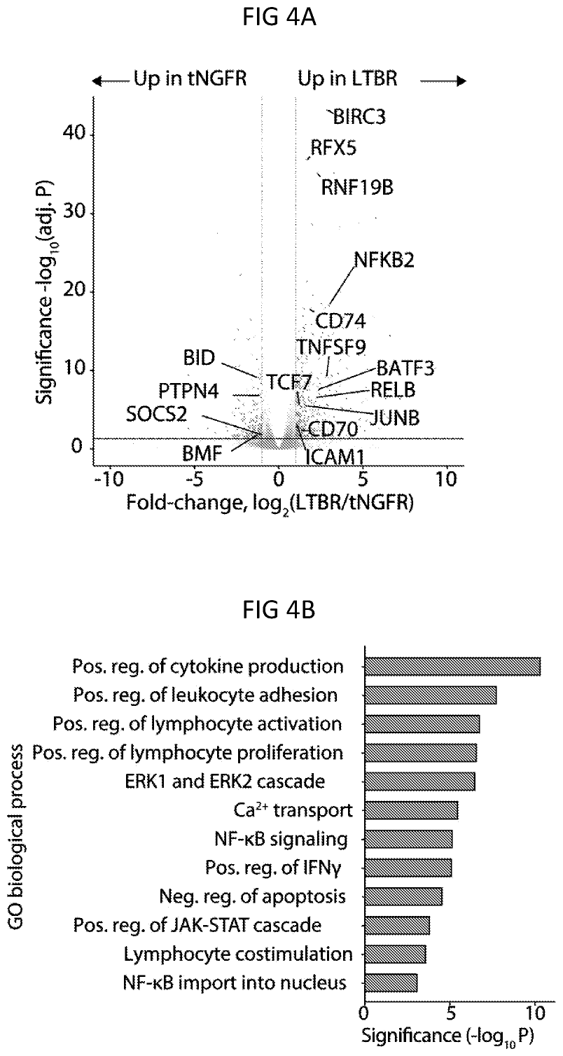

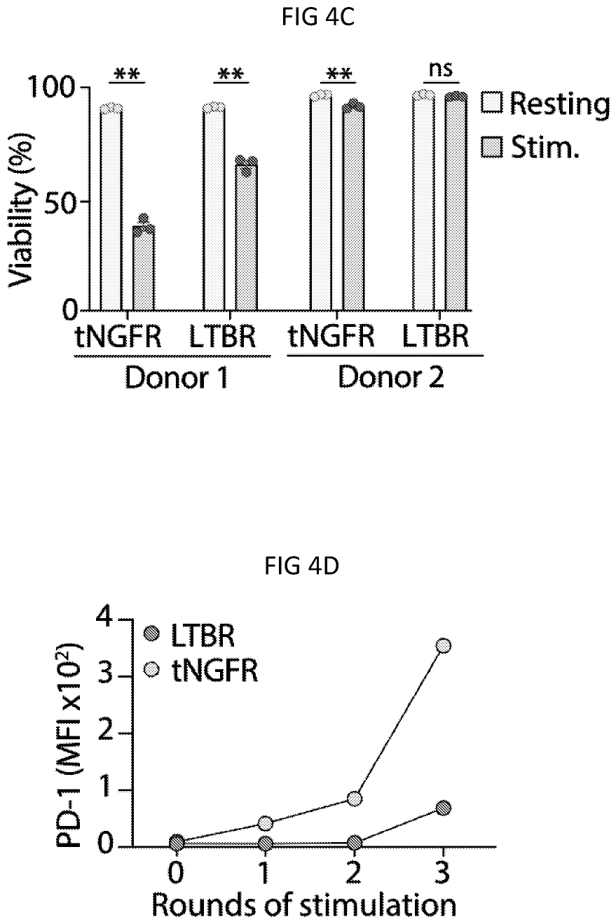

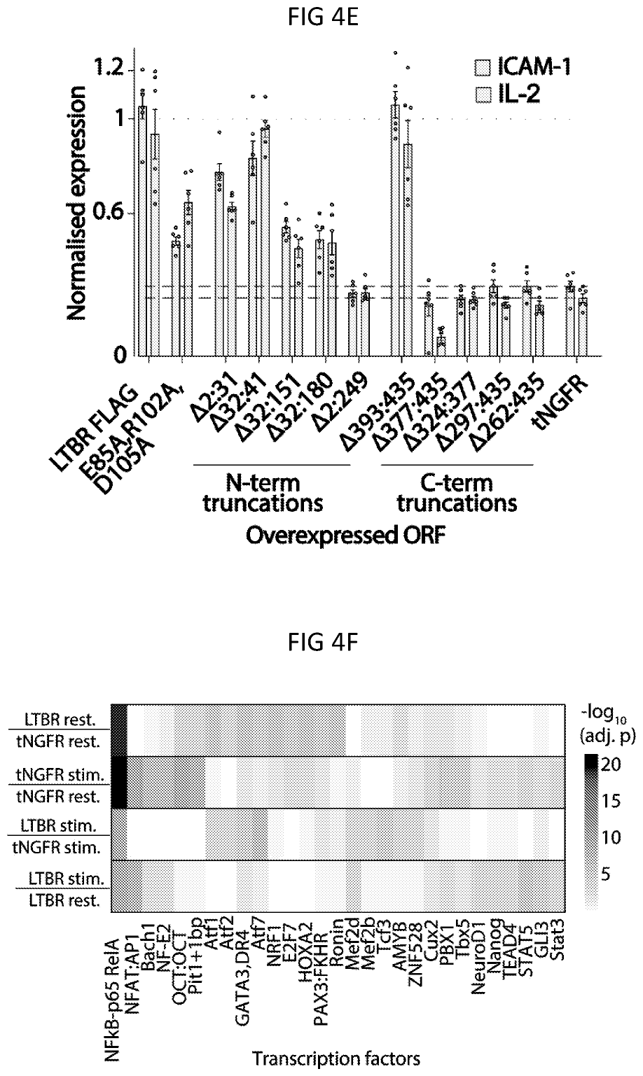

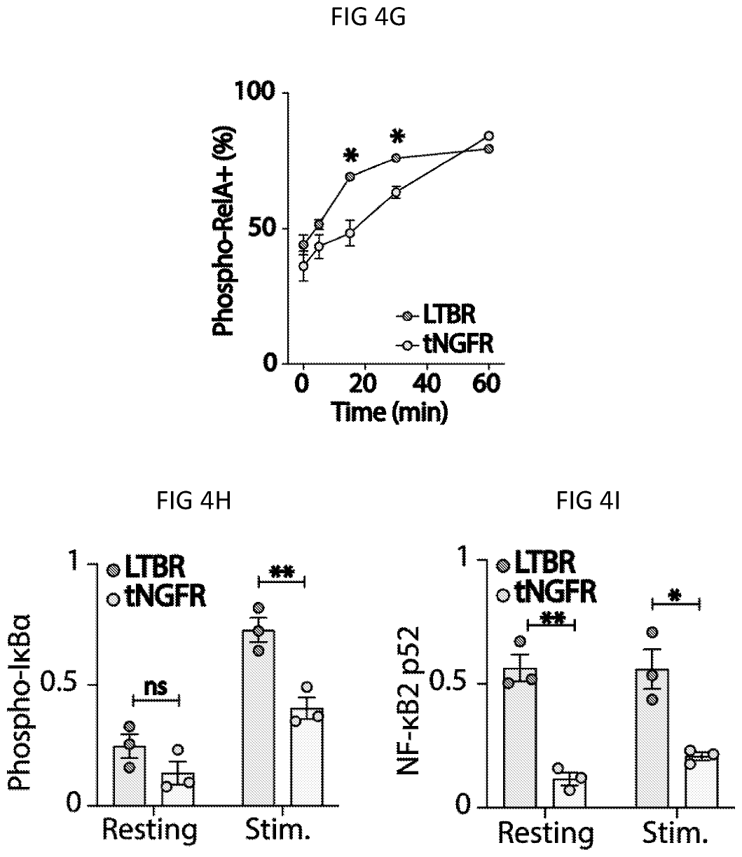

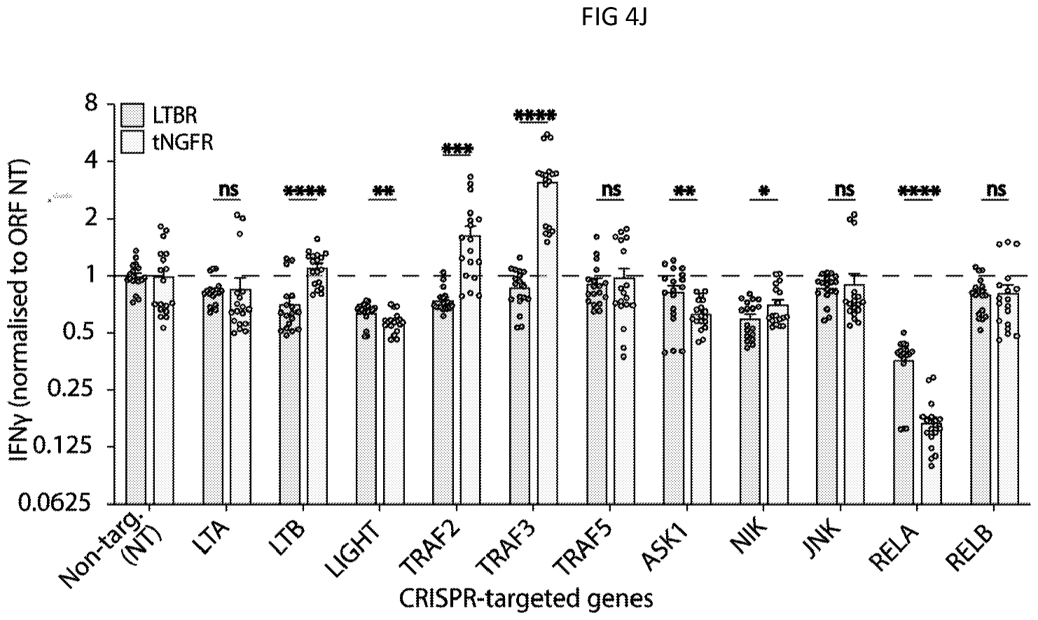

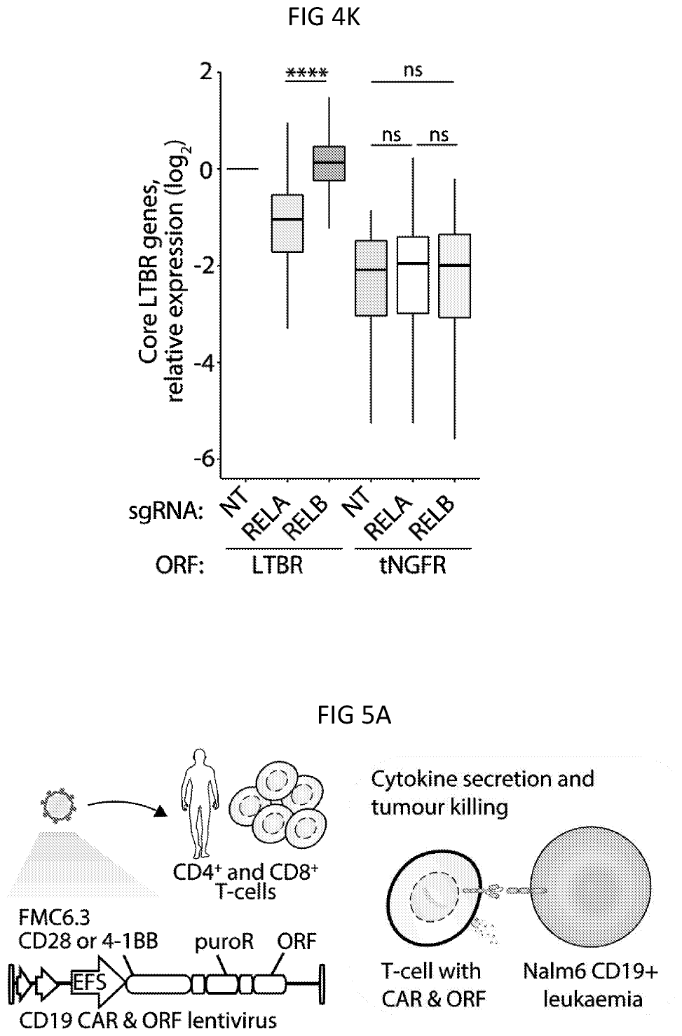

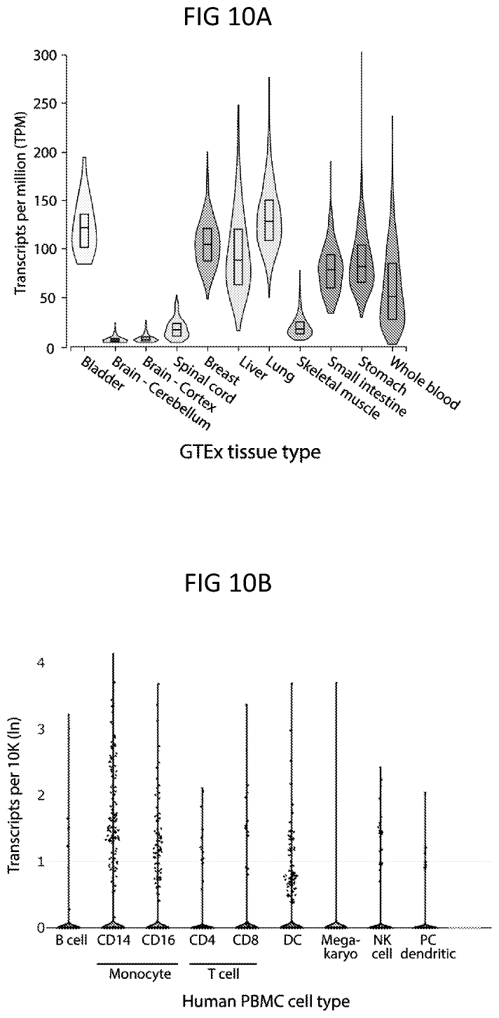

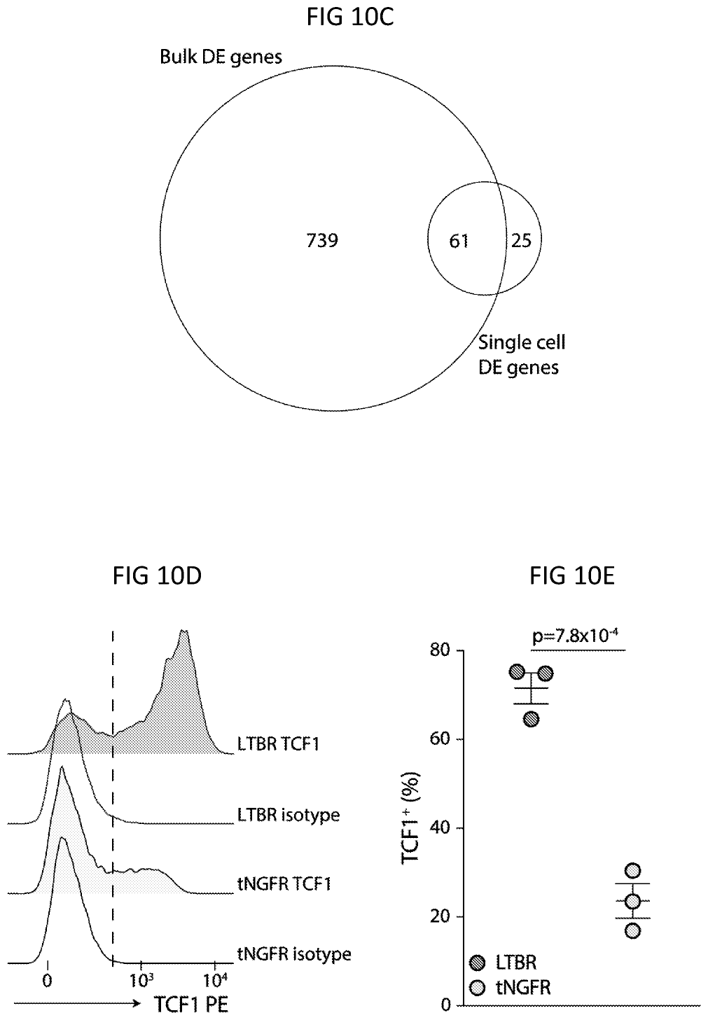

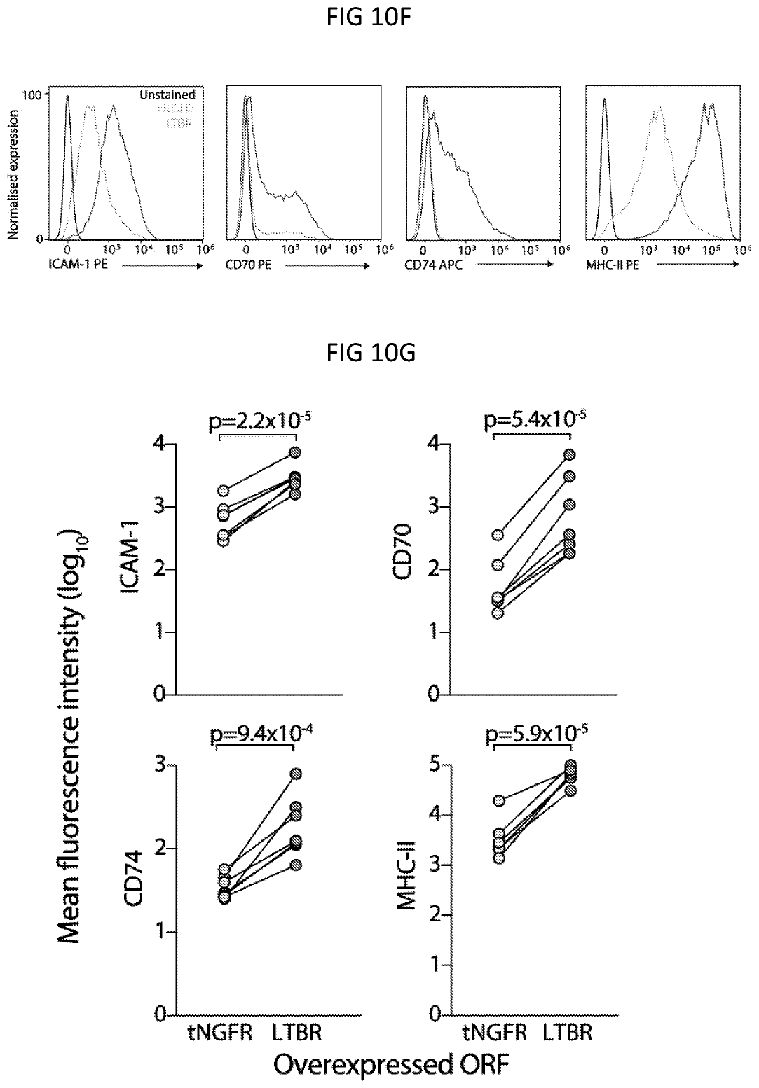

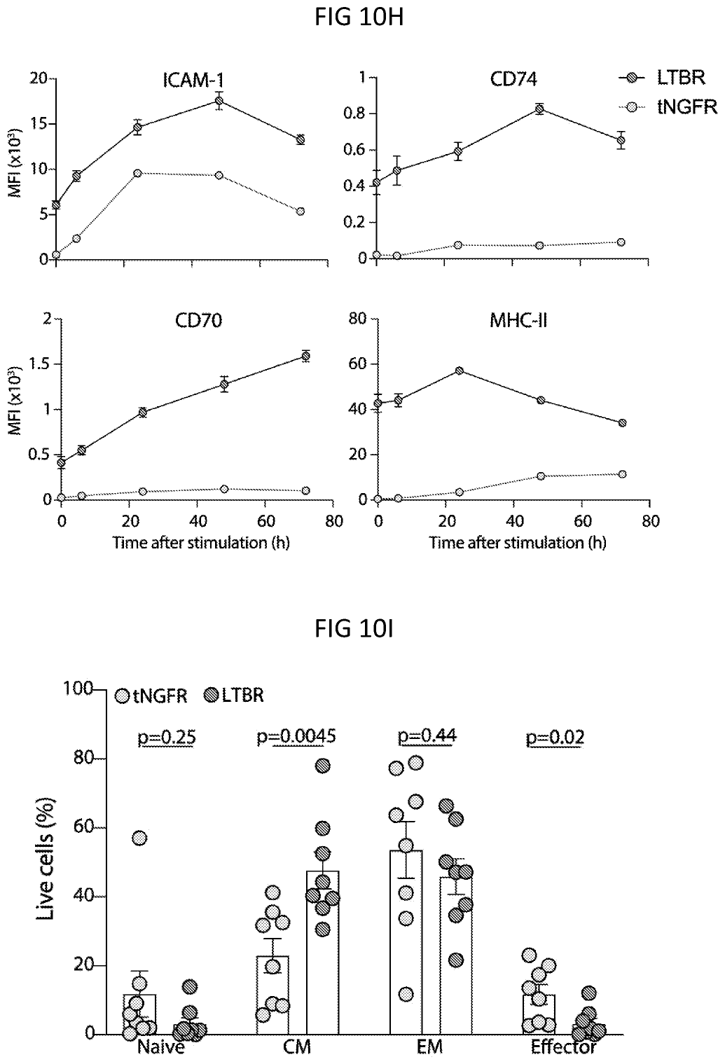

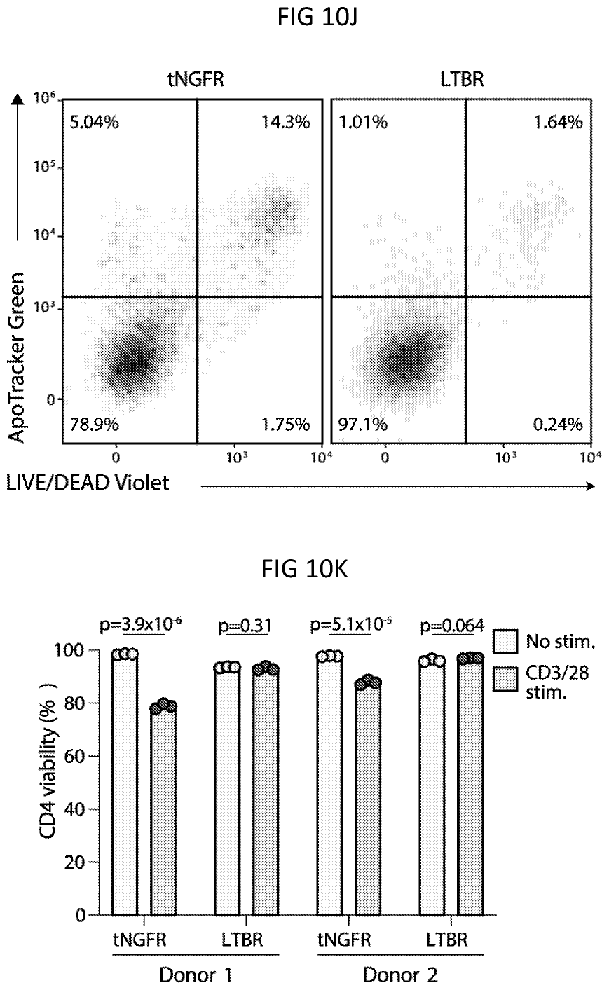

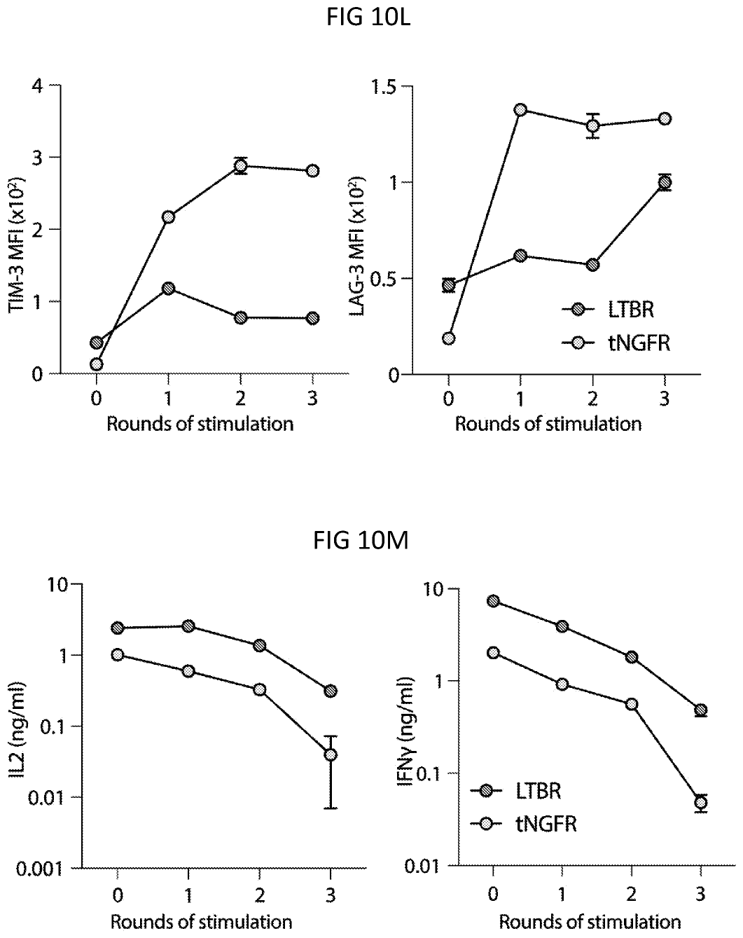

Having identified LTBR as a strong driver of proinflammatory cytokine secretion (FIG. 2E) and profound transcriptional remodeling (FIG. 3D, FIG. 3E), we decided to investigate its mechanisms of action in more detail. LTBR belongs to the tumor necrosis factor receptor superfamily (TNFRSF) and is expressed on a variety of non-immune cell types and on immune cells of myeloid origin, but is absent from lymphocytes (FIG. 10A, FIG. 10B). Using bulk RNA sequencing (RNA-seq), we compared global gene expression between LTBR- and tNGFR-transduced cells, with or without TCR stimulation (FIG. 4A, FIG. 4B, FIG. 10C). In addition to upregulation of MHC-I and II genes (HLA-C, HLA-B, HLA-DPB1, HLA-DPA1 and HLA-DRB6) and transcription factors necessary for MHC-II expression (RFX5 and CIITA), LTBR cells also expressed the MHC-II invariant chain (encoded by CD74). Notably, CD74 has been shown in B cells to activate the pro-survival NF-κB pathway, in particular through upregulation of the anti-apoptotic genes TRAF1 and BIRC3 (both of which are also upregulated in LTBR-overexpressing cells)²⁴. Similarly, LTBR cells strongly upregulated BATF3, which has been shown to promote the survival of CD8⁺ T cells²⁵. We also observed upregulation of JUNB, a transcription factor involved in IL-2 production²⁶, and TCF7 (encoding TCF1), a key transcription factor responsible for T cell self-renewal²⁷. We confirmed the RNA-seq results at the protein level (FIG. 10D-FIG. 10I). LTBR cells were also more resistant to activation-induced cell death and retained greater functionality after repeated stimulations (FIG. 4C, FIG. 4D, FIG. 10J-FIG. 10M).

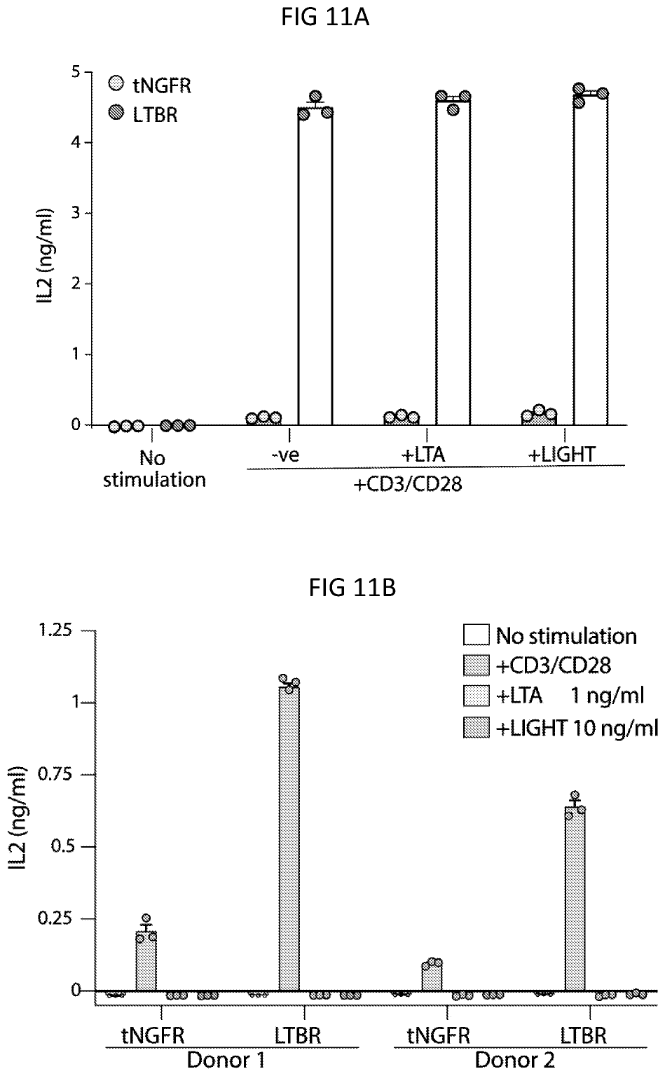

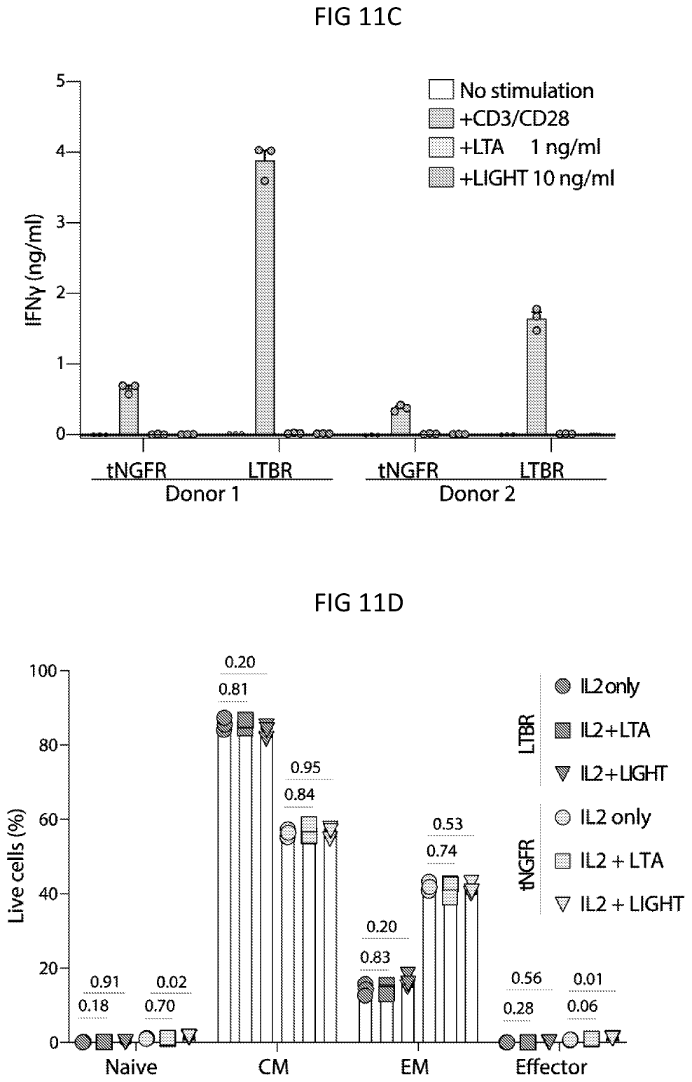

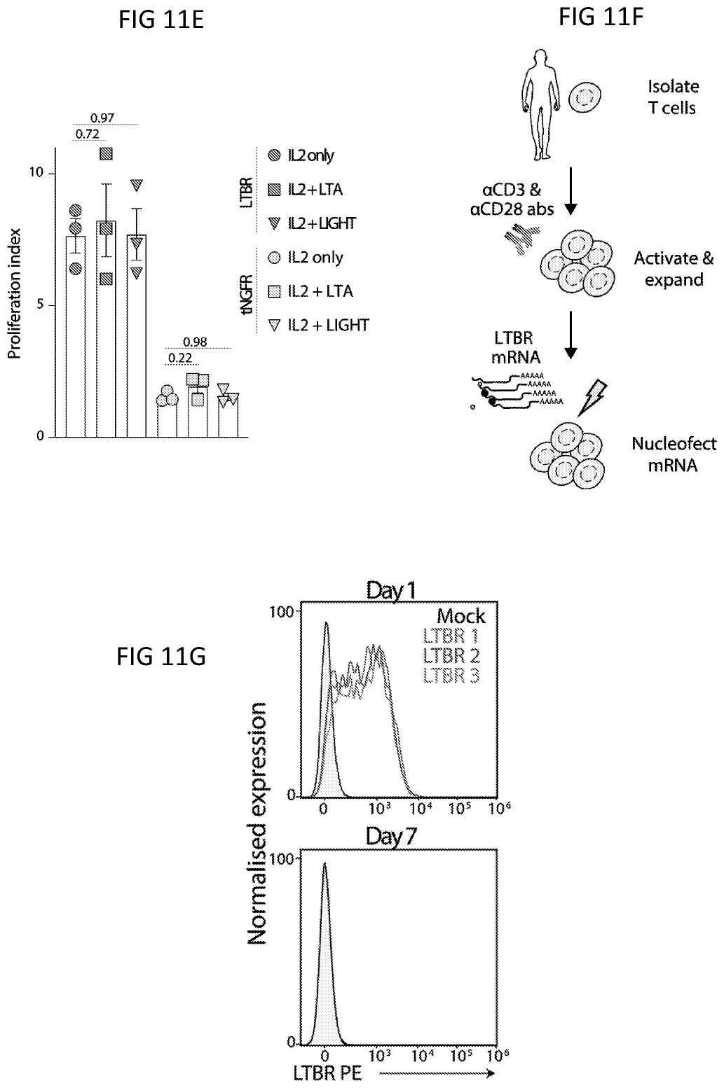

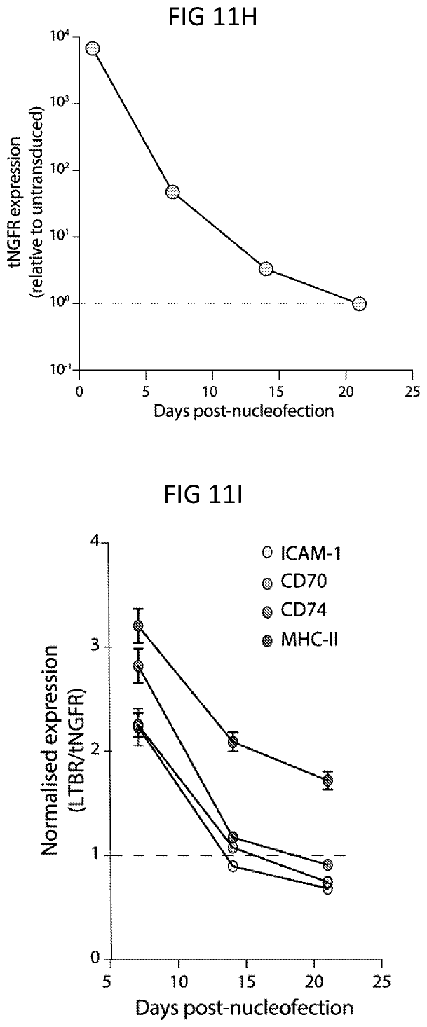

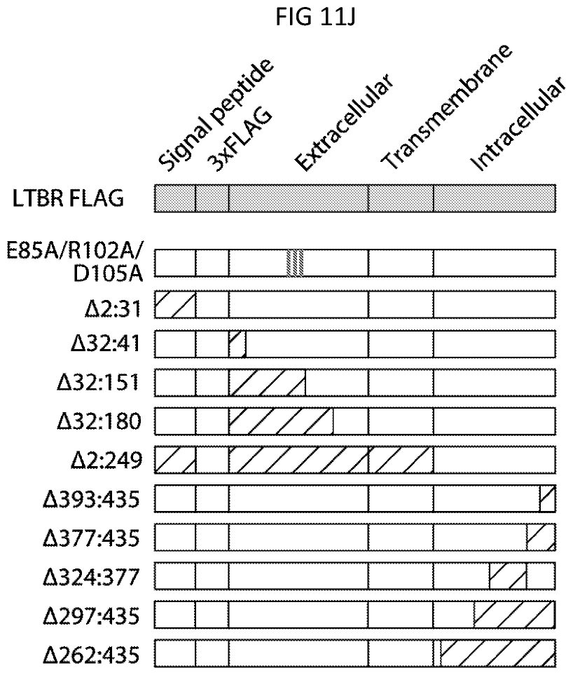

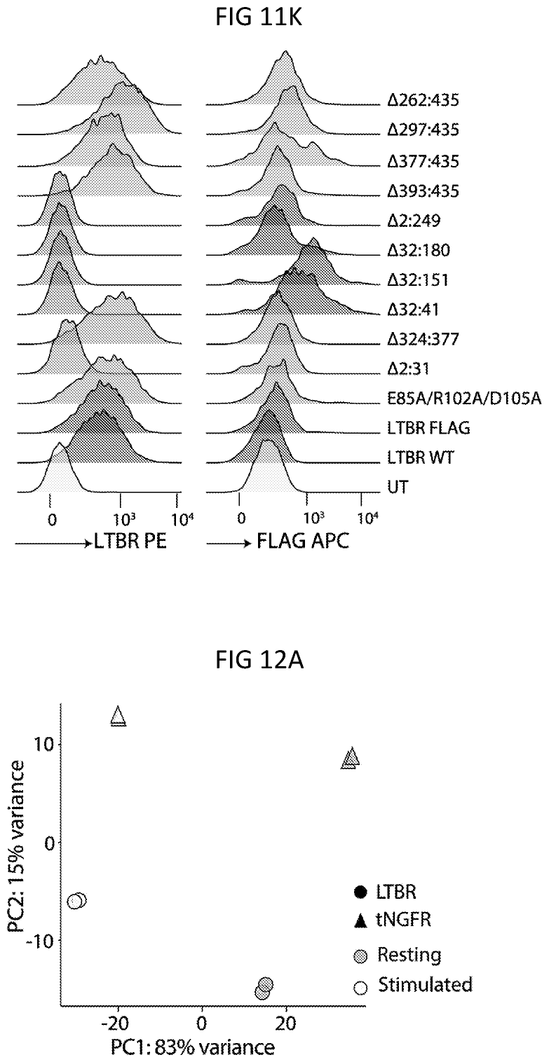

LTBR signaling in its endogenous context (in myeloid cells) is triggered either by a heterotrimer of lymphotoxin-α (LTA) and lymphotoxin-β (LTB) or by LIGHT (encoded by the TNFSF14 gene). As LTA, LTB and LIGHT are expressed by activated T cells, we sought to elucidate whether addition of exogenous LTA or LIGHT could modulate the cytokine secretion, differentiation or proliferation of CD3/CD28-stimulated LTBR-overexpressing T cells; however, we found no effect of exogenous ligands on LTBR T cell function (FIG. 11A-FIG. 11E). Thus, although LTBR could potentiate the TCR-driven T cell response, it does not drive activation on its own-which would be a potential safety issue and result in loss of antigen specificity of the engineered T cell response. We also determined that constitutive expression of LTBR is required for maintenance of its phenotype but that there is a substantial lag time between loss of detectable LTBR expression and loss of phenotype (FIG. 11F-FIG. 11I), indicating that transient expression of LTBR may be a safe avenue into a therapeutic application.

Finally, to identify the key domains of the LTBR protein that drive its activity in T cells, we designed a series of point or deletion mutants of LTBR (FIG. 4E, FIG. 11J). In general, we found that the N terminus of LTBR was less sensitive to deletions than the C terminus. Similarly, a partial reduction of the LTBR phenotype was achieved by introducing three alanine point mutations in the key residues for LTA and LTB binding²⁸, or by removal of the signal peptide. Using our C-terminal deletions, we found that a mutant version of LTBR that lacks residues 393-435 showed no difference compared with full-length LTBR, whereas the deletion of residues 377-435 completely abrogated the LTBR phenotype, despite being expressed at a comparable—if not higher—level (FIG. 11K), probably owing to the loss of a binding site for TRAF2, TRAF3 or TRAF5²⁹. Moreover, a deletion of the self association domain³⁰ (324-377) also completely abrogated the phenotype.

### LTBR Acts Through Canonical NF-κB in T Cells

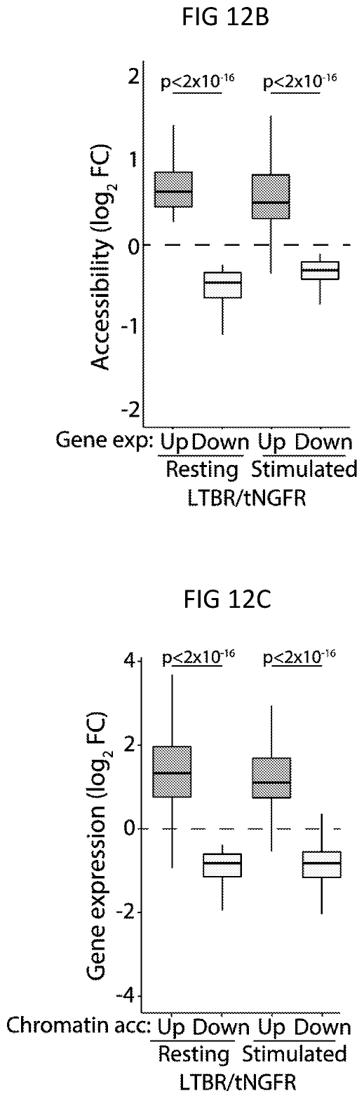

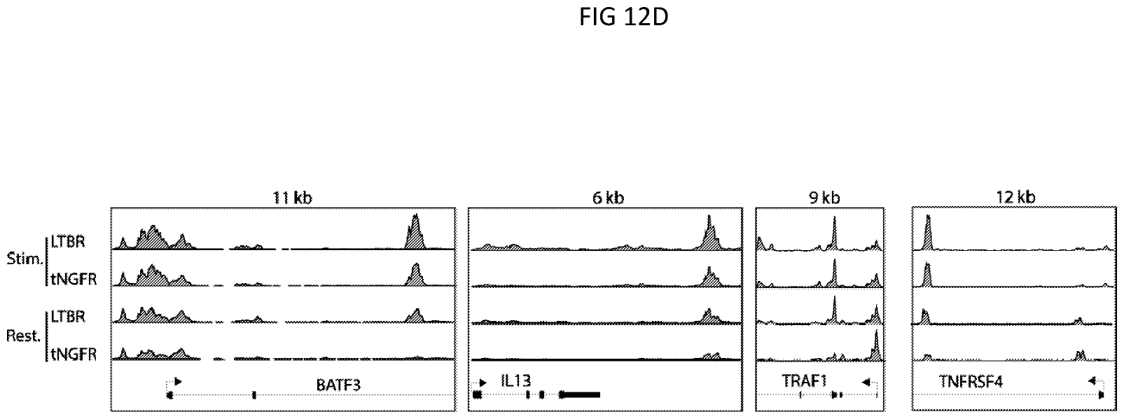

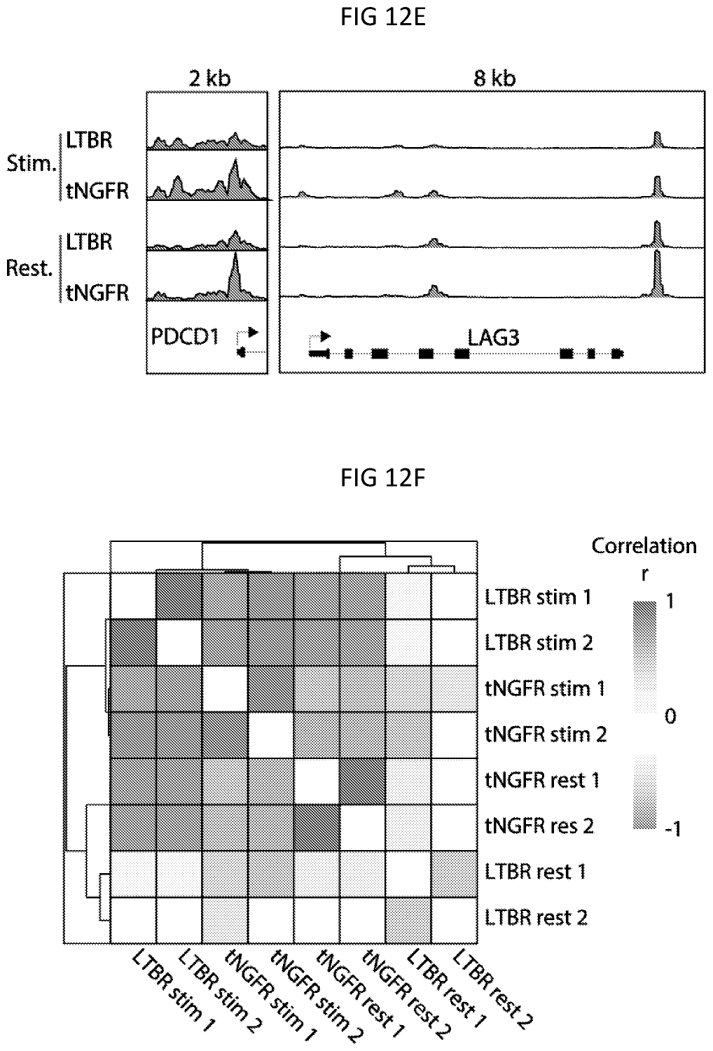

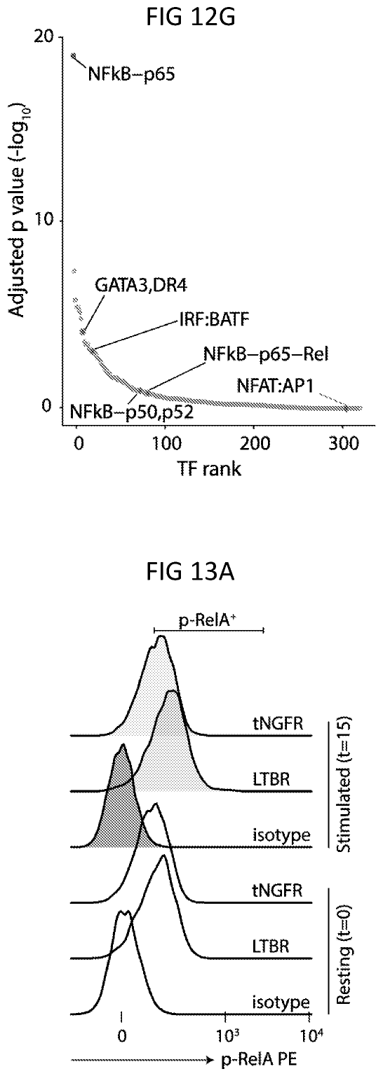

LTBR overexpression was shown to induce broad transcriptomic changes in T cells, accompanied by changes in T cell function (FIG. 4A, FIG. 4B). Thus, we sought to determine whether the perturbations in gene expression in LTBR cells were accompanied by epigenetic alterations, leveraging the assay for transposase-accessible chromatin by sequencing (ATAC-seq) (FIG. 12A-FIG. 12G). Comparing the enrichments of specific transcription factor motifs in differentially accessible chromatin regions, we identified NF-κB p65 (RELA) as the most enriched transcription factor in LTBR cells (FIG. 12H, FIG. 12I). Of note, NF-κB p65 and NFAT-AP-1 were the two most enriched transcription factors in open chromatin in stimulated versus resting T cells (both LTBR and tNGFR), in line with their well-established role in T cell activation³¹, but only NF-κB p65 showed strong enrichment in LTBR cells, with and without stimulation (FIG. 4F). This result suggests that LTBR induces a partial T cell activation state but still requires signal 1 (TCR stimulation) for full activation.

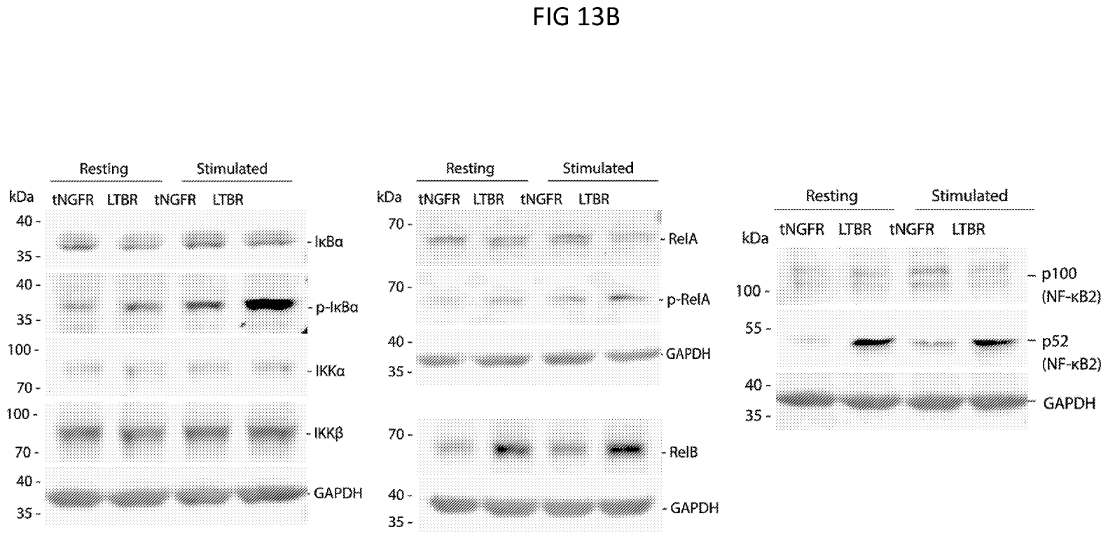

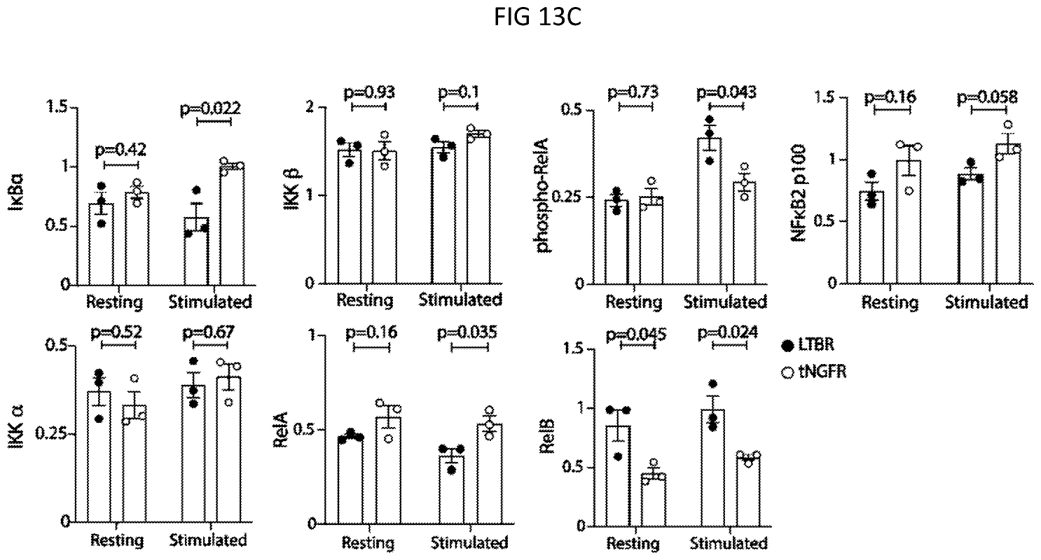

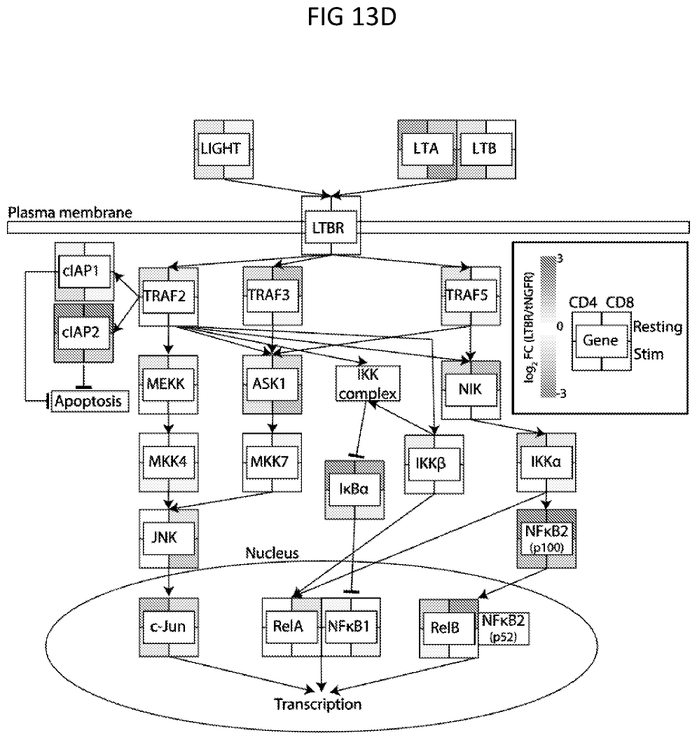

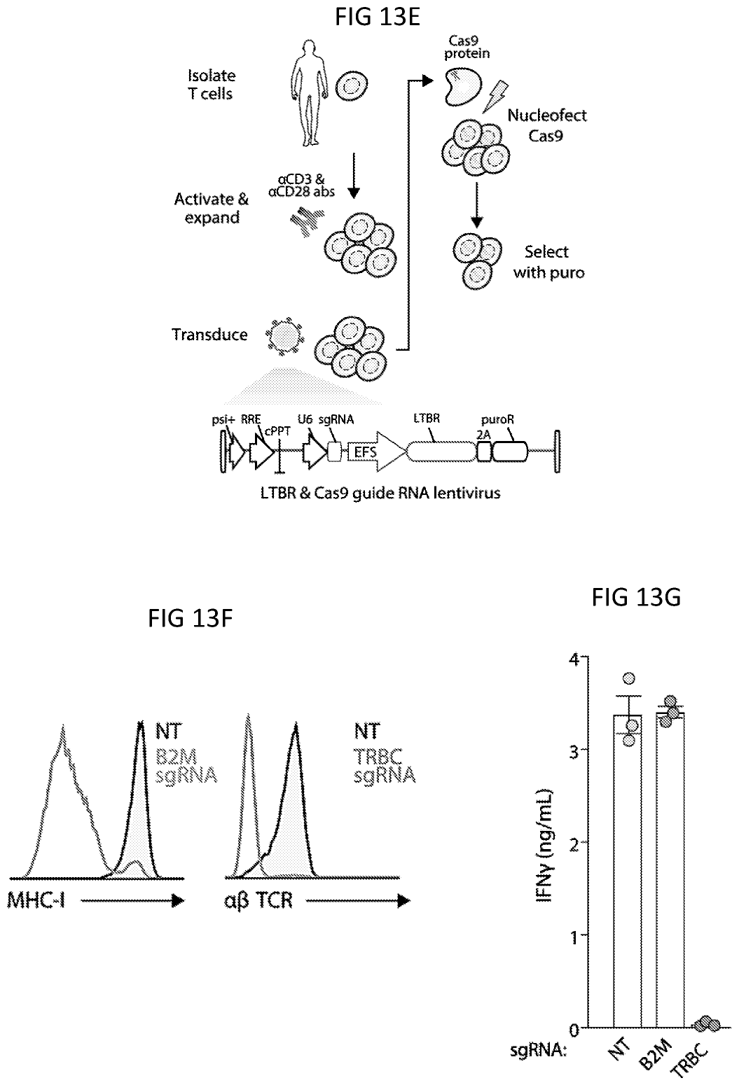

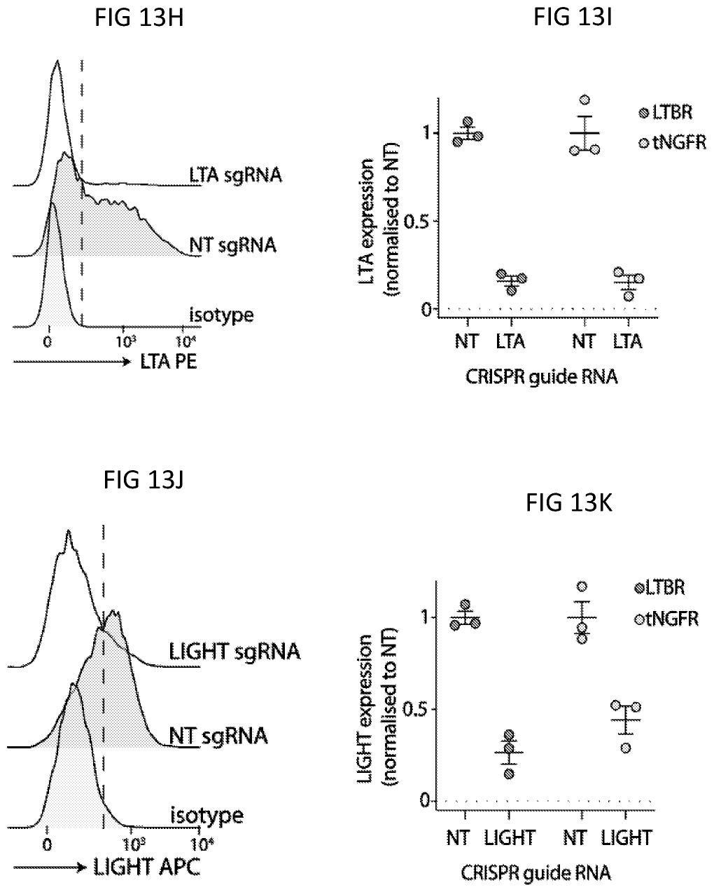

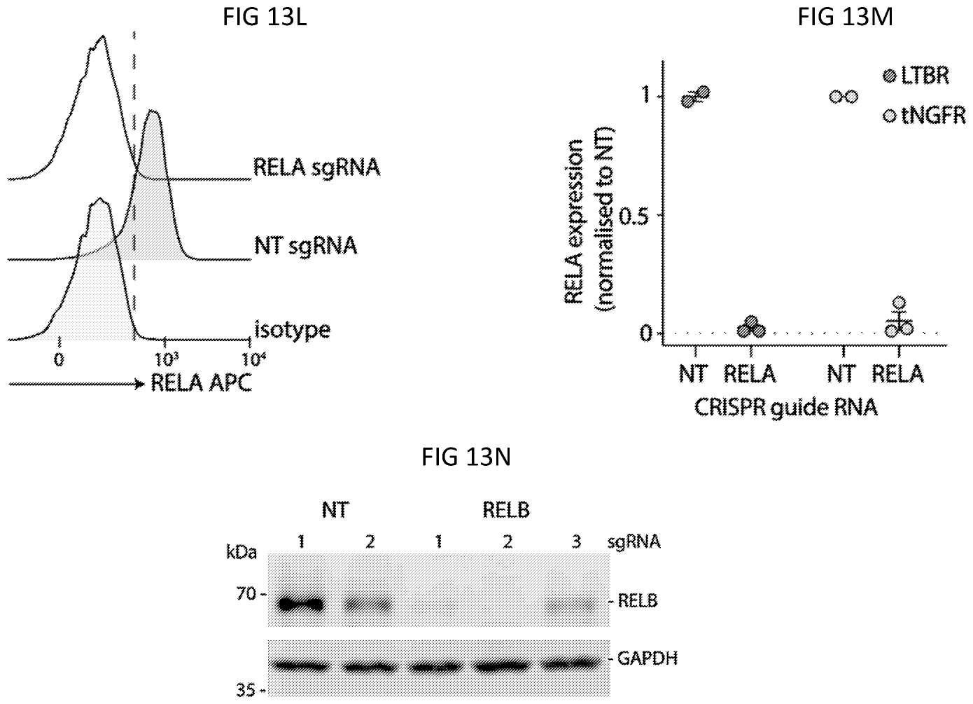

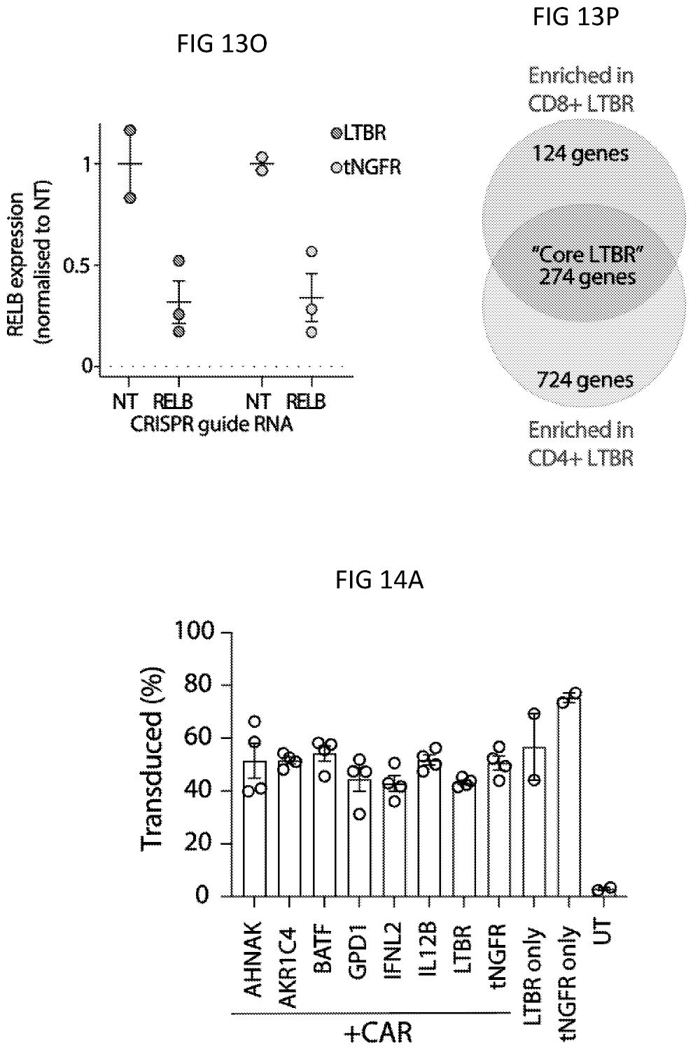

We then decided to investigate changes in protein expression and/or phosphorylation of the members of the NF-κB signaling pathway. We observed a more rapid phosphorylation of p65 (RELA) and a strong increase in phosphorylation of an NF-κB inhibitor, IκBα, targeting IκBα for degradation; both of these effects enhance NF-κB activation or transcription (FIG. 4G, FIG. 4H, FIG. 13A-FIG. 13C). In addition to changes in the canonical NF-κB pathway, we also detected an upregulation of key mediators of the non-canonical NF-κB pathway, RELB and NF-κB p52 (FIG. 4I, FIG. 13B, FIG. 13C).

Having established that LTBR activates both the canonical and the non-canonical NF-κB pathways, we sought to determine the molecular basis of this phenomenon by perturbing key genes in the LTBR and NF-κB pathways by co-delivery of LTBR or tNGFR and CRISPR constructs that target 11 genes involved in the LTBR signaling pathway³² (FIG. 4J, FIG. 13D—FIG. 13O). Knockout of LTB, TRAF2 and NIK (also known as MAP 3K14) significantly reduced the secretion of IFNγ from LTBR cells but not (or to a lesser extent) from control (tNGFR) cells, whereas perturbations of LIGHT (also known as TNFSF14), ASK1 (also known as MAP 3K5) and RELA had a stronger effect on control cells than on LTBR cells. The effect of LTB loss on T cell activation in LTBR cells supports the observation that alanine mutagenesis of key residues involved in LTA or LTB binding (FIG. 4E) partially reduced the LTBR phenotype. Notably, we observed that loss of either TRAF2 or TRAF3 boosted IFNγ secretion in tNGFR cells only, in line with previous findings that T cells from TRAF2, dominant negative mice are hyperresponsive to TCR stimulation³³.

To investigate the potential roles of canonical versus non-canonical NF-κB signaling in LTBR T cells, we decided to analyze the global effects of RELA or RELB loss on the LTBR-driven gene expression profiles. Using bulk RNA-seq on T cells overexpressing LTBR or tNGFR, we discovered that only the loss of RELA significantly downregulated the expression of ‘core’ LTBR genes, whereas loss of RELB had no effect (FIG. 4K, FIG. 13P).

### ORFs Enhance Antigen-Specific Responses

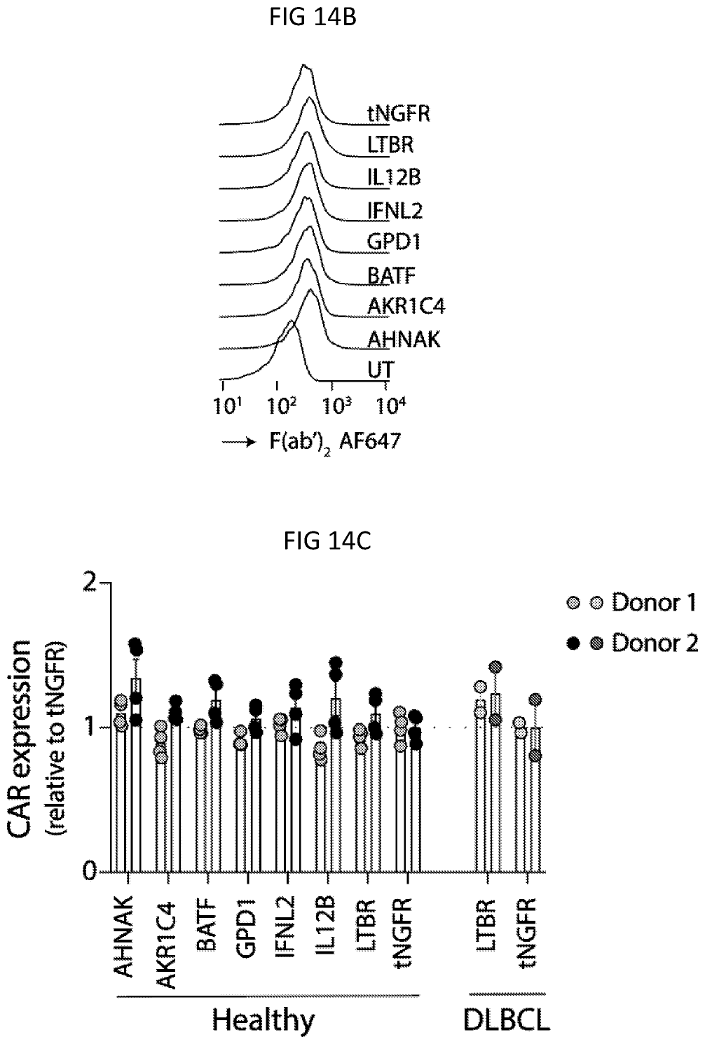

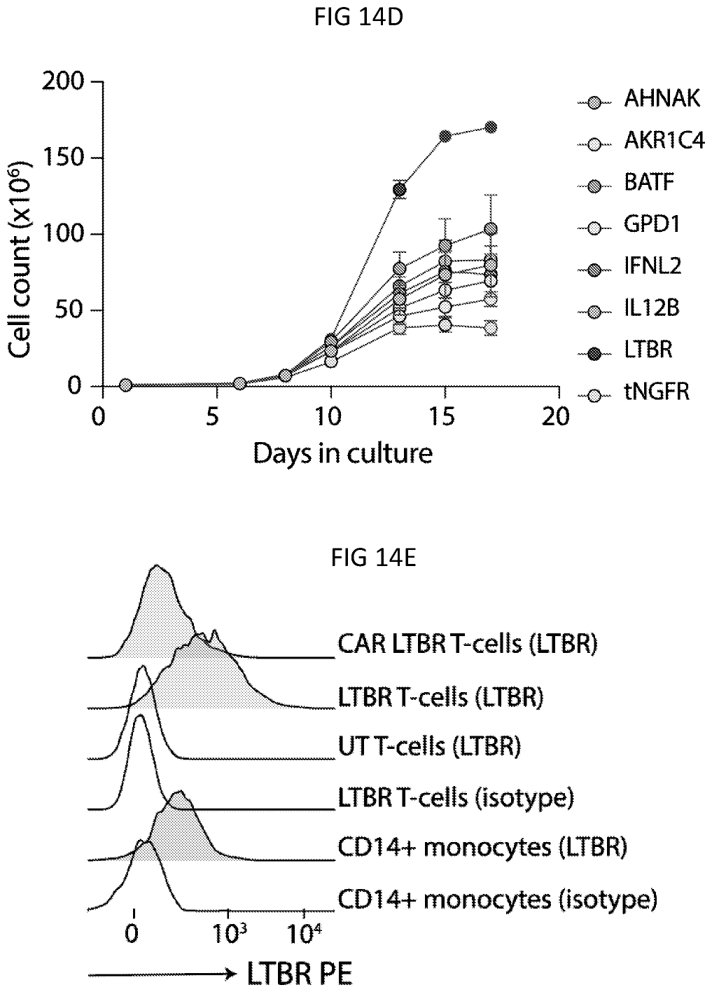

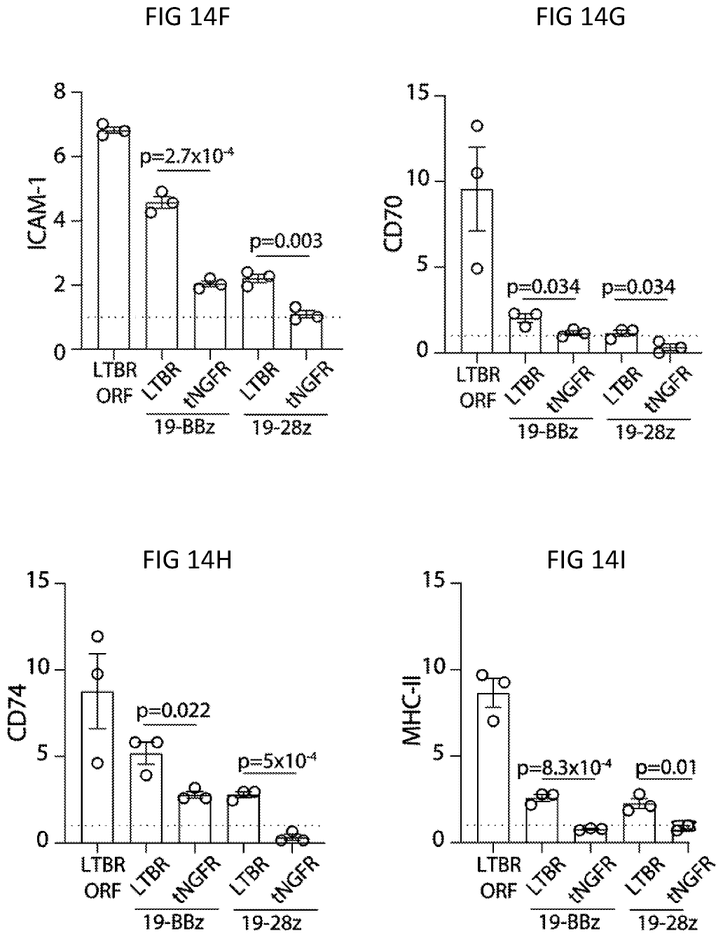

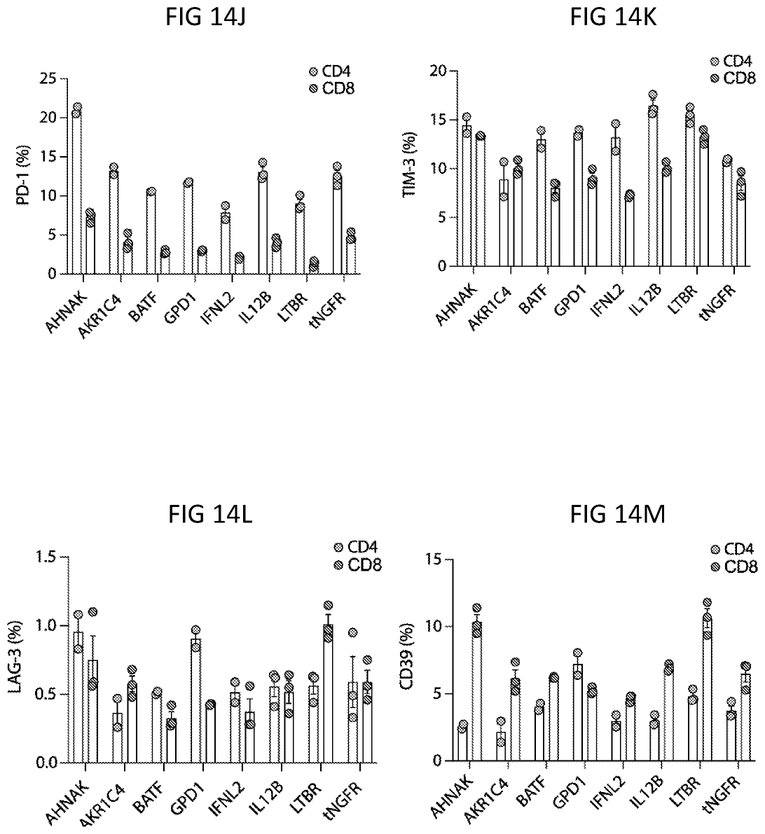

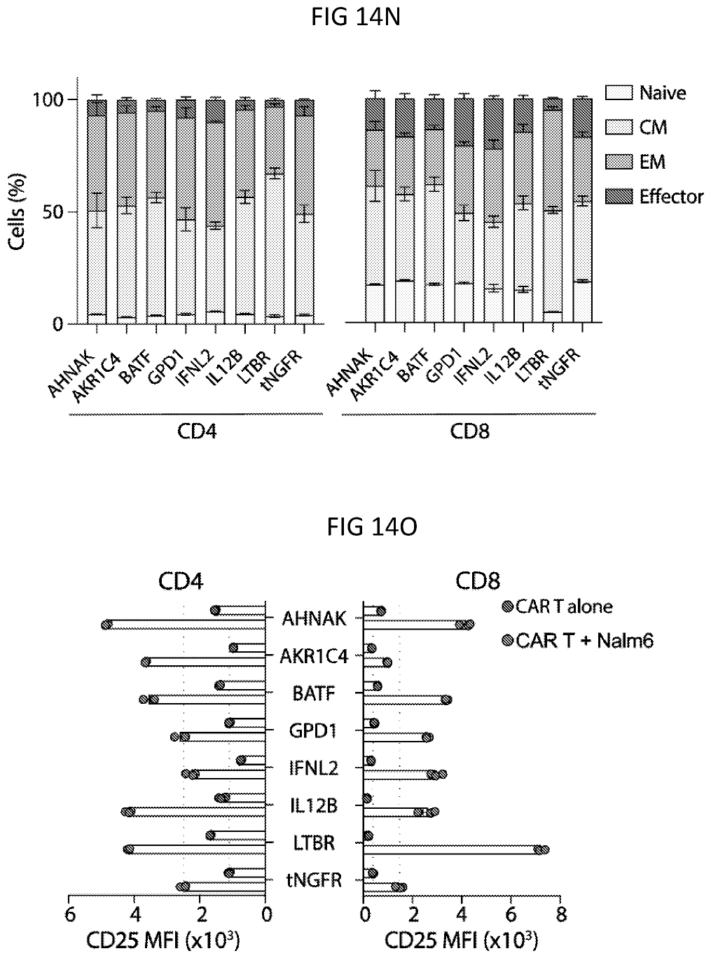

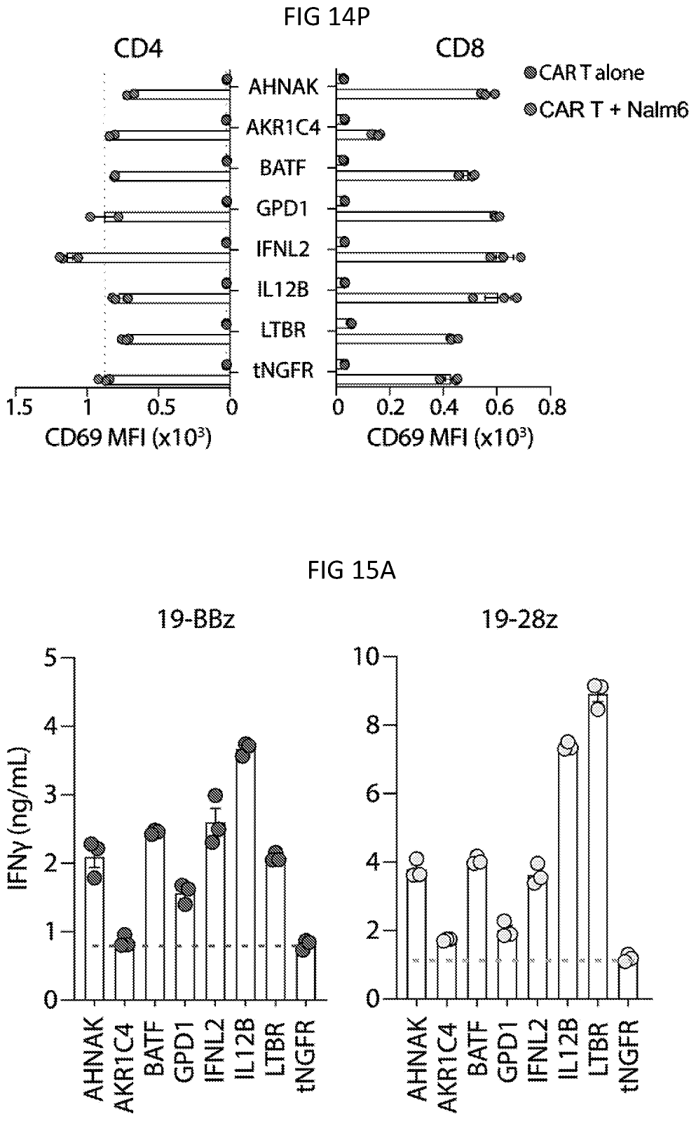

Thus far we have shown that top-ranked genes from the ORF screen improve T cell function using a non-specific, pan-TCR stimulation. We next sought to determine whether a similar improvement could be observed using antigen-specific stimulation (FIG. 5A). To that end, we co-expressed several top-ranked genes with two FDA-approved CARs that target CD19, a B cell marker (FIG. 14A-FIG. 14D). Using LTBR as an example, we demonstrated that ORF expression is achievable with this tricistronic vector (FIG. 14E-FIG. 14I). The sequences of the tricistronic vectors are provided in the sequence listing (with schematic provided in FIG. 5A):
19-28-z+LTBR protein: SEQ ID NO: 3
19-28-z+LTBR DNA: SEQ ID NO: 4
19-28-z+NGFR protein: SEQ ID NO: 5
19-28-z+NGFR DNA: SEQ ID NO: 6
19-BB-z+LTBR protein: SEQ ID NO: 7
19-BB-z+LTBR DNA: SEQ ID NO: 8
19-BB-z+NGFR protein: SEQ ID NO: 9
19-BB-z+NGFR DNA: SEQ ID NO: 10

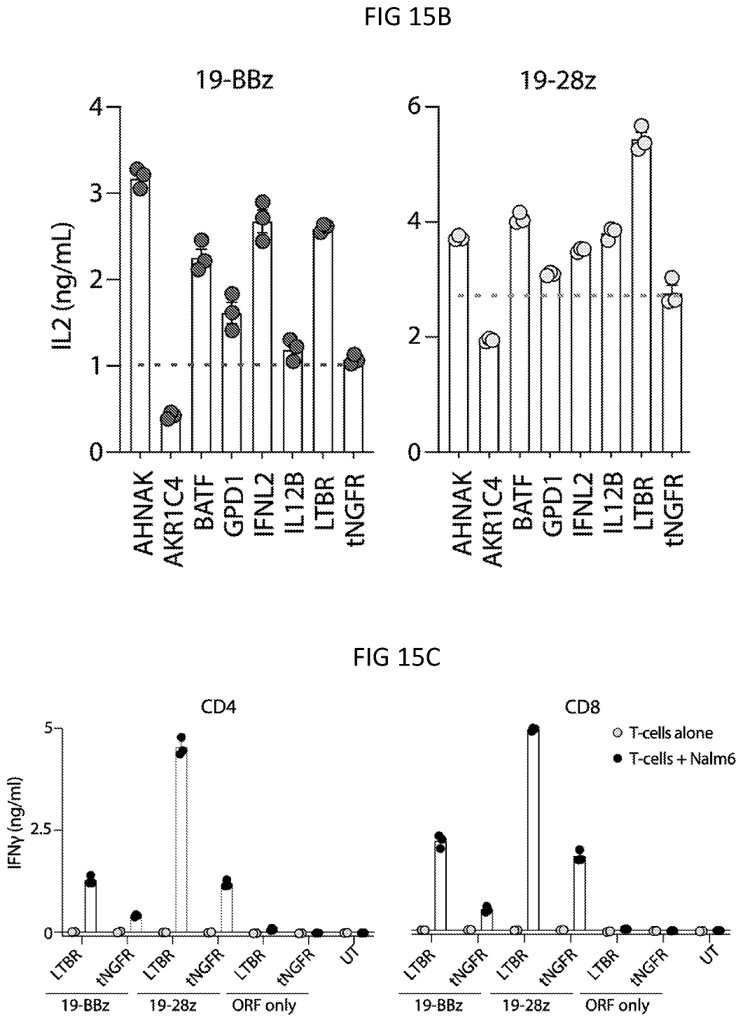

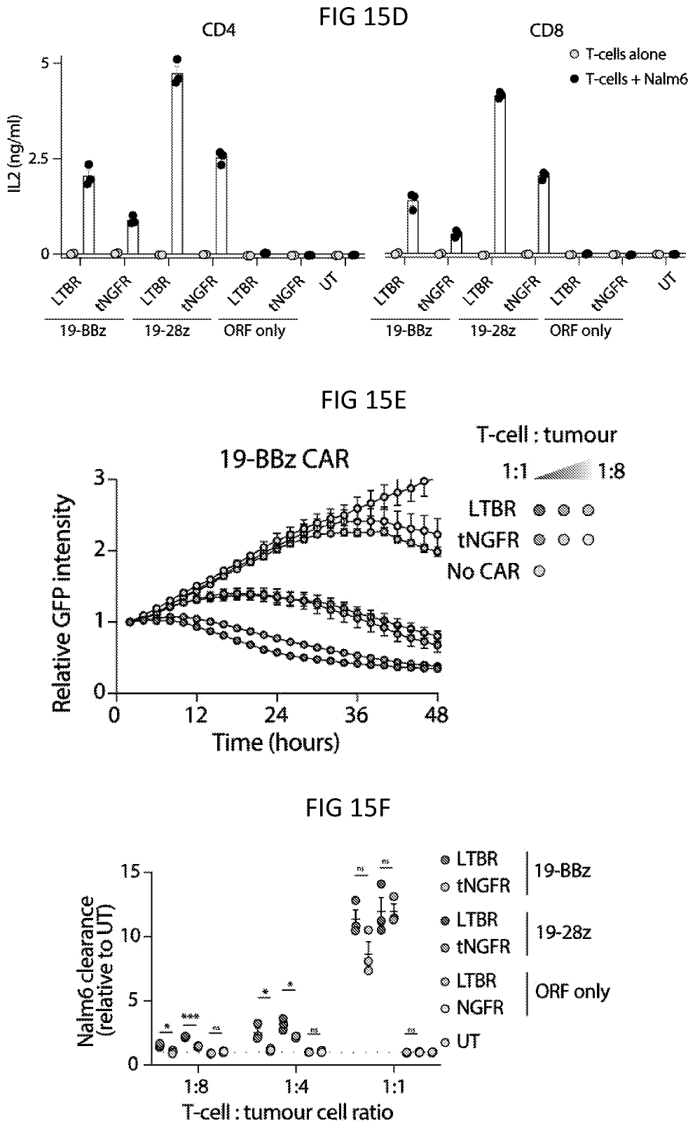

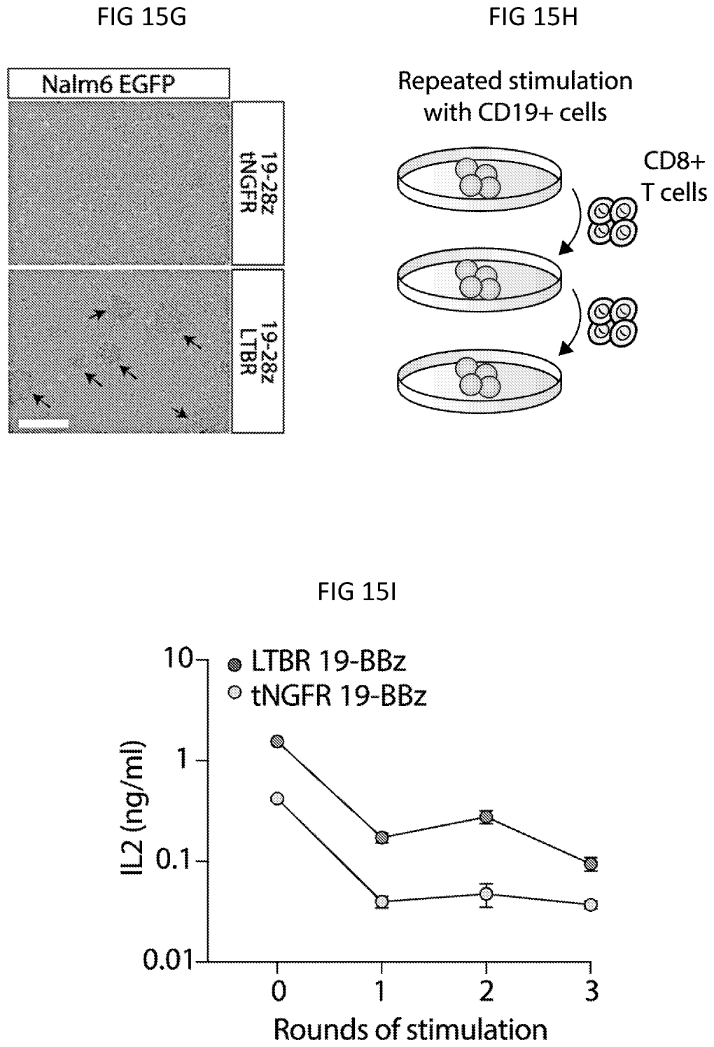

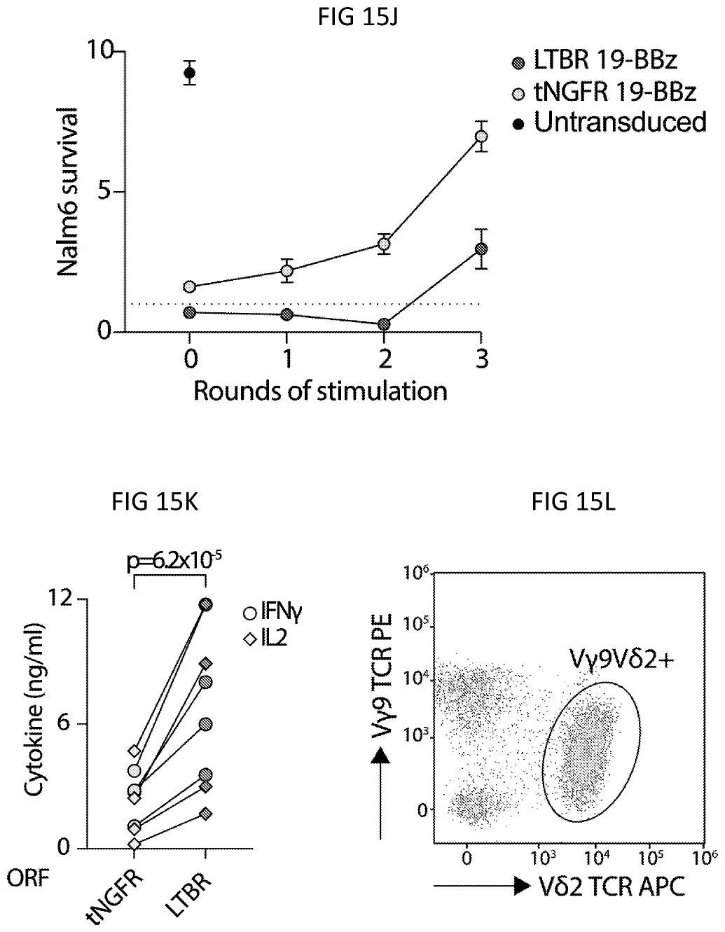

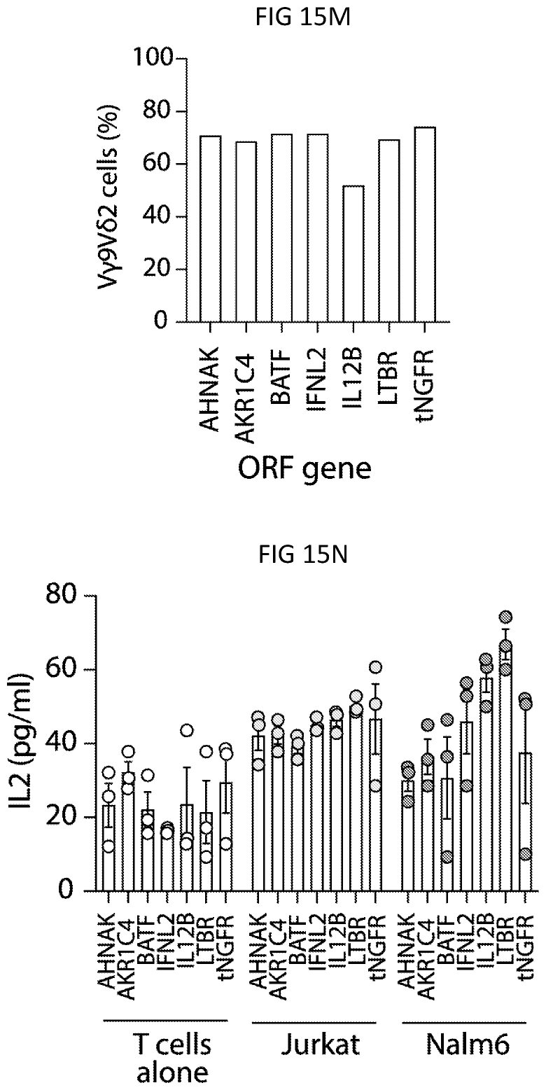

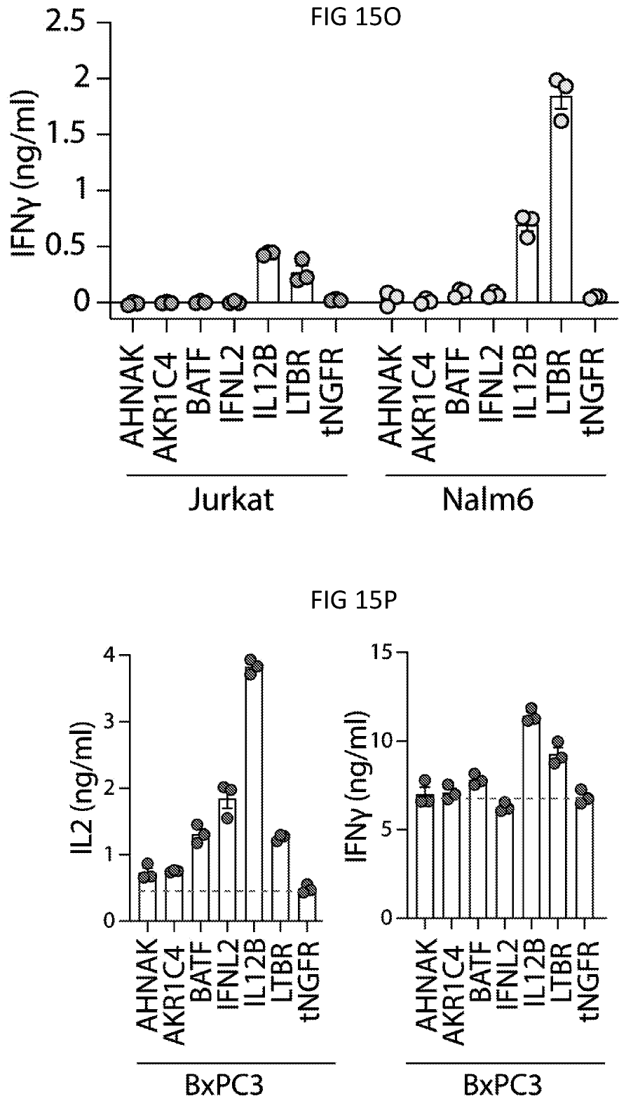

Since both CARs use different costimulatory domains, from CD28 or 4-1BB, we wanted to determine whether top-ranked genes that were selected using CD28 co-stimulation could also work in the context of 4-1BB co-stimulation. Nearly all of the top-ranked genes tested, with the exception of AKR1C4, improved upregulation of CD25 and antigen-specific cytokine secretion, with no major differences in the differentiation or exhaustion phenotype (FIG. 5B, FIG. 5C, FIG. 14J-FIG. 14P, FIG. 15A-FIG. 15D).

Although production of IL-2 and IFNγ is crucial for the clonal expansion and antitumor activity of T cells, another vital component of tumor immunosurveillance is direct cytotoxicity. Top-ranked genes had an overall stronger effect on the cytotoxicity of CD28 CAR T cells than 4-1BB CAR T cells (FIG. 5D-FIG. 5E, FIG. 15E, FIG. 15F). Notably, we observed that CAR T cells co-expressing LTBR tended to form large cell clusters; these clusters were typically absent in wells with control cells but are consistent with the overall higher expression of adhesion molecules such as ICAM-1 in LTBR-expressing cells (FIG. 15G). Another important feature of effective antitumor T cells is the ability to maintain functionality despite chronic antigen exposure. In line with our previous findings in the context of LTBR alone (FIG. 4D), CAR T cells expressing LTBR showed a better functionality than matched CAR T cells expressing tNGFR after repeated challenge with target cells (FIG. 5F, FIG. 15H-FIG. 15J).

T cells from healthy donors are relatively easy to engineer and rarely show signs of dysfunction in culture, whereas autologous T cells in patients with cancer are often dysfunctional, showing limited proliferation and effector functions³⁴. To investigate whether top-ranked genes can improve CAR T cell response not only in healthy T cells but also in potentially dysfunctional T cells derived from patients, we transduced CD19 CARs co-expressed with LTBR or a control gene into peripheral blood mononuclear cells (PBMCs) from patients with diffuse large B cell lymphoma. After co-incubation with CD19⁺ target cells, we observed a similar increase in the secretion of IL-2 and IFNγ from LTBR CAR T cells to that seen in healthy donors, indicating that identified ORFs can be successfully used to engineer T cells from patients with lymphoma ex vivo (FIG. 5G, FIG. 15K). Of note, there was no secretion of cytokines in response to CD19− cells, indicating that overexpression of LTBR does not induce a spurious, antigen-independent response.

The screen and subsequent validations were performed in αβ T cells, the predominant subset of T cells in human peripheral blood. Although immunotherapy based on αβ T cells has shown considerable potential in the clinic, γδ T cells present an attractive alternative, owing to their lack of MHC restriction, ability to target broadly expressed stress markers in a cancer-type-agnostic manner and more innate-like characteristics⁵. We therefore sought to determine whether the top genes validated in αβ T cells translated to γδ T cells. After co-incubation with leukemia or pancreatic ductal adenocarcinoma cancer cells, we observed an increase in IL-2 and IFNγ secretion from γδ T cells that were transduced with top-ranked genes (FIG. 5H, FIG. 15L-FIG. 15P). Thus, top-ranked genes from our screen can act on signaling pathways that are conserved between even highly divergent T cell subsets, highlighting their broad applicability for cancer immunotherapy.

### Discussion

In summary, here we developed a genome-scale gain-of-function screen in primary human T cells, in which we examined the effects of nearly 12,000 full-length genes on TCR-driven proliferation in a massively parallel manner. The largest—to our knowledge—a previously published gain-of-function study in primary T cells involved 36 constructs, including full-length genes and synthetic receptors³⁵. That approach relied on construct delivery via donor DNA and Cas9-mediated targeted insertion. Although using donor DNA for target gene delivery allows for more flexibility in terms of construct design, especially for engineering synthetic receptors, that method is less scalable and less accessible in terms of cost and complexity than the lentiviral library that we used here. Thus, ORF-based gain-of-function screens are readily applicable to a plethora of T cell phenotypes and settings, and that they offer the opportunity for clinical translation. In fact, all FDA-approved CAR therapies already rely on lentiviral or retroviral integration of a CAR transgene, and therefore an addition of an ORF to this system should pose no major manufacturing or regulatory challenges. The use of ORF-encoding mRNA delivered to CAR T cells before infusion is another translational route, especially if there are safety concerns about the mode of action of a particular ORF.

Gain-of-function screens have the potential to uncover regulators that are tightly controlled, restricted to a specific developmental stage or expressed only in certain circumstances. As shown here, LTBR is canonically absent from cells of lymphoid origin, but, owing to the intact signaling pathway, it can have a synthetic role when introduced to T cells. Although constitutive activation of other TNFRSF members might result in a similar phenotype, one of the features that distinguishes LTBR (and plausibly led to its enrichment, but not that of other TNFRSF members, in the screen) is the formation of an autocrine loop whereby the receptor and its ligands are present in the same cell. It is particularly noteworthy that expression of LTBR boosts IL-2 secretion, as this cytokine is produced exclusively by T cells and not by cell types that endogenously express LTBR. In addition to boosting cytokine secretion, overexpression of LTBR promoted stemness (expression of TCF1) and decreased activation-induced apoptosis, as well as offered a level of protection against phenotypic and functional hallmarks of T cell exhaustion-all of which are features not recapitulated by cell types that endogenously express LTBR. Previous work using overexpression of LTBR in cell lines showed that LTBR has a pro-apoptotic role³⁶, in direct contrast to the phenotype that we observed in primary T cells. Transcript- and protein-level analyses revealed that LTBR drives the constitutive activation of both canonical and non-canonical NF-κB pathways. However, using epigenomic profiling and CRISPR-based functional perturbations we showed that the phenotypic and functional changes resulting from LTBR expression are mediated primarily through activation of the canonical NF-κB pathway, whereas changes in the non-canonical pathway may not be essential for the observed phenotypes—in contrast to the well-established role of non-canonical NF-κB activation in cells that endogenously express LTBR37.

Gene overexpression has been used for pre-clinical enhancement of CAR T cell therapies in numerous studies. For example, armoring CAR T cells with cytokines such as IL-12 or IL-18, which are not typically produced by T cells but are known to improve T cell function when secreted by other cell types, was shown to improve their antitumor activity^{38,39}. Notably, a previous study found that CAR T cell exhaustion can be alleviated by overexpression of c-JUN, a transcription factor identified by RNA-seq as specifically depleted in exhausted cells⁴⁰. Future studies that adapt genome-wide gain-of-function screens to relevant models of immunotherapy will lead to advanced target selection for engineering synthetic cellular therapies that can overcome the immunosuppressive tumor microenvironment and eradicate established cancers.

### Example 3: Improved CAR Solid Tumor Responses

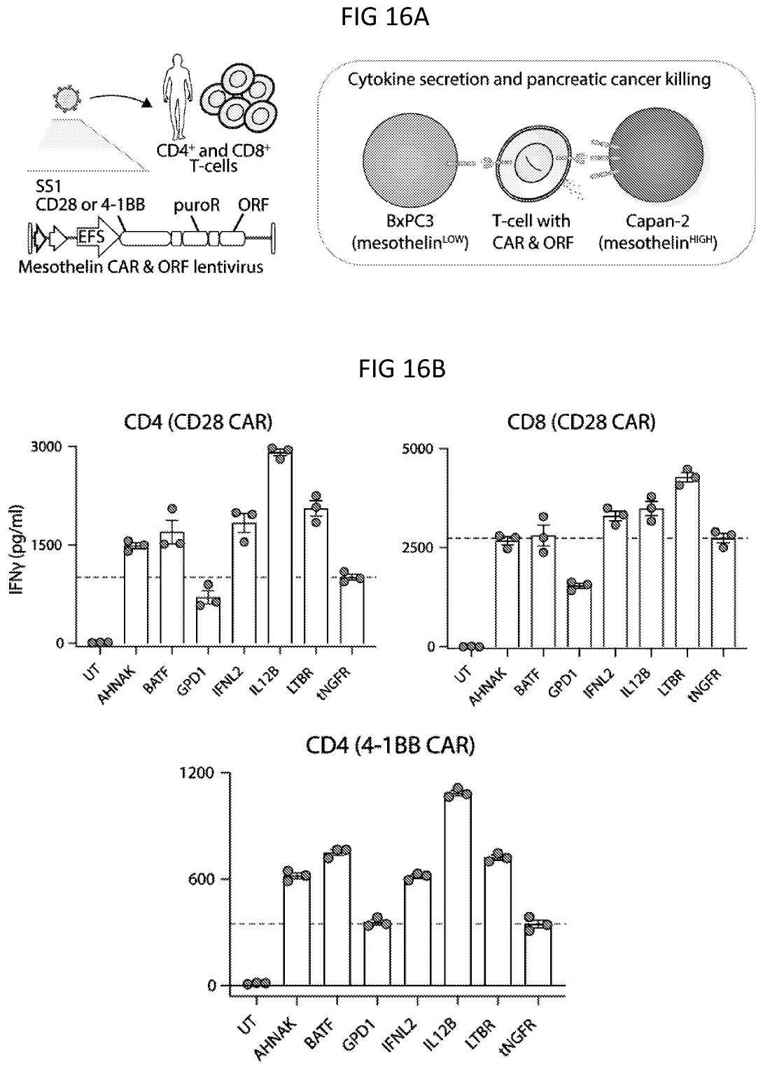

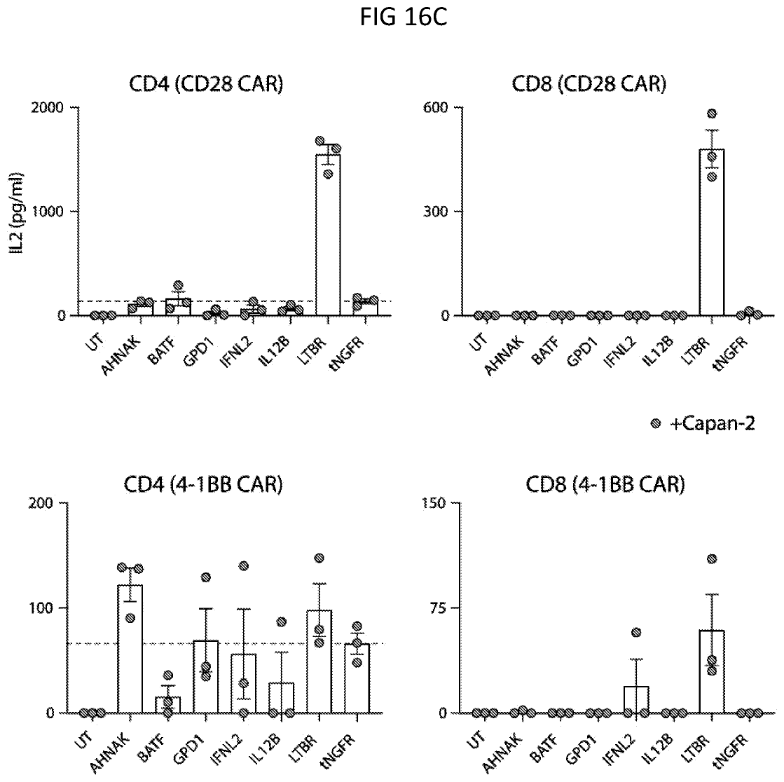

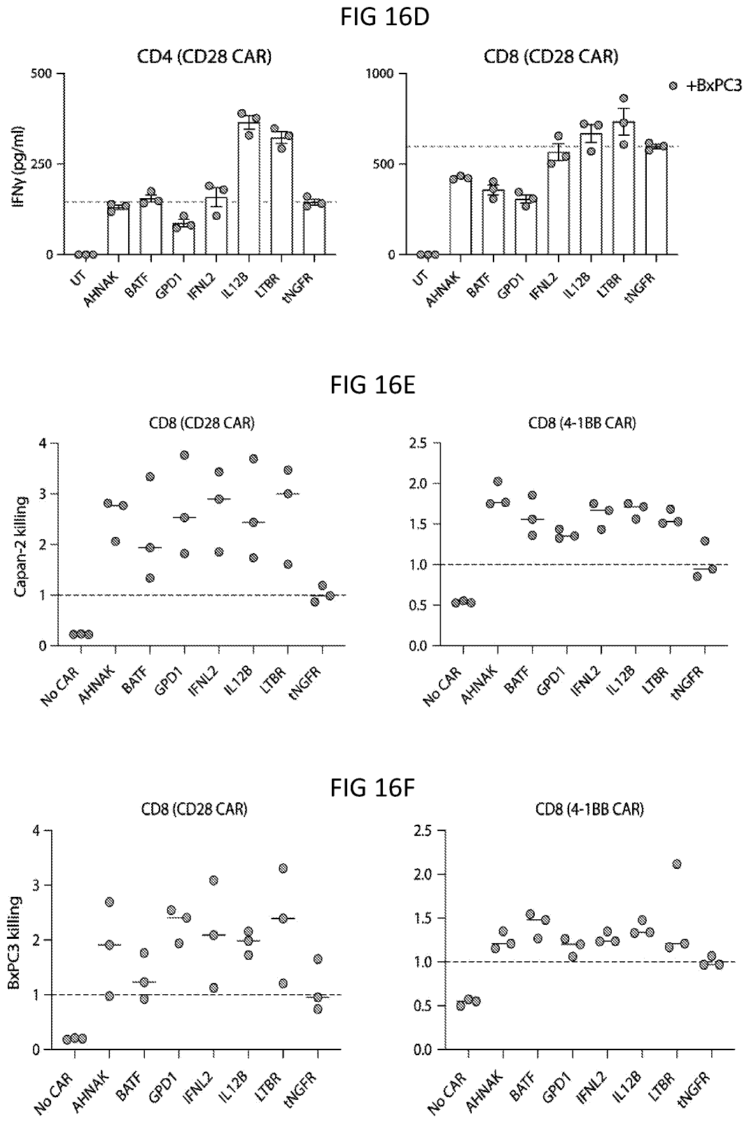

We have shown that LTBR and several other top-ranked genes (ORFs, open reading frames) identified in the screen boost the antitumor response of anti-CD19 CARs in context of a B cell leukemia. Here we tested whether a similar improvement of activity can be seen in conjunction with two clinically-tested anti-mesothelin CARs (using either 4-1BB or CD28 costimulatory domains) in the context of pancreatic cancers. We tested T cells co-expressing a CAR and an ORF against Capan-2, a pancreatic cancer line expressing high levels of mesothelin, the CAR target, and BxPC3, a pancreatic cancer line expressing low levels of mesothelin (FIG. 16A). The sequences of the tricistronic vectors are provided in the sequence listing (with schematic provided in FIG. 16A):
SS1-28-z+LTBR protein: SEQ ID NO: 11
SS1-28-z+LTBR DNA: SEQ ID NO: 12
SS1-BB-z+LTBR protein: SEQ ID NO: 13
SS1-BB-z+LTBR DNA: SEQ ID NO: 14

Following overnight co-incubation, we determined that all but one ORF tested (that is, AHNAK, BATF, IFNL2, IL12B, and LTBR) boosted antigen-specific secretion of IFNγ, when used with either CAR (either 41BB or CD28) against a mesothelin-high cell line Capan-2 (FIG. 16B). In terms of boosting IL-2 secretion, we observed a striking improvement over the negative (tNGFR) control when using LTBR, and to lesser extent AHNAK (FIG. 16C). In terms of reactivity against mesothelin-low line BxPC3, an improvement over the negative control was observed predominantly in T cells overexpressing IL12B or LTBR (FIG. 16D).

Cytokine secretion is one of the aspects of a productive antitumor response-another one is direct cytotoxicity. Therefore, we tested the ability of CAR T cells co-expressing top genes to kill GFP+ Capan2 or BxPC3 cells (FIG. 16E, FIG. 16F). While increased cytotoxicity against mesothelin-high Capan-2 exhibited by CAR T cells overexpressing any of the six top genes tested (including GPD1) was expected given the improvement in cytokine secretion, we also observed increased cytotoxicity against BxPC3 cells. Therefore, we concluded that the top-ranked genes identified in our screen (including but not limited to AHNAK, BATF, GPD1, IFNL2, IL12B, and LTBR) could boost reactivity of diverse CARs (anti-CD19 shown previously, anti-mesothelin shown here) using different costimulatory domains (CD28 or 4-1BB), in different cancer types (including liquid tumors such as B cell leukemia and solid tumors such as pancreatic cancer), and at different target antigen densities (mesothelin-high and mesothelin-low cell lines).

### Example 4: Improved Activity of a TCR in Solid Tumor

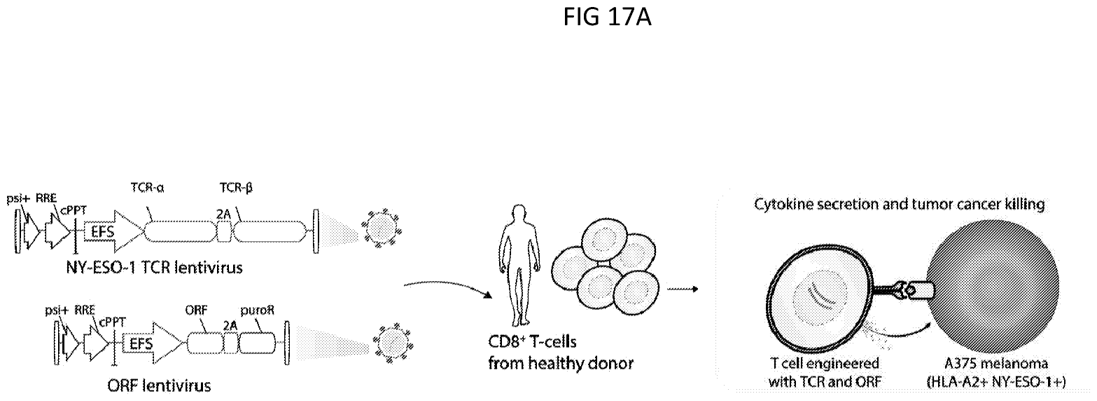

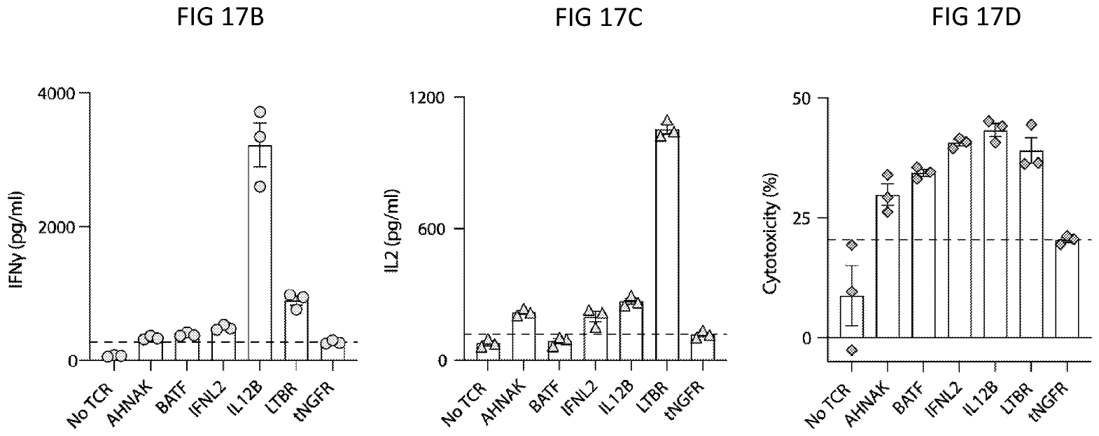

T cell therapies can rely on redirecting the cells to a given tumor target using either a CAR or a TCR. The former has the advantage of being able to target tumors in different patients, regardless of their HLA haplotype, while the latter can also target antigens that are intracellular (since epitopes from all cellular proteins are sampled by and displayed on the HLA molecules). Here we used a clinically-tested TCR directed against an epitope from NY-ESO-1, commonly expressed in many cancer histologies, including but not limited to melanoma, multiple myeloma, sarcoma, lung cancer. Due to size restrictions we delivered the TCR and the gene (ORF, open reading frame) on two separate lentiviruses that were used to co-transduce T cells (FIG. 17A). Then, the dual-transduced T cells were selected using puromycin (only T cells transduced with the ORF lentivirus would survive) and using antibody-based selection of NY-ESO-1 TCR positive cells (in presence of dasatinib to prevent T cell activation and thus activation-induced cell death during the selection process).

We then tested the engineered CD8+ T cells against an HLA-A2+NY-ESO-1+ melanoma line A375. Most genes tested increased the secretion one or both of cytokines IFNγ and IL2 (FIG. 17B, FIG. 17C). We also measured direct cytotoxicity against A375 cells and demonstrated that all genes tested showed superior cytotoxicity than the TCR-transduced T cells co-expressing the negative control gene tNGFR (FIG. 17D). Therefore, we concluded that the top-ranked genes identified in our screen (including but not limited to AHNAK, BATF, GPD1, IFNL2, IL12B, and LTBR) could boost reactivity of T cells engineered with a cancer-specific TCR.

### Example 5: LTBR Co-Delivery Improves Anti-CD19 4-1BB-z CAR Activity In Vivo

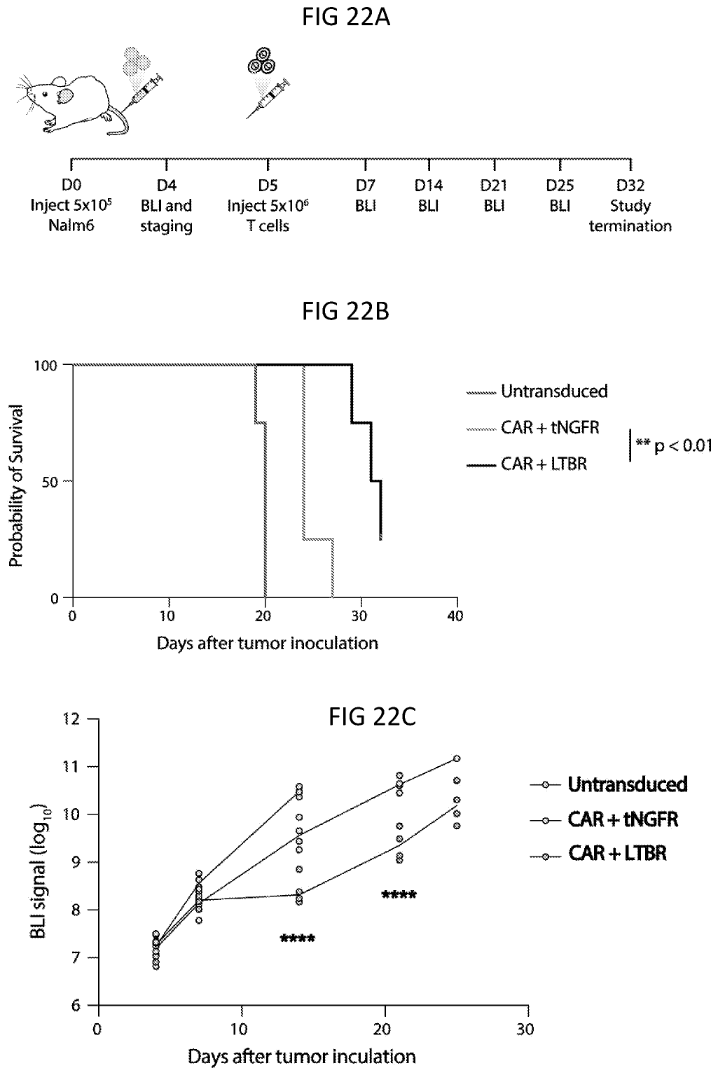

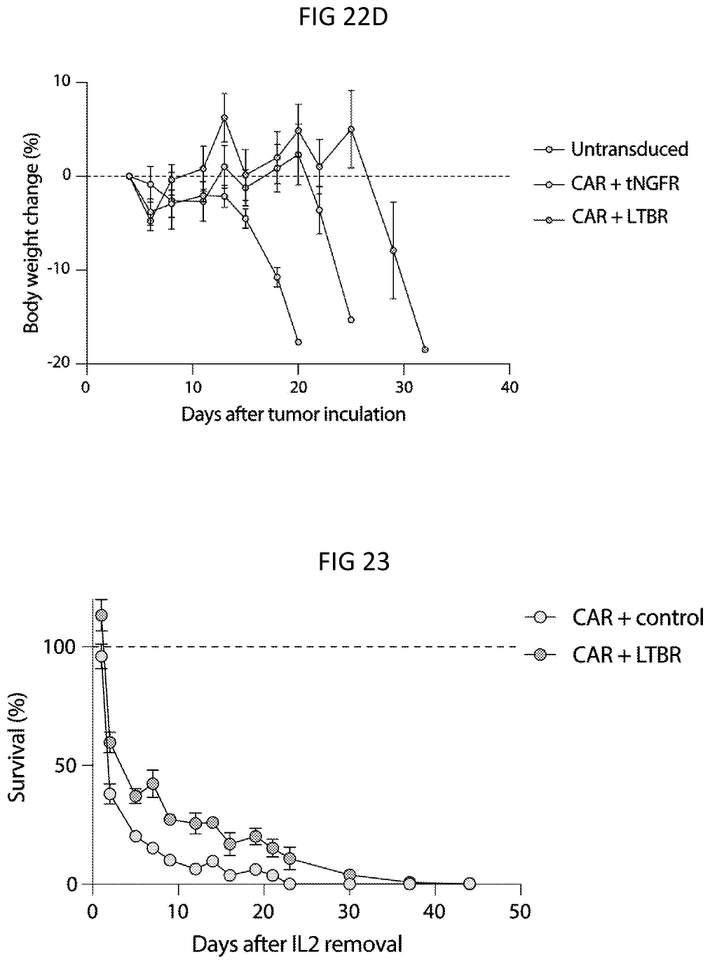

We have previously shown that co-delivery of LTBR on the same lentiviral vector as an anti-CD19 CAR results in superior antitumor activity in vitro (Legut et al, Nature 2022). Here we tested the efficacy of an anti-CD19 CAR (FMC6.3 scFv, CD8 stalk and transmembrane domain, 4-1BB and CD3z signaling domains: 19-BB-z), with or without LTBR, in an immunocompromised (NSG) mouse model of disseminated leukemia, Nalm6 (FIG. 22A). Mice treated with untransduced T cells (CD4 and CD8, 1:1 ratio) survived for a median of 19 days post tumor inoculation while mice treated with CAR T cells co-expressing an irrelevant gene tNGFR had their survival extended to a median of 23 days (20% increase over untransduced T cells). In contrast, mice treated with CAR T cells co-expressing LTBR survived for a median of 31 days (63% increase over untransduced T cells and 35% increase over control CAR T cells) (FIG. 22B). Furthermore, LTBR CAR T cells significantly reduced the tumor burden in treated mice, compared with control CAR T cells (FIG. 22C). Finally, no specific toxicities were observed in mice treated with either CAR T cell product, as determined by gross pathology examination at termination; the loss of body weight that was observed is typical for this model and is due to the tumor burden. In line with the improved antitumor efficacy of LTBR CAR T cells, the loss of body weight was substantially delayed in mice treated with LTBR CAR T cells, as compared to untransduced or control CAR T cells (FIG. 22D).

### Example 6: LTBR Co-Delivery Improves Anti-CD19 4-1BB-z CAR Survival in Absence of IL2 but does not Result in Leukemic Transformation

We have previously shown that LTBR boosts T cell proliferation and reduces apoptosis (Legut et al, Nature 2022). However, constitutive introduction of a gene capable of inducing T cell proliferation/evasion of apoptosis raises the possibility of malignant transformation. To test whether LTBR may allow T cells to survive and/or proliferate in absence of cytokines, we cultured T cells transduced with CAR+LTBR or a control gene tNGFR in media without any cytokines or with exogenous IL2, as per standard protocol. Both populations of CAR T cells, tNGFR and LTBR transduced, underwent rapid cell death after IL2 withdrawal (FIG. 23). In the initial phase, LTBR CAR T cells survived better than control CAR T cells in absence of IL2, in line with the antiapoptotic effect of LTBR-however, over the course of the experiment both T cell types reached complete loss of viability, without any evidence of outgrowth of an IL2-independent, potentially malignantly-transformed T cell population. Thus, we concluded that constitutive LTBR expression does not cause malignant transformation of transduced T cells.

### Example 7: LTBR Phenotype is Independent of Media Used

The composition of cell culture media used for ex vivo expansion of T cells, for either research or clinical applications, can have substantial impact on T cell phenotype, function and clinical efficacy (Sarah MacPherson et al, Clinically relevant T cell expansion media activate distinct metabolic programs uncoupled from cellular function, Molecular Therapy—Methods & Clinical Development, Volume 24, March 2022, Pages 380-393). Here we wanted to determine if media composition affects the phenotype induced by LTBR expression in T cells. Thus, we cultured freshly isolated T cells in five different media types (all supplemented with 10 ng/ml rhIL2), listed below:
1—AIM V+5% FBS+10 mM HEPES (Lynn et al, c-Jun overexpression in CAR T cells induces exhaustion resistance, Nature, 576, 293-300 (December 2019))
2—AIM V+5% human serum (Gurusamy et al, Multi-phenotype CRISPR-Cas9 Screen Identifies p38 Kinase as a Target for Adoptive Immunotherapies, Cancer Cell. 2020 Jun. 8; 37(6):818-833.e9)
3—RPMI 1640+10% FBS+10 mM HEPES+1 mM sodium pyruvate+1× non-essential amino acids (Legut et al, CRISPR-mediated TCR replacement generates superior anticancer transgenic T cells, Blood. 2018 Jan. 18; 131(3):311-322)
4—ImmunoCult-XF T Cell Expansion Medium (Legut et al, A genome-scale screen for synthetic drivers of T cell proliferation, Nature. 2022 March; 603(7902): 728-735)
5—X-Vivo 15+5% FBS+50 UM β-mercaptoethanol+10 mM N-acetyl-L-cysteine (Shifrut, et al, Genome-wide CRISPR Screens in Primary Human T Cells Reveal Key Regulators of Immune Function, Cell. 2018 Dec. 13; 175(7): 1958-1971.e15)

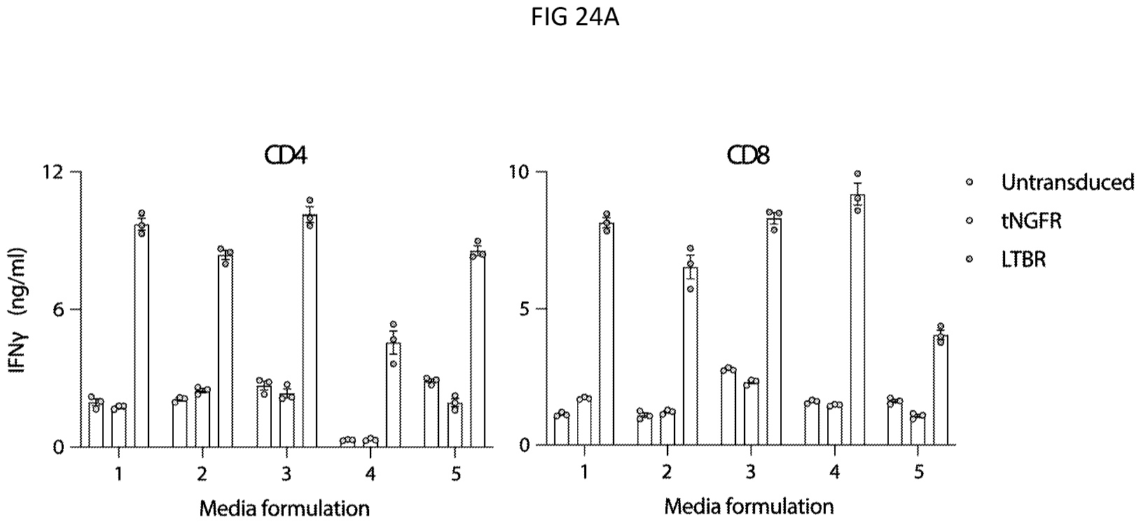

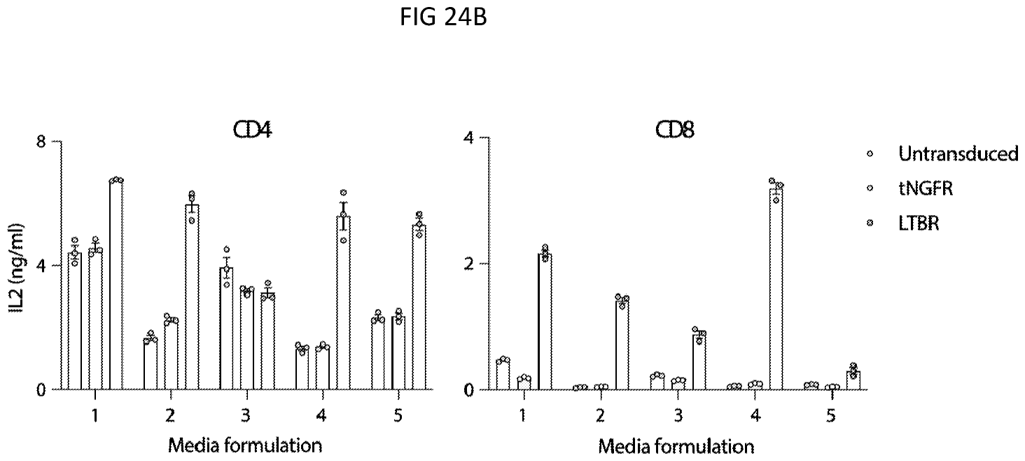

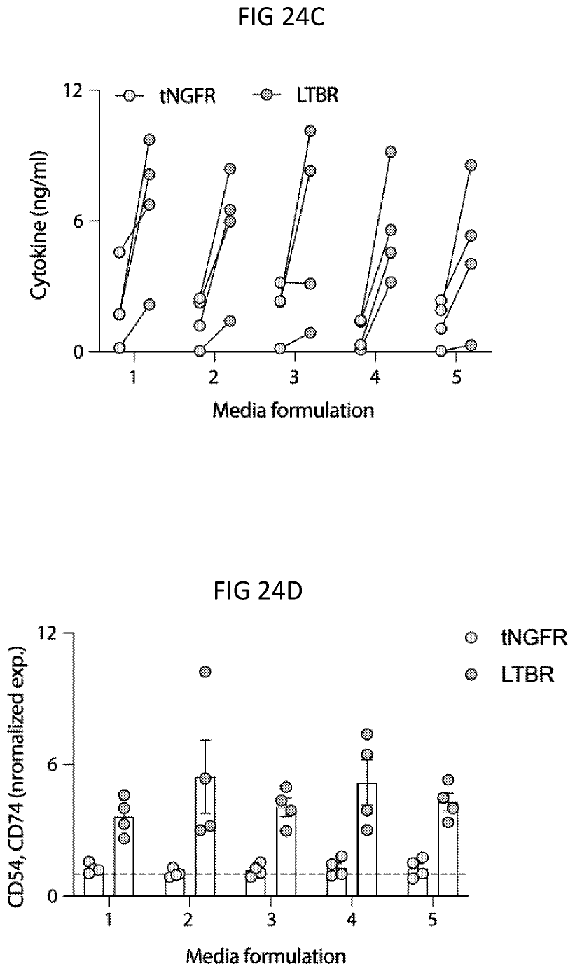

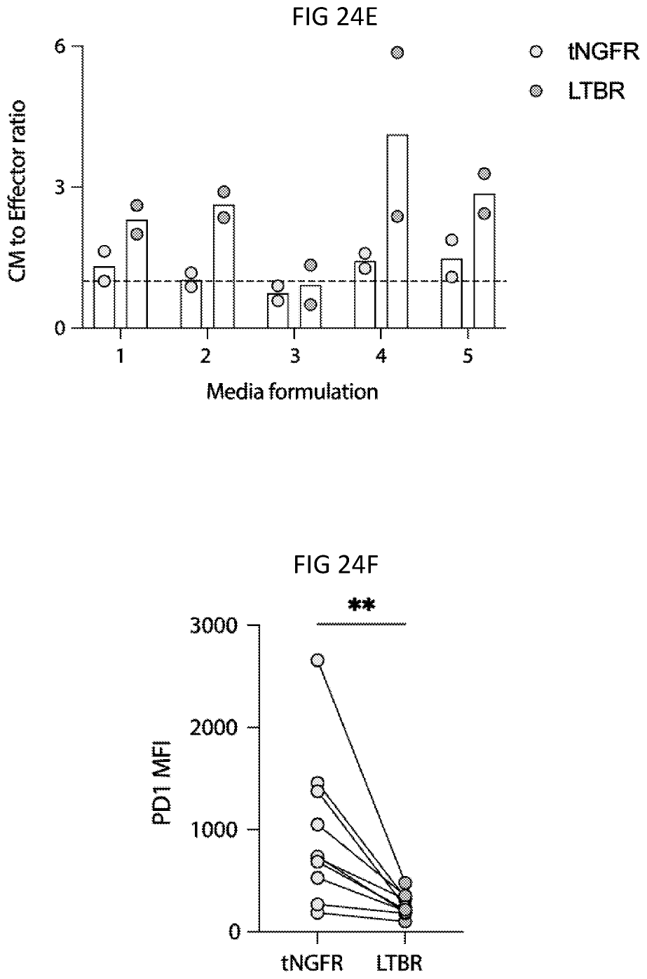

Following lentiviral transduction, selection and culture, T cells were restimulated and the quantity of secreted cytokines IFNγ (FIG. 24A) and IL2 (FIG. 24B) were measured (untreated, tNGFR, LTBR from left to right). As we showed before, LTBR overexpression strikingly increased the level of cytokines secreted upon stimulation. While the absolute quantities of cytokines differed between different media compositions, LTBR overexpression increased the cytokine levels above the ones observed with an irrelevant gene tNGFR in virtually all cases tested (FIG. 24C).

Another hallmark of LTBR overexpression in T cells is changes of expression of hundreds of genes, including CD54 and CD74 which are detectable on the protein level. In line with the data shown before, we observed an increase of CD54 and CD74 expression in LTBR T cells to a similar extent in all media tested (FIG. 24D). LTBR T cells also show preferential enrichment for the central memory phenotype, associated with improved clinical efficacy of the engineered T cell product—this preference for central memory phenotype was observed with LTBR T cells in all but one media type tested (FIG. 24E). Finally, LTBR alleviates hallmark of exhaustion in T cells, including expression on an inhibitory checkpoint PD-1. In all media compositions tested we observed a significant reduction in PD1 level in LTBR expressing T cells (FIG. 24F).

Thus, we conclude that while different media compositions do affect T cell phenotypes and functional responses, LTBR overexpression improves clinically-relevant T cell phenotypes regardless of the media used.

### Example 8: LTBR Phenotype is not Phenocopied by Other TNFRSF Members

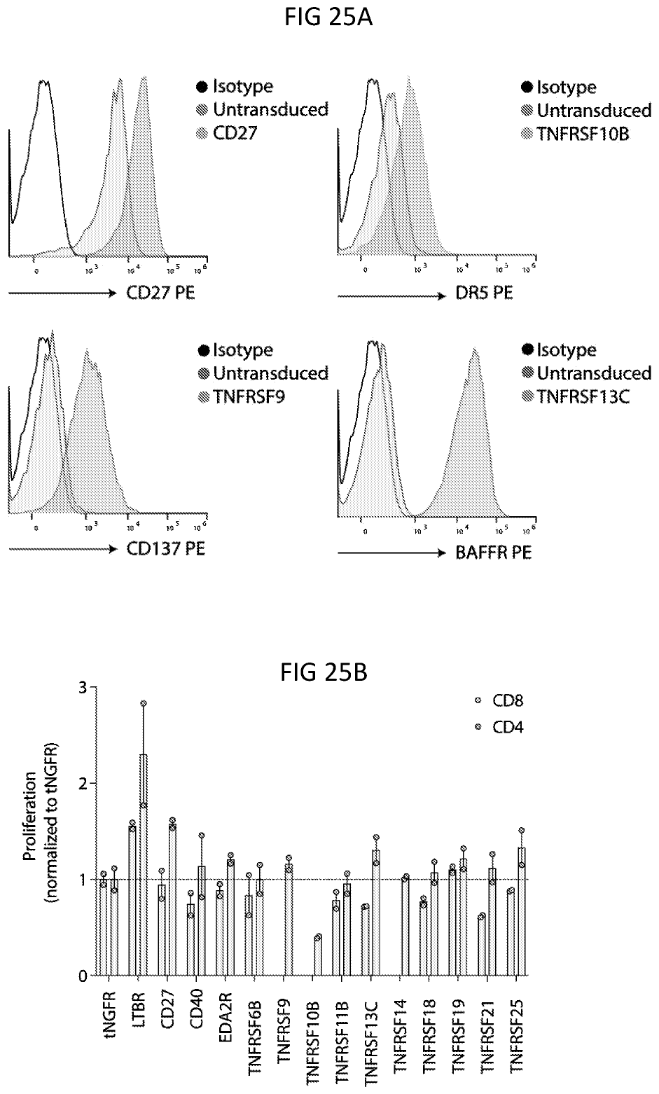

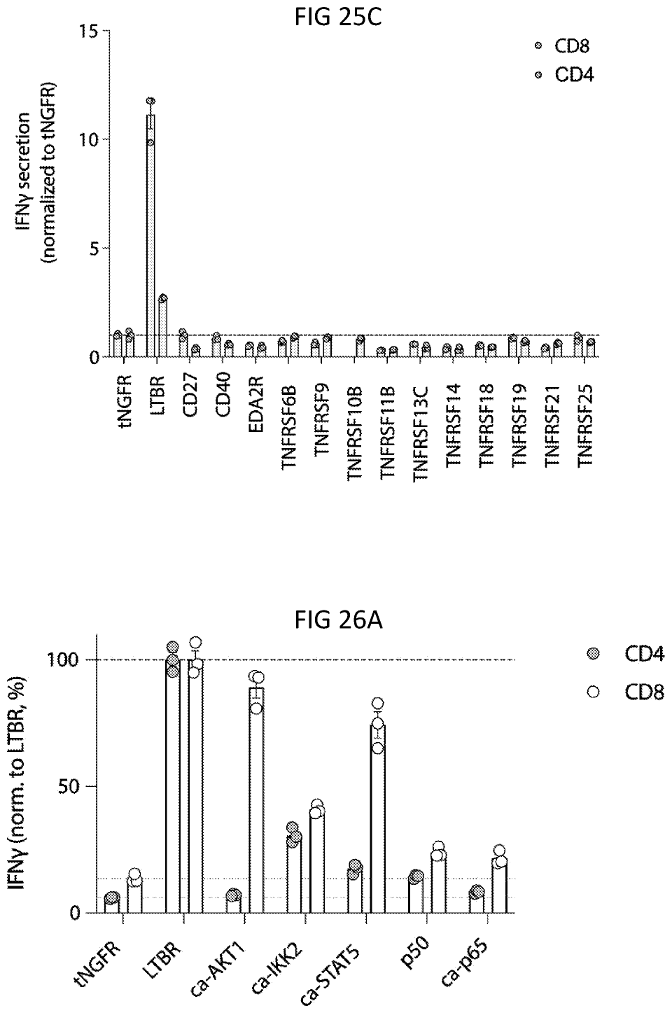

LTBR is a member of a protein family knows as the tumor necrosis factor receptor superfamily (TNFRSF). TNFRSF members are thought to act through similar molecular mechanisms and pathways (Dostert et al, The TNF Family of Ligands and Receptors: Communication Modules in the Immune System and Beyond, Physiol Rev99: 115-160, 2019, epublished October 2018)—therefore, we wanted to establish if the phenotype observed with LTBR overexpression in T cells could be replicated by overexpressing other TNFRSF members. We cloned and overexpressed 13 TNFRSF members alongside LTBR and irrelevant gene tNGFR in CD4 and CD8 T cells from a healthy donor. We achieved high levels of overexpression for TNFRSF members tested, regardless of the endogenous level of expression (FIG. 25A). We then stimulated the transduced T cells and looked at T cell proliferation after 4 days. As demonstrated before, LTBR drove strong increase in T cell proliferation for both CD4 and CD8 T cells (FIG. 25B). In case of other TNFRSFs tested, only a slight (at best) increase of CD4 proliferation, and no increase of CD8 proliferation, were observed. Another hallmark of LTBR overexpression in an increase in cytokine secretion after stimulation. However, out of 14 TNFRSF members tested, only LTBR induced a strong increase of IFNγ secretion, in both CD4 and CD8 (FIG. 25C). Thus, we concluded that LTBR drives a distinct program upon overexpression in T cells, compared to other TNFRSF members.

### Example 9: LTBR Phenotype is not Phenocopied by Overexpression of Constitutively Active Positive Regulators of the NFkB Pathway

We showed before that LTBR phenotype is dependent on constitutive activation of the NFκB pathway. Therefore, we sought to replicate the phenotype observed in T cells overexpressing LTBR by delivering constitutively active variants of the key mediators in the NFκB pathway, specifically mutants of:
AKT1 (Kim et al, Systematic Functional Interrogation of Rare Cancer Variants Identifies Oncogenic Alleles, Cancer Discov. 2016 July; 6(7):714-26. doi: 10.1158/2159-8290.CD-16-0160. Epub 2016 May 4)
IKK2 (Mercurio et al, IKK-1 and IKK-2: cytokine-activated IkappaB kinases essential for NF-kappaB activation, Science, 1997 Oct. 31; 278(5339): 860-6. doi: 10.1126/science.278.5339.860)
STAT5 (Richter, et al, Non-canonical H3K79me2-dependent pathways promote the survival of MLL-rearranged leukemia, Elife, 2021 Jul. 15; 10:e64960. doi: 10.7554/eLife.64960.)
p65 (He and Weber, Phosphorylation of NF-kappaB proteins by cyclic GMP-dependent kinase. A noncanonical pathway to NF-kappaB activation, Eur J Biochem. 2003 May; 270(10): 2174-85. doi: 10.1046/j. 1432-1033.2003.03574.x),
processed, active variant of NFKB1, p50 (Ballard et al, The 65-kDa subunit of human NF-kappa B functions as a potent transcriptional activator and a target for v-Rel-mediated repression, Proc Natl Acad Sci USA. 1992 Mar. 1; 89(5): 1875-9. doi: 10.1073/pnas.89.5.1875.).

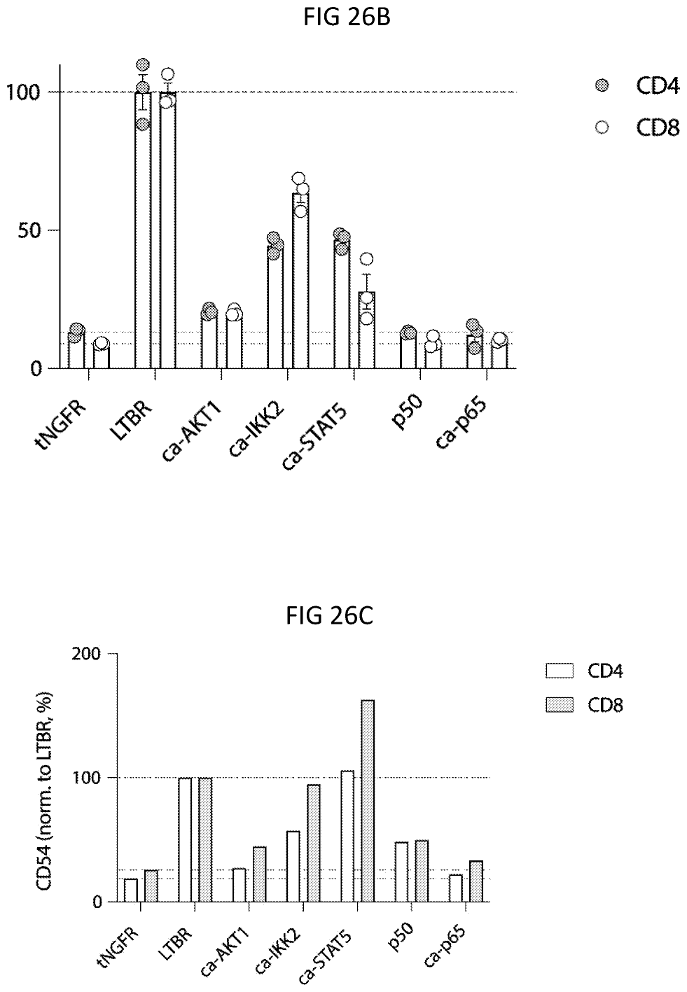

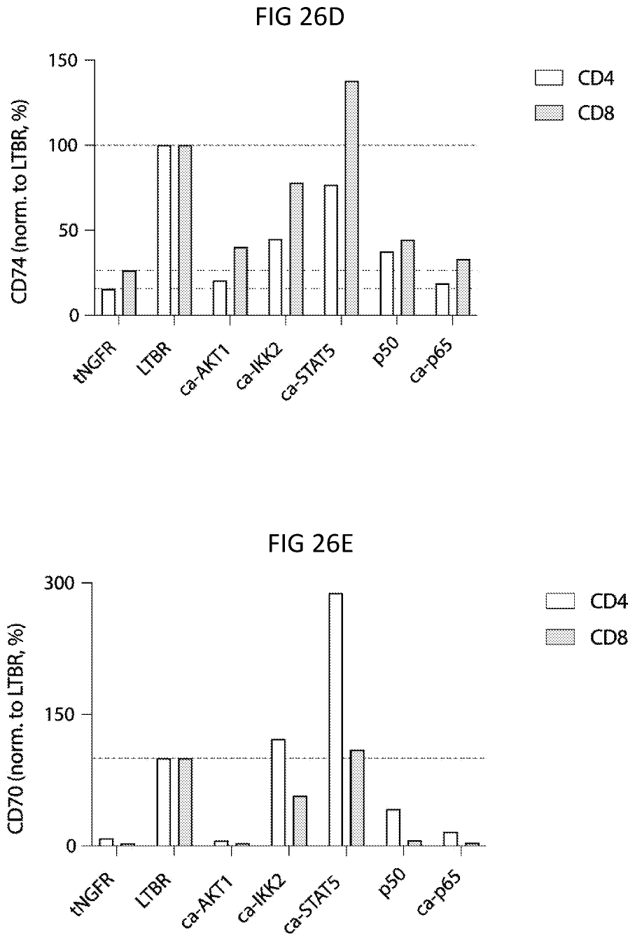

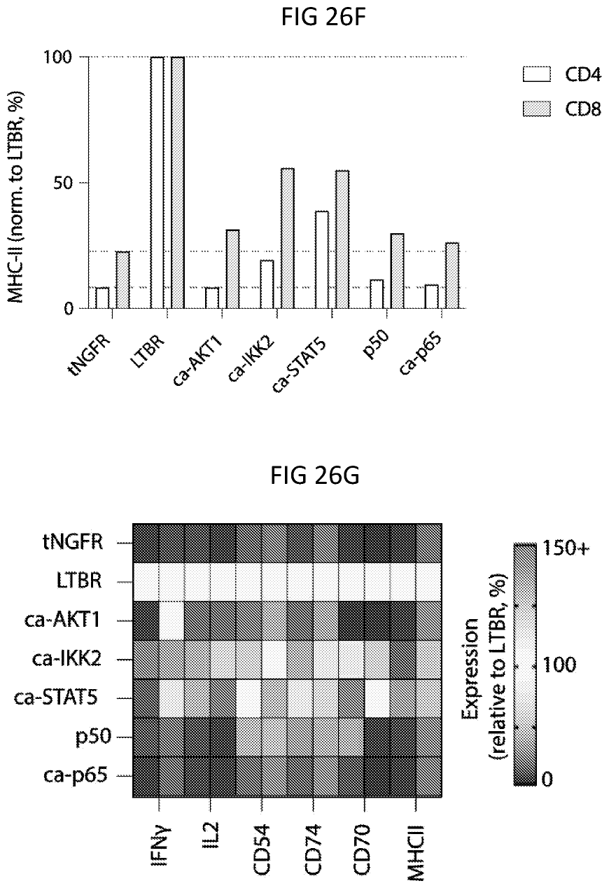

Two weeks post transduction and selection of primary human CD4 and CD8 T cells, we looked at hallmarks of LTBR overexpression phenotype: increased secretion of cytokines upon stimulation and increased expression of selected cell surface markers in resting T cells. In terms of cytokine secretion, we observed a striking increase of IFNγ in CD8, but not CD4, T cells expressing AKT1 or STAT5, and a moderate increase in T cells expressing IKK2 (FIG. 26A). Conversely, AKT1 expression in T cells did not increase the level of IL2 while STAT5 and IKK2 increased it slightly (FIG. 26B). None of the tested gene variants surpassed LTBR in terms of potentiating cytokine secretion.

LTBR overexpression has been shown to affect expression of hundreds of genes in T cells, including increased level of CD54, CD74, CD70 and MHC-II which can be detected on the protein level. STAT5, and IKK2 to a lesser extent, overexpression resulted in a considerable increased level of three of these markers, in some cases surpassing that of LTBR (FIG. 26C-F). Taken all together, overexpression of some constitutively active variants of the key mediators in the NFκB pathway can result in a phenotype resemblant of that of LTBR—but none of the proteins tested can fully phenocopy the breath and strength of LTBR-induced program in T cells (FIG. 26G).

### Example 10: LTBR Phenotype is not Phenocopied by Knockout of Negative Regulators of the NFkB Pathway but can be Further Enhanced by Said Knockouts

We showed before that LTBR phenotype is dependent on constitutive activation of the NFκB pathway. Therefore, we sought to replicate the phenotype observed in T cells overexpressing LTBR by knocking out two key inhibitors of the NFκB pathway, namely TNFAIP3 (also known as A20) and NFKBIA (also known as IκBα). To do so, we lentivirally delivered either LTBR or tNGFR to primary CD4 T cells, together with (on the same vector) sgRNAs targeting TNFAIP3, NFKBIA (3 independent sgRNAs each) or non-targeting (NT) sgRNAs (2 sgRNAs). Following lentiviral integration, T cells were electroporated with Cas9 protein as described before (Legut et al, Nature 2022).

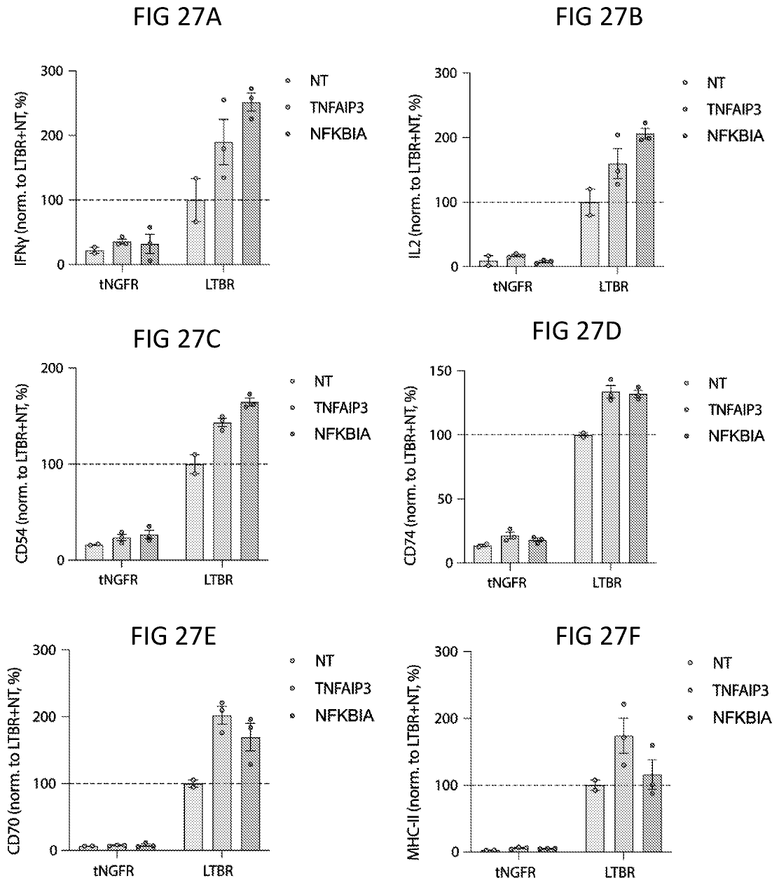

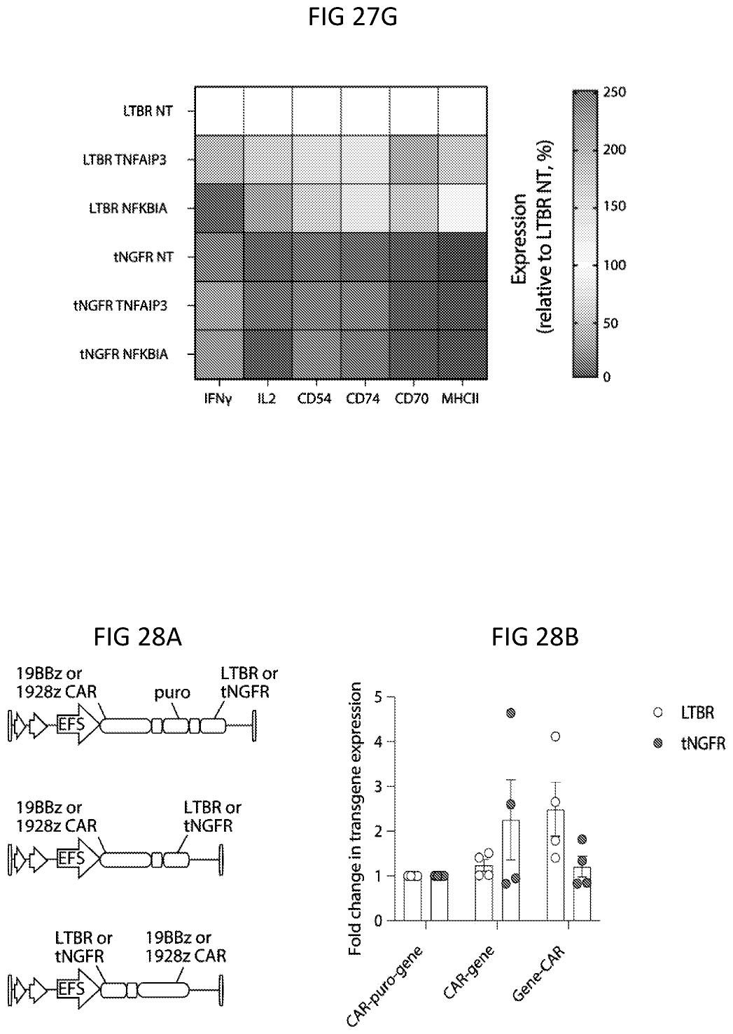

Two weeks post transduction and selection of primary human CD4 T cells, we looked at hallmarks of LTBR overexpression phenotype: increased secretion of cytokines upon stimulation and increased expression of selected cell surface markers in resting T cells. In terms of cytokine secretion, knocking out TNFAIP3 or NFKBIA showed hardly any effect in tNGFR T cells; conversely, knocking out either gene in LTBR T cells robustly increased secretion of IFNγ (FIG. 27A) and IL2 (FIG. 27B). A very similar phenotype was observed in resting T cells, looking at protein expression of representative genes (CD54, CD74, CD70 and MHC-II) upregulated upon LTBR overexpression—while knocking out TNFAIP3 or NFKBIA in tNGFR cells did not result in meaningful changes of expression of said genes, they became further upregulated in LTBR T cells (FIG. 27C-F). Taken together, knocking out negative regulators of the NFκB pathway by itself cannot replicate the phenotype seen in LTBR overexpression—but when LTBR is present, removing these negative regulators could further boost LTBR effects (FIG. 27G).

TABLE 4
sgRNA sequences.

| ID | Sequence | SEQ ID NO |
|---|---|---|
| NT sgRNA 1 | ACGGAGGCTAAGCGTCGCAA | 15 |
| NT sgRNA 2 | CGCTTCCGCGGCCCGTTCAA | 16 |
| TNFAIP3 sgRNA 1 | TTGCTCAAATACAAAGCCTG | 17 |
| TNFAIP3 sgRNA 2 | TGAGAGACTCCAGTTGCCAG | 18 |
| TNFAIP3 sgRNA 3 | GGCTTCCACAGACACACCCA | 19 |
| NFKBIA sgRNA 1 | CAACCAGCCAGAAATTGCTG | 20 |
| NFKBIA sgRNA 2 | TTCCAGGGCTCCGAGCCGCG | 21 |
| NFKBIA sgRNA 3 | GTTGTTCTGGAAGTTGAGGA | 22 |

### Example 11: Positioning of the LTBR Transgene within the Lentiviral CAR Expression Affects Expression Level and Functional Response

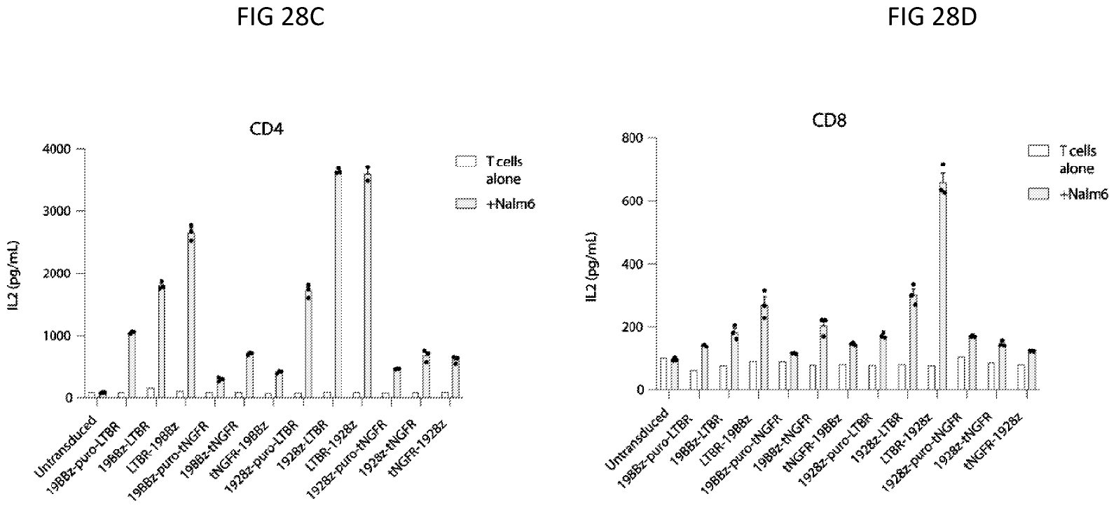

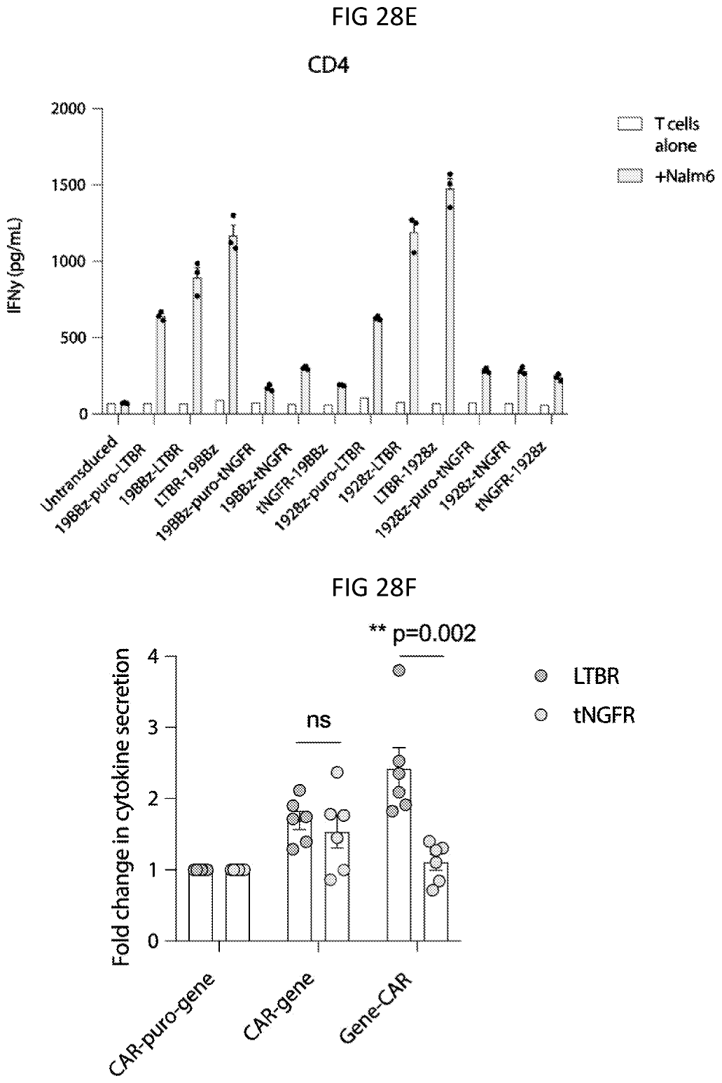

Previously, we observed that LTBR co-expressed from the same tricistronic vector as a CAR and puromycin resistance gene (CAR-puro-LTBR) is expressed at a lower level than when expressed from a bicistronic vector (LTBR-puro) (Legut et al, Nature 2022). Therefore, we sought to improve LTBR expression in CAR T cells by removing the puromycin resistance gene and testing different positions of LTBR within the vector (CAR-LTBR or LTBR-CAR) (FIG. 28A). To control for transduction efficiency and CAR expression level that could result from changes in the vector structure, we also generated vectors that included an irrelevant gene tNGFR in place of LTBR. The vectors used are disclosed in the sequence listing.
19BBz-P2A-puro-T2A-tNGFR—SEQ ID NO: 25 & 26
19BBz-P2A-puro-T2A-LTBR—SEQ ID NO: 27 & 28
1928z-P2A-puro-T2A-tNGFR—SEQ ID NO: 29 & 30
1928z-P2A-puro-T2A-LTBR—SEQ ID NO: 31 & 32
19BBz-P2A-tNGFR—SEQ ID NO: 33 & 34
1928z-P2A-tNGFR—SEQ ID NO: 35 & 36
19BBz-P2A-LTBR—SEQ ID NO: 37 & 38
1928z-P2A-LTBR—SEQ ID NO: 39 & 40
tNGFR-P2A-19BBz—SEQ ID NO: 41 & 42
tNGFR-P2A-1928z—SEQ ID NO: 43 & 44
LTBR-P2A-19BBz—SEQ ID NO: 45 & 46
LTBR-P2A-1928z—SEQ ID NO: 47 & 48
EFS-tNGFR PGK-puro—SEQ ID NO: 49
(no promoter)tNGFR PGK-puro—SEQ ID NO: 50
NFAT-tNGFR PGK-puro—SEQ ID NO: 51
NFkB-tNGFR PGK-puro—SEQ ID NO: 52
AP1-tNGFR PGK-puro—SEQ ID NO: 53
EFS-LTBR PGK-puro—SEQ ID NO: 54
(no promoter) LTBR PGK-puro—SEQ ID NO: 55
NFAT-LTBR PGK-puro—SEQ ID NO: 56
NFkB-LTBR PGK-puro—SEQ ID NO: 57
AP1-LTBR PGK-puro—SEQ ID NO: 58

Looking at the expression of the transgene (LTBR or tNGFR), normalized to the corresponding CAR-puro-gene vector, we observed that removing puromycin resistance gene (CAR-gene) had only a slight effect on LTBR level while a much stronger effect on tNGFR level; conversely, positioning the gene upstream of the CAR (Gene-CAR) resulted in much stronger expression of LTBR but not tNGFR (FIG. 28B).

To assess the functional impact of changes in the transgene expression level, we exposed the T cells engineered with different CAR vectors to target cells expressing the antigen, and measured the quantities of cytokines secreted in response to target engagement. Overall, across all the systems tested (4-1BB or CD28 costimulation in the CAR; CD4 or CD8 T cells; IL2 or IFNγ secretion), there was a clear dose-dependence between LTBR expression level and CAR functional response, with CAR-puro-LTBR showing the lowest response, CAR-LTBR a moderate one, and LTBR-CAR the highest one (FIG. 28C-E). Interestingly, in case of tNGFR, the CAR-tNGFR vector resulted in the strongest response while there was no meaningful difference between CAR-puro-tNGFR and tNGFR-CAR. In summary, the CAR-gene vectors resulted in stronger functional response to target cells than CAR-puro-gene vectors, in cases of both LTBR and tNGFR transgenes, presumably due to increased CAR expression level. Conversely, in the Gene-CAR design, only LTBR transgene, but not tNGFR, showed a striking increase in functional response over CAR-puro-gene vectors (FIG. 28F). A possible explanation would be that the CAR expression is similar between CAR-puro-gene and Gene-CAR (in the former case due to the increased size of the transgene, in the latter due to CAR positioning in the vector) and lower, in both cases, than in the CAR-gene vector. In context of LTBR, the gains from higher LTBR expression in the Gene-CAR vector offset the losses from overall lower CAR expression.

### Example 12: Inducible Expression of LTBR

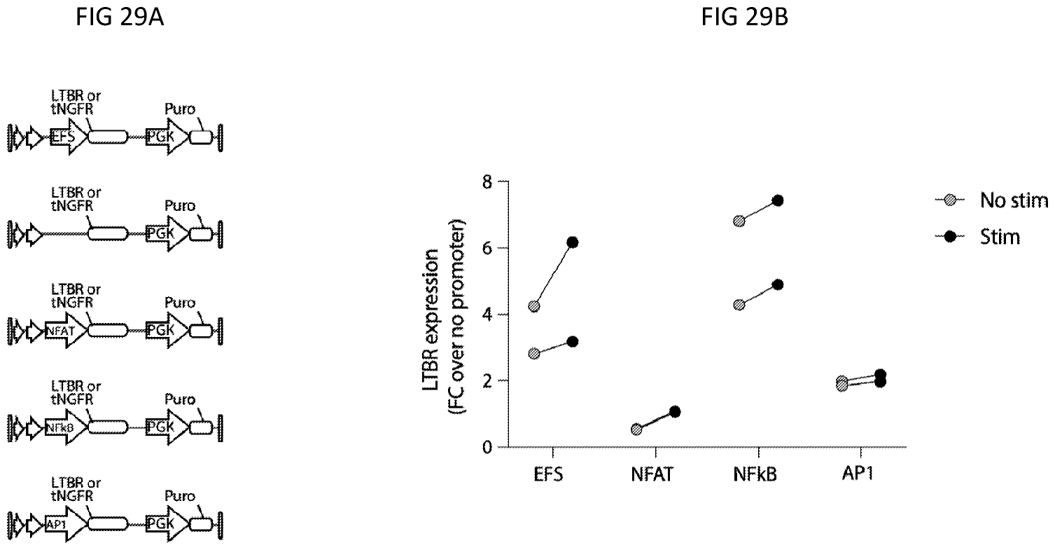

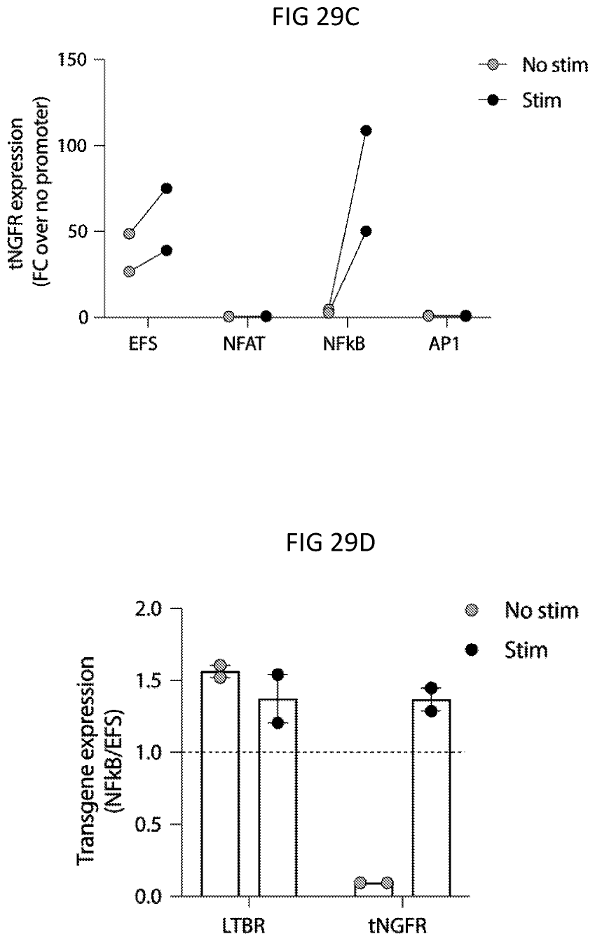

Controlling transgene expression offers an attractive possibility for improving the safety profile of engineered T cell therapies. Specifically, one could envision a system whereby LTBR (or other potentiator transgenes) are expressed only after a T cell encounters its target cell and receives signal through an antigen receptor (CAR or TCR); and once the target cells are cleared and antigen receptors no longer transmit signals, the potentiator gene expression decays back to the background level. In order to test that, we designed lentiviral vectors that drive LTBR (or control gene tNGFR) expression through inducible promoters that are activated by transcription factors upregulated upon T cell stimulation: NFAT, NFκB and AP1 (all from Jutz et al, Assessment of costimulation and coinhibition in a triple parameter T cell reporter line: Simultaneous measurement of NF-κB, NFAT and AP-1, J Immunol Methods, 2016 March; 430:10-20. doi: 10.1016/j.jim.2016.01.007. Epub 2016 Jan. 15). As a positive control, we included vectors driving transgene expression through a strong constitutive promoter (EFS); while for negative control we designed a vector lacking a promoter upstream of the transgene. All vectors also contained a selection marker (puromycin resistance) driven from a separate promoter, to allow for full selection of transduced cells regardless of the promoter tested (FIG. 29A).

Fourteen days post isolation, transduction and selection of primary human T cells (CD4 and CD8), we activated T cells transduced with each vector with anti-CD3/CD28 antibodies. 24h (FIG. 29B-D), 48h and 72h (data not shown) post re-stimulation we measured the expression of transgenes LTBR and tNGFR and compared it to the expression on unstimulated T cells as a control. In cases of NFAT and AP1 promoters, we observed no meaningful transgene expression, with or without stimulation. In case of constitutive promoter EFS, we observed an increase in transgene (both LTBR and tNGFR) expression after stimulation, presumably due to the increased rate of transcription and translation in activated/dividing T cells. Interestingly, the NFκB promoter drove a stronger expression of LTBR in both resting and stimulated T cells than that from the constitutive EFS promoter (FIG. 29B) which could be explained by LTBR's constitutive activation of the NFκB pathway (Legut et al, Nature 2022). Conversely, tNGFR was expressed from the NFκB promoter only after stimulation (FIG. 29C). Thus, we determined that the NFκB promoter can be used for inducible, stimulation-dependent transgene expression in T cells—but not in conjunction with NFκB-activating transgenes such as LTBR (FIG. 29D). For LTBR, the NFκB promoter offers an attractive alternative to the EFS promoter, given that it can drive higher expression of the transgene than EFS.

### REFERENCES

1. Abramson, J. S. et al. Transcend NHL 001: immunotherapy with the CD19-directed CAR T-cell product JCAR017 results in high complete response rates in relapsed or refractory B-cell non-Hodgkin lymphoma. Blood 128, 4192-4192 (2016).
2. Shifrut, E. et al. Genome-wide CRISPR screens in primary human T cells reveal key regulators of immune function. Cell 175, 1958-1971 (2018).
3. Dong. M. B. et al. Systematic immunotherapy target discovery using genome-scale in vivo CRISPR screens in CD8 T cells. Cell 178, 1189-1204 (2019).
4. Chen, Z. et al. In vivo CD8+ T cell CRISPR screening reveals control by Fli1 in infection and cancer. Cell 184, 1262-1280 (2021).
5. Kabelitz, D., Serrano, R., Kouakanou, L., Peters, C. & Kalyan, S. Cancer immunotherapy with γδ T cells: many paths ahead of us. Cell. Mol. Immunol. 17, 925-939 (2020).
6. Munshi, N. C. et al. Idecabtagene vicleucel in relapsed and refractory multiple myeloma. N. Engl. J. Med. 384, 705-716 (2021).
7. Fraietta, J. A. et al. Determinants of response and resistance to CD19 chimeric antigen receptor (CAR) T cell therapy of chronic lymphocytic leukemia. Nat. Med. 24, 563-571 (2018).
8. Singer, M. et al. A distinct gene module for dysfunction uncoupled from activation in tumor-infiltrating T cells. Cell 166, 1500-1511 (2016).
9. Gurusamy, D. et al. Multi-phenotype CRISPR-Cas9 screen identifies p38 kinase as a target for adoptive immunotherapies. Cancer Cell 37, 818-833 (2020).

10. Legut, M., Dolton. G., Mian, A. A., Ottmann. O. G. & Sewell. A. K. CRISPR-mediated TCR replacement generates superior anticancer transgenic T cells. Blood 131, 311-322 (2018).
11. Ren, J. et al. Multiplex genome editing to generate universal CAR T cells resistant to PD1 inhibition. Clin. Cancer Res. 23, 2255-2266 (2017).
12. Wagner, D. L. et al. High prevalence of Streptococcus pyogenes Cas9-reactive T cells within the adult human population. Nat. Med. 25, 242-248 (2019).
13. Wu, X. et al. Genome-wide binding of the CRISPR endonuclease Cas9 in mammalian cells. Nat. Biotechnol. 32, 670-676 (2014).
14. Sack, L. M. et al. Profound tissue specificity in proliferation control underlies cancer drivers and aneuploidy patterns. Cell 173, 499-514 (2018).
15. Yuan, J. et al. CTLA-4 blockade enhances polyfunctional NY-ESO-1 specific T cell responses in metastatic melanoma patients with clinical benefit. Proc. Natl Acad. Sci. USA 105, 20410-20415 (2008).
16. Sommermeyer, D. et al. Chimeric antigen receptor-modified T cells derived from defined CD8+ and CD4+ subsets confer superior antitumor reactivity in vivo. Leukemia 30, 492-500 (2016).
17. Fischer, A. M., Katayama. C. D., Pagès. G., Pouysségur. J. & Hedrick. S. M. The role of Erk1 and Erk2 in multiple stages of T cell development. Immunity 23, 431-443 (2005).
18. Lipp, A. M. et al. Lck mediates signal transmission from CD59 to the TCR/CD3 pathway in Jurkat T cells. PLOS One 9. e85934 (2014).
19. Ma, X. et al. Interleukin-23 engineering improves CAR T cell function in solid tumors. Nat. Biotechnol. 38, 448-459 (2020).
20. Seo, H. et al. BATF and IRF4 cooperate to counter exhaustion in tumor-infiltrating CAR T cells. Nat. Immunol. 22, 983-995 (2021).
21. Jamali, A. et al. Highly efficient and selective CAR-gene transfer using CD4- and CD8-targeted lentiviral vectors. Mol. Ther. Methods Clin. Dev. 13, 371-379 (2019).
22. Stoeckius, M. et al. Simultaneous epitope and transcriptome measurement in single cells. Nat. Methods 14, 865-868 (2017).
23. Mimitou, E. P. et al. Multiplexed detection of proteins, transcriptomes, clonotypes and CRISPR perturbations in single cells. Nat. Methods 16, 409-412 (2019).
24. Gil-Yarom, N. et al. CD74 is a novel transcription regulator. Proc. Natl Acad. Sci. USA 114, 562-567 (2017).
25. Ataide, M. A. et al. BATF3 programs CD8+ T cell memory. Nat. Immunol. 21, 1397-1407 (2020).
26. Katagiri, T., Kameda, H., Nakano. H. & Yamazaki. S. Regulation of T cell differentiation by the AP-1 transcription factor JunB. Immunol. Med. 44, 197-203 (2021).
27. Zhao, X . . . . Shan. Q. & Xuc. H.-H. TCF1 in T cell immunity: a broadened frontier. Nat. Rev. Immunol. https://doi.org/10.1038/s41577-021-00563-6 (2021).
28. Sudhamsu, J. et al. Dimerization of LTBR by LTα1β2 is necessary and sufficient for signal transduction. Proc. Natl Acad. Sci USA 110, 19896-19901 (2013).
29. Li, C. et al. Structurally distinct recognition motifs in lymphotoxin-receptor and CD40 for tumor necrosis factor receptor-associated factor (TRAF)-mediated signaling. J. Biol. Chem. 278, 50523-50529 (2003).
30. Wu, M.-Y., Wang. P.-Y., Han. S.-H. & Hsich. S.-L. The cytoplasmic domain of the lymphotoxin-β receptor mediates cell death in Hela cells. J. Biol. Chem. 274, 11868-11873 (1999).
31. Macian, F. NFAT proteins: key regulators of T-cell development and function. Nat. Rev. Immunol. 5, 472-484 (2005).
32. Dejardin, E. et al. The lymphotoxin-β receptor induces different patterns of gene expression via two NF-κB pathways. Immunity 17, 525-535 (2002).
33. Saoulli, K. et al. CD28-independent. TRAF2-dependent costimulation of resting T cells by 4-1BB ligand. J. Exp. Med. 187, 1849-1862 (1998).
34. Thommen, D. S. & Schumacher, T. N. T cell dysfunction in cancer. Cancer Cell 33, 547-562 (2018).
35. Roth, T. L. et al. Pooled knockin targeting for genome engineering of cellular immunotherapies. Cell 181, 728-744 (2020).
36. VanArsdale, T. L. et al. Lymphotoxin-receptor signaling complex: role of tumor necrosis factor receptor-associated factor 3 recruitment in cell death and activation of nuclear factor B. Proc. Natl Acad. Sci. USA 94, 2460-2465 (1997).
37. Yilmaz, Z. B. et al. Quantitative dissection and modeling of the NF-κB p100-p105 module reveals interdependent precursor proteolysis. Cell Rep. 9, 1756-1769 (2014).
38. Hu, B. et al. Augmentation of antitumor immunity by human and mouse CAR T cells secreting IL-18. Cell Rep. 20, 3025-3033 (2017).
39. Yeku, O. O., Purdon, T. J., Koneru, M., Spriggs, D. & Brentjens, R. J. Armored CAR T cells enhance antitumor efficacy and overcome the tumor microenvironment. Sci. Rep. 7, 10541 (2017).
40. Lynn, R. C. et al. c-Jun overexpression in CAR T cells induces exhaustion resistance. Nature 576, 293-300 (2019).
41. Schmiedel, B. J. et al. Impact of genetic polymorphisms on human immune cell gene expression. Cell 175, 1701-1715 (2018).

All publications cited in this specification are incorporated herein by reference. U.S. Provisional Patent Application No. 63/217,014, filed Jun. 30, 2021, U.S. Provisional Patent Application No. 63/287,389, filed Dec. 8, 2021, and U.S. Provisional Patent Application No. 63/320,101, filed Mar. 15, 2022 are incorporated herein by reference. While the invention has been described with reference to particular embodiments, it will be appreciated that modifications can be made without departing from the spirit of the invention. Such modifications are intended to fall within the scope of the appended embodiments.

What is claimed is:

1. A modified lymphocyte comprising an exogenous nucleic acid encoding LTBR.

2. The modified lymphocyte according to claim 1, wherein the nucleic acid encoding LTBR encodes an intracellular domain, or fragment or variant thereof.

3. The modified lymphocyte according to claim 2, wherein the LTBR intracellular domain comprises amino acids 249 to 435 of SEQ ID NO: 2, or a fragment, deletion, or variant thereof.

4. The modified lymphocyte according to claim 2 or 3, wherein the LTBR intracellular domain has a deletion in at least amino acids 393 to 435.

5. The modified lymphocyte according to any one of claims 1 to 4, wherein the lymphocyte comprises an expression cassette comprising an expression control sequence and the nucleic acid encoding LTBR.

6. The modified lymphocyte according to any one of claims 1 to 5, wherein the lymphocyte further comprises a nucleic acid encoding a chimeric antigen receptor (CAR).

7. The modified lymphocyte according to claim 1 or 5, wherein the lymphocyte further comprises a nucleic acid encoding a T cell receptor (TCR).

8. The modified lymphocyte according to claim 1, wherein the exogenous nucleic acid encoding LTBR is mRNA.

9. The modified lymphocyte according to any one of claims 1 to 8, wherein the lymphocyte is a T cell, an NK cell, or NK T cell.

10. The modified lymphocyte according to claim 6 or claim 8, wherein the CAR is Axicabtagene ciloleucel (Yescarta®), Brexucabtagene autoleucel (Tecartus™), Idecabtagene vicleucel (Abecma™), Lisocabtagene maraleucel (Breyanzi®), Tisagenlecleucel (Kyrmriah®), or one of those found in FIG. 19.

11. An expression cassette comprising a nucleotide sequence encoding a chimeric antigen receptor (CAR) and a nucleic acid encoding LTBR.

12. An expression cassette comprising a nucleic acid encoding a T cell receptor and a nucleic acid encoding LTBR.

13. An expression cassette comprising a nucleic acid encoding a viral protein and a nucleic acid encoding LTBR.

14. The expression cassette according to any one of claims 11 to 13, wherein the nucleic acid encoding LTBR encodes an LTBR intracellular domain, or fragment or variant thereof.

15. A method of producing a modified lymphocyte comprising introducing an exogenous nucleic acid encoding LTBR into the cell.

16. The method according to claim 15, wherein the nucleic acid encoding LTBR encodes an LTBR intracellular domain, or fragment or variant thereof.

17. The method according to claim 15 or 16, wherein the lymphocyte further comprises a nucleic acid encoding a chimeric antigen receptor (CAR).

18. The method according to any one of claims 15 to 17, wherein the lymphocyte further comprises a nucleic acid encoding an engineered T cell receptor (TCR).

19. A method of treating cancer in a subject in need thereof, the method comprising administering the modified lymphocyte according to any one of claims 1 to 10 or the expression cassette according to any one or claims 11 to 14 to the subject.

20. A method of treating a viral disease in a subject in need thereof, the method comprising administering a composition according to any one of claims 1 to 10 or the expression cassette according to any one or claims 11 to 14 to the subject.

21. A method of treating an autoimmune disorder in a subject in need thereof, the method comprising administering the modified lymphocyte according to any one of claims 1 to 10 or the expression cassette according to any one or claims 11 to 14 to the subject.

22. A method of increasing proliferation, or T cell effector function including cytokine production and/or secretion, the method comprising introducing the expression cassette according to any one of claims 1a to 14 into the T cell.

23. The method according to claim 22, wherein the T cell is obtained from a human prior to treating the T cell to overexpress LTBR, and the treated T cell is reintroduced into a human.

24. A method of increasing the response to a vaccine composition comprising co-administering to a subject a vaccine comprising a nucleic acid encoding LTBR.

25. The method according to claim 24, wherein the nucleic acid encoding LTBR encodes an LTBR intracellular domain, or fragment or variant thereof.

26. The method according to any one of claims 24 to 25, wherein the expression of LTBR is transient.

27. A modified lymphocyte comprising an exogenous nucleic acid encoding a gene of Table 1.

28. The modified lymphocyte according to claim 27, wherein the lymphocyte comprises an expression cassette comprising an expression control sequence and a nucleic acid encoding the gene of Table 1.

29. The modified lymphocyte according to claim 27 or 28, wherein the lymphocyte further comprises a nucleic acid encoding a chimeric antigen receptor (CAR).

30. The modified lymphocyte according to claim 27 or 28, wherein the lymphocyte further comprises a nucleic acid encoding a T cell receptor (TCR).

31. An expression cassette comprising a nucleotide sequence encoding a chimeric antigen receptor and a nucleic acid encoding a gene of Table 1.

32. An expression cassette comprising a nucleic acid encoding a T cell receptor and a nucleic acid encoding a gene of Table 1.

33. An expression cassette comprising a nucleic acid encoding a viral protein and a nucleic acid encoding a gene of Table 1.

34. A composition comprising a modified lymphocyte comprising the expression cassette of any one of claims 31 to 33.

35. A method of producing a modified lymphocyte comprising introducing an exogenous nucleic acid encoding a gene of Table 1 into the lymphocyte.

36. A method of treating cancer in a subject in need thereof, the method comprising administering the modified lymphocyte of any one of claims 27 to 30, the expression cassette according to any one of claims 31 to 33, or the composition according to claim 34 to the subject.

37. The modified lymphocyte, composition, expression cassette, or method according to any one of claims 27 to 36, wherein the gene of Table 1 is LTBR, ADA, IFNL2, IL12B CALML3 MRPL51, DBI GPN3, ITM2A, AHNAK, BATF, GPD1, ATF6B, AHCY, DUPD1, or AKR1C4.

38. A method of identifying a gene that alters the therapeutic function of a modified lymphocyte when exogenously expressed in the modified lymphocyte, the method comprising:
(a) obtaining a lymphocyte population;
(b) transducing the lymphocyte population with a plurality of viral vectors, each viral vector encoding a gene which may be linked to one or more barcodes;
(c) stimulating the transduced lymphocytes to induce activation, proliferation, and/or effector function;
(d) isolating a transduced lymphocyte from the lymphocyte population of (c); and
(e) detecting the presence of the gene and/or the linked barcodes in the isolated lymphocyte;
wherein the detected gene is effective to alter the therapeutic function of a modified lymphocyte that expresses the gene.

39. The method according to claim 38, wherein the gene is an open-reading frame (ORF) or a nucleotide sequence encoding a non-coding RNA, optionally a microRNA (miRNA) or long non-coding RNA (lncRNA, long ncRNA).

40. The method according to claim 38 or 39, wherein the lymphocyte population comprises a cell population that has been enriched for one or more of T cells, B cells, NK T cells, NK cells, or a subpopulation thereof, optionally wherein the cells are human.

41. The method according to any one of claims 38 to 40, wherein the lymphocyte population comprises a CAR T cell.

42. The method according to any one of claims 38 to 40, wherein the lymphocyte population comprises a lymphocyte comprising an engineered TCR expressed on its surface.

43. The method according to any one of claims 38 to 42, wherein the plurality of viral vectors comprises a library of open reading frames (ORFs).

44. The method according to any one of claims 38 to 43, wherein (e) comprises obtaining genomic DNA from the isolated lymphocyte and PCR amplification of the gene and/or barcode sequence.

45. The method according to any one of claims 38 to 44, wherein (e) further comprises single-cell transcriptome and/or proteome analysis.

46. A method of analyzing the effect on an individual cell of overexpression of an ORF of interest, comprising:
(a) introducing into the cell an expression cassette comprising a nucleic acid encoding the ORF of interest and overexpressing said ORF;
(b) providing a first set of nucleic acids derived from the individual cell and a first oligonucleotide having a first barcode sequence into a discrete partition, wherein the oligonucleotide is attached to a bead, wherein the first set of nucleic acids comprises endogenous transcriptome mRNA and ORF mRNA;
(c) performing RT-PCR to generate a second set of nucleic acids derived from the first set of nucleic acids, wherein said second set of nucleic acids within the partition have attached thereto first oligonucleotides that comprise the first nucleic acid barcode sequence, and wherein the RT-PCR is performed using RT-PCR reagents which comprise a primer which specifically anneals to a sequence on the ORF mRNA, that is not a poly A sequence, and wherein the second set of nucleic acids comprises endogenous transcriptome cDNA and ORF cDNA; and
(d) amplifying the second set of nucleic acids to generate a third set of nucleic acids using PCR reagents which comprise a second primer which specifically anneals to a sequence on the ORF cDNA, that is not a poly A sequence; and
(e) detecting and/or sequencing the barcode sequence, transcriptome cDNA, and/or ORF cDNA.

47. The method of claim 146, further comprising (d′) obtaining a portion of the third set of nucleic acids and amplifying the ORF cDNA using a second set of PCR reagents which comprise a third primer which specifically anneals to a sequence on the ORF cDNA, that is not a poly A sequence, to generate a fourth set of nucleic acids.

48. The method of claim 47, further comprising (d″) amplifying the ORF cDNA in the fourth set of nucleic acids using a third set of PCR reagents which comprise a fourth primer which specifically anneals to a sequence on the ORF cDNA, that is not a poly A sequence, to generate a fifth set of nucleic acids.

49. The method according to any one of claims 46 to 48, further comprising contacting the cell of (a) with a construct comprising an antibody or antibody fragment attached to the first oligonucleotide.

50. The method according to any one of claims 46 to 49, further comprising single-cell transcriptome and/or proteome analysis.
